SEQUENCE LISTING (56 sequences)

SEQ ID NO: 1 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 2 - protein, 955 aa, Homo sapiens [sequence omitted]

SEQ ID NO: 3 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 4 - protein, 180 aa, Homo sapiens
MLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 5 - protein, 267 aa, Homo sapiens
MHLLLFQLLVLLPLGKTTRHQDGRQNQSSLSPVLLPRNQRELPTGNHEEAEEKPDLFVAVPHLVATSPAGEGQRQREKMLSRFGRFWKKPEREMHPSRDSDSEPFPPGTQSLIQPIDGMKMEKSPLREEAKKFWHHFMFRKTPASQGVILPIKSHEVHWETCRTVPFSQTITHEGCEKVVVQNNLCFGKCGSVHFPGAAQHSHTSCSHCLPAKFTTMHLPLNCTELSSVIKVVMLVEECQCKVKTEHEDGHILHAGSQDSFIPGVSA

SEQ ID NO: 6 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 7 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 8 - protein, 70 aa, Homo sapiens
MKATIILLLLAQVSWAGPFQQRGLFDFMLEDEASGIGPEVPDDRDFEPSLGPVCPFRCQCHLRVVQCSDL

SEQ ID NO: 9 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 10 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 11 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 12 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 13 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 14 - protein, 172 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQERSPGQKKGRKDRRPRKDRKLDRRLDVRPRQPGLQP

SEQ ID NO: 15 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 16 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 17 - protein, 338 aa, Homo sapiens
AVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 18 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 19 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 20 - protein, 208 aa, Homo sapiens
MWKWILTHCASAFPHLPGCCCCCFLLLFLVSSVPVTCQALGQVMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 21 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 22 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 23 - protein, 159 aa, Homo sapiens
MVPSAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 24 - protein, 160 aa, Artificial Sequence
  Description of Artificial Sequence Synthetic polypeptide
MVPLAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVVL

SEQ ID NO: 25 - protein, 50 aa, Homo sapiens
VVSHFNDCPDSHTQFCFHGTCRFLVQEDKPACVCHSGYVGARCEHADLLA

SEQ ID NO: 26 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 27 - protein, 194 aa, Homo sapiens; identical to SEQ ID NO: 19

SEQ ID NO: 28 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 29 - protein, 169 aa, Homo sapiens
MRGSHHHHHHGSIEGRSAVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEI

SEQ ID NO: 30 - protein, 17 aa, Homo sapiens
CDDYYYGFGCNKFCRPR

SEQ ID NO: 31 - protein, 1238 aa, Homo sapiens
MRAQGRGRLPRRLLLLLALWVQAARPMGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTGLLVPVLCGAFSVLWLACVVLCVWWTRKRRKERERSRLPREESANNQWAPLNPIRNPIERPGGHKDVLYQCKNFTPPPRRADEALPGPAGHAAVREDEEDEDLGRGEEDSLEAEKFLSHKFTKDPGRSPGRPAHWASGPKVDNRAVRSINEARYAGKE

SEQ ID NO: 32 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 33 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 34 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 35 - protein, 587 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 36 - DNA, 1734 bp, Homo sapiens
ggtctgaggcctctgcctaaagacaaagcctgtgctggggtgtgcaggatataaggttggacttccagacccactgcccgggagaggagaggagcgggccgaggactccagcgtgcccaggtctggcatcctgcacttgctgccctctgacacctgggaagatggccggcccgtggaccttcacccttctctgtggtttgctggcagccaccttgatccaagccaccctcagtcccactgcagttctcatcctcggcccaaaagtcatcaaagaaaagctgacacaggagctgaaggaccacaacgccaccagcatcctgcagcagctgccgctgctcagtgccatgcgggaaaagccagccggaggcatccctgtgctgggcagcctggtgaacaccgtcctgaagcacatcatctggctgaaggtcatcacagctaacatcctccagctgcaggtgaagccctcggccaatgaccaggagctgctagtcaagatccccctggacatggtggctggattcaacacgcccctggtcaagaccatcgtggagttccacatgacgactgaggcccaagccaccatccgcatggacaccagtgcaagtggccccacccgcctggtcctcagtgactgtgccaccagccatgggagcctgcgcatccaactgctgcataagctctccttcctggtgaacgccttagctaagcaggtcatgaacctcctagtgccatccctgcccaatctagtgaaaaaccagctgtgtcccgtgatcgaggcttccttcaatggcatgtatgcagacctcctgcagctggtgaaggtgcccatttccctcagcattgaccgtctggagtttgaccttctgtatcctgccatcaagggtgacaccattcagctctacctgggggccaagttgttggactcacagggaaaggtgaccaagtggttcaataactctgcagcttccctgacaatgcccaccctggacaacatcccgttcagcctcatcgtgagtcaggacgtggtgaaagctgcagtggctgctgtgctctctccagaagaattcatggtcctgttggactctgtgcttcctgagagtgcccatcggctgaagtcaagcatcgggctgatcaatgaaaaggctgcagataagctgggatctacccagatcgtgaagatcctaactcaggacactcccgagttttttatagaccaaggccatgccaaggtggcccaactgatcgtgctggaagtgtttccctccagtgaagccctccgccctttgttcaccctgggcatcgaagccagctcggaagctcagttttacaccaaaggtgaccaacttatactcaacttgaataacatcagctctgatcggatccagctgatgaactctgggattggctggttccaacctgatgttctgaaaaacatcatcactgagatcatccactccatcctgctgccgaaccagaatggcaaattaagatctggggtcccagtgtcattggtgaaggccttgggattcgaggcagctgagtcctcactgaccaaggatgcccttgtgcttactccagcctccttgtggaaacccagctctcctgtctcccagtgaagacttggatggcagccatcagggaaggctgggtcccagctgggagtatgggtgtgagctctatagaccatccctctctgcaatcaataaacacttgcctgtgatgcctgcaaaaaaa

SEQ ID NO: 37 - DNA, 1561 bp, Homo sapiens
gcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataataaaaaaaaa

SEQ ID NO: 38 - DNA, 3698 bp, Homo sapiens
ggaagagggagtgttcccgggggagatactccagtcgtagcaagagtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccataaaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttg

SEQ ID NO: 39 - DNA, 3699 bp, Homo sapiens
aatcacggtggaagtatgatattttggctgtggatctgagttgatcaatctgcttagtggacttgagtccccccacccccgcttgtctgattggggctcctgggaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccataaaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttg

SEQ ID NO: 40 - DNA, 8571 bp, Homo sapiens
ctttaacaaagtcctcctctctttgctccctcccacttcattcacttgcaaatcagtgtgtgcccacaagagccagctctcccgagcccgtaaccttcgcatcccaagagctgcagtttcagccgcgacagcaagaacggcagagccggcgaccgcggcggcggcggcggcggaggcaggagcagcctgggcgggtcgcagggtctccgcgggcgcaggaaggcgagcagagatatcctctgagagccaagcaaagaacattaaggaaggaaggaggaatgaggctggatacggtgcagtgaaaaaggcacttccaagagtggggcactcactacgcacagactcgacggtgccatcagcatgagaacttaccgctacttcttgctgctcttttgggtgggccagccctacccaactctctcaactccactatcaaagaggactagtggtttcccagcaaagaaaagggccctggagctctctggaaacagcaaaaatgagctgaaccgttcaaaaaggagctggatgtggaatcagttctttctcctggaggaatacacaggatccgattatcagtatgtgggcaagttacattcagaccaggatagaggagatggatcacttaaatatatcctttcaggagatggagcaggagatctcttcattattaatgaaaacacaggcgacatacaggccaccaagaggctggacagggaagaaaaacccgtttacatccttcgagctcaagctataaacagaaggacagggagacccgtggagcccgagtctgaattcatcatcaagatccatgacatcaatgacaatgaaccaatattcaccaaggaggtttacacagccactgtccctgaaatgtctgatgtcggtacatttgttgtccaagtcactgcgacggatgcagatgatccaacatatgggaacagtgctaaagttgtctacagtattctacagggacagccctatttttcagttgaatcagaaacaggtattatcaagacagctttgctcaacatggatcgagaaaacagggagcagtaccaagtggtgattcaagccaaggatatgggcggccagatgggaggattatctgggaccaccaccgtgaacatcacactgactgatgtcaacgacaaccctccccgattcccccagagtacataccagtttaaaactcctgaatcttctccaccggggacaccaattggcagaatcaaagccagcgacgctgatgtgggagaaaatgctgaaattgagtacagcatcacagacggtgaggggctggatatgtttgatgtcatcaccgaccaggaaacccaggaagggattataactgtcaaaaagctcttggactttgaaaagaagaaagtgtatacccttaaagtggaagcctccaatccttatgttgagccacgatttctctacttggggcctttcaaagattcagccacggttagaattgtggtggaggatgtagatgagccacctgtcttcagcaaactggcctacatcttacaaataagagaagatgctcagataaacaccacaataggctccgtcacagcccaagatccagatgctgccaggaatcctgtcaagtactctgtagatcgacacacagatatggacagaatattcaacattgattctggaaatggttcgatttttacatcgaaacttcttgaccgagaaacactgctatggcacaacattacagtgatagcaacagagatcaataatccaaagcaaagtagtcgagtacctctatatattaaagttctagatgtcaatgacaacgccccagaatttgctgagttctatgaaacttttgtctgtgaaaaagcaaaggcagatcagttgattcagaccctgcatgctgttgacaaggatgacccttatagtggacaccaattttcgttttccttggcccctgaagcagccagtggctcaaactttaccattcaagacaacaaagacaacacggcgggaatcttaactcggaaaaatggctataatagacacgagatgagcacctatctcttgcctgtggtcatttcagacaacgactacccagttcaaagcagcactgggacagtgactgtccgggtctgtgcatgtgaccaccacgggaacatgcaatcctgccatgcggaggcgctcatccaccccacgggactgagcacgggggctctggttgccatccttctgtgcatcgtgatcctactagtgacagtggtgctgtttgcagctctgaggcggcagcgaaaaaaagagcctttgatcatttccaaagaggacatcagagataacattgtcagttacaacgacgaaggtggtggagaggaggacacccaggcttttgatatcggcaccctgaggaatcctgaagccatagaggacaacaaattacgaagggacattgtgcccgaagcccttttcctaccccgacggactccaacagctcgcgacaacaccgatgtcagagatttcattaaccaaaggttaaaggaaaatgacacggaccccactgccccgccatacgactccttggccacttacgcctatgaaggcactggctccgtggcggattccctgagctcgctggagtcagtgaccacggatgcagatcaagactatgattaccttagtgactggggacctcgattcaaaaagcttgcagatatgtatggaggagtggacagtgacaaagactcctaatctgttgcctttttcattttccaatacgacactgaaatatgtgaagtggctatttctttatatttatccactactccgtgaaggcttctctgttctacccgttccaaaagccaatggctgcagtccgtgtggatccaatgttagagacttttttctagtacacttttatgagcttccaaggggcaaatttttattttttagtgcatccagttaaccaagtcagcccaacaggcaggtgccggaggggaggacagggaacagtatttccacttgttctcagggcagcgtgcccgcttccgctgtcctggtgttttactacactccatgtcaggtcagccaactgccctaactgtacatttcacaggctaatgggataaaggactgtgctttaaagataaaaatatcatcatagtaaaagaaatgagggcatatcggctcacaaagagataaactacataggggtgtttatttgtgtcacaaagaatttaaaataacacttgcccatgctatttgttcttcaagaactttctctgccatcaactactattcaaaacctcaaatccacccatatgttaaaattctcattactcttaaggaatagaagcaaattaaacggtaacatccaaaagcaaccacaaacctagtacgacttcattccttccactaactcatagtttgttatatcctagactagacatgcgaaagtttgcctttgtaccatataaagggggagggaaatagctaataatgttaaccaaggaaatatattttaccatacatttaaagttttggccaccacatgtatcacgggtcacttgaaattctttcagctatcagtaggctaatgtcaaaattgtttaaaaattcttgaaagaattttcctgagacaaattttaacttcttgtctatagttgtcagtattattctactatactgtacatgaaagtagcagtgtgaagtacaataattcatattcttcatatccttcttacacgactaagttgaattagtaaagttagattaaataaaacttaaatctcactctaggagttcagtggagaggttagagccagccacacttgaacctaataccctgcccttgacatctggaaacctctacatatttatataacgtgatacatttggataaacaacattgagattatgatgaaaacctacatattccatgtttggaagacccttggaagaggaaaattggattcccttaaacaaaagtgtttaagattgtaattaaaatgatagttgattttcaaaagcattaattttttttcattgtttttaactttgctttcatgaccatcctgccatccttgactttgaactaatgataaagtaatgatctcaaactatgacagaaaagtaatgtaaaatccatccaatctattatttctctaattatgcaattagcctcatagttattatccagaggacccaactgaactgaactaatccttctggcagattcaaatcgtttatttcacacgctgttctaatggcacttatcattagaatcttaccttgtgcagtcatcagaaattccagcgtactataatgaaaacatccttgttttgaaaacctaaaagacaggctctgtatatatatatacttaagaatatgctgacttcacttattagtcttagggatttattttcaattaatattaattttctacaaataattttagtgtcatttccatttggggatattgtcatatcagcacatattttctgtttggaaacacactgttgtttagttaagttttaaataggtgtattacccaagaagtaaagatggaaacgttaaaagaagagaaatgtagtattttgggttacctgattagagtgaaaattttttacaatcatattattccttgtgtcttctgaatggtttccgattttataatggactgccctatatagtaacaagtatttcatgcttgagctatttcctgctttcagggtttcttttttctagttcttcatacacacacatacacacacacacacacacacacacacacacacacgaatgcaaacaaaaggctatatgaggtcttcactctaatgaattgatatgtatcatagtcacaggtaagtgttgaaaaaagcttagtaaagttagaagctacttactcatagcaatagaacagcaccttaatcacacgatttactgtaaaattaaagaggtctctatctgtatgtttcatgtcacgtaacaaattgaatcaaggaagatagtcctgtaaaaagaaaggtatcatctgaagttgaggattgacactagcagtttccaatgtttaaaggtaagatctgagttctcctaataagtaaaagtaagtagttctatagcagaatatctgagatgtaattggcaaggtattttatccctccctgcagatgacacagcataccaagaacaggttaatatgattacttatggaaataactttaatctcttatcataaaagctgatgatgaagtaaatttataggaaattggataatttgagactggggctaaatatttagtaccagggtactgtaagtatcaagttggagtgacgttttcctataattcagactctttgacatcgtggaaccaataagagtcatagttccatcattctccagcttcgtctcacttccttcccaccccacctgagtatcaggtcaaacatcattgcatgcgcaggttttttttttaattgctaggtcccaggcaacatgaaagattattggagaaaaaaataattttcagcccagttttttcattgtctgtttcctaattttagatgttggtgatgggaaagatggaaggagagtgggaagaagtaaaattttaatatttgtttcaatcactttgaaactaaaattcattaagcataaccagattgcttttgtgggttgtttcaaggacattgagagctttctgatgatatgtttttgccctctattcaaaagcaagagttcctttaaactactaagatattccctagaataagctgaatttaaaaaaacattaagccattgtttaaagccccttcacttcctggccacttacttctgaaaggcctaaaaaacatttgtgcccaaataagtaaataaaccaaatgggaaagaagcaaagattattccatagaaccacaagagagggaatgtgggcacagtaaatagatgtttctttcagaactttcctgcctttacagtttgtgtccataaagggatgttcagcaatgaaattactcccttttcagatggaacaaaacctgcccatttaattttaacgcagtataaaaaacgtgtggtttagtttttattttcagctcccaaagagttgtgcagaaaatcttaaaattttttttttttttttttttttttttttgagacagaatctcgctctgtcgcccaggctggaatgcagtggcgcgatctctgctcactacaagctccgcctcccgggttcacgccattctcctgcctcagcgcccccagtagctgggactacaggcacacaccaccacgcccggctaatttttgttgtattttttagtggagacagggtttcaccatgttagccaggatgatctccatctcctgacctcgtggtccgcccgcctcggcctcccaaagtgctgggattacaggcgtgagccaccgcgcccggccttaaaaattgaatctgtagcttaggccatccaaattttataaatccaaattaactttagaatgtttctattactttcacttttacatatataaattttaagtgtcctgattggctgaacaatatctcacatcaaatgctttgcctggaaatagatatcccactggggatagtggtgtgtaaactatgacttggacaattctatatactcaagcaccataaaaagtatgcagttgaaaagaaaatcaaagttgattcctgggtgccaactaaatattcaaatcaggtactcatccttatcagctaaattcattttcaccaggaacagaccaccaaataaattattttatcctaataactagttttgaagcagtgtaattactctggaagaaggctctaaaaagtcatgattcccccactattttgaaatgtatcctctaacaaggatcattatagtgtaatcttaatttttatgttttatcaagatgaaatcttgtttgaattgtgatattataaaaggggactcaaaaatccaagcagtctactgtgtttaaattaacaccacaaccttccttatcagattataagagtagaaaaattaacacttggtgtgtgaatcttcaggaaaatgagctatttcataagctcaaacaagcagcttccttttccagagaatatagaattatattatggtctccttaaatgtttagtagctcttatggtcacagcatttttaatctccctatggcatctttatggaataattttctaaagggtaattctctactaaaaatatcagaccccgaccatatttaatgtggagagcaataccctcttagaaagaaaatacattgactcatacacttgttaaaagttaataaagaaatagctcatttttaaagccggaagtttatggtctctgcatcgtcaatttaatttaagcattgctgagacaatctttaatctactcccctttttgtaataccttatttatggtgcattttcatttttatttgggggaaacgttagcccaacagagccggcagatgaaagtgttgaaaagaggtcaaatggaaacaaaggctcttacccgctgtatttcagacaggactgaggcacttagccgaggagccactgggttattagattaatttcaaaagagcttttacaagttgcttaattcctttttttttttttttttttttcaaaaacccatgaaccacaaactcaaatttctcctcaaatggggttaatctgacaaacgaggcatggacccagccttgtggaaaaagcattccacgctaatgagatcttggtctttcttgtgaggctacgttatttatgtaaatatgtctggaggcaccttctctaagcttttagttttctatgatctattagtttagtgtttattaaagaatcaaatgtatagaattaccaggcattcgtggggaatgctgtgtagcaaatgtaaaactgacctgctcggaagaaacgtaggaacgcttcaaacccactgtaatgtttggtttgagattattttcattgctttgagagtgaactgcctaagagtaggccttataataaatgctatgtgcgtcttcagtagttccaagctaaagcaatttggcattctcccactgtgatttgtgacttttaaacccacaaaataaaagctttttggtattgattgtttttaattaaaaatacttccaagtataaattgaaacggatgccacccttgaagatttactggcgggaatgctcactcttgtcgttttcctcagtatcgttcatgtctttggcaacaagaacacctgatgaaagcaagcaatgctcagttcccatcaacatttctagttagggggattctcataaccccacagtttacctgagaaagttttctgtgttagaagaatggggtcgagagtattaccttttagctcagtgtggccgggccttttgttgcagtcaaatggcaaatacgcactccttgaaatggcttcttttatttggttttgttttcttagacttataaatttgaaaagaatgcaatttaaaaagtgatttctcacaaagagtaaatatgccttttgcaaatcaatttttgtaacaagttatttatatgatattacttaataaactggtttttttctaa

SEQ ID NO: 41 - DNA, 3359 bp, Homo sapiens
cacaccttcggcagcaggagggcggcagcttctcgcaggcggcagggcgggcggccaggatcatgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaatgctctaagacctctcagcacgggcggaagaaactcccggagagctcacccaaaaaacaaggagatcccatctagatttcttcttgcttttgactcacagctggaagttagaaaagcctcgatttcatctttggagaggccaaatggtcttagcctcagtctctgtctctaaatattccaccataaaacagctgagttatttatgaattagaggctatagctcacattttcaatcctctatttctttttttaaatataactttctactctgatgagagaatgtggttttaatctctctctcacattttgatgatttagacagactccccctcttcctcctagtcaataaacccattgatgatctatttcccagcttatccccaagaaaacttttgaaaggaaagagtagacccaaagatgttattttctgctgtttgaattttgtctccccacccccaacttggctagtaataaacacttactgaagaagaagcaataagagaaagatatttgtaatctctccagcccatgatctcggttttcttacactgtgatcttaaaagttaccaaaccaaagtcattttcagtttgaggcaaccaaacctttctactgctgttgacatcttcttattacagcaacaccattctaggagtttcctgagctctccactggagtcctctttctgtcgcgggtcagaaattgtccctagatgaatgagaaaattattttttttaatttaagtcctaaatatagttaaaataaataatgttttagtaaaatgatacactatctctgtgaaatagcctcacccctacatgtggatagaaggaaatgaaaaaataattgctttgacattgtctatatggtactttgtaaagtcatgcttaagtacaaattccatgaaaagctcactgatcctaattctttccctttgaggtctctatggctctgattgtacatgatagtaagtgtaagccatgtaaaaagtaaataatgtctgggcacagtggctcacgcctgtaatcctagcactttgggaggctgaggaggaaggatcacttgagcccagaagttcgagactagcctgggcaacatggagaagccctgtctctacaaaatacagagagaaaaaatcagccagtcatggtggcctacacctgtagtcccagcattccgggaggctgaggtgggaggatcacttgagcccagggaggttggggctgcagtgagccatgatcacaccactgcactccagccaggtgacatagcgagatcctgtctaaaaaaataaaaaataaataatggaacacagcaagtcctaggaagtaggttaaaactaattctttaaaaaaaaaaaaaagttgagcctgaattaaatgtaatgtttccaagtgacaggtatccacatttgcatggttacaagccactgccagttagcagtagcactttcctggcactgtggtcggttttgttttgttttgctttgtttagagacggggtctcactttccaggctggcctcaaactcctgcactcaagcaattcttctaccctggcctcccaagtagctggaattacaggtgtgcgccatcacaactagctggtggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctacagtt

SEQ ID NO: 42 - DNA, 3350 bp, Homo sapiens
agaattgcgctgtccacttgtcgtgtggctctgtgtcgacactgtgcgccaccatggccgtgactgcctgtcagggcttggggttcgtggtttcactgattgggattgcgggcatcattgctgccacctgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaatgctctaagacctctcagcacgggcggaagaaactcccggagagctcacccaaaaaacaaggagatcccatctagatttcttcttgcttttgactcacagctggaagttagaaaagcctcgatttcatctttggagaggccaaatggtcttagcctcagtctctgtctctaaatattccaccataaaacagctgagttatttatgaattagaggctatagctcacattttcaatcctctatttctttttttaaatataactttctactctgatgagagaatgtggttttaatctctctctcacattttgatgatttagacagactccccctcttcctcctagtcaataaacccattgatgatctatttcccagcttatccccaagaaaacttttgaaaggaaagagtagacccaaagatgttattttctgctgtttgaattttgtctccccacccccaacttggctagtaataaacacttactgaagaagaagcaataagagaaagatatttgtaatctctccagcccatgatctcggttttcttacactgtgatcttaaaagttaccaaaccaaagtcattttcagtttgaggcaaccaaacctttctactgctgttgacatcttcttattacagcaacaccattctaggagtttcctgagctctccactggagtcctctttctgtcgcgggtcagaaattgtccctagatgaatgagaaaattattttttttaatttaagtcctaaatatagttaaaataaataatgttttagtaaaatgatacactatctctgtgaaatagcctcacccctacatgtggatagaaggaaatgaaaaaataattgctttgacattgtctatatggtactttgtaaagtcatgcttaagtacaaattccatgaaaagctcactgatcctaattctttccctttgaggtctctatggctctgattgtacatgatagtaagtgtaagccatgtaaaaagtaaataatgtctgggcacagtggctcacgcctgtaatcctagcactttgggaggctgaggaggaaggatcacttgagcccagaagttcgagactagcctgggcaacatggagaagccctgtctctacaaaatacagagagaaaaaatcagccagtcatggtggcctacacctgtagtcccagcattccgggaggctgaggtgggaggatcacttgagcccagggaggttggggctgcagtgagccatgatcacaccactgcactccagccaggtgacatagcgagatcctgtctaaaaaaataaaaaataaataatggaacacagcaagtcctaggaagtaggttaaaactaattctttaaaaaaaaaaaaaagttgagcctgaattaaatgtaatgtttccaagtgacaggtatccacatttgcatggttacaagccactgccagttagcagtagcactttcctggcactgtggtcggttttgttttgttttgctttgtttagagacggggtctcactttccaggctggcctcaaactcctgcactcaagcaattcttctaccctggcctcccaagtagctggaattacaggtgtgcgccatcacaactagctggtggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctacagtt

SEQ ID NO: 43 - DNA, 584 bp, Homo sapiens
agacactctccaaaaagcagagacagcaggaagaggggagtggaggcagcccattcacctggggaaatgactgggttgtcgatggacggtggcggcagccccaagggggacgtggacccgttctactatgactatgagaccgttcgcaatgggggcctgatcttcgctggactggccttcatcgtggggctcctcatcctcctcagcagaagattccgctgtgggggcaataagaagcgcaggcaaatcaatgaagatgagccgtaacagcagcctcggcggtgccacccactgcactggggccagctgggaagccaagcatggccctgcctctggcgcctccccttcttccctgggctttagacctttgtccccgtcactgccagcgcttgggctgaaggaagctccagactcaatgtgacccccaggtggcatcgccaactcctgcctcgtgccacctcatgcttataataaagccggcgtcagagaccgctgcttccctcacctgcctgcctgtctccctcctctgtcaccaccagcctctccaagctcaagtacaaatacagccgggaaaaaaaaaaaaa

SEQ ID NO: 44 - DNA, 591 bp, Homo sapiens
gccactctccatccaggccccaggcaagcagcacctccctgctctcctgcactcctggacacaaccagcagctcctgccatggacaggtggtacctgggcggcagccccaagggggacgtggacccgttctactatgactatgagaccgttcgcaatgggggcctgatcttcgctggactggccttcatcgtggggctcctcatcctcctcagcagaagattccgctgtgggggcaataagaagcgcaggcaaatcaatgaagatgagccgtaacagcagcctcggcggtgccacccactgcactggggccagctgggaagccaagcatggccctgcctctggcgcctccccttcttccctgggctttagacctttgtccccgtcactgccagcgcttgggctgaaggaagctccagactcaatgtgacccccaggtggcatcgccaactcctgcctcgtgccacctcatgcttataataaagccggcgtcagagaccgctgcttccctcacctgcctgcctgtctccctcctctgtcaccaccagcctctccaagctcaagtacaaatacagccgggaaaaaaaaaaaaa

SEQ ID NO: 45 - DNA, 4509 bp, Homo sapiens
actgagcatttctaagggagttgaggctggtggctcctccttccttcctactggtgcttccacctgccttggtctgagttgcagtccatggggcagcgcctaagtgtctgagcacacttaagaatctctagtggtttatgacccagactttgccctaccacctcagtcttctgaatgttctcttccctggaccctgctccagacactttaaattcagaagaggaaaatgtgcccagcctgcctggagaaaagtgtctgctcctagccaagatctcctcatcacaaaagtaatgtgggccatggagtcaggccacctcctctgggctctgctgttcatgcagtccttgtggcctcaactgactgatggagccactcgagtctactacctgggcatccgggatgtgcagtggaactatgctcccaagggaagaaatgtcatcacgaaccagcctctggacagtgacatagtggcttccagcttcttaaagtctgacaagaaccggatagggggaacctacaagaagaccatctataaagaatacaaggatgactcatacacagatgaagtggcccagcctgcctggttgggcttcctggggccagtgttgcaggctgaagtgggggatgtcattcttattcacctgaagaattttgccactcgtccctataccatccaccctcatggtgtcttctacgagaaggactctgaaggttccctatacccagatggctcctctgggccactgaaagctgatgactctgttcccccggggggcagccatatctacaactggaccattccagaaggccatgcacccaccgatgctgacccagcgtgcctcacctggatctaccattctcatgtagatgctccacgagacattgcaactggcctaattgggcctctcatcacctgtaaaagaggagccctggatgggaactcccctcctcaacgccaggatgtagaccatgatttcttcctcctcttcagtgtggtagatgagaacctcagctggcatctcaatgagaacattgccacttactgctcagatcctgcttcagtggacaaagaagatgagacatttcaggagagcaataggatgcatgcaatcaatggctttgtttttgggaatttacctgagctgaacatgtgtgcacagaaacgtgtggcctggcacttgtttggcatgggcaatgaaattgatgtccacacagcatttttccatggacagatgctgactacccgtggacaccacactgatgtggctaacatctttccagccacctttgtgactgctgagatggtgccctgggaacctggtacctggttaattagctgccaagtgaacagtcactttcgagatggcatgcaggcactctacaaggtcaagtcttgctccatggcccctcctgtggacctgctcacaggcaaagttcgacagtacttcattgaggcccatgagattcaatgggactatggcccgatggggcatgatgggagtactgggaagaatttgagagagccaggcagtatctcagataagtttttccagaagagctccagccgaattgggggcacttactggaaagtgcgatatgaagcctttcaagatgagacattccaagagaagatgcatttggaggaagataggcatcttggaatcctggggccagtgatccgggctgaggtgggtgacaccattcaggtggtcttctacaaccgtgcctcccagccattcagcatgcagccccatggggtcttttatgagaaagactatgaaggcactgtgtacaatgatggctcatcttaccctggcttggttgccaagccctttgagaaagtaacataccgctggacagtcccccctcatgccggtcccactgctcaggatcctgcttgtctcacttggatgtacttctctgctgcagatcccataagagacacaaattctggcctggtgggcccgctgctggtgtgcagggctggtgccttgggtgcagatggcaagcagaaaggggtggataaagaattctttcttctcttcactgtgttggatgagaacaagagctggtacagcaatgccaatcaagcagctgctatgttggatttccgactgctttcagaggatattgagggcttccaagactccaatcggatgcatgccattaatgggtttctgttctctaacctgcccaggctggacatgtgcaagggtgacacagtggcctggcacctgctcggcctgggcacagagactgatgtgcatggagtcatgttccagggcaacactgtgcagcttcagggcatgaggaagggtgcagctatgctctttcctcatacctttgtcatggccatcatgcagcctgacaaccttgggacatttgagatttattgccaggcaggcagccatcgagaagcagggatgagggcaatctataatgtctcccagtgtcctggccaccaagccacccctcgccaacgctaccaagctgcaagaatctactatatcatggcagaagaagtagagtgggactattgccctgaccggagctgggaacgggaatggcacaaccagtctgagaaggacagttatggttacattttcctgagcaacaaggatgggctcctgggttccagatacaagaaagctgtattcagggaatacactgatggtacattcaggatccctcggccaaggactggaccagaagaacacttgggaatcttgggtccacttatcaaaggtgaagttggtgatatcctgactgtggtattcaagaataatgccagccgcccctactctgtgcatgctcatggagtgctagaatctactactgtctggccactggctgctgagcctggtgaggtggtcacttatcagtggaacatcccagagaggtctggccctgggcccaatgactctgcttgtgtttcctggatctattattctgcagtggatcccatcaaggacatgtatagtggcctggtggggcccttggctatctgccaaaagggcatcctggagccccatggaggacggagtgacatggatcgggaatttgcattgttgttcttgatttttgatgaaaataagtcttggtatttggaggaaaatgtggcaacccatgggtcccaggatccaggcagtattaacctacaggatgaaactttcttggagagcaataaaatgcatgcaatcaatgggaaactctatgccaaccttaggggtcttaccatgtaccaaggagaacgagtggcctggtacatgctggccatgggccaagatgtggatctacacaccatccactttcatgcagagagcttcctctatcggaatggcgagaactaccgggcagatgtggtggatctgttcccagggacttttgaggttgtggagatggtggccagcaaccctgggacatggctgatgcactgccatgtgactgaccatgtccatgctggcatggagaccctcttcactgttttttctcgaacagaacacttaagccctctcaccgtcatcaccaaagagactgaaaaagcagtgccccccagagacattgaagaaggcaatgtgaagatgctgggcatgcagatccccataaagaatgttgagatgctggcctctgttttggttgccattagtgtcacccttctgctcgttgttctggctcttggtggagtggtttggtaccaacatcgacagagaaagctacgacgcaataggaggtccatcctggatgacagcttcaagcttctgtctttcaaacagtaacatctggagcctggagatatcctcaggaagcacatctgtagtgcactcccagcaggccatggactagtcactaaccccacactcaaaggggcatgggtggtggagaagcagaaggagcaatcaagcttatctggatatttctttctttatttattttacatggaaataatatgatttcactttttctttagtttctttgctctacgtgggcacctggcactaagggagtaccttattatcctacatcgcaaatttcaacagctacattatatttccttctgacacttggaaggtattgaaatttctagaaatgtatccttctcacaaagtagagaccaagagaaaaactcattgattgggtttctacttctttcaaggactcaggaaatttcactttgaactgaggccaagtgagctgttaagataacccacacttaaactaaaggctaagaatataggcttgatgggaaattgaaggtaggctgagtattgggaatccaaattgaattttgattctccttggcagtgaactactttgaagaagtggtcaatgggttgttgctgccatgagcatgtacaacctctggagctagaagctcctcaggaaagccagttctccaagttcttaacctgtggcactgaaaggaatgttgagttacctcttcatgttttagacagcaaaccctatccattaaagtacttgttagaacactgaaaaaaaaaaaaaaaaaaa

SEQ ID NO: 46 - DNA, 4231 bp, Homo sapiens
ggaaaagagggcacccagcccttccccctccctcatcctcccatcccagtaaaccctgccaaattggaatcctggacttaatttaggagaaaggccctgtaaccaagatactgactgaacatggctggcggactcaggctggggtctgcagtgcagcattaatgggccgctgacatgaatatggagtagttttctctagcaaagagtggcttccagcttcttaaagtctgacaagaaccggatagggggaacctacaagaagaccatctataaagaatacaaggatgactcatacacagatgaagtggcccagcctgcctggttgggcttcctggggccagtgttgcaggctgaagtgggggatgtcattcttattcacctgaagaattttgccactcgtccctataccatccaccctcatggtgtcttctacgagaaggactctgaaggttccctatacccagatggctcctctgggccactgaaagctgatgactctgttcccccggggggcagccatatctacaactggaccattccagaaggccatgcacccaccgatgctgacccagcgtgcctcacctggatctaccattctcatgtagatgctccacgagacattgcaactggcctaattgggcctctcatcacctgtaaaagaggagccctggatgggaactcccctcctcaacgccaggatgtagaccatgatttcttcctcctcttcagtgtggtagatgagaacctcagctggcatctcaatgagaacattgccacttactgctcagatcctgcttcagtggacaaagaagatgagacatttcaggagagcaataggatgcatgcaatcaatggctttgtttttgggaatttacctgagctgaacatgtgtgcacagaaacgtgtggcctggcacttgtttggcatgggcaatgaaattgatgtccacacagcatttttccatggacagatgctgactacccgtggacaccacactgatgtggctaacatctttccagccacctttgtgactgctgagatggtgccctgggaacctggtacctggttaattagctgccaagtgaacagtcactttcgagatggcatgcaggcactctacaaggtcaagtcttgctccatggcccctcctgtggacctgctcacaggcaaagttcgacagtacttcattgaggcccatgagattcaatgggactatggcccgatggggcatgatgggagtactgggaagaatttgagagagccaggcagtatctcagataagtttttccagaagagctccagccgaattgggggcacttactggaaagtgcgatatgaagcctttcaagatgagacattccaagagaagatgcatttggaggaagataggcatcttggaatcctggggccagtgatccgggctgaggtgggtgacaccattcaggtggtcttctacaaccgtgcctcccagccattcagcatgcagccccatggggtcttttatgagaaagactatgaaggcactgtgtacaatgatggctcatcttaccctggcttggttgccaagccctttgagaaagtaacataccgctggacagtcccccctcatgccggtcccactgctcaggatcctgcttgtctcacttggatgtacttctctgctgcagatcccataagagacacaaattctggcctggtgggcccgctgctggtgtgcagggctggtgccttgggtgcagatggcaagcagaaaggggtggataaagaattctttcttctcttcactgtgttggatgagaacaagagctggtacagcaatgccaatcaagcagctgctatgttggatttccgactgctttcagaggatattgagggcttccaagactccaatcggatgcatgccattaatgggtttctgttctctaacctgcccaggctggacatgtgcaagggtgacacagtggcctggcacctgctcggcctgggcacagagactgatgtgcatggagtcatgttccagggcaacactgtgcagcttcagggcatgaggaagggtgcagctatgctctttcctcatacctttgtcatggccatcatgcagcctgacaaccttgggacatttgagatttattgccaggcaggcagccatcgagaagcagggatgagggcaatctataatgtctcccagtgtcctggccaccaagccacccctcgccaacgctaccaagctgcaagaatctactatatcatggcagaagaagtagagtgggactattgccctgaccggagctgggaacgggaatggcacaaccagtctgagaaggacagttatggttacattttcctgagcaacaaggatgggctcctgggttccagatacaagaaagctgtattcagggaatacactgatggtacattcaggatccctcggccaaggactggaccagaagaacacttgggaatcttgggtccacttatcaaaggtgaagttggtgatatcctgactgtggtattcaagaataatgccagccgcccctactctgtgcatgctcatggagtgctagaatctactactgtctggccactggctgctgagcctggtgaggtggtcacttatcagtggaacatcccagagaggtctggccctgggcccaatgactctgcttgtgtttcctggatctattattctgcagtggatcccatcaaggacatgtatagtggcctggtggggcccttggctatctgccaaaagggcatcctggagccccatggaggacggagtgacatggatcgggaatttgcattgttgttcttgatttttgatgaaaataagtcttggtatttggaggaaaatgtggcaacccatgggtcccaggatccaggcagtattaacctacaggatgaaactttcttggagagcaataaaatgcatgcaatcaatgggaaactctatgccaaccttaggggtcttaccatgtaccaaggagaacgagtggcctggtacatgctggccatgggccaagatgtggatctacacaccatccactttcatgcagagagcttcctctatcggaatggcgagaactaccgggcagatgtggtggatctgttcccagggacttttgaggttgtggagatggtggccagcaaccctgggacatggctgatgcactgccatgtgactgaccatgtccatgctggcatggagaccctcttcactgttttttctcgaacagaacacttaagccctctcaccgtcatcaccaaagagactgaaaaagcagtgccccccagagacattgaagaaggcaatgtgaagatgctgggcatgcagatccccataaagaatgttgagatgctggcctctgttttggttgccattagtgtcacccttctgctcgttgttctggctcttggtggagtggtttggtaccaacatcgacagagaaagctacgacgcaataggaggtccatcctggatgacagcttcaagcttctgtctttcaaacagtaacatctggagcctggagatatcctcaggaagcacatctgtagtgcactcccagcaggccatggactagtcactaaccccacactcaaaggggcatgggtggtggagaagcagaaggagcaatcaagcttatctggatatttctttctttatttattttacatggaaataatatgatttcactttttctttagtttctttgctctacgtgggcacctggcactaagggagtaccttattatcctacatcgcaaatttcaacagctacattatatttccttctgacacttggaaggtattgaaatttctagaaatgtatccttctcacaaagtagagaccaagagaaaaactcattgattgggtttctacttctttcaaggactcaggaaatttcactttgaactgaggccaagtgagctgttaagataacccacacttaaactaaaggctaagaatataggcttgatgggaaattgaaggtaggctgagtattgggaatccaaattgaattttgattctccttggcagtgaactactttgaagaagtggtcaatgggttgttgctgccatgagcatgtacaacctctggagctagaagctcctcaggaaagccagttctccaagttcttaacctgtggcactgaaaggaatgttgagttacctcttcatgttttagacagcaaaccctatccattaaagtacttgttagaacactgaaa

SEQ ID NO: 47 - DNA, 4454 bp, Homo sapiens
ggaacaggacattccagtagttttgtttctggaaaagagggcacccagcccttccccctccctcatcctcccatcccagtaaaccctgccaaattggaatcctggacttaatttaggagaaaggccctgtaaccaagatactgactgaacatggctggcggactcaggctggggtctgcagtgcagcattaatgggccgctgacatgaatatggagtagttttctctagcaaagagtaatgtgggccatggagtcaggccacctcctctgggctctgctgttcatgcagtccttgtggcctcaactgactgatggagccactcgagtctactacctgggcatccgggatgtgcagtggaactatgctcccaagggaagaaatgtcatcacgaaccagcctctggacagtgacatagtggcttccagcttcttaaagtctgacaagaaccggatagggggaacctacaagaagaccatctataaagaatacaaggatgactcatacacagatgaagtggcccagcctgcctggttgggcttcctggggccagtgttgcaggctgaagtgggggatgtcattcttattcacctgaagaattttgccactcgtccctataccatccaccctcatggtgtcttctacgagaaggactctgaaggttccctatacccagatggctcctctgggccactgaaagctgatgactctgttcccccggggggcagccatatctacaactggaccattccagaaggccatgcacccaccgatgctgacccagcgtgcctcacctggatctaccattctcatgtagatgctccacgagacattgcaactggcctaattgggcctctcatcacctgtaaaagaggagccctggatgggaactcccctcctcaacgccaggatgtagaccatgatttcttcctcctcttcagtgtggtagatgagaacctcagctggcatctcaatgagaacattgccacttactgctcagatcctgcttcagtggacaaagaagatgagacatttcaggagagcaataggatgcatgcaatcaatggctttgtttttgggaatttacctgagctgaacatgtgtgcacagaaacgtgtggcctggcacttgtttggcatgggcaatgaaattgatgtccacacagcatttttccatggacagatgctgactacccgtggacaccacactgatgtggctaacatctttccagccacctttgtgactgctgagatggtgccctgggaacctggtacctggttaattagctgccaagtgaacagtcactttcgagatggcatgcaggcactctacaaggtcaagtcttgctccatggcccctcctgtggacctgctcacaggcaaagttcgacagtacttcattgaggcccatgagattcaatgggactatggcccgatggggcatgatgggagtactgggaagaatttgagagagccaggcagtatctcagataagtttttccagaagagctccagccgaattgggggcacttactggaaagtgcgatatgaagcctttcaagatgagacattccaagagaagatgcatttggaggaagataggcatcttggaatcctggggccagtgatccgggctgaggtgggtgacaccattcaggtggtcttctacaaccgtgcctcccagccattcagcatgcagccccatggggtcttttatgagaaagactatgaaggcactgtgtacaatgatggctcatcttaccctggcttggttgccaagccctttgagaaagtaacataccgctggacagtcccccctcatgccggtcccactgctcaggatcctgcttgtctcacttggatgtacttctctgctgcagatcccataagagacacaaattctggcctggtgggcccgctgctggtgtgcagggctggtgccttgggtgcagatggcaagcagaaaggggtggataaagaattctttcttctcttcactgtgttggatgagaacaagagctggtacagcaatgccaatcaagcagctgctatgttggatttccgactgctttcagaggatattgagggcttccaagactccaatcggatgcatgccattaatgggtttctgttctctaacctgcccaggctggacatgtgcaagggtgacacagtggcctggcacctgctcggcctgggcacagagactgatgtgcatggagtcatgttccagggcaacactgtgcagcttcagggcatgaggaagggtgcagctatgctctttcctcatacctttgtcatggccatcatgcagcctgacaaccttgggacatttgagatttattgccaggcaggcagccatcgagaagcagggatgagggcaatctataatgtctcccagtgtcctggccaccaagccacccctcgccaacgctaccaagctgcaagaatctactatatcatggcagaagaagtagagtgggactattgccctgaccggagctgggaacgggaatggcacaaccagtctgagaaggacagttatggttacattttcctgagcaacaaggatgggctcctgggttccagatacaagaaagctgtattcagggaatacactgatggtacattcaggatccctcggccaaggactggaccagaagaacacttgggaatcttgggtccacttatcaaaggtgaagttggtgatatcctgactgtggtattcaagaataatgccagccgcccctactctgtgcatgctcatggagtgctagaatctactactgtctggccactggctgctgagcctggtgaggtggtcacttatcagtggaacatcccagagaggtctggccctgggcccaatgactctgcttgtgtttcctggatctattattctgcagtggatcccatcaaggacatgtatagtggcctggtggggcccttggctatctgccaaaagggcatcctggagccccatggaggacggagtgacatggatcgggaatttgcattgttgttcttgatttttgatgaaaataagtcttggtatttggaggaaaatgtggcaacccatgggtcccaggatccaggcagtattaacctacaggatgaaactttcttggagagcaataaaatgcatgcaatcaatgggaaactctatgccaaccttaggggtcttaccatgtaccaaggagaacgagtggcctggtacatgctggccatgggccaagatgtggatctacacaccatccactttcatgcagagagcttcctctatcggaatggcgagaactaccgggcagatgtggtggatctgttcccagggacttttgaggttgtggagatggtggccagcaaccctgggacatggctgatgcactgccatgtgactgaccatgtccatgctggcatggagaccctcttcactgttttttctcgaacagaacacttaagccctctcaccgtcatcaccaaagagactgaaaaagtgccccccagagacattgaagaaggcaatgtgaagatgctgggcatgcagatccccataaagaatgttgagatgctggcctctgttttggttgccattagtgtcacccttctgctcgttgttctggctcttggtggagtggtttggtaccaacatcgacagagaaagctacgacgcaataggaggtccatcctggatgacagcttcaagcttctgtctttcaaacagtaacatctggagcctggagatatcctcaggaagcacatctgtagtgcactcccagcaggccatggactagtcactaaccccacactcaaaggggcatgggtggtggagaagcagaaggagcaatcaagcttatctggatatttctttctttatttattttacatggaaataatatgatttcactttttctttagtttctttgctctacgtgggcacctggcactaagggagtaccttattatcctacatcgcaaatttcaacagctacattatatttccttctgacacttggaaggtattgaaatttctagaaatgtatccttctcacaaagtagagaccaagagaaaaactcattgattgggtttctacttctttcaaggactcaggaaatttcactttgaactgaggccaagtgagctgttaagataacccacacttaaactaaaggctaagaatataggcttgatgggaaattgaaggtaggctgagtattgggaatccaaattgaattttgattctccttggcagtgaactactttgaagaagtggtcaatgggttgttgctgccatgagcatgtacaacctctggagctagaagctcctcaggaaagccagttctccaagttcttaacctgtggcactgaaaggaatgttgagttacctcttcatgttttagacagcaaaccctatccattaaagtacttgttagaacactgaaaaaaaaaaaaaaaaaaa

SEQ ID NO: 48 - DNA, 1490 bp, Homo sapiens
agatatccgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaa

SEQ ID NO: 49 - DNA, 1753 bp, Homo sapiens
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 50 - DNA, 2880 bp, Homo sapiens
tgctgctctccgcccgcgtccggctcgtggccccctacttcgggcaccatggacacctcccggctcggtgtgctcctgtccttgcctgtgctgctgcagctggcgaccgggggcagctctcccaggtctggtgtgttgctgaggggctgccccacacactgtcattgcgagcccgacggcaggatgttgctcagggtggactgctccgacctggggctctcggagctgccttccaacctcagcgtcttcacctcctacctagacctcagtatgaacaacatcagtcagctgctcccgaatcccctgcccagtctccgcttcctggaggagttacgtcttgcgggaaacgctctgacatacattcccaagggagcattcactggcctttacagtcttaaagttcttatgctgcagaataatcagctaagacacgtacccacagaagctctgcagaatttgcgaagccttcaatccctgcgtctggatgctaaccacatcagctatgtgcccccaagctgtttcagtggcctgcattccctgaggcacctgtggctggatgacaatgcgttaacagaaatccccgtccaggcttttagaagtttatcggcattgcaagccatgaccttggccctgaacaaaatacaccacataccagactatgcctttggaaacctctccagcttggtagttctacatctccataacaatagaatccactccctgggaaagaaatgctttgatgggctccacagcctagagactttagatttaaattacaataaccttgatgaattccccactgcaattaggacactctccaaccttaaagaactaggatttcatagcaacaatatcaggtcgatacctgagaaagcatttgtaggcaacccttctcttattacaatacatttctatgacaatcccatccaatttgttgggagatctgcttttcaacatttacctgaactaagaacactgactctgaatggtgcctcacaaataactgaatttcctgatttaactggaactgcaaacctggagagtctgactttaactggagcacagatctcatctcttcctcaaaccgtctgcaatcagttacctaatctccaagtgctagatctgtcttacaacctattagaagatttacccagtttttcagtctgccaaaagcttcagaaaattgacctaagacataatgaaatctacgaaattaaagttgacactttccagcagttgcttagcctccgatcgctgaatttggcttggaacaaaattgctattattcaccccaatgcattttccactttgccatccctaataaagctggacctatcgtccaacctcctgtcgtcttttcctataactgggttacatggtttaactcacttaaaattaacaggaaatcatgccttacagagcttgatatcatctgaaaactttccagaactcaaggttatagaaatgccttatgcttaccagtgctgtgcatttggagtgtgtgagaatgcctataagatttctaatcaatggaataaaggtgacaacagcagtatggacgaccttcataagaaagatgctggaatgtttcaggctcaagatgaacgtgaccttgaagatttcctgcttgactttgaggaagacctgaaagcccttcattcagtgcagtgttcaccttccccaggccccttcaaaccctgtgaacacctgcttgatggctggctgatcagaattggagtgtggaccatagcagttctggcacttacttgtaatgctttggtgacttcaacagttttcagatcccctctgtacatttcccccattaaactgttaattggggtcatcgcagcagtgaacatgctcacgggagtctccagtgccgtgctggctggtgtggatgcgttcacttttggcagctttgcacgacatggtgcctggtgggagaatggggttggttgccatgtcattggttttttgtccatttttgcttcagaatcatctgttttcctgcttactctggcagccctggagcgtgggttctctgtgaaatattctgcaaaatttgaaacgaaagctccattttctagcctgaaagtaatcattttgctctgtgccctgctggccttgaccatggccgcagttcccctgctgggtggcagcaagtatggcgcctcccctctctgcctgcctttgccttttggggagcccagcaccatgggctacatggtcgctctcatcttgctcaattccctttgcttcctcatgatgaccattgcctacaccaagctctactgcaatttggacaagggagacctggagaatatttgggactgctctatggtaaaacacattgccctgttgctcttcaccaactgcatcctaaactgccctgtggctttcttgtccttctcctctttaataaaccttacatttatcagtcctgaagtaattaagtttatccttctggtggtagtcccacttcctgcatgtctcaatccccttctctacatcttgttcaatcctcactttaaggaggatctggtgagcctgagaaagcaaacctacgtctggacaagatcaaaacacccaagcttgatgtcaattaactctgatgatgtcgaaaaacagtcctgtgactcaactcaagccttggtaacctttaccagctccagcatcacttatgacctgcctcccagttccgtgccatcaccagcttatccagtgactgagagctgccatctttcctctgtggcatttgtcccatgtctctaattaatatgtgaaggaaaatgttttcaaaggttgagaacctgaaaatgtgagattgagtatatcagagcagtaattaataagaagagctgaggtgaaactcggtttaaa

SEQ ID NO: 51 - DNA, 2935 bp, Homo sapiens
ttttcctacatgctggccatggggaaatcaccactgggcactataagaagcccctgggctctctgcagagccagcggctccagctaagaggacaagatgaggcccggcctctcatttctcctagcccttctgttcttccttggccaagctgcaggggatttgggggatgtgggacctccaattcccagccccggcttcagctctttcccaggtgttgactccagctccagcttcagctccagctccaggtcgggctccagctccagccgcagcttaggcagcggaggttctgtgtcccagttgttttccaatttcaccggctccgtggatgaccgtgggacctgccagtgctctgtttccctgccagacaccacctttcccgtggacagagtggaacgcttggaattcacagctcatgttctttctcagaagtttgagaaagaactttccaaagtgagggaatatgtccaattaattagtgtgtatgaaaagaaactgttaaacctaactgtccgaattgacatcatggagaaggataccatttcttacactgaactggacttcgagctgatcaaggtagaagtgaaggagatggaaaaactggtcatacagctgaaggagagttttggtggaagctcagaaattgttgaccagctggaggtggagataagaaatatgactctcttggtagagaagcttgagacactagacaaaaacaatgtccttgccattcgccgagaaatcgtggctctgaagaccaagctgaaagagtgtgaggcctctaaagatcaaaacacccctgtcgtccaccctcctcccactccagggagctgtggtcatggtggtgtggtgaacatcagcaaaccgtctgtggttcagctcaactggagagggttttcttatctatatggtgcttggggtagggattactctccccagcatccaaacaaaggactgtattgggtggcgccattgaatacagatgggagactgttggagtattatagactgtacaacacactggatgatttgctattgtatataaatgctcgagagttgcggatcacctatggccaaggtagtggtacagcagtttacaacaacaacatgtacgtcaacatgtacaacaccgggaatattgccagagttaacctgaccaccaacacgattgctgtgactcaaactctccctaatgctgcctataataaccgcttttcatatgctaatgttgcttggcaagatattgactttgctgtggatgagaatggattgtgggttatttattcaactgaagccagcactggtaacatggtgattagtaaactcaatgacaccacacttcaggtgctaaacacttggtataccaagcagtataaaccatctgcttctaacgccttcatggtatgtggggttctgtatgccacccgtactatgaacaccagaacagaagagattttttactattatgacacaaacacagggaaagagggcaaactagacattgtaatgcataagatgcaggaaaaagtgcagagcattaactataacccttttgaccagaaactttatgtctataacgatggttaccttctgaattatgatctttctgtcttgcagaagccccagtaagctgtttaggagttagggtgaaagagaaaatgtttgttgaaaaaatagtcttctccacttacttagatatctgcaggggtgtctaaaagtgtgttcattttgcagcaatgtttaggtgcatagttctaccacactagagatctaggacatttgtcttgatttggtgagttctcttgggaatcatctgcctcttcaggcgcattttgcaataaagtctgtctagggtgggattgtcagaggtctaggggcactgtgggcctagtgaagcctactgtgaggaggcttcactagaagccttaaattaggaattaaggaacttaaaactcagtatggcgtctagggattctttgtacaggaaatattgcccaatgactagtcctcatccatgtagcaccactaattcttccatgcctggaagaaacctggggacttagttaggtagattaatatctggagctcctcgagggaccaaatctccaacttttttttcccctcactagcacctggaatgatgctttgtatgtggcagataagtaaatttggcatgcttatatattctacatctgtaaagtgctgagttttatggagagaggcctttttatgcattaaattgtacatggcaaataaatcccagaaggatctgtagatgaggcacctgctttttcttttctctcattgtccaccttactaaaagtcagtagaatcttctacctcataacttccttccaaaggcagctcagaagattagaaccagacttactaaccaattccaccccccaccaacccccttctactgcctactttaaaaaaattaatagttttctatggaactgatctaagattagaaaaattaattttctttaatttcattatgaacttttatttacatgactctaagactataagaaaatctgatggcagtgacaaagtgctagcatttattgttatctaataaagaccttggagcatatgtgcaacttatgagtgtatcagttgttgcatgtaatttttgcctttgtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctataggattggcagtttaaatgctttactcccccttttaaaataaatgattaaaatgtgctttgaaaaaagtcaaaaaaaaaaaaaaaaa

SEQ ID NO: 52 - DNA, 6318 bp, Homo sapiens
tagtaagacaggtgccttcagttcactctcagtaaggggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacagaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacgcttaaaacctagaacttcaaaatgttcgtattctattcaaaaggaaatatatatatatatatatatatatatatatatatatataaattaaaaaggaaagaaaactaacaaccaaccaaccaaccaaccaaccacaaaccaccctaaaatgacagccgctgatgtctgggcatcagcctttgtactctgtttttttaagaaagtgcagaatcaacttgaagcaagctttctctcataacgtaatgattatatgacaatcctgaagaaaccacaggttccatagaactaatatcctgtctctctctctctctctctctctctcttttttttttctttttccttttgccatggaatctgggtgggagaggatactgcgggcaccagaatgctaaagtttcctaacattttgaagtttctgtagttcatccttaatcctgacacccatgtaaatgtccaaaatgttgatcttccactgcaaatttcaaaagccttgtcaatggtcaagcgtgcagcttgttcagcggttctttctgaggagcggacaccgggttacattactaatgagagttgggtagaactctctgagatgtgttcagatagtgtaattgctacattctctgatgtagttaagtatttacagatgttaaatggagtatttttattttatgtatatactatacaacaatgttcttttttgttacagctatgcactgtaaatgcagccttcttttcaaaactgctaaatttttcttaatcaagaatattcaaatgtaattatgaggtgaaacaattattgtacactaacatatttagaagctgaacttactgcttatatatatttgattgtaaaaacaaaaagacagtgtgtgtgtctgttgagtgcaacaagagcaaaatgatgctttccgcacatccatcccttaggtgagcttcaatctaagcatcttgtcaagaaatatcctagtcccctaaaggtattaaccacttctgcgatatttttccacattttcttgtcgcttgtttttctttgaagttttatacactggatttgttaggggaatgaaattttctcatctaaaatttttctagaagatatcatgattttatgtaaagtctctcaatgggtaaccattaagaaatgtttttattttctctatcaacagtagttttgaaactagaagtcaaaaatctttttaaaatgctgttttgttttaatttttgtgattttaatttgatacaaaatgctgaggtaataattatagtatgatttttacaataattaatgtgtgtctgaagactatctttgaagccagtatttctttcccttggcagagtatgacgatggtatttatctgtattttttacagttatgcatcctgtataaatactgatatttcattcctttgtttactaaagagacatatttatcagttgcagatagcctatttattataaattatgagatgatgaaaataataaagccagtggaaattttctacctaggatgcatgacaattgtcaggttggagtgtaagtgcttcatttgggaaattcagcttttgcagaagcagtgtttctacttgcactagcatggcctctgacgtgaccatggtgttgttcttgatgacattgcttctgctaaatttaataaaaacttcagaaaaacctccattttgatcatcaggatttcatctgagtgtggagtccctggaatggaattcagtaacatttggagtgtgtattcaagtttctaaattgagattcgattactgtttggctgacatgacttttctggaagacatgatacacctactactcaattgttcttttcctttctctcgcccaacacgatcttgtaagatggatttcacccccaggccaatgcagctaattttgatagctgcattcatttatcaccagcatattgtgttctgagtgaatccactgtttgtcctgtcggatgcttgcttgattttttggcttcttatttctaagtagatagaaagcaataaaaatactatgaaatgaaagaacttgttcacaggttctgcgttacaacagtaacacatctttaatccgcctaattcttgttgttctgtaggttaaatgcaggtattttaactgtgtgaacgccaaactaaagtttacagtctttctttctgaattttgagtatcttctgttgtagaataataataaaaagactattaagagcaataaattatttttaagaaatcgagatttagtaaatcctattatgtgttcaaggaccacatgtgttctctattttgcctttaaatttttgtgaaccaattttaaatacattctcctttttgccctggattgttgacatgagtggaatacttggtttcttttcttacttatcaaaagacagcactacagatatcatattgaggattaatttatcccccctacccccagcctgacaaatattgttaccatgaagatagttttcctcaatggacttcaaattgcatctagaattagtggagcttttgtatcttctgcagacactgtgggtagcccatcaaaatgtaagctgtgctcctctcatttttatttttatttttttgggagagaatatttcaaatgaacacgtgcaccccatcatcactggaggcaaatttcagcatagatctgtaggatttttagaagaccgtgggccattgccttcatgccgtggtaagtaccacatctacaattttggtaaccgaactggtgctttagtaatgtggatttttttcttttttaaaagagatgtagcagaataattcttccagtgcaacaaaatcaattttttgctaaacgactccgagaacaacagttgggctgtcaacattcaaagcagcagagagggaactttgcactattggggtatgatgtttgggtcagttgataaaaggaaaccttttcatgcctttagatgtgagcttccagtaggtaatgattatgtgtcctttcttgatggctgtaatgagaacttcaatcactgtagtctaagacctgatctatagatgacctagaatagccatgtactataatgtgatgattctaaatttgtacctatgtgacagacattttcaataatgtgaactgctgatttgatggagctactttaagatttgtaggtgaaagtgtaatactgttggttgaactatgctgaagagggaaagtgagcgattagttgagcccttgccgggccttttttccacctgccaattctacatgtattgttgtggttttattcattgtatgaaaattcctgtgattttttttaaatgtgcagtacacatcagcctcactgagctaataaagggaaacgaatgtttcaaatcta

SEQ ID NO: 53 - DNA, 12844 bp, Homo sapiens
agactccgcccttgggcggggcctggatgcggccggagcggagcagtgctggagcgggagcctcagccctcaggcgccactgtgaggacctgaccggaccagaccatcccgcagcgccccgccccggccccctccgcgccctcccgacgccaggtcctgccgtcccgccgaccgtccgggagcgaacccgtcgtcccgcactcggagtccgcgatggcttcagtgacagatggtaaaactggagtcaaagatgcctctgaccagaattttgactacatgtttaaactgcttatcattggcaacagcagtgttggcaagacctccttcctcttccgctatgctgatgacacgttcaccccagccttcgttagcaccgtgggcatcgacttcaaggtgaagacagtctaccgtcacgagaagcgggtgaaactgcagatctgggacacagctgggcaggagcggtaccggaccatcacaacagcctattaccgtggggccatgggcttcattctgatgtatgacatcaccaatgaagagtccttcaatgctgtccaagactgggctactcagatcaagacctactcctgggacaatgcacaagttattctggtggggaacaagtgtgacatggaggaagagagggttgttcccactgagaagggccagctccttgcagagcagcttgggtttgatttctttgaagccagtgcaaaggagaacatcagtgtaaggcaggcctttgagcgcctggtggatgccatttgtgacaagatgtctgattcgctggacacagacccgtcgatgctgggctcctccaagaacacgcgtctctcggacaccccaccgctgctgcagcagaactgctcatgctaggcaaggcccaccttcctgacctcccctcattgtggccccacacccagtctgcttctccctgttacacactgtccgctctcagcccactctccctgttacacactgcccacactcagagcaagatgagttgctgctattctttgcctgcccctggggttctctgcagatggtcccagtaatagatactcagcactagactaacataacaggtcactacacgggtgcagaatcactttacaaaagaagactctgttttacgaaggggattcactacagggacttagagaacagtctcttttctgcctttaaaatgagagttcctccatttaccaaaatttgacacgcacacattcttcaggggcatgccaattgcgtaaagtgaggctcgcctgcatagctaatcctgttaaagacaacttctcaaagcacaacgtgcttgtttcctatcgggctccctgcggggctttctctcactacaagtcaagcttgggctctcaaagccctgcgcctgttaccacggatgcccacagggcctgggcagttgctgtggcgacaggaagagctaatcttcagagagctcagactctctaatgatgctgaaggagcaaaggctgagtcagaaacacacttaagagaaaaggattggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccggcctgggcgacagagcgagactccgtctcaaaaaaaaaaaaaaaaaaaaaaagagaaaaggattatcccctacaaaatgtcagaggttcctgctatatgaaagagcaagtaggtatgctcaagaaagacaaacagagaaaaagagaaacaggcaagatcaagaaacagatcatgagtttctgattttgctgctttccagttggttcttaactgtgggaacttagtgaaattggttattagttcttagactcctagaacctgaggattttagatttgacgggatgcccaaatttacctagtctgactagtcagttctaaccttcctttttctgacaagtgactgtcaagcctaacaatcaaatctctttcttttaaagcacaccttctaggcagggacaggagctcattttccacaccatctttgtcaactctcatagaaagttttccttgtatcgagctcaaatctgcctcctggaaattcttcttcttcttccctccctgttggtaccagctctgctgtcagagacttcacagtctgtgctccctctgccctgtgacgtcttcagactatttgagaacaggaatcatgactcctgggacttgccttttctctaggtcaaatacctctataattccatctgctgttcttcatagggtcttctccctatcctgcccttttcctccaatccatcttttaactgctcttgagcagtctaactgagaagtatgattcaaagcaaaataaatcttaaggtggcatgactctgaaaaaattgagaaaattgaactcagagatcccgatcccaacccctttctcctgggagtgaaaccttagtttctaccagagagtgtgggaaaccacttctggtggaagccccttaattaaatacctgaggaaaaaaataaaagaaactcagagaccagaataaattagctcattattctagcttgcttggccacagggacatattttgttttggctgaaataatgacatggaactggcagtgattccagaaaacctttcttctctatcatggcctgaatcctcagccacctcaaaagtcagcgggcaggaggagtctctcgccagttttcttttcatttcaaatgaggctcattgtcctagaaaagtaattaactagcaaccagtccaatgactaaataaaaggaccatccagctgtggctcacacctgtaatcccagcactttggaaagccaaggcaggaggaacacttgaggccaggagtttgagaccagcctgggcaatgtggtcaaattctatccctacaaaaaaaaaaattagccagatgtgatggtgcatgcctgtagtcccagctacttgggaggctgaggcaggaggattgcttgagcccaagaatttgaggttgcagtgagctgtaattatgccactgcattccagtctgggtgacagagtaagaataagaccctgtctctctgtctctctttctctctttttttttttaaaggagtcagctctacaaagatgttgctttctttgatgcaatgcagagagcagagctttggacttggaatcaggagacccggactctgtcattaaatcaactgtgactctgggccagttactttccacttttgagtcttgatttcctacttataaaatgagggagcttatttggatgatctttaaggtctcttttggcactaataactcggtgtctcttttttttcaccttcaccatttcagttgatccaccaaacaaacctgagagatcaggattggcatccaagagttgtctcggccaactctgatgtcatgcttactctgtactagacattgttccaagcattttacgtgcattaactcatttatcttcccaacatcttgtgagggaggcactatagtgagcctcatttgaagatgaggaaacaaaggtacaaagaggttctagctggacctctaaagtcacataataagtaagtggtagagctggagttcacatccaggcagtaggctccaaggtctgtgctcttaaccacattctgggctgcatcttttatagacaaactatgatccagagagattacgagacttggatcacataccaagagagtgttaaagccacattaggattcaattccagggccatcagattccaagtccactggagaaaagatgtatatctctaatctgttaacaaattgctcaactactcagactaatcccaggtgatggatgtctaatgctcaggaaaggcgagtcagtctctgaggcaacagatcccatgggcctgggtagaaaatgcccagtgcttcccagtcccaagtgctggctttccctgtatctgcctctgccaggcaacacttatcaggctcccaatcagcaggagcctccatgctccactttgaacagcctctatgctccagcaatggggcatttgtgaagagtgacttgattaacttttctgaccatgggtataatacagttgcttcagagggcagtggttctgggtgtgatttttacactgtaacattgtatacagtgtcatggataattactatttttttctggtcattaacactcacctactctagtactaggatttcagaccaaggtcctcatgacgcctggatattttagtatctatatccaataatcttttctctcctactgaatatccaggcaaagatgaaatcgttttctttaaaactgtcaaattctgtaaaactcaggagccagttcaagggaacaagcatcttcacaatagatggaatcaagagttaaatgttatagtggcaagcttgtctactgggcaacagacaaccagacctgcttgtgagatggcagctccccagccctgctctgtgacctcatttctgtcaaatgaaaggcagcagcttccagctgattgcagcatagtgttcatcaatcacagtaatagcgcaattagccaccaaggttcaagctgtgtaatatgtgttagtggcaacttgtcctggatttaatcttcctcaacaatccaaataaaatatttaaaaactcttgacttctggctgggcgcagcagctcatgcctgtaatcccagcactttgggaggccgaggtgggcagatcacctgaggtcaggagttcaagaccagcctgggcaacatggtgaaacccccatctctactaaaaatacaaaaattagccaggcgtggtggtgggcacctgaaatccctactcaggaggctgaggcagagaatcgcttgaacctgggaggcagaggttgcagtgagccgagatcgtgccactgcacttcagcctgggtgacagagcgagactccatctcaaaacaaaacaagcaaacaaacaacaacaacaaaaaacacctcttgacttctaaagacgcaaaagtggccaaaagtgcaatacagtattgtgtttatttacatctattttaaatgcatgtgtatctgtaaatacaaagtgattcgtgactcattgtctcctcagtctatagcattattaacttctaggagcagcagtggagtagagtgtactgaatggtcacagactcatcgattatcagatctggaaaggagcttagagaagatctgttccaggctcctattttatagaagggaaggttgacatccaaagaatggaaggaaatctcctaattattctgagagtatcacagtgatggagccaggactaggtcctggatcacctctaagaagacacttagctatttgactatcgactagggcctagcattattaagcactagataaatacagatgaaaaaaaaaatgatccctgcctgcaaggtcctatgatctaatggagatgctgtttctaaaatattattatcccaatttggcagtcaaggaaacagccctggaaaagttaacatgctcaagtcacccactagcatcatttgaaccctcctctgtctgactcatgctctttcaaatttttttcttcagattgtcttagcagaagggtagatgggatataccctctggtagtaccaggctcccaaggattcttagagttaaataacctcagttaattaaatagccacaattgcttggtgaccgaagccttataacatccacagaataagaccattctccagacctgactccccaactcatatcacctgctcctgccggccactaagctccttgcttggatatcgagttttctggagtatcctgaggaatgtttgtttgactttgtttgccaacagtttaggggaaggggaaagaactacaataaccagtgtcctgggatctcattgatttcagattccctgccccaagcctacacccaattacctgccatagttggggaatcaagtagcatcctgtggctggaagtaaatgcaaaacactagtccgtgagatataaatactgttaaatgatggttttttaaggtcctgatccattatatgaagtagacaaaattcaaatttatttattcatttattttctcaacaaatgaatatatattatgtgccaggatacaagtagtggcaaattagacacagttcttgctttcatgaaacgtatagcttcatgatttagtatagacattgtcaaatcatcacccaaatataattacaaagtactctaaaggaaaggcacgtgatgctgtgagaacactcaactgggaaaccggaatcacctttgagaaactgtttcaggggctcttggaagagtctactgctcccaaatatctctgctacccactggccattgctttacattcctcaactaagctttcaccttttagtactaacctttgatgactgatcaaatacaaatgccccaagaagactgaggataggagaaagaatatctctacctgtgaaacattgttagactgcctggctaggagttcattgttgttttctgaaggacgtaaccaaccactccaaaacttacaggcttaaaacaacaaacatgtatcatttcttatgattctgtgggttggctgggtggttcttctggctgaggcaggatggtctaggatagctacatccacatgtctggggtcccagctgagatgactggggctgttgaggcctttctccctgtggtgtcatcctccagaaggctgcccagatttgtccatatggtagcaggagtttcctcgaagcaagagagggcaagatccaacacagaagcacttttcaagctctgtttccatcacatttgccaatgtctcactgatgaacacaagttccatggccaagtccagttttaagaaatggagaaatagggcttggctcagtggctcatgtctgtaatcccagcactttgggaggccaaggcatgcggatcatttgaggtcaggagttccagaccagcctggccaacatggtgaaaacccatctctactaaaaatacaaaaattagctgggtgtggtggcgggcatctgtaatcccagctatttgggaggctgaagcacaagaattgcttgaacccaggaggaggaggttgcaatgagcctaaatcgcaccactgcacttcagcctgggcgatagagccagactcagtctcaaaaaaaaaaaaggggagggggaaatagatgccatctctttatgggaggagctacaaaatatggtgaccaatttttcaatctaccacaggaagcaccctcagtcctctgaaactaagtctggtagatgtcctggggtcttaaaacatggctccgatgatatcaccaaagacaagtggcaaaactgtatagggcagggcagtcttatcatttgtttaatagtgatccaaaggatttactttggaggaatcaagacactcgagatgaagaagttttgatgcttgttaaacagtccatttggatacctcttagctatccccgagggatgaatctgacttctcatttcacaggattcaccgtagataatggttgtaattcctaccggaagttcctggccagaagcccagcagaaagattcagtatatatagaaaagatggctccaagaacagttgggccttctgttctaactgtacttccttctttgatgtactcgtctagtcccgaggctttagatgccaagtctttgataataacgtgtatctaagtgcctactggacattttcatgtctcaaacttaacatgtccaaattgaaactcttgattctgcccccaaacttgtttgaaccccagtcttcacagaaaactcatccttaattctttgatttttctctttttctcagcctccttgtctaatctagcagcagatcctagggttttacttctaaatatatctcaaatctgatcatttttctccattttcattggcatgaccttggtccaggccaccattgttttctgccctagagagctaccacagagttcctaacatttccctacttacgtaattactccactctagtccattctgtctcacaggagtaacattttttatatatatatatatatatatatatatatatatatatatatatatatttttttttttttaatagagacggtcttgctatgttgcccaagctggtttcaaattctggcctcaagcgatcctcttgccttggcctcctgagtcactaggattgtacgtatgagccaccgcatccagcctcaatggcaatctcttaaaaatctaaataaatgaacggctcagtaacactgaggtttacttcacacaaaaacaatccaaaccttggcaagacggtgaaaccctgtctctacaaaaaatacaaaaaattaactgggcaaagtagcctgcacctatagtcccagctacttgggaggctgaggtgggaggatcgattgagccctggaggtcaagaatacagtgagccatagccatgattgtacactgtgccactccagcctgggtgacagagcaagaccctgtccccctctcaaaaaaaaaaaaaaaagaaagaaagaaagaaaaaaaagaagaaaaggaaagaaatgaagagaattcagagacttccattattattaatacctattttattgattctgtttctagccctgagtccgctcctaacttgctataggatctctggtaaatcatttcctgtaataagcagctgtcacctctctccttgtttcttccagaaatagtaatctcttctttagtagtactactactccctaacccaaaccaggtgattctagtgaagactgtcaataaacggagcatgtgatcaagcagggcccatcagaatccttccctaagatttttataaaaagctggacctattctttttccatttgagtggcaaatatttgaagatatgaggtctaaagctgtgatggcttattctccatccctgtgtaaattctggtctatagtaagcgaaaacaaggccattaggcagagggcagcagagacataaggtgagaaagagtgtggtctctggttttctagaccctgattctggtttggaggcttggctgatcacctcttcctttgattctgatagaaagctcaatgtatctttctaataaaacccccctttgctttgcttgttggagttaggttcttatcccttgcaaccaaaaatatattgtctcttcttttgttctcagttttctcatttatatatccttctagctccaaagcacagaaattctaaaacaaacaaacaaacaaacaaaaacaaacaaaaaaaacctgggtcattcagaaaatcccactgatatagactttctgatccagaatgtataatctgaaaagaagcctaccctcgtctccatcctctcttcttgtacctgaaggaacgaagaagagggatttctcaaggtgagaagcagttctccatggacactgatgacagcacaggcaaagtttcctatgactagggatcactgtccacacagagtctggcttcccaggtatccagcaggtagacaaaacagctaactccactgccactcctttctccacatccgttcctatttctcagccatctcagtgacatccgccatcttgagagtcaactactgactggactgagttgtgtggtatatgcttctgtttacttctcttctgtcttttttaagtggccaaatagcaaacgcttaaataggaaatctctgggagacttgaataaaagactttgcttggtagaaaatcatgtcacagaaaggctaatagacagcaaagtaaatcagcaagtccctgagcagtaggattaggattcctgtctcctttcagattcaaatgcatctgtttctggggttaacagtggactgttaagaggctgtgcagcttgggttaagtcattcttatctctgggcttcaggagcttagaccagatagtttctacaggctctcttggtgctgatgccttgggattctgtggctgttttctgtaagatctgcaagggggaaacaggattttggcagcaatcctttcattactaaagcttcctttcttttcgggtacagtgaaaagagccaaggctgtgtgaccccctcatcacttagccaggcgtatggtcctggtttctgaggctgccagaaagcatcttagcaatttgtgtttggatggtccatgcctgactattctaggctggaggttcctaaagagtaacaagaggaagagaaacaagaatctctgacacttgttgagaatagagcacagtcccatttgtttgaaaagagacaccaggcagccatgtttatgtgccagaaatgcattccacctcaaggaggacttaatttatggacccgtgtgtgccaggctgagctgggcaagatctttctcaggacaaactctgccatgcagctaaaagcctggaaactaaaggatttcatgtagtaaactatcttccaacccctgtagacatcagaccacaggatgaggtttcagaaggtcataaggcagaatagttaagcctacagggcttacagtctgacagacctgggttcagttcttgggtcttcatcactagttttgtgacttcgggaagatgactcccggagcctcagtgagcctcagttacttcatatgtaaatgaagtaatactatctacttcacaaggctgttgaaaggattaaatggagaatgggtgtaaaacccttagtgcagtgccgtgcacacacagtagatgccgaacgtgtgatgttggcactacacaatgtgtaatcccaatcaggcagagctaggcaggcaaatctaatccaggatctttgtaaggggactgagaaccagagactggagaaagccagtgtaaacaccatgagcaaaggagcaagagaaggggcattgtgtaagtaggagatggagcttgaacttactaagtggatcagggtagaagaatccagtcaggaccaagggaggagagtccaggaaaatgccatgagcagctctgtagcatgaccttgttgggctgggttaaagtagggtctgccaccagtcatgtgacagaaaggtacctcatgcacttcctccttcccccagaaatcagcctccaggagtgaggaatgagcccagaatgagagtttagagtgctccagagcctttgttagaggtgccctccgacattcagaaaaccaggattccagagacctgggtttgagtcctgactttgcagcatactaactgtgtgatcttgaaccaacatattttcacctaatgaggctgacaatcttccctacttcacaaaatagttatgagagtcaaataaaagtacattttagaaagtgaaatgctgtggacatttaaggtggagccactgtgagagtctagggggatagatggtattcgtctcagaatgaaacgaatacacccctctcagagccctttccaaggatcccctccttctttcagctccttccctccacctcaatacacactcctgtcccaggaacctaacctcatctagaaataccagggccagcatgccttacacctagaggtttggttggcttcagagaaacttctggaggctaaaagcagccaagaagaatcagccactacatgctgggcctggatgaacagagcagtgagctgtgatggggctggggctggggcccaggaggagcaggcaggagagtttgtatgcaccgtgattcaaatattataacaaaaatcatcgatcatgtgttaggcactttacagttcccaaagcactttcccatccatgccctgatgatctttgacacaacactgtgatgtgggttttattatttccagtacagatgaggaagactgaggcctgcatcagtgaagcaacctatccaagactacatagagaaggcagtaaatggcagggttagtctcagaacaggggagggtctgttccccccgcagtgggcagtcctaattctgaacttcacctatctgggggtgatagaggggaacaagaggaagcctgctgaagagaaaacctaaacatctgttttgtctacgtatgacttcctctgcttgtgggagagaaggaaggaaaggaacacattgttgtcagccccacaaccccaacagaattaaaccctggagcaggttgaacagcagaggcttccctcagatcaaggagccaggagcagatgatctatctctgtggccacacagagagatgtcaccttatgcaatttgcatatcatattcaattcccccaactgctctttctaatttattcaactggggaccaggctggtctcatgccaacctaggagatgtaccatagcagtatgagcagaattcctcaggaggaacaattagcaaaaactgcagttgcctctcgataggcctgagcagagagaggaacaatagctctcacgtctctcctcatcagattctaactaagcagatgttctcatgcttttttcttcttcctatgttctgtatactgacacctcttctcagtggcatatgaaatatgaaatgtcatgtgttgtgagtttgtataaatataaaggaatatatatacacagtagcaaaagagaagatctcatttacaaatatctatggtgtttccttgttctgtgttgatctgttttattgatacaaactgaattttcttaatgtatcttctatctctattatagtggcaatgatggtatatgcattaaagttcttctgaattgtg

SEQ ID NO: 54 - DNA, 2520 bp, Homo sapiens
ggatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaa

SEQ ID NO: 55 - DNA, 3963 bp, Homo sapiens
ggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgaggaggcctcccacgaagggcgaagatggccgagatgatcctaaaaataaccgaagaaagagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagtatttttgtacagatttgctttctcttacaaaaagaaaaaaaaaatcctgttgtattaacatttaaaaacagaattgtgttatgtgatcagttttgggggttaactttgcttaattcctcaggctttgcgatttaaggaggagctgccttaaaaaaaaataaaggccttattttgcaattatgggagtaaacaatagtctagagaagcatttggtaagctttatcatatatatattttttaaagaagagaaaaacaccttgagccttaaaacggtgctgctgggaaacatttgcactcttttagtgcatttcctcctgcctttgcttgttcactgcagtcttaagaaagaggtaaaaggcaagcaaaggagatgaaatctgttctgggaatgtttcagcagccaataagtgcccgagcacactgcccccggttgcctgcctgggccccatgtggaaggcagatgcctgctcgctctgtcacctgtgcctctcagaacaccagcagttaaccttcaagacattccacttgctaaaattatttattttgtaaggagaggttttaattaaaacaaaaaaaaattcttttttttttttttttccaattttaccttctttaaaataggttgttggagctttcctcaaagggtatggtcatctgttgttaaattatgttcttaactgtaaccagtttttttttatttatctctttaatctttttttattattaaaagcaagtttctttgtattcctcaccctagatttgtataaatgcctttttgtccatcccttttttctttgttgtttttgttgaaaacaaactggaaacttgtttctttttttgtataaatgagagattgcaaatgtagtgtatcactgagtcatttgcagtgttttctgccacagacctttgggctgccttatattgtgtgtgtgtgtgggtgtgtgtgtgttttgacacaaaaacaatgcaagcatgtgtcatccatatttctctgcatcttctcttggagtgagggaggctacctggaggggatcagcccactgacagaccttaatcttaattactgctgtggctagagagtttgaggattgctttttaaaaaagacagcaaacttttttttttatttaaaaaaagatatattaacagttttagaagtcagtagaataaaatcttaaagcactcataatatggcatccttcaatttctgtataaaagcagatctttttaaaaagatacttctgtaacttaagaaacctggcatttaaatcatattttgtctttaggtaaaagctttggtttgtgttcgtgttttgtttgtttcacttgtttccctcccagccccaaaccttttgttctctccgtgaaacttacctttccctttttctttctcttttttttttttgtatattattgtttacaataaatatacattgcattaaaaagaaaaaaaaaaaaaaaaaaaaaaaaaaaaaaa

SEQ ID NO: 56 - DNA, 1159 bp, Homo sapiens
agtgccccaggagctatgacaagcaaaggaacatacttgcctggagatagcctttgcgatatttaaatgtccgtggatacagaaatctctgcaggcaagttgctccagagcatattgcaggacaagcctgtaacgaatagttaaattcacggcatctggattcctaatccttttccgaaatggcaggtgtgagtgcctgtataaaatattctatgtttaccttcaacttcttgttctggctatgtggtatcttgatcctagcattagcaatatgggtacgagtaagcaatgactctcaagcaatttttggttctgaagatgtaggctctagctcctacgttgctgtggacatattgattgctgtaggtgccatcatcatgattctgggcttcctgggatgctgcggtgctataaaagaaagtcgctgcatgcttctgttgtttttcataggcttgcttctgatcctgctcctgcaggtggcgacaggtatcctaggagctgttttcaaatctaagtctgatcgcattgtgaatgaaactctctatgaaaacacaaagcttttgagcgccacaggggaaagtgaaaaacaattccaggaagccataattgtgtttcaagaagagtttaaatgctgcggtttggtcaatggagctgctgattggggaaataattttcaacactatcctgaattatgtgcctgtctagataagcagagaccatgccaaagctataatggaaaacaagtttacaaagagacctgtatttctttcataaaagacttcttggcaaaaaatttgattatagttattggaatatcatttggactggcagttattgagatactgggtttggtgttttctatggtcctgtattgccagatcgggaacaaatgaatctgtggatgcatcaacctatcgtcagtcaaacccctttaaaatgttgctttggctttgtaaatttaaatatgtaagtgctatataagtcaggagcagctgtctttttaaaatgtctcggctagctagaccacagatatcttctagacatattgaacacatttaagatttgagggatataagggaaaatgatatgaatgtgtatttttactcaaaataaaagtaactgtttacgttaaaaaaaaaaaaaaaaaaaaaaaaaaaaa

(19) United States
(12) Patent Application Publication
(10) Pub. No.: US 2016/0060594 A1
(43) Pub. Date: Mar. 3, 2016

(54) ISOLATION OF NON-EMBRYONIC STEM CELLS AND USES THEREOF

(71) Applicant: THE JACKSON LABORATORY, Bar Harbor, ME (US)

(72) Inventor: Wa Xian, Unionville, CT (US)

(73) Assignee: THE JACKSON LABORATORY, Bar Harbor, ME (US)

(21) Appl. No.: 14/853,210

(22) Filed: Sep. 14, 2015

Related U.S. Application Data
(63) Continuation of application No. PCT/US2014/027207, filed on Mar. 14, 2014.
(60) Provisional application No. 61/912,795, filed on Dec. 6, 2013.
(60) Provisional application No. 61/792,027, filed on Mar. 15, 2013.

(51) Int. Cl. C12N 5/074; A61K 35/407; A61K 35/42; A61K 35/48; A61K 35/30; A61K 35/22; A61K 35/38; A61K 35/39

(52) U.S. Cl. CPC C12N 5/0607; A61K 35/38; A61K 35/407; A61K 35/39; A61K 35/48; A61K 35/30; A61K 35/22; A61K 35/42; C12N 2501/42; C12N 2501/155; C12N 2501/415; C12N 2501/105; C12N 2501/15; C12N 2533/52

(57) ABSTRACT

The invention described herein relates to methods of isolating non-embryonic stem cell, e.g., adult stem cell, from a non-embryonic tissue, e.g., an adult tissue or organ. Non-embryonic stem cells (e.g., adult stem cells) thus isolated from the various tissues or organs can self-renew or propagate indefinitely in vitro, are multipotent and can differentiate into the various differentiated cell types normally found within the tissue or organ from which the stem cells are isolated. In addition, the isolated stem cells can be propagated through clonal expansion of a single isolated stem cell, to produce a clone of which at least about 40%, 70%, or 90% or more cells within the clone can be further passaged as single cell originated clones.

### REFERENCE TO RELATED APPLICATIONS

This application is a continuation application of International Application No. PCT/US2014/027207, filed on Mar. 14, 2014, which claims the benefit of the filing dates under 35 U.S.C. §119(e) to U.S. Provisional Application No. 61/792,027, filed on Mar. 15, 2013, and U.S. Provisional Application No. 61/912,795, filed on Dec. 6, 2013, the entire content of each of which applications is incorporated herein by reference.

### BACKGROUND OF THE INVENTION

Embryonic stem cells (ES cells) are multipotent stem cells derived from the inner cell mass of a blastocyst—an early-stage embryo. For example, in human, embryos reach the blastocyst stage at around 4-5 days post fertilization, at which time they typically consist of about 50-150 cells. Isolating the embryoblast or inner cell mass (ICM) results in destruction of the fertilized human embryo, which raises ethical issues and legal issues.

In contrast, non-embryonic stem cells (such as adult stem cells) are stem cells not of embryonic origin, and the isolation of which does not involve the destruction of a mammalian embryo. For example, adult stem cells, also known as somatic stem cells, are undifferentiated stem cells that can be found throughout the body of juvenile as well as adult animals and human bodies. These stem cells, on the one hand, are capable of self-renewal or self-regeneration virtually indefinitely, and on the other hand, are capable of differentiating into various mature or differentiated cell types, thus replenishing dying cells and regenerating damaged tissues.

Numerous adult stem cells have been identified so far.

For example, hematopoietic stem cells are found in the bone marrow and give rise to all the blood cell types.

Mammary stem cells provide the source of cells for growth of the mammary gland during puberty and gestation, and play an important role in carcinogenesis of the breast. Mammary stem cells have been isolated from human and mouse tissue as well as from cell lines derived from the mammary gland. Such stem cells can give rise to both the luminal and myoepithelial cell types of the gland, and have been shown to have the ability to regenerate the entire organ in mice (Liu et al., Breast Cancer Research 7(3):86-95, 2005).

Intestinal stem cells divide continuously throughout life, and use a complex genetic program to produce the cells lining the surface of the small and large intestines (Van Der Flier and Clevers, Annual Review of Physiology 71:241-260, 2009). Intestinal stem cells reside near the base of the stem cell niche, called the crypts of Lieberkuhn. Intestinal stem cells are probably the source of most cancers of the small intestine and colon (Barker et al., Nature 457(7229):608-611, 2008).

Mesenchymal stem cells (MSCs) are of stromal origin and may differentiate into a variety of tissues. MSCs have been isolated from placenta, adipose tissue, lung, bone marrow and blood, Wharton's jelly from the umbilical cord (Phinney and Prockop, Stem Cells 25(11):2896-2902, 2007), and teeth (perivascular niche of dental pulp and periodontal ligament) (Shi et al., Orthod. Craniofac. Res. 8(3):191-199, 2005). MSCs are attractive for clinical therapy due to their ability to differentiate, provide trophic support, and modulate innate immune response (Phinney and Prockop, supra).

Endothelial Stem Cells are one of the three types of Multipotent stem cells found in the bone marrow. They are a rare and controversial group with the ability to differentiate into endothelial cells, the cells that line blood vessels.

The existence of stem cells in the adult brain has been postulated following the discovery that the process of neurogenesis, the birth of new neurons, continues into adulthood in rats (Altman and Das, The Journal of Comparative Neurology 124 (3):319-335, 1965). The presence of stem cells in the mature primate brain was first reported in 1967 (Lewis, Nature 217(5132):974-975, 1968). It has since been shown that new neurons are generated in adult mice, songbirds and primates, including humans. Normally, adult neurogenesis is restricted to two areas of the brain—the subventricular zone, which lines the lateral ventricles, and the dentate gyrus of the hippocampal formation (Alvarez-Buylla et al., Brain Research Bulletin 57(6):751-758, 2002). Although the generation of new neurons in the hippocampus is well established, the presence of true self-renewing stem cells there has been debated (Bull and Bartlett, The Journal of Neuroscience 25(47):10815-10821, 2005). Under certain circumstances, such as following tissue damage in ischemia, neurogenesis can be induced in other brain regions, including the neocortex.

Neural stem cells are commonly cultured in vitro as so called neurospheres—floating heterogeneous aggregates of cells, containing a large proportion of stem cells (Reynolds and Weiss, Science 255 (5052):1707-1710, 1992). They can be propagated for extended periods of time and differentiated into both neuronal and glia cells, and therefore behave as stem cells. However, some recent studies suggest that this behavior is induced by the culture conditions in progenitor cells, the progeny of stem cell division that normally undergo a strictly limited number of replication cycles in vivo (Doetsch et al., Neuron 36(6):1021-1034, 2002). Furthermore, neurosphere-derived cells do not behave as stem cells when transplanted back into the brain (Marshall et al., Stem Cells 24(3):731-738, 2006).

Neural stem cells share many properties with haematopoietic stem cells (HSCs). Remarkably, when injected into the blood, neurosphere-derived cells differentiate into various cell types of the immune system (Bjornson et al., Science 283(5401):534-537, 1999).

Olfactory adult stem cells have been successfully harvested from the human olfactory mucosa cells, which are found in the lining of the nose and are involved in the sense of smell (Murrell et al., Developmental Dynamics 233(2):496-515, 2005). If they are given the right chemical environment, these cells have the same ability as embryonic stem cells to develop into many different cell types. Olfactory stem cells hold the potential for therapeutic applications and, in contrast to neural stem cells, can be harvested with ease without harm to the patient. This means that they can be easily obtained from all individuals, including older patients who might be most in need of stem cell therapies.

Hair follicles contain two types of stem cells, one of which appears to represent a remnant of the stem cells of the embryonic neural crest. Similar cells have been found in the gastrointestinal tract, sciatic nerve, cardiac outflow tract and spinal and sympathetic ganglia. These cells can generate neurons, Schwann cells, myofibroblast, chondrocytes and melanocytes (Sieber-Blum and Hu, Stem Cell Rev. 4(4):256-260, 2008; Kruger et al., Neuron 35(4):657-669, 2002).

Multipotent stem cells with a claimed equivalency to embryonic stem cells have been derived from spermatogonial progenitor cells found in the testicles of laboratory mice by scientists in Germany and the United States. Researchers from Germany and the United Kingdom has confirmed the same capability using cells from the testicles of humans. The extracted stem cells are known as human adult germline stem cells (GSCs). Multipotent stem cells have also been derived from germ cells found in human testicles.

Since adult stem cells have the ability to divide or self-renew indefinitely, and the ability to generate all the cell types of the organ from which they originate, potentially regenerating the entire organ from a few cells, adult stem cells hold great potential for personalized and regenerative medicine. In addition, unlike embryonic stem cells, the use of adult stem cells in research and therapy is not considered to be controversial, because they are derived from adult tissue samples rather than destroyed human embryos.

### SUMMARY OF THE INVENTION

In one aspect, the invention provides a method for isolating a non-embryonic stem cell (e.g., a fetal stem cell or an adult stem cell) from a non-embryonic tissue (e.g., a fetal tissue or an adult tissue), the method comprising: (1) culturing dissociated epithelial cells from the non-embryonic tissue, in contact with a first population of lethally irradiated feeder cells and a basement membrane matrix, to form epithelial cell clones, in a medium comprising: (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF; the medium optionally further comprising at least one of: (g) a TGFβ signaling pathway inhibitor (e.g., a TGFβ inhibitor or a TGFβ receptor inhibitor); and, (h) nicotinamide or an analog, precursor, or mimic thereof; (2) isolating single cells from the epithelial cell clones, and, (3) culturing isolated single cells from step (2) individually to form single cell clones, in contact with a second population of lethally irradiated feeder cells and a second basement membrane matrix in the medium; wherein each of the single cell clones represents a clonal expansion of the non-embryonic stem cell, thereby isolating the non-embryonic stem cell.

In a related aspect, the invention provides a method for isolating a non-embryonic stem cell (e.g., a fetal stem cell or an adult stem cell) from a non-embryonic tissue (e.g., a fetal tissue or an adult tissue), the method comprising: (1) culturing dissociated epithelial cells from the non-embryonic tissue, in contact with a first population of lethally irradiated feeder cells and a basement membrane matrix, to form epithelial cell clones, in a medium comprising: (a) a Notch agonist; (b) a ROCK (Rho Kinase) inhibitor; (c) a TGFβ signaling pathway inhibitor, such as TGFβ inhibitor, or a TGFβ receptor inhibitor); (d) a Wnt agonist; (e) nicotinamide or an analog, precursor, or mimic thereof, (f) a mitogenic growth factor; and, (g) insulin or IGF; the medium optionally further comprising (h) a Bone Morphogenetic Protein (BMP) antagonist; (2) isolating single cells from the epithelial cell clones, and, (3) culturing isolated single cells from step (2) individually to form single cell clones, in contact with a second population of lethally irradiated feeder cells and a second basement membrane matrix in the medium; wherein each of the single cell clones represents a clonal expansion of the non-embryonic stem cell, thereby isolating the non-embryonic stem cell.

In certain embodiments, the non-embryonic tissue is a cuboidal or columnar epithelial tissue. In certain embodiments, the non-embryonic tissue is an adult cuboidal or columnar epithelial tissue. In certain embodiments, the non-embryonic tissue is not a stratified epithelial tissue, such as skin or other epithelial tissues similar to skin.

In certain embodiments, the non-embryonic stem cell is an adult stem cell that substantially lacks expression of p63, or does not detectably express p63. In other embodiments, the non-embryonic stem cell is an adult stem cell that does express p63 (e.g., certain adult stem cell from lung, esophagus, or bladder).

As used herein, “p63” refers to a member of the tumor suppressor p53 family (for review, see Yang et al., Trends Genet. 18:90-95, 2002; and McKeon, Genes & Dev. 18:465-469, 2004).

In certain embodiments, the non-embryonic stem cell is an adult lung stem cell isolated from an adult lung tissue.

In certain embodiments, the method further comprises isolating single non-embryonic stem cell from the single cell clones.

In certain embodiments, the method further comprises culturing one of the single cell clones to generate a pedigree cell line of the non-embryonic stem cell.

In certain embodiments, the non-embryonic tissue is an adult tissue.

In other embodiments, the non-embryonic tissue is a fetal tissue.

In certain embodiments, the non-embryonic tissue is a mammalian tissue (e.g., a human tissue).

In certain embodiments, the non-embryonic tissue is obtained from or originates in lung, esophagus, stomach, small intestine, colon, intestinal metaplasia, fallopian tube, kidney, pancreas, bladder, or liver, or a portion/section thereof.

In certain embodiments, the non-embryonic tissue is a disease tissue, a disorder tissue, an abnormal condition tissue, or a tissue from a patient having the disease, disorder, or abnormal condition.

In certain embodiments, the disease, disorder, or abnormal condition comprises an adenoma, a carcinoma, an adenocarcinoma, a cancer, a solid tumor, an inflammatory bowel disease (e.g., Crohn's disease, ulcerative colitis), ulcer, gastropathy, gastritis, oesophagitis, cystitis, glomerulonephritis, polycystic kidney disease, hepatitis, pancreatitis, an inflammatory disorder (e.g., type I diabetes, diabetic nephropathy) and autoimmune disorder.

In certain embodiments, the cancer is ovarian cancer, pancreatic cancer (such as pancreatic ductal carcinoma), lung cancer (such as lung adenocarcinoma), or gastric cancer (such as gastric adenocarcinoma). In certain embodiments, the cancer is from a human patient (e.g., surgically removed cancer from patient, or a biopsy from patient), or is from a xenograft tumor grown in an immunosuppressed animal (e.g., mouse) using human cancer cell line or primary cancer cells.

In certain embodiments, the tissue from the patient having the disease, disorder, or abnormal condition is inflicted by the disease, disorder, or abnormal condition.

In certain embodiments, the non-embryonic stem cell is an adult stem cell.

In certain embodiments, in step (1), the (epithelial) cells are dissociated from the non-embryonic tissue through enzymatic digestion with an enzyme. For example, the enzyme may comprise collagenase, protease, dispase, pronase, elastase, hyaluronidase, accutase or trypsin.

In certain embodiments, in step (1), the (epithelial) cells are dissociated from the non-embryonic tissue through dissolving extracellular matrix surrounding the (epithelial) cells.

In certain embodiments, the feeder cells comprise 3T3-J2 cells (e.g., those forming a feeder cell layer).

In certain embodiments, the basement membrane matrix is a laminin-containing basement membrane matrix (e.g., MATRIGEL™ basement membrane matrix (BD Biosciences)), preferably growth factor-reduced.

In certain embodiments, the basement membrane matrix does not support 3-dimensional growth, or does not form a 3-dimensional matrix necessary to support 3-dimensional growth.

In certain embodiments, the medium further comprises 10% FBS that is not heat inactivated.

In certain embodiments, the Notch agonist comprises Jagged-1.

In certain embodiments, the ROCK inhibitor comprises Rho Kinase Inhibitor VI (Y-27632, (R)-(+)-trans-N-(4-Pyridyl)-4-(1-aminoethyl)-cyclohexanecarboxamide)), Fasudil or HA1071 (5-(1,4-diazepan-1-ylsulfonyl)isoquinoline), or H1152 ((S)-(+)-2-methyl-1-[(4-methyl-5-isoquinolinyl)sulfonyl]-hexahydro-1H-1,4-diazepine dihydrochloride).

In certain embodiments, the BMP antagonist comprises Noggin, DAN, a DAN-like proteins comprising a DAN cystine-knot domain (e.g., Cerberus and Gremlin), Chordin, a chordin-like protein comprising a chordin domain, Follistatin, a follistatin-related protein comprising a follistatin domain, sclerostin/SOST, decorin, or α-2 macroglobulin.

In certain embodiments, the Wnt agonist comprises R-spondin 1, R-spondin 2, R-spondin 3, R-spondin 4, an R-spondin mimic, a Wnt family protein (e.g., Wnt-3a, Wnt 5, Wnt-6a), Norrin, or a GSK-inhibitor (e.g., CHIR99021).

In certain embodiments, the mitogenic growth factor comprises EGF, Keratinocyte Growth Factor (KGF), TGFα, BDNF, HGF, and/or bFGF (e.g., FGF7 or FGF10).

In certain embodiments, the TGFβ receptor inhibitor comprises SB431542 (4-(4-(benzo[d][1,3]dioxol-5-yl)-5-(pyridin-2-yl)-1H-imidazol-2-yl)benzamide), A83-01, SB-505124, SB-525334, LY 364947, SD-208, or SJN 2511.

In certain embodiments, the TGFβ (signaling) inhibitor binds to and reduces the activity of one or more serine/threonine protein kinases selected from the group consisting of ALK5, ALK4, TGF-beta receptor kinase 1 and ALK7.

In certain embodiments, the TGFβ (signaling) inhibitor is added at a concentration of between 1 nM and 100 μM, between 10 nM and 100 μM, between 100 nM and 10 μM, or approximately 1 μM.

In certain embodiments, the medium comprises: 5 μg/mL insulin; 2 nM of (3,3′,5-Triiodo-L-Thyronine); 400 ng/mL hydrocortisone; 24.3 μg/mL adenine; 10 ng/mL EGF; 10% fetal bovine serum (without heat inactivation); 1 μM Jagged-1; 100 ng/mL noggin; 125 ng/mL R-spondin 1; 2.5 μM Y-27632; and 1.35 mM L-glutamine in DMEM:F12 3:1 medium, optionally the medium further comprises 0.1 nM cholera enterotoxin.

In certain embodiments, the medium further comprises 2 μM SB431542.

In certain embodiments, the medium further comprises 10 mM nicotinamide.

In certain embodiments, the medium further comprises 2 μM SB431542 and 10 mM nicotinamide.

In certain embodiments, the medium comprises: 5 μg/mL insulin; 2 nM of (3,3′,5-Triiodo-L-Thyronine); 400 ng/mL hydrocortisone; 24.3 μg/mL adenine; 10 ng/mL EGF; 10% fetal bovine serum (without heat inactivation); 1 μM Jagged-1; 125 ng/mL R-spondin 1; 2.5 μM Y-27632; 2 μM SB431542; 10 mM nicotinamide; and 1.35 mM L-glutamine in DMEM:F12 3:1 medium. Optionally, the medium further comprises 100 ng/mL noggin. Optionally the medium further comprises 0.1 nM cholera enterotoxin.

In certain embodiments, the non-embryonic tissue is adult small intestine, and the medium further comprises 10 mM nicotinamide.

In certain embodiments, the non-embryonic tissue is adult small intestine, and the medium further comprises 2 μM SB431542 and 10 mM nicotinamide.

In certain embodiments, the non-embryonic tissue is adult small intestine, and the medium further comprises (1) 2 μM SB431542, and one of Gastrin, PGE2, Wnt3a; or (2) 10 mM nicotinamide, and a GSK3 inhibitor.

In certain embodiments, the non-embryonic tissue is fetal small intestine, and the medium further comprises 10 mM nicotinamide.

In certain embodiments, the non-embryonic tissue is fetal small intestine, and the medium further comprises: FGF receptor inhibitor; N-Acetyl-L-cysteine; a p38 inhibitor (e.g., SB-202190, SB-203580, VX-702, VX-745, PD-169316, RO-4402257 and BIRB-796); Gastrin; PGE2; an FGF receptor inhibitor; Shh; TGFβ; 10 mM nicotinamide and TGFβ; 10 mM nicotinamide and Wnt3a; 10 mM nicotinamide and GSK3 inhibitor; or 10 mM nicotinamide and 2 μM SB431542.

In certain embodiments, the medium lacks at least one of: Wnt3a, p38 inhibitor (e.g., SB-202190, SB-203580, VX-702, VX-745, PD-169316, RO-4402257 and BIRB-796), N-Acetyl-L-cysteine, Gastrin, HGF, testosterone (e.g., (dihydro)testosterone), and PGE2.

In certain embodiments, at least about 40%, 50%, 60%, 70%, 80%, 85%, or about 90% of cells within each of the single cell clones, when isolated as single cell, is capable of proliferation as a single cell clone. In certain embodiments, a sing cell clone has at least about 300, 400, 450, 500, 550, 600 or more cells. In certain embodiments, cells in the single cell clone have substantially the same morphology or substantially homogeneous. In certain embodiments, the single cell clone grow substantially as a flat cell layer (e.g., a cell layer on top of the feeder layer and basement membrane matrix). In certain embodiments, the single cell clone does not form a three-dimensional structure, such as an organoid.

In certain embodiments, the non-embryonic stem cell, when isolated as single cell, is capable of self-renewal for greater than about 50 generations, 70 generations, 100 generations, 150 generations, 200 generations, 250 generations, 300 generations, 350 generations, or about 400 or more generations. In certain embodiments, the non-embryonic stem cell is capable of dividing once every about 25 hrs, 30 hrs, or 35 hrs.

In certain embodiments, the cloned stem cells can be frozen and stored short term at about −80° C. (e.g., on dry ice), or long term at about −200° C. (e.g., in liquid nitrogen), and subsequently thawed for culturing using standard tissue culture methods. Frozen cells can be thawed and put into culture according to the methods of the invention without losing their characteristics as stem cells (e.g., long-term renewability, and ability to differentiate, etc.), and without significant cell death. Therefore, in one embodiment, the invention provides frozen cloned stem cells stored at below −5° C., below −10° C., below −20° C., below −40° C., below −60° C., below −80° C., below −90° C., below −100° C., below −190° C., below −200° C., below −210° C., or below −220° C.

In certain embodiments, the non-embryonic stem cell is capable of differentiating into a differentiated cell type of the non-embryonic tissue.

In certain embodiments, the non-embryonic stem cell is a small intestine stem cell, and is capable of differentiating into a differentiated small intestine cell that (1) expresses a marker selected from MUC or PAS (goblet cell markers), CHGA (neuroendocrine cell marker), LYZ (Paneth cell marker), MUC7, MUC13, and KRT20; and/or (2) absorbs water and nutrients (such as by differentiated enterocytes), secretes mucus (such as by differentiated goblet cells), secretes intestinal hormones (such as by differentiated enteroendocrine cells), or secreting antibacterial substances (such as by differentiated Paneth cells).

As used herein, “expresses (certain) marker” includes the situation where a specific cell or cell type expresses a gene product (mRNA or protein) that can be readily detected and/or quantitated using an art recognized method for RNA or protein detection, such as in situ hybridization or immunostaining with antibody, or any other methods known in the art or described hereinbelow. The term may also include the situation where the gene product is preferentially expressed, such as expressing at a level significantly higher (e.g., 2-fold, 3-fold, 5-fold, 10-fold, 20-fold, 30-fold, 50-fold, 100-fold, 200-fold, 500-fold, 1000-fold or more) compared to that a relevant control cell.

Conversely, “does not express (certain) marker” includes the situation where a specific cell or cell type does not express a gene product (mRNA or protein) that can be readily detected and/or quantitated using an art recognized method for RNA or protein detection. The term may also include the situation where the gene product is expressed at a level significantly lower (e.g., 2-fold, 3-fold, 5-fold, 10-fold, 20-fold, 30-fold, 50-fold, 100-fold, 200-fold, 500-fold, 1000-fold or more) compared to that a relevant control cell.

For example, an undifferentiated stem cell (such as a small intestine stem cell) may not “express” a marker associated with a cell differentiated therefrom (e.g., a goblet cell), which may mean that the undifferentiated stem cell has undetectable level of expression of the marker, or may mean that the expression level of a marker in the undifferentiated stem cell is so low compared to that in the differentiated cell (e.g., goblet cell) such that the expression level in the undifferentiated stem cell is practicably negligible.

In certain embodiments, the non-embryonic stem cell expresses one or more stem cell markers selected from: SOX9, KRT19, KRT7, LGR5, CA9, FXYD2, CDH6, CLDN18, TSPAN8, BPIFB1, OLFM4, CDH17, and PPARGC1A.

In certain embodiments, the non-embryonic stem cell is a small intestine stem cell, and expresses one or more markers selected from: OLFM4, SOX9, LGR5, CLDN18, CA9, BPIFB1, KRT19, CDH17, and TSPAN8.

In certain embodiments, the non-embryonic stem cell substantially lacks expression of marker(s) associated with differentiated cell types in the non-embryonic tissue.

In certain embodiments, the non-embryonic stem cell is a small intestine stem cell, and lacks expression of markers associated with differentiated small intestine cells selected from MUC or PAS (goblet cell markers), CHGA (neuroendocrine cell marker), LYZ (Paneth cell marker), MUC7, MUC13, and KRT20.

In certain embodiments, the non-embryonic stem cell has an immature, undifferentiated morphology characterized by small round cell shape with high nucleus to cytoplasm ratio.

In another aspect, the invention provides a non-embryonic stem cell (e.g., a fetal stem cell or an adult stem cell) isolated according to any of the methods of the invention, or an in vitro culture thereof, such as one comprising a subject medium.

In certain embodiments, the non-embryonic stem cell is isolated from a cuboidal or columnar epithelial tissue. In certain embodiments, the non-embryonic stem cell is isolated from an adult cuboidal or columnar epithelial tissue. In certain embodiments, the non-embryonic stem cell is not isolated from a stratified epithelial tissue, such as skin or other tissues similar to skin.

In certain embodiments, the non-embryonic stem cell is an adult stem cell that substantially lacks p63 expression, or does not detectably express p63. In other embodiments, the non-embryonic stem cell is an adult stem cell that does express p63 (e.g., certain adult stem cell from lung, esophagus, or bladder).

In certain embodiments, the non-embryonic stem cell is isolated from an adult lung tissue (e.g., an adult lung tissue that is distinct from the upper airway tissue).

In certain embodiments, the medium does not comprise cholera enterotoxin.

In another aspect, the invention provides a single cell clone of a non-embryonic stem cell, or an in vitro culture thereof, such as one comprising a subject medium, wherein at least about 40%, 50%, 60%, 70%, or about 80% of cells within the single cell clone, when isolated as single cell, is capable of proliferation to produce single cell clone.

In another aspect, the invention provides a single cell clone of a non-embryonic stem cell, or an in vitro culture thereof, such as one comprising a subject medium, wherein the non-embryonic stem cell, when isolated as single cell, is capable of self-renewal for greater than about 50 generations, 70 generations, 100 generations, 150 generations, 200 generations, 250 generations, 300 generations, 350 generations, or about 400 or more generations.

In another aspect, the invention provides a single cell clone of a non-embryonic stem cell, or an in vitro culture thereof, such as one comprising a subject medium, wherein the non-embryonic stem cell is capable of differentiating into a differentiated cell type of a non-embryonic tissue from which the non-embryonic stem cell is isolated, or in which the non-embryonic stem cell resides.

In another aspect, the invention provides a single cell clone of a non-embryonic stem cell, or an in vitro culture thereof, such as one comprising a subject medium, wherein the non-embryonic stem cell expresses one or more stem cell markers selected from: SOX9, KRT19, KRT7, LGR5, CA9, FXYD2, CDH6, CLDN18, TSPAN8, BPIFB1, OLFM4, CDH17, and PPARGC1A.

In another aspect, the invention provides a single cell clone of a small intestine stem cell, or an in vitro culture thereof, such as one comprising a subject medium, which expresses one or more markers selected from: OLFM4, SOX9, LGR5, CLDN18, CA9, BPIFB1, KRT19, CDH17, and TSPAN8.

In another aspect, the invention provides a single cell clone of a non-embryonic stem cell, or an in vitro culture thereof, such as one comprising a subject medium, wherein the non-embryonic stem cell substantially lacks expression of marker(s) associated with differentiated cell types in the non-embryonic tissue.

In another aspect, the invention provides a single cell clone of a non-embryonic stem cell, or an in vitro culture thereof, such as one comprising a subject medium, wherein the non-embryonic stem cell substantially lacks expression of p63 (or does not detectably express p63). In a related aspect, the invention provides a single cell clone of a non-embryonic stem cell, or an in vitro culture thereof, such as one comprising a subject medium, wherein the non-embryonic stem cell expresses p63.

In another aspect, the invention provides a single cell clone of a non-embryonic stem cell, or an in vitro culture thereof, such as one comprising a subject medium, wherein the non-embryonic stem cell has an immature, undifferentiated morphology characterized by small round cell shape with high nucleus to cytoplasm ratio.

In a related aspect, the invention also provides a library or collection of the subject single cell clone, or in vitro culture (such as one comprising a subject medium) thereof. In certain embodiments, the library or collection may comprise single cell clones from the same tissue/organ type. In certain embodiments, the library or collection may comprise single cell clones isolated from the same type of tissue/organ type, but from different members of a population. In certain embodiments, one or more (preferably each) member of the population are homozygous across at least one tissue typing locus (such as HLA-A, HLA-B, and HLA-D). In certain embodiments, at least one tissue typing locus (e.g., the HLA loci above) is engineered in the cloned stem cells via, for example, TALEN or CRISPR technologies (see below) to generate universal donor cell lines (e.g. liver cells) lacking tissue antigens encode by the tissue typing locus (e.g., HLA-A, HLA-B, and HLA-D, etc.). See Torikai et al. (Blood, 122(8):1341-1349, 2013, incorporated by reference). In certain embodiments, the population may be defined by ethnic group, age, gender, disease status, or any common characteristics of a population. The library or collection may have at least about 20, 30, 40, 50, 60, 70, 80, 90, 100, 120, 150, 180, 200, 250, 300 or more members.

The availability of MHC haplotype data across populations has enabled the “banking” or creating a library or collection of stem cells (iPSC or adult stem cells, such as the subject stem cells) from relatively small numbers of homozygous individuals for use across the population. For instance, in an analysis of 10,000 individuals in the UK, it was determined that only 10 individuals homozygous across the key tissue typing loci on chromosome 6 could provide matches for 37.5% of the UK population at HLA-A, HLA-B, and HLA-DR. Stem cells derived from 150 individuals could provide even closer matches across these loci which would further enhance the success of transplants and perhaps obviate the need of immunosuppression therapy. Thus in the case of liver transplants for which the patient's stem cells might be limited by disease or infection, one could create such libraries or collections from donors, and have unlimited sources for transplantation of tissue-matched cells for a large segment of the population.

In another aspect, the invention provides a medium for isolating and/or culturing non-embryonic stem cell, the medium comprising: (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF.

In certain embodiments, the medium further comprises at least one of: (g) a TGFβ signaling pathway inhibitor (e.g., a TGFβ inhibitor or a TGFβ receptor inhibitor); and, (h) nicotinamide or a precursor, analog, or mimic thereof.

In a related aspect, the invention provides a medium for isolating and/or culturing non-embryonic stem cell, the medium comprising: (a) a Notch agonist; (b) a ROCK (Rho Kinase) inhibitor; (c) a TGFβ signaling pathway inhibitor (e.g., a TGFβ inhibitor or a TGFβ receptor inhibitor); (d) a Wnt agonist; (e) nicotinamide or a precursor, analog, or mimic thereof; (f) a mitogenic growth factor; and, (g) insulin or IGF.

In certain embodiments, the medium further comprises (h) a Bone Morphogenetic Protein (BMP) antagonist.

In another aspect, the invention provides a method of treating a subject having a disease, a disorder, or an abnormal condition and in need of treatment, comprising: (1) using any of the subject method, isolating an adult stem cell from a tissue corresponding to a tissue affected by the disease, disorder, or abnormal condition in the subject; (2) optionally, altering the expression of at least one gene in the adult stem cell to produce an altered adult stem cell; (3) reintroducing the isolated adult stem cell or altered adult stem cell, or a clonal expansion thereof, into the subject, wherein at least one adverse effect or symptom of the disease, disorder, or abnormal condition is alleviated in the subject.

In certain embodiments, the expression of at least one gene in the adult stem cell is altered to produce an altered adult stem cell.

In certain embodiments, the tissue from which the adult stem cell is isolated is from a healthy subject.

In certain embodiments, the tissue from which the adult stem cell is isolated is from the subject.

In certain embodiments, the tissue from which the adult stem cell is isolated is an affected tissue affected by the disease, disorder, or abnormal condition.

In certain embodiments, the tissue from which the adult stem cell is isolated is adjacent to an affected tissue affected by the disease, disorder, or abnormal condition.

In certain embodiments, the at least one gene is under-expressed in the tissue affected by the disease, disorder, or abnormal condition in the subject, and expression of the at least one gene is enhanced in the altered adult stem cell.

In certain embodiments, the at least one gene is over-expressed in the tissue affected by the disease, disorder, or abnormal condition in the subject, and expression of the at least one gene is reduced in the altered adult stem cell.

In certain embodiments, step (2) is effected by introducing into the adult stem cell an exogenous DNA or RNA.

In yet another aspect, the invention provides a method of screening for a compound, the method comprising: (1) using any of the methods of the invention, isolating an adult stem cell from a subject; (2) producing a cell line of the adult stem cell via single cell clonal expansion; (3) contacting test cells from the cell line with a plurality of candidate compounds; and, (4) identifying one or more compounds that produces a pre-determined phenotype change in the test cells.

It is contemplated that any embodiments described herein, including embodiments described in the examples and figures/drawings, and embodiments described under different aspects of the invention, can be combined with any one or more other embodiments where applicable.

### BRIEF DESCRIPTION OF THE DRAWINGS

FIG. 1 is a schematic diagram illustrating a general non-embryonic (e.g., adult) epithelial stem cell cloning technology. Pedigree stem cell lines derived from single stem cells of various adult (human) epithelial tissues can be established using the methods described herein. The epithelial stem cell lines can be cultured in vitro indefinitely. They can also be characterized by a number of sophisticated in vitro differentiation assays, in vivo xenograft experiments using “humanized” mouse models, as well as genomic profiling methods, such as gene expression array, genomic sequencing etc. The cell lines can be cryo preserved for long-term storage. The stem cells thus isolated have numerous practical utilities ranging from serving as international standard for drug screening and biomarkers discovery, to utility in regenerative medicine in patients from which they are originally isolated or derived from.

FIG. 2 shows representative undifferentiated morphology of the various columnar epithelial stem cell clones isolated from various tissue types from human, including stomach, small intestine, colon, intestinal metaplasia, Fallopian tube (oviducts), kidney, pancreas, liver, tracheal/upper airway (not shown), distal airway (not shown), bladder (both human and mouse), hippocampus, and lung (both human and mouse). Note that the individual cells in the shown single cell clones all have similar small, round morphology, with relatively large nucleus and high nuclear/cytoplasm ratio.

FIG. 3A shows that the pedigree cell lines from cloned human liver stem cells can propagate in vitro for more than 100 (e.g., 135) divisions while still maintaining the immature cell morphology (see FIG. 2). Note the same small, round morphology, with relatively large nucleus and high nuclear/cytoplasm ratio. FIG. 3B further shows that the immature cell morphology is maintained after 400 cell divisions.

FIG. 4 shows that the isolated liver stem cells can differentiate in vitro and highly express the differentiated liver cell markers such as ALB (Albumin), HNF4α (Hepatic Nuclear Factor 4, alpha) and AFP (alpha-fetoprotein). The fold change compares expression level of the marker genes in the differentiated liver cells vs. the undifferentiated stem cells. The presented real time PCR data was obtained using specific primers for each individual gene.

FIG. 5 shows that the pedigree cell lines from cloned human small intestine stem cells can propagate in vitro for more than 400 times while still maintaining the immature cell morphology (see FIG. 2).

FIG. 6 shows that a pedigree cell line derived from a single isolated human small intestine stem cell can differentiate into intestine-tissue-like structures in the air-liquid interface (ALI) cell culture system. One single intestine stem cell can differentiate into goblet cells (PAS staining and 5F4G1 antibody staining positive); Paneth cells (LYZ or lysozyme positive) and neuroendocrine cells (CHGA positive). 5F4G1 is an antibody that specifically stains the goblet cells. The intestine-tissue-like structure also expresses Villin that stains the microvilli-covered surface of small intestine tract where absorption takes place.

FIG. 7 shows that the stratified epithelial stem cells (from human upper airway) and the columnar epithelial stem cells (from small intestine) look similar morphologically when cultured in SCM medium in the feeder system, but display distinct differentiation capacity in the air-liquid interface (ALI) culture system. The small intestine stem cells differentiated into mature intestine-like structures (upper panel), while the upper airway stem cells differentiated into mature upper airway epithelium in the same differentiation system (Mucin5AC stains goblet cells in an isolated pattern; tubulin stains ciliated cells in a relatively continuous pattern surrounding the Mucin5AC stained goblet cells). Specifically, the upper airway stem cells differentiate into trachea like epithelium consisting ciliated cells and goblet cells. This data supports that tissue specific epithelial stem cells are intrinsically committed. Long term culturing is not affecting their commitment to respective tissue types.

FIG. 8 shows a gene expression comparison between intestine epithelial stem cells and upper airway epithelial stem cells. It shows that intestinal stem cells highly expressed markers such as OLFM4, CD133, ALDH1A1, LGR5 and LGR4, while upper airway stem cells highly expressed a distinct set of markers such as Krt14, Krt5, p63, Krt15 and SOX2.

FIG. 9A shows that cloned human stomach epithelial stem cells display the typical immature morphology (small, round cells with relatively large nucleus and high nuclear/cytoplasm ratio). The cells are positively stained for E-Cadherin (epithelial cell origin), SOX2 and SOX9 (stem cells marker for gastric epithelial stem cells). Occasionally, a couple of cells in culture express GKN1 which is a typical gastric epithelium differentiation marker, suggesting the cells are derived from the stomach.

FIG. 9B shows that the pedigree cell line derived from a single cloned human stomach stem cell can differentiate in vitro to form columnar epithelium expressing mature gastric epithelium markers such as GKN1, Gastric mucin, H⁺K⁺ ATPase and Muc5Ac.

FIG. 10 shows that the cloned intestinal metaplasia stem cells residing at the gastroesophageal junction express the columnar epithelial stem cell markers such as SOX9, and also express intestinal metaplasia specific markers such as CDH17. However, they don't express esophagus squamous stem cell marker, p63 or gastric epithelial stem cell marker, SOX2. The pedigree cell line derived from one single stem cell of Barrett's esophagus, the intestinal metaplasia can differentiate into columnar epithelium that mimic the mature intestinal metaplasia expressing the markers such as Cdx2 and Villin. However, they don't express gastric epithelium markers such as GKN1.

FIG. 11A shows a schematic diagram for isolating epithelial stem cells from human Fallopian Tube. Specifically, the tissue was enzymatically digested and cells were seeded on the feeder layer to form colonies consisting of hundreds of epithelial stem cells.

FIG. 11B shows that the isolated stem cells can divide more than 70 times in vitro without differentiation or senescence. FIG. 11C shows that the cloned cells are PAX8 positive (typical markers for Fallopian Tube epithelium stem cells), E-Cadherin positive (Epithelial cell marker), and Ki67 positive (proliferation marker).

FIG. 12A shows that the cloned human pancreatic stem cells express putative stem cell markers such as SOX9, Pdx1 and ALDH1A1.

FIG. 12B shows that the isolated stem cells can differentiate into tubal structures in vitro. The real time PCR data using gene specific primers shows that Pdx1 and SOX9 expressions are dramatically down-regulated when these cells differentiate.

FIG. 13 shows the organized structure formed by cells differentiated from liver stem cells (left panel), and the expression of several liver cell marker genes in the differentiated structure, such as albumin, HNF1α, and AFP.

FIG. 14 shows, in the left panel, hysterectomy of a patient with high-grade ovarian cancer showing bilateral involvement of ovaries and fallopian tube; and in the right panel, clones of cancer stem cells (CSCs) on irradiated 3T3 feeder cells derived from the tumor from the patient having the high-grade ovarian cancer.

FIG. 15 shows rodamine stain of colonies of CSCs derived from plating equal numbers of tumor cells in different media as described in Table 3. Plate 2 is supported by the six-factor media or SCM media.

FIG. 16 shows that the cloned CSCs from the high-grade ovarian cancer can be passaged indefinitely.

FIG. 17 shows that the CSCs derived from high-grade ovarian cancer can be used to generate tumors in immunosuppressed mice, and such tumors have the same histology as the tumors from which they were derived.

FIG. 18 shows examples of CSCs derived from pancreatic ductal carcinoma (upper panel) and lung adenocarcinoma (lower panel) grown on 3T3 feeder cells, and tumors from immunosuppressed mice generated from these CSCs.

FIG. 19 shows example of CSCs isolated from gastric adenocarcinoma.

FIG. 20 shows cloning of CSCs from human lung adenocarcinoma and ovarian cancer first grown in immunosuppressed mice.

FIG. 21 is a general scheme for identifying clones of CSCs resistant to standard chemotherapeutics. Top (left, center, and right) panels, 30,000 CSC clones on 3T3 cells are treated with cancer drugs such as cisplatin, paclitaxel, or a combination thereof such that less than 0.1% survive. Surviving clones (Right) are then retested for resistance and stably resistant clones selected for expansion and analysis. The bottom panel shows heat map of gene expression differences between three pedigrees of chemotherapy sensitive (left) and three resistant CSCs (right) from the same patient, showing 100-200 genes with significantly different expression profiles.

FIG. 22, top panel, shows principal component analysis of gene expression differences between sensitive, cisplatin-resistant, and paclitaxel-resistant CSCs. The bottom panel of FIG. 22 shows Venn diagram of cisplatin- and paclitaxel-resistant CSCs showing overlap of genes with altered expression.

FIG. 23A shows a representative image of liver stem cells infected with GFP retroviral vector. Note the relatively sparsely populated bright cells that express the heterologous gene GFP. FIG. 23B shows the FACS profile of sorting GFP-expressing cells. FIG. 23C shows images of sorted GFP-expressing liver stem cell clones growing on feeder. The left panel shows phase-contrast image of a large bright clone with relatively uniform high level of GFP expression, immediately adjacent to a slightly smaller 2^{nd} clone with uniform yet lower level of GFP expression. The partial image of a 3^{rd} clone having similar (relatively low) GFP expression level as the 2^{nd} clone is also visible at the lower right corner of the left panel. The right panel shows dark-field view of cloned liver stem cells with strong GFP expression level (bright patches of cell clones) against a mostly black background representing feeders without GFP expression.

FIGS. 24A and 24B show the results of intrasplenic injection of GFP-labeled liver stem cells. FIG. 24A shows a colony of human liver stem cells engineered to express a heterologous gene (GFP), and method of intrasplenic cell injection through the peritoneum of a mouse host. FIG. 24B shows an excised liver of the NSG mouse 7 days post injection of the GFP-expressing cloned liver stem cells. Note the already visible bright GFP-expressing tissue patch towards the bottom side of the liver. The two right panels of FIG. 24B show the enlarged images of the bright GPF signals in the excised liver.

### DETAILED DESCRIPTION OF THE INVENTION

### 1. Overview

The invention described herein relates to methods of isolating and/or maintaining in culture non-embryonic stem cell, e.g., adult stem cell, from a non-embryonic tissue, e.g., an adult tissue or organ. Non-embryonic stem cells (e.g., adult stem cells) thus isolated from the various tissues or organs can self-renew or propagate indefinitely in vitro, are multipotent and can differentiate into the various differentiated cell types normally found within the tissue or organ from which the stem cells are isolated. Cultures (including in vitro cultures) comprising the non-embryonic stem cells (e.g., adult stem cells) thus isolated are also within the scope of the invention.

In addition, the isolated stem cells can be propagated through clonal expansion of a single isolated stem cell, to produce a clone (e.g., as an in vitro culture) of which at least about 40%, 70%, or 90% or more cells within the clone can be further passaged as single cell originated clones. Thus the stem cells isolated using the methods of the invention are uniquely capable of being manipulated in vitro through standard molecular biology techniques, such as introduction of exogenous genetic materials through infection or transfection.

Thus in one aspect, the invention provides a method for isolating a non-embryonic stem cell from a non-embryonic tissue, the method comprising: (1) culturing dissociated epithelial cells from the non-embryonic tissue, in contact with a first population of lethally irradiated feeder cells and a basement membrane matrix, to form epithelial cell clones, in a medium comprising: (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF; the medium optionally further comprising at least one of: (g) a TGFβ signaling pathway inhibitor (such as a TGFβ inhibitor or a TGFβ receptor inhibitor); and, (h) nicotinamide or an analog, precursor, or mimic thereof; (2) isolating single cells from the epithelial cell clones, and, (3) culturing isolated single cells from step (2) individually to form single cell clones, in contact with a second population of lethally irradiated feeder cells and a second basement membrane matrix in the medium; wherein each of the single cell clones represents a clonal expansion of the non-embryonic stem cell, thereby isolating the non-embryonic stem cell.

Alternatively, the invention provides a method for isolating a non-embryonic stem cell from a non-embryonic tissue, the method comprising: (1) culturing dissociated epithelial cells from the non-embryonic tissue, in contact with a first population of lethally irradiated feeder cells and a basement membrane matrix, to form epithelial cell clones, in a medium comprising: (a) a Notch agonist; (b) a ROCK (Rho Kinase) inhibitor; (c) a TGFβ signaling pathway inhibitor, such as TGFβ inhibitor, or a TGFβ receptor inhibitor); (d) a Wnt agonist; (e) nicotinamide or an analog, precursor, or mimic thereof, (f) a mitogenic growth factor; and, (g) insulin or IGF; the medium optionally further comprising (h) a Bone Morphogenetic Protein (BMP) antagonist; (2) isolating single cells from the epithelial cell clones, and, (3) culturing isolated single cells from step (2) individually to form single cell clones, in contact with a second population of lethally irradiated feeder cells and a second basement membrane matrix in the medium; wherein each of the single cell clones represents a clonal expansion of the non-embryonic stem cell, thereby isolating the non-embryonic stem cell.

In a related aspect, the invention provides a method for culturing a non-embryonic stem cell obtained using the isolation method of the invention, comprising culturing isolated single cells or single cell clones in contact with a population of lethally irradiated feeder cells and a basement membrane matrix in the subject medium, such as a medium comprising: (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF; the medium optionally further comprising at least one of: (g) a TGFβ signaling pathway inhibitor (such as a TGFβ inhibitor or a TGFβ receptor inhibitor); and, (h) nicotinamide or an analog, precursor, or mimic thereof.

Alternatively, the invention provides a method for culturing a non-embryonic stem cell obtained using the isolation method of the invention, comprising culturing isolated single cells or single cell clones in contact with a population of lethally irradiated feeder cells and a basement membrane matrix in the subject medium, such as a medium comprising: (a) a Notch agonist; (b) a ROCK (Rho Kinase) inhibitor; (c) a TGFβ signaling pathway inhibitor (such as a TGFβ inhibitor or a TGFβ receptor inhibitor); (d) a Wnt agonist; (e) nicotinamide or an analog, precursor, or mimic thereof; (f) a mitogenic growth factor; and, (g) insulin or IGF; the medium optionally further comprising (h) a Bone Morphogenetic Protein (BMP) antagonist.

In yet another related aspect, the invention provides an in vitro culture of the non-embryonic stem cell obtained using the isolation method of the invention. In certain embodiments, the in vitro culture comprises isolated single cells or single cell clones in contact with a population of lethally irradiated feeder cells and a basement membrane matrix in the subject medium, such as a medium comprising: (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF; the medium optionally further comprising at least one of: (g) a TGFβ signaling pathway inhibitor (such as a TGFβ inhibitor or a TGFβ receptor inhibitor); and, (h) nicotinamide or an analog, precursor, or mimic thereof.

Alternatively, the invention provides an in vitro culture of the non-embryonic stem cell obtained using the isolation method of the invention. In certain embodiments, the in vitro culture comprises isolated single cells or single cell clones in contact with a population of lethally irradiated feeder cells and a basement membrane matrix in the subject medium, such as a medium comprising: (a) a Notch agonist; (b) a ROCK (Rho Kinase) inhibitor; (c) a TGFβ signaling pathway inhibitor (such as a TGFβ inhibitor or a TGFβ receptor inhibitor); (d) a Wnt agonist; (e) nicotinamide or an analog, precursor, or mimic thereof; (f) a mitogenic growth factor; and, (g) insulin or IGF; the medium optionally further comprising (h) a Bone Morphogenetic Protein (BMP) antagonist.

In certain embodiments, the non-embryonic tissue is a cuboidal or columnar epithelial tissue. In certain embodiments, the non-embryonic tissue is not a stratified epithelial tissue such as skin. In certain embodiments, the non-embryonic tissue is from an adult lung.

The methods of the invention for isolating and culturing non-embryonic stem cells are described in further detail below in Section 2 (Methods for Obtaining and/or Culturing Stem Cells).

As used herein, “non-embryonic stem cell” includes adult stem cell isolated from an adult tissue or organ, and fetal stem cell isolated from prenatal tissue or organ.

In certain embodiments, the methods of the invention described herein isolate adult stem cell from an adult tissue or organ.

In a related embodiment, the methods of the invention described herein isolate fetal stem cell from a fetal or prenatal tissue or organ. In certain embodiments, when fetal tissue or organ is the source of the stem cell, the methods of the invention do not destroy the fetus or otherwise impair the normal development of the fetus, especially when the fetus is a human fetus. In other embodiments, the source of the fetal tissue is obtained from aborted fetus, dead fetus, macerated fetal material, or cell, tissue or organs excised therefrom.

Methods to obtain fetal tissue is well known in the art. For example, in human, human fetal tissue transplants have been attempted in a number of human disorders including Parkinson's disease, diabetes, severe combined immunodeficiency disease, DiGeorge syndrome, aplastic anemia, leukemia, thalassemia, Fabry's disease, and Gaucher's disease. With the immunodeficient disorders, restoration of immune function and long-term patient survival have been achieved (see Joint Report of the Council on Ethical and Judicial Affairs and the Council on Scientific Affairs, A-89, Medical Applications of Fetal Tissue Transplantation).

The methods of the invention is applicable to any animal tissue containing non-embryonic stem cells, including tissues from human, non-human mammal, non-human primate, rodent (including but not limited to mouse, rat, ferret, hamster, guinea pig, rabbit), livestock animals (including but not limited to pig, cattle, sheep, goat, horse, camel), bird, reptile, fish, pet or other companion animals (e.g., cat, dog, bird) or other vertebrates, etc.

The non-embryonic tissue may be obtained from or originates in any parts of the animal, including but not limited to stomach, small intestine, colon, intestinal metaplasia, fallopian tube, kidney, pancreas, bladder, esophagus, or liver, or a portion/section thereof.

In certain embodiments, the non-embryonic tissue is obtained from a tissue comprising epithelial tissue. In certain embodiments, the non-embryonic tissue is obtained from GI tract.

In certain embodiments, the non-embryonic tissue is obtained from a portion of a tissue or organ. For example, the non-embryonic tissue may be isolated from the duodenum portion of the small intestine, or the jejunum portion of the small intestine, or the ileum portion of the small intestine. The non-embryonic tissue may also be isolated from the cecum portion of the large intestine, or the colon portion of the large intestine, or the sigmoid colon of the large intestine, or the rectum portion of the large intestine. The non-embryonic tissue may be isolated from the greater curvature, the lesser curvature, the angular incisure, the cardia, the body, the fundus, the pylorus, the pyloric antrum, or the pyloric canal of the stomach. The non-embryonic tissue may further be isolated from the upper airway, or the distal airway of the lung.

In certain embodiments, the non-embryonic tissue is isolated from a healthy or normal individual.

In certain embodiments, the non-embryonic tissue is isolated from a disease tissue (e.g., a tissue affected by a disease), a disorder tissue (e.g., a tissue affected by a disorder), or a tissue otherwise have an abnormal condition.

As used herein, the term “disease” includes an abnormal or medical condition that affects the body of an organism, and is usually associated with specific symptoms and signs. The disease may be caused by external factors (such as infectious disease), or by internal dysfunctions (such as autoimmune diseases). In a broad sense, “disease” may also include any condition that causes pain, dysfunction, distress, social problems, or death to the person afflicted, or similar problems for those in contact with the person. In this broader sense, it may include injuries, disabilities, disorders, syndromes, infections, isolated symptoms, deviant behaviors, and atypical variations of structure and function, while in other contexts and for other purposes these may be considered distinguishable categories.

The term “disorder” includes a functional abnormality or disturbance, such as mental disorders, physical disorders, genetic disorders, emotional and behavioral disorders, and functional disorders, or physical disorders that are not caused by infectious organisms, such as metabolic disorders. Thus the concepts of disease, disorder, and other abnormal condition are not necessarily mutually exclusive.

In certain embodiments, the non-embryonic tissue is isolated from an individual having a disease, disorder, or otherwise abnormal condition, although the non-embryonic tissue itself may not have been inflicted with the disease, disorder, or abnormal condition. For example, the non-embryonic tissue may be isolated from a patient having lung cancer, but from a healthy portion of the lung not already inflicted with the lung cancer. In certain embodiments, the non-embryonic tissue may be nearby or distant from the disease, disorder, or abnormal tissue.

In certain embodiments, the non-embryonic tissue is isolated from an individual pre-disposed to develop a disease, disorder, or otherwise abnormal condition, or in high risk of developing the disease, disorder, or otherwise abnormal condition, based on, for example, genetic composition, family history, life style choice (e.g., smoking, diet, exercise habit) of the individual, although the individual has not yet developed the disease, disorder, or otherwise abnormal condition, or displayed a detectable symptom of the disease, disorder, or otherwise abnormal condition.

The methods of the invention can be used to isolate non-embryonic stem cells from a tissue or organ of a subject having any disease, disorder, or abnormal condition, without regarding to the type, severity, degree or stage of the disease, disorder, or abnormal condition. A representative list of disease, disorder, or abnormal condition comprises, without limitation, infectious disease, contagious disease, foodborne illness, foodborne illness or food poisoning, disease caused by pathogenic bacteria, toxins, viruses, prions or parasites, communicable disease, non-communicable disease, airborne disease, lifestyle disease, mental disorder, organic disease, an adenoma, a carcinoma, an adenocarcinoma, a cancer, a solid tumor, a blood disease, an inflammatory bowel disease (e.g., Crohn's disease, ulcerative colitis), ulcer, gastropathy, gastritis, oesophagitis, cystitis, glomerulonephritis, polycystic kidney disease, pancreatitis, hepatitis, an inflammatory disorder (e.g., type I diabetes, diabetic nephropathy), cystic fibrosis, and autoimmune disorder.

In certain embodiments, the cancer is ovarian cancer, pancreatic cancer (such as pancreatic ductal carcinoma), lung cancer (such as lung adenocarcinoma), or gastric cancer (such as gastric adenocarcinoma). In certain embodiments, the cancer is from a human patient (e.g., surgically removed cancer from patient, or a biopsy from patient), or is from a xenograft tumor grown in an immunosuppressed animal (e.g., mouse) using human cancer cell line or primary cancer cells.

Another aspect of the invention provides a non-embryonic stem cell isolated according to any one of the methods of the invention, or an in vitro culture thereof.

For example, the non-embryonic stem cell may be an adult or fetal stem cell. The non-embryonic stem cell may be isolated from a human, or from any of the non-human animals, mammals, vertebrates described above. The non-embryonic stem cell may be isolated from any parts of the animal, including but not limited to stomach, small intestine, colon, intestinal metaplasia, fallopian tube, kidney, pancreas, bladder, esophagus, or liver, or a portion/section thereof, including those described above. The non-embryonic stem cell may be isolated from a healthy individual, or an individual inflicted with or predisposed to develop a high risk of developing a disease, disorder, or otherwise abnormal condition.

In yet another aspect, the invention further provides a single cell clone of an isolated non-embryonic stem cell, or an in vitro culture thereof, wherein at least about 40%, 50%, 60%, 70%, or about 80% of cells within the single cell clone, when isolated as single cell, is capable of proliferation to produce single cell clone.

Each single cell clone, depending on stages of growth and other growth conditions, may comprise at least about 10, 100, 10³, 10⁴, 10⁵, 10⁶ or more cells.

In a related aspect, the invention provides a single cell clone of an isolated non-embryonic stem cell, or an in vitro culture thereof, wherein the non-embryonic stem cell, when isolated as single cell, is capable of self-renewal for greater than about 50 generations, 70 generations, 100 generations, 150 generations, 200 generations, 250 generations, 300 generations, 350 generations, or about 400 or more generations.

In a related aspect, the invention provides a single cell clone of an isolated non-embryonic stem cell, or an in vitro culture thereof, wherein the non-embryonic stem cell is capable of differentiating into a differentiated cell type of a non-embryonic tissue from which the non-embryonic stem cell is isolated, or in which the non-embryonic stem cell resides.

In a related aspect, the invention provides a single cell clone of an isolated non-embryonic stem cell, or an in vitro culture thereof, wherein the non-embryonic stem cell expresses one or more stem cell markers selected from: SOX9, KRT19, KRT7, LGR5, CA9, FXYD2, CDH6, CLDN18, TSPAN8, BPIFB1, OLFM4, CDH17, and PPARGC1A.

In a related aspect, the invention provides a single cell clone of a small intestine stem cell, or an in vitro culture thereof, which expresses one or more markers selected from: OLFM4, SOX9, LGR5, CLDN18, CA9, BPIFB1, KRT19, CDH17, and TSPAN8.

In a related aspect, the invention provides a single cell clone of a stomach stem cell, or an in vitro culture thereof, which expresses one or more markers selected from: SOX9, SOX2, CLDN18, TSPAN8, KRT7, KRT19, BPIFB1, and PPARGC1A.

In a related aspect, the invention provides a single cell clone of a colon stem cell, or an in vitro culture thereof, which expresses one or more markers selected from: SOX9, OLFM4, LGR5, CLDN18, CA9, BPIFB1, KRT19, and PPARGC1A.

In a related aspect, the invention provides a single cell clone of a intestinal metaplasia stem cell, or an in vitro culture thereof, which expresses one or more markers selected from: SOX9, CDH17, HEPH and RAB3B.

In a related aspect, the invention provides a single cell clone of a liver stem cell, or an in vitro culture thereof, which expresses one or more markers selected from: SOX9, KRT19, KRT7, FXYD2, and TSPAN8.

In a related aspect, the invention provides a single cell clone of a pancreatic stem cell, or an in vitro culture thereof, which expresses one or more markers selected from: SOX9, KRT19, KRT7, FXYD2, CA9, CDH6, PDX1 and ALDH1A1.

In a related aspect, the invention provides a single cell clone of a kidney stem cell, or an in vitro culture thereof, which expresses one or more markers selected from: KRT19, KRT7, FXYD2, and CDH6.

In a related aspect, the invention provides a single cell clone of a Fallopian tube stem cell, or an in vitro culture thereof, which expresses one or more markers selected from: ZFPM2, CLDN10, and PAX8.

In certain embodiments, the in vitro culture comprises a medium of the invention (e.g., a modified medium of the invention as described below). See section below describing the medium of the invention, each medium described therein is incorporated herein by reference.

In certain embodiments, the non-embryonic stem cell is capable of differentiating into a differentiated cell type of the non-embryonic tissue. For example, the isolated small intestine stem cell of the invention may differentiate into one or more cell types normally found in small intestine, such as enterocytes (the most abundant cell type, absorbing water and nutrients), goblet cells (the second major cell type and secreting mucus), enteroendocrine cells (secreting intestinal hormones), and Paneth cells (secreting, antibacterial substances). The isolated upper airway stem cell of the invention may differentiate into one or more cell types normally found in upper airway of the lung, such as ciliated cells and goblet cells. The isolated lung stem cell of the invention may differentiate into one or more cell types normally found in lung epithelium, such as type I and type II pneumocytes.

In certain embodiments, the non-embryonic stem cell is capable of differentiating into organized structures resembling the structure or substructures found in the tissue from which such non-embryonic stem cell originates. For example, the isolated small intestine stem cell of the invention may differentiate into intestine-tissue-like structure that resembles the microvilli-covered surface of small intestine tract. One characteristic function of the intestine-tissue-like structure is that these differentiated intestine cells can form brush border expressing Villin protein and multiple enzymes involved in absorption functions, including sucrase-isomaltase, lactase, maltase-glucoamylase, alanyl aminopeptidase.

In certain embodiments, the non-embryonic stem cell substantially lacks expression of marker(s) associated with differentiated cell types in the non-embryonic tissue. For example, In certain embodiments, the non-embryonic stem cell is a small intestine stem cell, and lacks expression of certain protein markers associated with differentiated small intestine cells selected from mucin/MUC or PAS (goblet cell markers), Chromogranin A/CHGA (neuroendocrine cell marker), lysozyme/LYZ (Paneth cell marker), MUC7, MUC13, and KRT20.

In certain embodiments, the non-embryonic stem cell has an immature, undifferentiated morphology characterized by small round cell shape with high nucleus to cytoplasm ratio. See, for example, the various isolated adult stem cell clones displaying similar morphology in culture.

In still another aspect, the invention provides a medium for isolating and/or culturing non-embryonic stem cell, the medium comprising: (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF.

In certain embodiments, the medium further comprises at least one of: (g) a TGFβ signaling pathway inhibitor, such as a TGFβ inhibitor or a TGFβ receptor inhibitor; and, (h) nicotinamide or a precursor, analog, or mimic thereof.

In a related aspect, the invention provides a medium for isolating and/or culturing non-embryonic stem cell, the medium comprising: (a) a Notch agonist; (b) a ROCK (Rho Kinase) inhibitor; (c) a TGFβ signaling pathway inhibitor (e.g., a TGFβ inhibitor or a TGFβ receptor inhibitor); (d) a Wnt agonist; (e) nicotinamide or a precursor, analog, or mimic thereof; (f) a mitogenic growth factor; and, (g) insulin or IGF.

In certain embodiments, the medium further comprises (h) a Bone Morphogenetic Protein (BMP) antagonist.

The various media of the invention and the components thereof are described in Section 3 (Medium) and the related Section 4 (Protein Sequences of the Representative Medium Factors). The various embodiments of the media of the invention specifically include any of the embodiments described in detail in these sections and other parts of the specification.

A further aspect of the invention provides a method of treating a subject having a disease, a disorder, or an abnormal condition and in need of treatment, comprising: (1) using any of the methods of the invention to isolate a non-embryonic (e.g., an adult) stem cell from a tissue corresponding to a tissue affected by the disease, disorder, or abnormal condition in the subject; (2) altering the expression of at least one gene in the adult stem cell to produce an altered adult stem cell; (3) reintroducing the altered adult stem cell or a clonal expansion thereof into the subject, wherein at least one adverse effect or symptom of the disease, disorder, or abnormal condition is alleviated in the subject.

For example, step (2) of the method may be effected by introducing into the adult stem cell an exogenous DNA or RNA that either increases or decreases the expression of a target gene in the isolated adult stem cell. Any of the art-recognized molecular biology techniques can be used to alter gene expression in a cell, e.g., in vitro or ex vivo. Such methods may include, without limitation, transfection or infection by a viral or non-viral based vector, which may encode the coding sequence of a protein or functional fragments thereof that is dysfunctional or deficient in the target cell, or may encode an RNA (antisense RNA, siRNA, miRNA, shRNA, ribozyme, etc.) that disrupts the function of a target gene.

In a recent study, Marvilio et al. (Nature Medicine 12(12): 1397-1402, 2006) reported that junctional epidermolysis bullosa (a nonlethal skin disorder) in a patient was treated by transplantation of genetically modified adult epidermal stem cells isolated from the same patient. The adult stem cell was isolated (using a different method) from a relatively healthy area (i.e., the palm) of the patient where adult stem cell can still be recovered. The genetic modification involved infecting the isolated adult stem cell with a retroviral vector that exogenously expresses a gene defective in the patient. Genetically corrected cultured epidermal grafts so prepared were then transplanted onto surgically prepared regions of the patient's body. Synthesis and proper assembly of normal levels of functional transgene were observed, together with the development of a firmly adherent epidermis that remained stable for the duration of the follow-up (1 year) in the absence of blisters, infections, inflammation or immune response.

In certain embodiments, the tissue from which the adult stem cell is isolated is from a healthy subject. Preferably, the healthy subject is HLA-type matched with the subject in need of treatment.

In certain embodiments, the tissue from which the adult stem cell is isolated is from the subject, and the isolated adult stem cell is autologous with respect to the subject.

In certain embodiments, the tissue from which the adult stem cell is isolated is an affected tissue affected by the disease, disorder, or abnormal condition.

In certain embodiments, the tissue from which the adult stem cell is isolated is adjacent to an affected tissue affected by the disease, disorder, or abnormal condition.

In certain embodiments, at least one gene is under-expressed in the tissue affected by the disease, disorder, or abnormal condition in the subject, and expression of the at least one gene is enhanced in the altered adult stem cell.

In certain embodiments, at least one gene is over-expressed in the tissue affected by the disease, disorder, or abnormal condition in the subject, and expression of the at least one gene is reduced in the altered adult stem cell.

In another aspect, the invention also provides a method of screening for a compound, the method comprising: (1) using any of the methods of the invention to isolate an adult stem cell (including a cancer stem cell) from a subject; (2) producing a cell line of the adult stem cell via single cell clonal expansion; (3) contacting test cells from the cell line with a plurality of candidate compounds; and, (4) identifying one or more compounds that produces a pre-determined phenotype change in the test cells.

This screening method of the invention may be used for target identification and validation. For example, a potential target gene in an adult stem cell isolated from a patient in need of treatment may functional abnormally (either over-expression or under-expression) to cause a phenotype associated with a disease, disorder, or abnormal condition. A clonal expansion of the adult stem cell isolated using the method of the invention may be subject to the screening method of the invention to test an array of potential compounds (small molecule compounds, etc.) to identify one or more compounds that can correct, alleviate, or reverse the phenotype.

In another embodiment, an adult stem cell may be isolated from a patient in need of treatment, such as from the a tissue affected by a disease, disorder, or abnormal condition. A clonal expansion of the adult stem cell isolated using the method of the invention may be subject to the screening method of the invention to test an array of potential compounds (small molecule compounds, or any RNA-based antagonists such as library of siRNA, etc.) to identify one or more compounds that can correct, alleviate, or reverse the phenotype. The affected target gene by an effective compound may be further identified by, for example, microarray, RNA-Seq, or PCR based expression profile analysis.

The adult stem cell isolated using the methods of the invention and clonal expansion thereof may be further useful for toxicology screens or studies such that any toxicology analysis and test can be tailored to individual patients set to receive a certain medicine or medical intervention.

The adult stem cell isolated using the methods of the invention and clonal expansion thereof may also be useful for regenerative medicine, where either autologous stem cells or stem cells isolated from HLA-type matched healthy donor can be induced to differentiate into tissues or organs in vitro, ex vivo, or in vivo to treat an existing condition or prevent/delay such a condition from developing. Such stem cells may be genetically manipulated prior to induced differentiation.

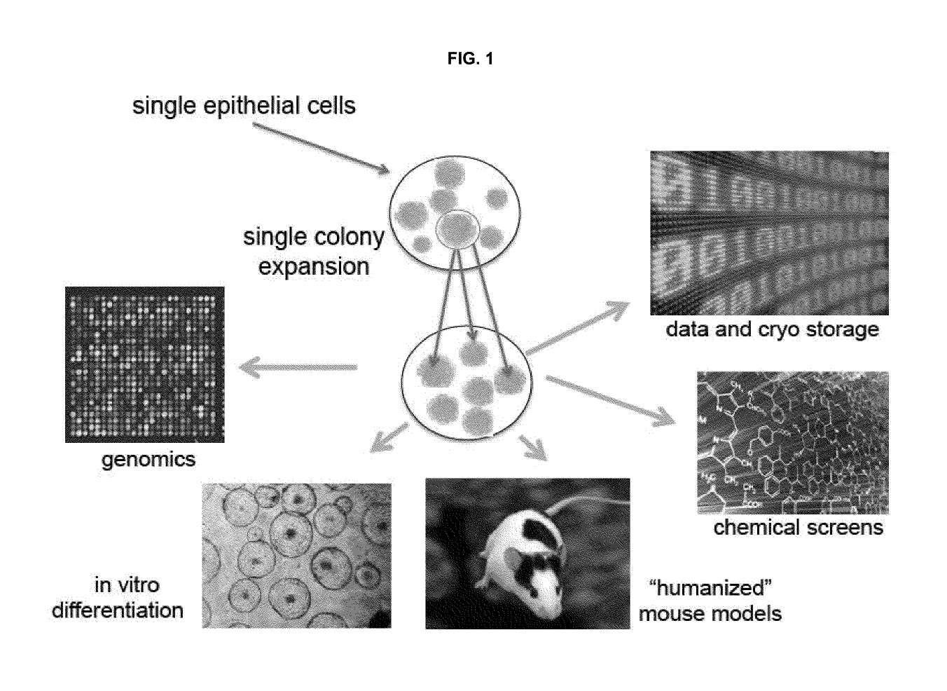

The adult stem cell isolated using the methods of the invention and clonal expansion thereof may be used in an in vitro or in vivo disease model. For example, isolated upper airway stem cells may be induced to differentiate in an air-liquid interface (ALI) to produce upper airway epithelia like structure, which may be used in any of the screening methods described herein. The isolated adult stem cells (e.g., those from human) may also be introduced into SCID or nude mice or rat to establish humanized disease model suitable for carrying out in vivo methods, such as the screening methods of the invention. See FIG. 1 for a representative number of uses of the subject stem cells.

### 2. Methods for Obtaining and/or Culturing Stem Cells

One aspect of the invention relates to a method for isolating a non-embryonic stem cell from a non-embryonic tissue, as generally described above.

Specifically, one step of the method comprises culturing dissociated cells (such as dissociated cuboidal epithelial cells) from the non-embryonic tissue, in contact with a first population of lethally irradiated feeder cells and an extracellular matrix, e.g., a basement membrane matrix, to form epithelial cell clones.

In certain embodiments, the (epithelial) cells are dissociated from the non-embryonic tissue through enzymatic digestion with an enzyme, including, without limitation, any one or more of collagenase, protease, dispase, pronase, elastase, hyaluronidase, accutase and/or trypsin.

These enzymes or functional equivalents are well known in the art, and in almost all cases are commercially available.

In other embodiments, the (epithelial) cells may be dissociated from the non-embryonic tissue through dissolving extracellular matrix surrounding the (epithelial) cells. One reagent suitable for this embodiment of the invention include a non-enzymatic proprietary solution marketed by BD Biosciences (San Jose, Calif.) as the BD™ Cell Recovery Solution (BD Catalog No. 354253), which allows for the recovery of cells cultured on BD MATRIGEL™ Basement Membrane Matrix for subsequent biochemical analyses.

In certain embodiments, the feeder cells may comprise certain lethally irradiated fibroblast, such as the murine 3T3-J2 cells. The feeder cells may form a feeder cell layer on top of the basement membrane matrix.

A suitable 3T3-J2 cell clone is well known in the art (see, for example, Todaro and Green, “Quantitative studies of the growth of mouse embryo cells in culture and their development into established lines.” J. Cell Biol. 17: 299-313, 1963), and is readily available to the public. For example, Waisman Biomanufacturing (Madison, Wis.) sells irradiated 3T3-J2 feeder cells produced and tested according to cGMP guidelines. These cells were originally obtained from Dr. Howard Green's laboratory under a material transfer agreement, and according to the vender, are of the quality sufficient to support, for example, skin gene therapy and wound healing clinical trials. Also according to the vender, each vial of the 3T3 cells contains a minimum of 3×10⁶ cells that have been manufactured in fully compliant cleanrooms, and are certified mycoplasma free and low endotoxin. In addition, the cell bank has been fully tested for adventitious agents, including murine viruses. These cells have been screened for keratinocyte culture support and do not contain mitomycin C.

The method of the invention provides the use of feeder cells, such as the murine 3T3-J2 clone of fibroblasts. In general, without being limited to any particular phenotype, feeder cell layers are often used to support the culture of stem cells, and/or to inhibit their differentiation. A feeder cell layer is generally a monolayer of cells that is co-cultured with, and which provides a surface suitable for growth of, the cells of interest. The feeder cell layer provides an environment in which the cells of interest can grow. Feeder cells are often mitotically inactivated (e.g., by (lethal) irradiation or treatment with mitomycin C) to prevent their proliferation.

In certain embodiments, the feeder cells are appropriately screened and GMP-grade human feeder cells, e.g., those sufficient to support clinical-grade stem cell of the invention. See Crook et al. (Cell Stem Cell 1(5):490-494, 2007, incorporated by reference), for GMP-grade human feeder cells grown in medium with GMP-quality FBS.

In certain embodiments, the feeder cells can be labeled by a marker that is lacking in the stem cells, such that the stem cells can be readily distinguished and isolated from the feeder cells. For example, the feeder cells can be engineered to express a fluorescent marker, such as GFP or other similar fluorescent markers. The fluorescent-labeled feeder cells can be isolated from the stem cells using, for example, FACS sorting.

Any one of a number of physical methods of separation known in the art may be used to separate the stem cells of the invention from the feeder cells. Such physical methods, other than FACS, may include various immuno-affinity methods based upon specifically expressed makers. For example, the stem cells of the invention can be isolated based on the specific stem cell markers they express, using antibodies specific for such markers.

In one embodiment, the stem cells of the invention may be isolated by FACS utilizing an antibody, for example, against one of these markers. Fluorescent activated cell sorting (FACS) can be used to detect markers characteristic of a particular cell type or lineage. As will be apparent to one skilled in the art, this may be achieved through a fluorescent labeled antibody, or through a fluorescent labeled secondary antibody with binding specificity for the primary antibody. Examples of suitable fluorescent labels includes, but is not limited to, FITC, Alexa Fluor® 488, GFP, CFSE, CFDA-SE, DyLight 488, PE, PerCP, PE-Alexa Fluor® 700, PE-Cy5 (TRI-COLOR®), PE-Cy5.5, PI, PE-Alexa Fluor® 750, and PE-Cy7. The list of fluorescent markers is provided by way of example only, and is not intended to be limiting.

It will be apparent to a person skilled in the art that FACS analysis using, for example, an antibody specific for stem cell will provide a purified stem cell population. However, in some embodiments, it may be preferable to purify the cell population further by performing a further round of FACS analysis using one or more of the other identifiable markers, such as one that select against the feeders.

The use of feeder cells is considered undesirable for certain competing methods, because the presence of feeders may complicate passaging of the cells in those competing methods. For example, the cells must be separated from the feeder cells at each passage, and new feeder cells are required at each passage. In addition, the use of feeder cells may lead to contamination of the desired cells by the feeder cells.

Use of feeder layer, however, is not necessarily a disadvantage of the present invention, since the isolated stem cells of the invention are capable of being passaged as single cell, and are in fact preferably passaged as single cell clones. Thus the potential risk of contamination by the feeders during passaging is minimized, if not eliminated.

In certain embodiments, the basement membrane matrix is a laminin-containing basement membrane matrix (e.g., MATRIGEL™ basement membrane matrix (BD Biosciences)), preferably growth factor-reduced.

In certain embodiments, the basement membrane matrix does not support 3-dimensional growth, or does not form a 3-dimensional matrix necessary to support 3-dimensional growth. Thus when plating the basement membrane matrix, it is usually not required to deposit the basement membrane matrix in a specific shape or form on a support, such as forming a dome shape or form and maintaining such shape or form after solidification, which shape or form may be required to support 3-dimensional growth. In certain embodiments, the basement membrane matrix is evenly distributed or spread out on a flat surface or supporting structure (such as a flat bottom tissue culture dish or well).

In certain embodiments, the basement membrane matrix is first thawed and diluted in cold (e.g., about 0-4° C.) feeder cell growth medium to a proper concentration (e.g., 10%), and plated and solidified on a flat surface, such as by warming up to 37° C. in a tissue culture incubator with appropriate CO₂ content (e.g., about 5%). Lethally irradiated feeder cells are then plated on top of the solidified basement membrane matrix at a proper density such that settled feeder cells forms a subconfluent or confluent feeder cell layer overnight on top of the basement membrane matrix. The feeder cells are cultured in feeder cell medium, such as a medium (e.g., 3T3-J2 growth medium) comprising: a base tissue culture medium that preferably has high glucose (e.g., about 4.5 g/L), no L-glutamine, and no sodium pyruvate (e.g., DMEM (Invitrogen cat. no. 11960; high glucose (4.5 g/L), no L-glutamine, no sodium pyruvate), 10% bovine calf serum (not heat inactivated), one or more antibiotics (e.g., 1% penicillin-streptomycin), and L-glutamine (e.g., about 1.5 mM, or 1-2 mM, or 0.5-5 mM, or 0.2-10 mM, or 0.1-20 mM).

In certain embodiments, the dissociated cells from the non-embryonic tissue are first plated in contact with the lethally irradiated feeder cells and the basement membrane matrix, in a medium of the invention (a “modified growth medium,” or “modified medium” for short) that promotes the growth of the non-embryonic stem cells. In certain embodiments, the modified medium of the invention comprises a Notch agonist, a ROCK (Rho Kinase) inhibitor, a Bone Morphogenetic Protein (BMP) antagonist, a Wnt agonist, a mitogenic growth factor; and, insulin or IGF; in a base medium, and optionally, the medium further comprises at least one (either one or both) of: a TGFβ signaling pathway inhibitor (e.g., a TGFβ inhibitor or a TGFβ receptor inhibitor); and, nicotinamide or an analog, precursor (such as niacin), or mimic thereof. Alternatively, in other embodiments, the modified medium of the invention comprises a Notch agonist; a ROCK (Rho Kinase) inhibitor; a Wnt agonist; a TGFβ signaling pathway inhibitor (e.g., a TGFβ inhibitor or a TGFβ receptor inhibitor); nicotinamide or an analog, precursor (such as niacin), or mimic thereof; a mitogenic growth factor; and, insulin or IGF in a base medium, and optionally, the medium further comprises a Bone Morphogenetic Protein (BMP) antagonist.

Illustrative (non-limiting) basal and modified medium, including compositions or factors therein, concentration ranges thereof, specific combinations of factors, or variations thereof are described in further detail in Section 3 below.

According to the methods of the invention, epithelial cell colonies becomes detectable after a few days (e.g., 3-4 days, or about 10 days) of culturing the dissociated cells from the source tissue in the subject modified medium.

In certain embodiments, single cells may be isolated from these epithelial cell colonies by, for example, enzyme digestion. Suitable enzymes for this purpose include trypsin, such as warm 0.25% trypsin (Invitrogen, cat. no 25200056). In certain embodiments, the enzyme digestion is substantially complete such that essentially all cells in the epithelial cell clones becomes dissociated from other cells and becomes single cells.

In certain embodiments, the method comprises culturing the isolated single cells (preferably after washing and resuspending the single cells) in the modified growth medium in contact with a second population of lethally irradiated feeder cells and a second basement membrane matrix in the modified growth medium. Optionally, the isolated single cells may be passed through a cell strainer of proper size (e.g., 40 micron), before the single cells are plated on the feeder cells and basement membrane matrix.

In certain embodiments, the modified growth medium is changed periodically (e.g., once every day, once every 2, 3, or 4 days, etc.) till single cell clones or clonal expansion of the isolated single stem cells form.

In certain embodiments, a single colony of the stem cell can be isolated using, for example, a cloning ring. The isolated stem cell clone can be expanded to develop a pedigree cell line, i.e., a cell line that has been derived from a single stem cell.

In certain embodiments, single stem cells can be isolated from the clonal expansion of the single stem cell, and can be passaged again as single stem cells.

It has been shown that more than 70% or even 90% of the isolated intestine stem cells in culture maintain the clonogenic ability, indicating that they are stem cells. Furthermore, after more than 400 cell divisions, these intestine epithelial stem cells maintain their ability for multipotent differentiation, and can form intestine-like structures in the air-liquid interface assay.

More detailed description of the methods for isolating non-embryonic stem cells has been described in further detail below in illustrative Example 1-5. Details in these examples also constitute part of this section relating to the general description of the subject isolation methods.

### 3. Medium

The invention provides various cell culture media for isolating, culturing, and/or differentiation of the subject stem cells, comprising a base medium to which a number of factors are added to produce a modified medium. The factors that may be added to the base medium or the modified medium are first described below. Several exemplary base media and modified media of the invention are then described with further details to illustrate specific non-limiting embodiments of the invention.

BMP Inhibitor

Bone Morphogenetic Proteins (BMPs) bind as a dimeric ligand to a receptor complex consisting of two different receptor serine/threonine kinases, type I and type II receptors. The type II receptor phosphorylates the type I receptor, resulting in the activation of this receptor kinase. The type I receptor subsequently phosphorylates specific receptor substrates (such as SMAD), resulting in a signal transduction pathway leading to transcriptional activity.

A BMP inhibitor as used herein includes an agent that inhibits BMP signaling through its receptors. In one embodiment, a BMP inhibitor binds to a BMP molecule to form a complex such that BMP activity is neutralized, for example, by preventing or inhibiting the binding of the BMP molecule to a BMP receptor. Examples of such BMP inhibitors may include an antibody specific for the BMP ligand, or an antigen-binding portion thereof. Other examples of such BMP inhibitors include a dominant negative mutant of a BMP receptor, such as a soluble BMP receptor that binds the BMP ligand and prevents the ligand from binding to the natural BMP receptor on the cell surface.

Alternatively, the BMP inhibitor may include an agent that acts as an antagonist or reverse agonist. This type of inhibitor binds with a BMP receptor and prevents binding of a BMP to the receptor. An example of such an agent is an antibody that specifically binds a BMP receptor and prevents binding of BMP to the antibody-bound BMP receptor.

In certain embodiments, the BMP inhibitor inhibits a BMP-dependent activity in a cell to at most 90%, at most 80%, at most 70%, at most 50%, at most 30%, at most 10%, or about 0% (near complete inhibition), relative to a level of a BMP activity in the absence of the inhibitor. As is known to one of skill in the art, a BMP activity can be determined by, for example, measuring the transcriptional activity of BMP as exemplified in Zilberberg et al. (“A rapid and sensitive bioassay to measure bone morphogenetic protein activity,” BMC Cell Biology 8:41, 2007, incorporated herein by reference).

Several classes of natural BMP-binding proteins are known, including Noggin (Peprotech), Chordin, and chordin-like proteins comprising a chordin domain (R&D systems) comprising chordin domains, Follistatin and follistatin-related proteins comprising a follistatin domain (R&D systems) comprising a follistatin domain, DAN and DAN-like proteins comprising a DAN Cystine-knot domain (e.g., Cerberus and Gremlin) (R&D systems), sclerostin/SOST (R&D systems), decorin (R&D systems), and alpha-2 macroglobulin (R&D systems) or as described in U.S. Pat. No. 8,383,349.

An exemplary BMP inhibitor for use in a method of the invention is selected from Noggin, DAN, and DAN-like proteins including Cerberus and Gremlin (R&D systems). These diffusible proteins are able to bind a BMP ligand with varying degrees of affinity, and inhibit BMPs' access to their signaling receptors.

Any of the above-described BMP inhibitors may be added either alone or in combination to the subject culture medium when desirable.

In certain embodiments, the BMP inhibitor is Noggin. Noggin may be added to the respective culture medium at a concentration of at least about 10 ng/mL, or at least about 20 ng/mL, or at least about 50 ng/mL, or at least about 100 ng/mL (e.g., 100 ng/mL).

In certain embodiments, any of the specific BMP inhibitors referenced herein, such as Noggin, Chordin, Follistatin, DAN, Cerberus, Gremlin, sclerostin/SOST, decorin, and alpha-2 macroglobulin may be replaced by a natural, synthetic, or recombinantly produced homologs or fragments thereof that retain at least about 80%, 85%, 90%, 95%, 99% of the respective BMP inhibiting activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm).

The sequences of the representative BMP inhibitors referenced herein are represented in SEQ ID NOs. 1-9.

During culturing of the subject stem cells, the BMP inhibitor may be added to the culture medium every day, every 2^{nd} day, every 3^{rd} day, or every 4^{th} day, while the culture medium is refreshed every day, every second day, every third day, or every fourth day as appropriate.

Wnt Agonist

The Wnt signaling pathway is defined by a series of events that occur when a Wnt protein ligand binds to a cell-surface receptor of a Frizzled receptor family member. This results in the activation of Dishevelled (Dsh) family proteins which inhibit a complex of proteins that includes axin, GSK-3, and the protein APC to degrade intracellular β-catenin. The resulting enriched nuclear β-catenin enhances transcription by TCF/LEF family of transcription factors.

A “Wnt agonist” as used herein includes an agent that directly or indirectly activates TCF/LEF-mediated transcription in a cell, such as through modulating the activity of any one of the proteins/genes in the Wnt signaling cascade (e.g., enhancing the activity of a positive regulator of the Wnt signaling pathway, or inhibiting the activity of a negative regulator of the Wnt signaling pathway).

Wnt agonists are selected from true Wnt agonists that bind and activate a Frizzled receptor family member including any and all of the Wnt family proteins, an inhibitor of intracellular β-catenin degradation, and activators of TCF/LEF. The Wnt agonist may stimulate a Wnt activity in a cell by at least about 10%, at least about 20%, at least about 30%, at least about 50%, at least about 70%, at least about 90%, at least about 100%, at least about 2-fold, 3-fold, 5-fold, 10-fold, 20-fold, 50-fold, 100-fold, 200-fold, 500-fold, or 1000-fold or more relative to a level of the Wnt activity in the absence of the Wnt agonist. As is known to a person of skill in the art, a Wnt activity can be determined by measuring the transcriptional activity of Wnt, for example by pTOPFLASH and pFOPFLASH Tcf luciferase reporter constructs (see Korinek et al., Science 275:1784-1787, 1997, incorporated herein by reference).

Representative Wnt agonist may comprise a secreted glycoprotein including Wnt-1/Int-1, Wnt-2/Irp (Int-1-related Protein), Wnt-2b/13, Wnt-3/Int-4, Wnt-3a (R&D systems), Wnt-4, Wnt-5a, Wnt-5b, Wnt-6 (Kirikoshi et al., Biochem. Biophys. Res. Com., 283:798-805, 2001), Wnt-7a (R&D systems), Wnt-7b, Wnt-8a/8d, Wnt-8b, Wnt-9a/14, Wnt-9b/14b/15, Wnt-10a, Wnt-10b/12, Wnt-11, and Wnt-16. An overview of human Wnt proteins is provided in “The Wnt Family of Secreted Proteins,” R&D Systems Catalog, 2004 (incorporated herein by reference).

Further Wnt agonists include the R-spondin family of secreted proteins, which is implicated in the activation and regulation of Wnt signaling pathway, and which comprises at least 4 members, namely R-spondin 1 (NU206, Nuvelo, San Carlos, Calif.), R-spondin 2 (R&D systems), R-spondin 3, and R-spondin 4. Wnt agonists also include Norrin (also known as Norrie Disease Protein or NDP) (R&D systems), which is a secreted regulatory protein that functions like a Wnt protein in that it binds with high affinity to the Frizzled-4 receptor and induces activation of the Wnt signaling pathway (Kestutis Planutis et al., BMC Cell Biol. 8:12, 2007).

Wnt agonists further include a small-molecule agonist of the Wnt signaling pathway, an aminopyrimidine derivative (N⁴-(benzo[d][1,3]dioxol-5-ylmethyl)-6-(3-methoxyphenyl)pyrimidine-2,4-diamine) of the following structure, as described in Liu et al. (Angew Chem. Int. Ed. Engl. 44(13): 1987-1990, 2005, incorporated herein by reference).

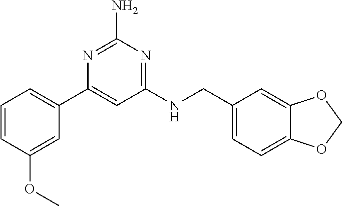 [SMILES: COc1cccc(-c2cc(NCc3ccc4c(c3)OCO4)nc(N)n2)c1]

GSK-inhibitors comprise small-interfering RNAs (siRNA, Cell Signaling), lithium (Sigma), kenpaullone (Biomol International, Leost et al., Eur. J. Biochem. 267:5983-5994, 2000), 6-Bromoindirubin-30-acetoxime (Meyer et al., Chem. Biol. 10:1255-1266, 2003), SB 216763, and SB 415286 (Sigma-Aldrich), and FRAT-family members and FRAT-derived peptides that prevent interaction of GSK-3 with axin. An overview is provided by Meijer et al. (Trends in Pharmacological Sciences 25:471-480, 2004, incorporated herein by reference). Methods and assays for determining a level of GSK-3 inhibition are known in the art, and may comprise, for example, the methods and assay as described in Liao et al. (Endocrinology 145(6):2941-2949, 2004, incorporated herein by reference).

In certain embodiments, Wnt agonist is selected from: one or more of a Wnt family member, R-spondin 1-4 (such as R-spondin 1), Norrin, Wnt3a, Wnt-6, and a GSK-inhibitor.

In certain embodiments, the Wnt agonist comprises or consists of R-spondin 1. R-spondin 1 may be added to the subject culture medium at a concentration of at least about 50 ng/mL, at least about 75 ng/mL, at least about 100 ng/mL, at least about 125 ng/mL, at least about 150 ng/mL, at least about 175 ng/mL, at least about 200 ng/mL, at least about 300 ng/mL, at least about 500 ng/mL. In certain embodiments, R-spondin 1 is about 125 ng/mL.

In certain embodiments, any of the specific protein-based Wnt agonist referenced herein, such as R-spondin 1 to R-spondin 4, any Wnt family member, etc. may be replaced by a natural, synthetic, or recombinantly produced homologs or fragments thereof that retain at least about 80%, 85%, 90%, 95%, 99% of the respective Wnt agonist activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm).

The sequences of the representative Wnt agonist referenced herein are represented in SEQ ID NOs. 10-17.

During culturing of the subject stem cells, the Wnt family member may be added to the medium every day, every second day, every third day, while the medium is refreshed, e.g., every 1, 2, 3, 4, 5, or more days.

In certain embodiments, a Wnt agonist is selected from the group consisting of: an R-spondin, Wnt-3a and Wnt-6, or combinations thereof. In certain embodiments, an R-spondin and Wnt-3a are used together as Wnt agonist. In certain embodiments, R-spondin concentration is about 125 ng/mL, and Wnt3a concentration is about 100 ng/mL.

Mitogenic Growth Factor

Mitogenic growth factors suitable for the invention may include a family of growth factors comprising epidermal growth factor (EGF) (Peprotech), Transforming Growth Factor-α (TGFα, Peprotech), basic Fibroblast Growth Factor (bFGF, Peprotech), brain-derived neurotrophic factor (BDNF, R&D Systems), and Keratinocyte Growth Factor (KGF, Peprotech).

EGF is a potent mitogenic factor for a variety of cultured ectodermal and mesodermal cells, and has a profound effect on the differentiation of specific cells in vivo and in vitro, and of some fibroblasts in cell culture. The EGF precursor exists as a membrane-bound molecule, which is proteolytically cleaved to generate the 53-amino acid peptide hormone that stimulates cells. EGF may be added to the subject culture medium at a concentration of between 1-500 ng/mL. In certain embodiments, final EGF concentration in the medium is at least about 1, 2, 5, 10, 20, 25, 30, 40, 45, or 50 ng/mL, and is not higher than about 500, 450, 400, 350, 300, 250, 200, 150, 100, 50, 30, 20 ng/mL. In certain embodiments, final EGF concentration is about 1-50 ng/mL, or about 2-50 ng/mL, or about 5-30 ng/mL, or about 5-20 ng/mL, or about 10 ng/mL.

The same concentrations may be used for an FGF, such as FGF10 or FGF7. If more than one FGF is used, for example FGF7 and FGF10, the concentration of FGF above may refer to the total concentration of all FGF used in the medium.

In certain embodiments, any of the specific mitogenic growth factors referenced herein, such as EGF, TGFα, bFGF, BDNF, KGF, etc. may be replaced by a natural, synthetic, or recombinantly produced homologs or fragments thereof that retain at least about 80%, 85%, 90%, 95%, 99% of the respective mitogenic growth factor activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm).

The sequences of the representative mitogenic growth factors referenced herein are represented in SEQ ID NOs. 18-27.

During culturing of the subject stem cells, the mitogenic growth factor may be added to the culture medium every day, every 2^{nd} day, while the culture medium is refreshed, e.g., every 1^{st}, 2^{nd}, 3^{rd}, 4^{th} or 5^{th} day.

Any member of the bFGF family may be used. In certain embodiments, FGF7 and/or FGF10 is used. FGF7 is also known as KGF (Keratinocyte Growth Factor). In certain embodiments, a combination of mitogenic growth factors, such as EGF and KGF, or EGF and BDNF, is added to the subject culture medium. In certain embodiments, a combination of mitogenic growth factors, such as EGF and KGF, or EGF and FGF10, is added to the subject culture medium.

Rock (Rho-Kinase) Inhibitor

While not wishing to be bound by any particular theory, the addition of a Rock inhibitor may prevent anoikis, especially when culturing single stem cells. The Rock inhibitor may be (R)-(+)-trans-4-(1-aminoethyl)-N-(4-Pyridyl)cyclohexanecarboxamide dihydrochloride monohydrate (Y-27632, Sigma-Aldrich), 5-(1,4-diazepan-1-ylsulfonyl)isoquinoline (fasudil or HA1077, Cayman Chemical), (S)-(+)-2-methyl-1-[(4-methyl-5-isquinolinyl)sulfonyl]-hexahydro-1H-1,4-diazepine dihydrochloride (H-1152, Tocris Bioscience), and N-(6-fluoro-1H-indazol-5-yl)-2-methyl-6-oxo-4-(4-(trifluoromethyl)phenyl)-1,4,5,6-tetrahydropyridine-3-carboxamide (GSK429286A, Stemgent).

In certain embodiments, the final concentration for Y27632 is about 1-5 μM, or 2.5 μm.

The Rho-kinase inhibitor, e.g., Y-27632, may be added to the culture medium every 1, 2, 3, 4, 5, 6, or 7 days during the first seven days of culturing the stem cells.

Notch Agonist

Notch signaling has been shown to play an important role in cell-fate determination, as well as in cell survival and proliferation. Notch receptor proteins can interact with a number of surface-bound or secreted ligands, including but not limited to Jagged-1, Jagged-2, Delta-1 or Delta-like 1, Delta-like 3, Delta-like 4, etc. Upon ligand binding, Notch receptors are activated by serial cleavage events involving members of the ADAM protease family, as well as an intramembranous cleavage regulated by the gamma secretase presinilin. The result is a translocation of the intracellular domain of Notch to the nucleus, where it transcriptionally activates downstream genes.

A “Notch agonist” as used herein includes a molecule that stimulates a Notch activity in a cell by at least about 10%, at least about 20%, at least about 30%, at least about 50%, at least about 70%, at least about 90%, at least about 100%, at least about 3-fold, 5-fold, 10-fold, 20-fold, 50-fold, 100-fold, 200-fold, 500-fold, 1000-fold or more, relative to a level of a Notch activity in the absence of the Notch agonist. As is known in the art, Notch activity can be determined by, for example, measuring the transcriptional activity of Notch, by a 4xwtCBF1-luciferase reporter construct described by Hsieh et al. (Mol. Cell. Biol. 16:952-959, 1996, incorporated herein by reference).

In certain embodiments, the Notch agonist is selected from: Jagged-1, Delta-1 and Delta-like 4, or an active fragment or derivative thereof. In certain embodiments, the Notch agonist is DSL peptide (Dontu et al., Breast Cancer Res., 6:R605-R615, 2004), having the amino acid sequence CDDYYYGFGCNKFCRPR (SEQ ID NO: 36). The DSL peptide (ANA spec) may be used at a concentration between 10 μM and 100 nM, or at least 10 μM and not higher than 100 nM. In certain embodiments, the final concentration of Jagged-1 is about 0.1-10 μM; or about 0.2-5 μM; or about 0.5-2 μM; or about 1 μM.

In certain embodiments, any of the specific Notch agonist referenced herein, such as Jagged-1, Jagged-2, Delta-1 and Delta-like 4 may be replaced by a natural, synthetic, or recombinantly produced homologs or fragments thereof that retain at least about 80%, 85%, 90%, 95%, 99% of the respective Notch agonist activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm).

The sequences of the representative Notch agonists referenced herein are represented in SEQ ID NOs. 28-35.

The Notch agonist may be added to the culture medium every 1, 2, 3, or 4 days during the first 1-2 weeks of culturing the stem cells.

Nicotinamide

The culture medium of the invention may additionally be supplemented with nicotinamide or its analogs, precursors, or mimics, such as methyl-nicotinamid, benazamid, pyrazinamide, thymine, or niacin. Nicotinamide may be added to the culture medium to a final concentration of between 1 and 100 mM, between 5 and 50 mM, or preferably between 5 and 20 mM. For example, nicotinamide may be added to the culture medium to a final concentration of approximately 10 mM. The similar concentrations of nicotinamide analogs, precursors, or mimics can also be used alone or in combination.

In certain stem cell cultures, adding TGFβ receptor inhibitor (see below) and/or nicotinamide (alone or in combination) greatly increases the self-renewal ability of the stem cells in culture. The number of the cells in each colony may be significantly increased, and the size of the cells dramatically reduced in the presence of Nicotinamide and/or TGFβ receptor inhibitor.

THG-β or TGF-β Receptor Inhibitor

TGF-β signaling is involved in many cellular functions, including cell growth, cell fate and apoptosis. Signaling typically begins with binding of a TGF-β superfamily ligand to a Type II receptor, which recruits and phosphorylates a Type I receptor. The Type 1 receptor then phosphorylates SMADs, which act as transcription factors in the nucleus and regulate target gene expression. Alternatively, TGF-β signaling can activate MAP kinase signaling pathways, for example, via p38 MAP kinase.

The TGF-β superfamily ligands comprise bone morphogenetic proteins (BMPs), growth and differentiation factors (GDFs), anti-Mullerian hormone (AMH), activin, nodal and TGF-βs.

A TGF-β inhibitor as used herein include an agent that reduces the activity of the TGF-β signaling pathway. There are many different ways of disrupting the TGF-β signaling pathway known in the art, any of which may be used in conjunction with the subject invention. For example, TGF-β signaling may be disrupted by: inhibition of TGF-β expression by a small-interfering RNA strategy; inhibition of furin (a TGF-β activating protease); inhibition of the pathway by physiological inhibitors, such as inhibition of BMP by Noggin, DAN or DAN-like proteins; neutralization of TGF-β with a monoclonal antibody; inhibition with small-molecule inhibitors of TGF-β receptor kinase 1 (also known as activin receptor-like kinase, ALK5), ALK4, ALK6, ALK7 or other TGF-β-related receptor kinases; inhibition of Smad 2 and Smad 3 signaling by overexpression of their physiological inhibitor, Smad 7, or by using thioredoxin as an Smad anchor disabling Smad from activation (Fuchs, Inhibition of TGF-β Signaling for the Treatment of Tumor Metastasis and Fibrotic Diseases. Current Signal Transduction Therapy 6(1):29-43(15), 2011).

For example, a TGF-β inhibitor may target a serine/threonine protein kinase selected from: TGF-β receptor kinase 1, ALK4, ALK5, ALK7, or p38. ALK4, ALK5 and ALK7 are all closely related receptors of the TGF-β superfamily. ALK4 has GI number 91; ALK5 (also known as TGF-β receptor kinase 1) has GI number 7046; and ALK7 has GI number 658. An inhibitor of any one of these kinases is one that effects a reduction in the enzymatic activity of any one (or more) of these kinases. Inhibition of ALK and p38 kinase has previously been shown to be linked in B-cell lymphoma (Bakkebo et al., “TGF-β-induced growth inhibition in B-cell lymphoma correlates with Smad 1/5 signaling and constitutively active p38 MAPK,” BMC Immunol. 11:57, 2010).

In certain embodiments, a TGF-β inhibitor may bind to and inhibit the activity of a Smad protein, such as R-SMAD or SMAD1-5 (i.e., SMAD1, SMAD2, SMAD3, SMAD4 or SMAD5).

In certain embodiments, a TGF-β inhibitor may bind to and reduces the activity of Ser/Thr protein kinase selected from: TGF-β receptor kinase 1, ALK4, ALK5, ALK7, or p38.

In certain embodiments, the medium of the invention comprises an inhibitor of ALK5.

In certain embodiments, the TGF-β inhibitor or TGF-β receptor inhibitor does not include a BMP antagonist (i.e., is an agent other than BMP antagonist).

Various methods for determining if a substance is a TGF-β inhibitor are known. For example, a cellular assay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al., Br. J. Pharmacol. 145(2): 166-177, 2005, incorporated herein by reference). Another example is the ALPHASCREEN® phosphosensor assay for measurement of kinase activity (Drew et al., J. Biomol. Screen. 16(2):164-173, 2011, incorporated herein by reference).

A TGF-β inhibitor useful for the present invention may be a protein, a peptide, a small-molecule, a small-interfering RNA, an antisense oligonucleotide, an aptamer, an antibody or an antigen-binding portion thereof. The inhibitor may be naturally occurring or synthetic. Examples of small-molecule TGF-β inhibitors that can be used in the context of this invention include, but are not limited to, the small molecule inhibitors listed in Table 1 below:

TABLE 1
Small-molecule TGF-β inhibitors targeting receptor kinases

| | | IC50 | | | |
|---|---|---|---|---|---|
| Inhibitor | Targets | (nm) | Mol Wt | Name | Formula |
| A83-01 | ALK5 | 12 | 421.52 | 3-(6-Methyl-2- | C25H19N5S |
| | (TGP-βR1) | | | pyridinyl)-N- | |
| | ALK4 | 45 | | phenyl-4- | |
| | ALK7 | 7.5 | | (4-quinolinyl)- | |
| | | | | 1H-pyrazole-1- | |
| | | | | carbothioamide | |
| SB-431542 | ALK5 | 94 | 384.39 | 4-[4-(1,3- | C22H16N4O3 |
| | ALK4 | | | benzodioxol- | |
| | ALK7 | | | 5-yl)-5- | |
| | | | | (2-pyridinyl)- | |
| | | | | 1H-imidazol-2- | |
| | | | | yl]benzamide | |
| SB-505124 | ALK5 | 47 | 335.4 | 2-(5- | C20H21N3O2 |
| | ALK4 | 129 | | benzo[1,3]dioxol- | |
| | | | | 5-yl-2-tert-butyl- | |
| | | | | 3Himidazol- | |
| | | | | 4-yl)-6- | |
| | | | | methylpyridine | |
| | | | | hydrochloride | |
| | | | | hydrate | |
| SB-525334 | ALK5 | 14.3 | 343.42 | 6-[2-(1,1- | C21H21N5 |
| | | | | Dimethylethyl)- | |
| | | | | 5-(6-methyl- | |
| | | | | 2-pyridinyl)- | |
| | | | | 1H-imidazol-4- | |
| | | | | yl]quinoxaline | |
| SD-208 | ALK5 | 49 | 352.75 | 2-(5-Chloro-2- | C17H10ClFN6 |
| | | | | fluorophenyl)-4- | |
| | | | | [(4-pyridyl) | |
| | | | | amino]pteridine | |
| LY-36494 | TGR-βRI | 59 | 272.31 | 4-[3-(2-Pyridinyl)- | C17H12N4 |
| | TGF-βRII | 400 | | 1H-pyrazol-4- | |
| | MLK-7K | 1400 | | yl]-quinoline | |
| SJN-2511 | ALK5 | 23 | 287.32 | 2-(3-(6- | C17H13N5 |
| | | | | Methylpyridine- | |
| | | | | 2-yl)-1H- | |
| | | | | pyrazol-4-yl)-1,5- | |
| | | | | naphthyridine | |

One or more of any of the inhibitors listed in Table 1 above, or a combination thereof, may be used as a TGF-β inhibitor in the subject invention. In certain embodiments, the combination may include: SB-525334 and SD-208 and A83-01; SD-208 and A83-01; or SD-208 and A83-01.

One of skill in the art will appreciate that a number of other small-molecule inhibitors exist that are primarily designed to target other kinases, but at high concentrations may also inhibit TGF-β receptor kinases. For example, SB-203580 is a p38 MAP kinase inhibitor that, at high concentrations (for example, approximate 10 μM or more) may inhibit ALK5. Any such inhibitor that inhibits the TGF-β signaling pathway may also be used in this invention.

In certain embodiments, A83-01 may be added to the culture medium at a concentration of between 10 nM and 10 μM, or between 20 nM and 5 μM, or between 50 nM and 1 μM. In certain embodiments, A83-01 may be added to the medium at about 500 nM. In certain embodiments, A83-01 may be added to the culture medium at a concentration of between 350-650 nM, 450-550 nM, or about 500 nM. In certain embodiments, A83-01 may be added to the culture medium at a concentration of between 25-75 nM, 40-60 nM, or about 50 nM.

SB-431542 may be added to the culture medium at a concentration of between 80 nM and 80 μM, or between 100 nM and 40 μM, or between 500 nM and 10 μM, or between 1-5 μM. For example, SB-431542 may be added to the culture medium at about 2 μM.

SB-505124 may be added to the culture medium at a concentration of between 40 nM and 40 μM, or between 80 nM and 20 μM, or between 200 nM and 1 μM. For example, SB-505124 may be added to the culture medium at about 500 nM.

SB-525334 may be added to the culture medium at a concentration of between 10 nM and 10 μM, or between 20 nM and 5 μM, or between 50 nM and 1 μM. For example, SB-525334 may be added to the culture medium at about 100 nM.

LY 364947 may be added to the culture medium at a concentration of between 40 nM and 40 μM, or between 80 nM and 20 μM, or between 200 nM and 1 μM. For example, LY 364947 may be added to the culture medium at about 500 nM.

SD-208 may be added to the culture medium at a concentration of between 40 nM and 40 μM, or between 80 nM and 20 μM, or between 200 nM and 1 μM. For example, SD-208 may be added to the culture medium at abut 500 nM.

SJN 2511 may be added to the culture medium at a concentration of between 20 nM and 20 μM, or between 40 nM and 10 μM, or between 100 nM and 1 μM. For example, A83-01 may be added to the culture medium at approximately 200 nM.

p38 Inhibitor

A “p38 inhibitor” may include an inhibitor that, directly or indirectly, negatively regulates p38 signaling, such as an agent that binds to and reduces the activity of at least one p38 isoform. p38 protein kinases (see, GI number 1432) are part of the family of mitogen-activated protein kinases (MAPKs). MAPKs are serine/threonine-specific protein kinases that respond to extracellular stimuli, such as environmental stress and inflammatory cytokines, and regulate various cellular activities, such as gene expression, differentiation, mitosis, proliferation, and cell survival/apoptosis. The p38 MAPKs exist as α, β, β2, γ and δ isoforms.

Various methods for determining if a substance is a p38 inhibitor are known, such as: phospho-specific antibody detection of phosphorylation at Thr180/Tyr182, which provides a well-established measure of cellular p38 activation or inhibition; biochemical recombinant kinase assays; tumor necrosis factor alpha (TNFα) secretion assays; and DiscoverRx high throughput screening platform for p38 inhibitors. Several p38 activity assay kits also exist (e.g. Millipore, Sigma-Aldrich).

In certain embodiments, high concentrations (e.g., more than 100 nM, or more than 1 μM, more than 10 μM, or more than 100 μM) of a p38 inhibitor may have the effect of inhibiting TGF-β. In other embodiments, the p38 inhibitor does not inhibit TGF-β signaling.

Various p38 inhibitors are known in the art (for example, see Table 1). In some embodiments, the inhibitor that directly or indirectly negatively regulates p38 signaling is selected from the group consisting of SB-202190, SB-203580, VX-702, VX-745, PD-169316, RO-4402257 and BIRB-796.

In certain embodiments, the medium comprises both: a) an inhibitor that binds to and reduces the activity of any one or more of the kinases from the group consisting of: ALK4, ALK5 and ALK7; and b) an inhibitor that binds to and reduces the activity of p38.

In certain embodiments, the medium comprises an inhibitor that binds to and reduces the activity of ALK5 and an inhibitor that binds to and reduces the activity of p38.

In one embodiment, the inhibitor binds to and reduces the activity of its target (for example, TGF-β and/or p38) by more than 10%; more than 30%; more than 60%; more than 80%; more than 90%; more than 95%; or more than 99% compared to a control, as assessed by a cellular assay. Examples of cellular assays for measuring target inhibition are well known in the art as described above.

An inhibitor of TGF-β and/or p38 may have an IC₅₀ value equal to or less than 2000 nM; less than 1000 nM; less than 100 nM; less than 50 nM; less than 30 nM; less than 20 nM or less than 10 mM. The IC₅₀ value refers to the effectiveness of an inhibitor in inhibiting its target's biological or biochemical function. The IC₅₀ indicates how much of a particular inhibitor is required to inhibit a kinase by 50%. IC₅₀ values can be calculated in accordance with the assay methods set out above.

An inhibitor of TGF-β and/or p38 may exist in various forms, including natural or modified substrates, enzymes, receptors, small organic molecules, such as small natural or synthetic organic molecules of up to 2000 Da, preferably 800 Da or less, peptidomimetics, inorganic molecules, peptides, polypeptides, antisense oligonucleotides aptamers, and structural or functional mimetics of these including small molecules.

In certain embodiments, the inhibitor of TGF-β and/or p38 may also be an aptamer. As used herein, the term “aptamer” refers to strands of oligonucleotides (DNA or RNA) that can adopt highly specific three-dimensional conformations. Aptamers are designed to have high binding affinities and specificities towards certain target molecules, including extracellular and intracellular proteins. Aptamers may be produced using, for example, Systematic Evolution of Ligands by Exponential Enrichment (SELEX) process (see, for example, Tuerk and Gold, Systematic evolution of ligands by exponential enrichment: RNA ligands to bacteriophage T4 DNA Polymerase. Science 249:505-510, 1990, incorporated herein by reference).

In certain embodiments, the TGF-β and/or p38 inhibitor may be a small synthetic molecule with a molecular weight of between 50 and 800 Da, between 80 and 700 Da, between 100 and 600 Da, or between 150 and 500 Da.

In certain embodiments, the TGF-β and/or p38 inhibitor comprises a pyridinylimidazole or a 2,4-disubstituted pteridine or a quinazoline, for example comprises:

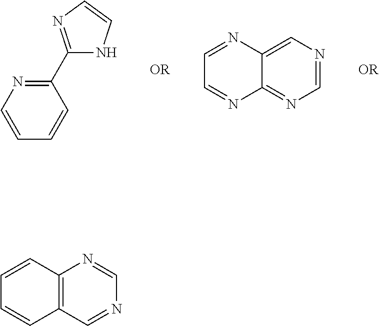 [SMILES: c1ccc(-c2ncc[nH]2)nc1 ; c1cnc2ncncc2n1 ; c1ccc2ncncc2c1]

Particular examples of TGF-β and/or p38 inhibitors that may be used in accordance with the invention include, but are not limited to: SB-202190, SB-203580, SB-206718, SB-227931, VX-702, VX-745, PD-169316, RO-4402257, BIRB-796, A83-01 SB-431542, SB-505124, SB-525334, LY 364947, SD-208, SJ 2511 (see Table 2).

A culture medium of the invention may comprise one or more of any of the inhibitors listed in Table 2. A culture medium of the invention may comprise any combination of one inhibitor with another inhibitor listed. For example, a culture medium of the invention may comprise SB-202190 or SB-203580 or A83-01; or SB-202190 and A83-01; or SB-203580 and A83-01. The skilled person will appreciate that other inhibitors and combinations of inhibitors which bind to and reduce the activity of the targets (e.g., TGF-β and/or p38), may be included in a culture medium or a culture medium supplement in accordance with the invention.

Inhibitors according to the invention may be added to the culture medium to a final concentration that is appropriate, taking into account the IC₅₀ value of the inhibitor.

For example, SB-202190 may be added to the culture medium at a concentration of between 50 nM and 100 μM, or between 100 nM and 50 μM, or between 1 μM and 50 μM. For example, SB-202190 may be added to the culture medium at approximately 10 μM.

SB-203580 may be added to the culture medium at a concentration of between 50 nM and 100 μM, or between 100 nM and 50 μM, or between 1 μM and 50 μM. For example, SB-203580 may be added to the culture medium at approximately 10 μM.

VX-702 may be added to the culture medium at a concentration of between 50 nM and 100 μM, or between 100 nM and 50 μM, or between 1 μM and 25 μM. For example, VX-702 may be added to the culture medium at approximately 5 μM.

VX-745 may be added to the culture medium at a concentration of between 10 nM and 50 μM, or between 50 nM and 50 μM, or between 250 nM and 10 μM. For example, VX-745 may be added to the culture medium at approximately 1 μM.

PD-169316 may be added to the culture medium at a concentration of between 100 nM and 200 μM, or between 200 nM and 100 μM, or between 1 μM and 50 μM. For example, PD-169316 may be added to the culture medium at approximately 20 μM.

RO-4402257 may be added to the culture medium at a concentration of between 10 nM and 50 μM, or between 50 nM and 50 μM, or between 500 nM and 10 μM. For example, RO-4402257 may be added to the culture medium at approximately 1 μM.

BIRB-796 may be added to the culture medium at a concentration of between 10 nM and 50 μM, or between 50 nM and 50 μM, or between 500 nM and 10 μM. For example, BIRB-796 may be added to the culture medium at approximately 1 μM.

See Table 1 and associated text above for the applicable concentrations for the other factors in Table 2.

TABLE 2
Exemplary TGF-β and/or p38 Inhibitors

| | | IC50 | | | |
|---|---|---|---|---|---|
| Inhibitor | Targets | (nM) | Mol Wt | Name | Formula |
| A83-01 | ALK5 | 12 | 421.52 | 3-(6-Methyl-2- | C25H19N5S |
| | (TGF-βR1) | | | pyridinyl)-N-phenyl-4- | |
| | ALK4 | 45 | | (4-quinolinyl)-1H- | |
| | ALK7 | 7.5 | | pyrazole-1- | |
| | | | | carbothioamide | |
| SB-431542 | ALK | 94 | 384.39 | 4-[4-(1,3-benzodioxol- | C22H16N4O3 |
| | ALK4 | | | 5-yl)-5-(2-pyridinyl)- | |
| | ALK7 | | | 1H-imidazol-2- | |
| | | | | yl]benzamide | |
| SB-505124 | ALK5 | 47 | 335.4 | 2-(5-benzo[1,3]dioxol- | C20H21N3O2 |
| | ALK4 | 129 | | 5-yl-2-tert-butyl- | |
| | | | | 3Himidazol- | |
| | | | | 4-yl)-6-methylpyridine | |
| | | | | hydrochloride hydrate | |
| SB-525334 | ALK5 | 14.3 | 343.42 | 6-[2-(1,1- | C21H21N5 |
| | | | | Dimethylethyl)-5-(6- | |
| | | | | methyl-2-pyridinyl)- | |
| | | | | 1H-imidazol-4- | |
| | | | | yl]quinoxaline | |
| SD-208 | ALK5 | 49 | 352.75 | 2-(5-Chloro-2- | C17H10ClFN6 |
| | | | | fluorophenyl)-4-[(4- | |
| | | | | pyridyl)amino]pteridine | |
| LY-36494 | TGR-βRI | 59 | 272.31 | 4-[3-(2-Pyridinyl)-1H- | C17H12N4 |
| | TGF-βRII | 400 | | pyrazol-4-yl]-quinoline | |
| | MLK-7K | 1400 | | | |
| LY364947 | ALK5 | 59 | 272.30 | 4-[3-(2-pyridinyl)-1H- | C₁₇H₁₂N₄ |
| | | | | pyrazol-4-yl]-quinoline | |
| SJN-2511 | ALK5 | 23 | 287.32 | 2-(3-(6- | C17H13N5 |
| | | | | Methylpyridine-2-yl)- | |
| | | | | 1H-pyrazol-4-yl)-1,5- | |
| | | | | naphthyridine | |
| SB-202190 | p38 MAP | 38 | 331.35 | 4-[4-(4-Fluorophenyl)- | C20H14N3OF |
| | kinase | | | 5-(4-pyridinyl)-1H- | |
| | p38α | 50 | | imidazol-2-yl]phenol | |
| | p38β | 100 | | | |
| SB-203580 | p38 | 50 | 377.44 | 4-[5-(4-Fluorophenyl)- | C21H16FN3OS |
| | p38β2 | 500 | | 2-[4- | |
| | | | | (methylsulfonyl)phenyl]- | |
| | | | | 1H-imidazol-4- | |
| | | | | yl]pyridine | |
| VX-702 | p38α | 4-20; | 404.32 | 6- | C19H12F4N4O2 |
| | | (Kd = 3.7) | | [(Aminocarbonyl)(2,6- | |
| | p38β | Kd = 17 | | difluorophenyl)amino]- | |
| | | | | 2-(2,4-difluorophenyl)- | |
| | | | | 3-pyridinecarboxamide | |
| VX-745 | p38α | 10 | 436.26 | 5-(2,6-Dichlorophenyl)- | C19H9Cl2F2N3OS |
| | | | | 2-[2,4- | |
| | | | | difluorophenyl)thio]- | |
| | | | | 6H-pyrimido[1,6- | |
| | | | | b]pyridazin-6-one | |
| PD-169316 | p38 | 89 | 360.3 | 4-[5-(4-fluorophenyl)- | C20H13FN4O |
| | | | | 2-(4-nitrophenyl)-1H- | |
| | | | | imidazol-4-yl]-pyridine | |
| RO- | p38α | 14 | | Pyrido[2,3-d]pyrimidin- | |
| 4402257 | | | | 7(8H)-one,6-(2,4- | |
| | | | | difluorophenoxy)-2-[[3- | |
| | | | | hydroxy-1-(2- | |
| | | | | hydroxyethyl)propyl] | |
| | | | | amino]-8-methyl- | |
| | p38β | 480 | | | |
| BIRB-796 | p38 | 4 | 527.67 | 1-[2-(4-methylphenyl)- | C31H37N5O3 |
| | | | | 5-tert-butyl-pyrazol-3- | |
| | | | | yl]-3-[4-(2-morpholin- | |
| | | | | 4-ylethoxy)naphthalen- | |
| | | | | 1-yl]urea::3-[2-(4- | |
| | | | | methylphenyl)-5-tert- | |
| | | | | butyl-pyrazol-3-yl]-1- | |
| | | | | [4-(2-morpholin-4- | |
| | | | | ylethoxy)naphthalen-1- | |
| | | | | yl]urea ::3-[3-tert-butyl- | |
| | | | | 1-(4-methylphenyl)- | |
| | | | | 1H-pyrazol-5-yl]-1-{4- | |
| | | | | [2-(morpholin-4- | |
| | | | | yl)ethoxy]naphthalen- | |
| | | | | 1-yl}urea | |

Thus, in some embodiments, the inhibitor that directly or indirectly, negatively regulates TGF-β and/or p38 signaling is added to the culture medium at a concentration of between 1 nM and 100 μM, between 10 nM and 100 μM, between 100 nM and 10 μM, or about 1 μM. For example, wherein the total concentration of the one or more inhibitor is between 10 nM and 100 μM, between 100 nM and 10 μM, or about 1 μM.

Extracellular Matrix (ECM)

Extracellular matrix (ECM), used interchangeably herein with “basement membrane matrix,” is secreted by connective tissue cells, and comprises a variety of polysaccharides, water, elastin, and proteins that may comprise proteoglycans, collagen, entactin (nidogen), fibronectin, fibrinogen, fibrillin, laminin, and hyaluronic acid. ECM may provide the suitable substrate and microenvironment conductive for selecting and culturing the subject stem cells.

In certain embodiments, the subject stem cells are attached to or in contact with an ECM. Different types of ECM are known in the art, and may comprise different compositions including different types of proteoglycans and/or different combination of proteoglycans. The ECM may be provided by culturing ECM-producing cells, such as certain fibroblast cells. Examples of extracellular matrix-producing cells include chondrocytes that mainly produce collagen and proteoglycans; fibroblast cells that mainly produce type IV collagen, laminin, interstitial procollagens, and fibronectin; and colonic myofibroblasts that mainly produce collagens (type I, III, and V), chondroitin sulfate proteoglycan, hyaluronic acid, fibronectin, and tenascin-C.

In certain embodiments, at least some ECM is produced by the murine 3T3-J2 clone, which may be grown on top of the MATRIGEL™ basement membrane matrix (BD Biosciences) as feeder cell layer.

Alternatively, the ECM may be commercially provided. Examples of commercially available extracellular matrices are extracellular matrix proteins (Invitrogen) and MATRIGEL™ basement membrane matrix (BD Biosciences). The use of an ECM for culturing stem cells may enhance long-term survival of the stem cells and/or the continued presence of undifferentiated stem cells. An alternative may be a fibrin substrate or fibrin gel—or a scaffold, such as glycerolized allografts that are depleted from the original cells.

In certain embodiments, the ECM for use in a method of the invention comprises at least two distinct glycoproteins, such as two different types of collagen or a collagen and laminin. The ECM may be a synthetic hydrogel extracellular matrix, or a naturally occurring ECM. In certain embodiments, the ECM is provided by MATRIGEL™ basement membrane matrix (BD Biosciences), which comprises laminin, entactin, and collagen IV.

Medium

A cell culture medium that is used in a method of the invention may comprise any cell culture medium, such as culture medium buffered at about pH 7.4 (e.g., between about pH 7.2-7.6) with a carbonate-based buffer. Many commercially available tissue culture media are potentially suitable for the methods of the invention, including, but are not limited to, Dulbecco's Modified Eagle Media (DMEM, e.g., DMEM without L-glutamine but with high glucose), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

The cells may be cultured in an atmosphere comprising between 5-10% CO₂ (e.g., at least about 5% but no more than 10% CO₂, or about 5% CO₂).

In certain embodiments, the cell culture medium is DMEM/F12 (e.g., 3:1 mixture) or RPMI 1640, supplemented with L-glutamine, insulin, Penicillin/streptomycin, and/or transferrin. In certain embodiments, Advanced DMEM/F12 or Advanced RPMI is used, which is optimized for serum free culture and already includes insulin. The Advanced DMEM/F12 or Advanced RPMI medium may be further supplemented with L-glutamine and Penicillin/streptomycin. In certain embodiments, the cell culture medium is supplemented with one or more a purified, natural, semi-synthetic and/or synthetic factors described herein. In certain embodiments, the cell culture medium is supplemented by about 10% fetal bovine serum (FBS) that is not heat inactivated prior to use. Additional supplements, such as, for example, B-27® Serum Free Supplement (Invitrogen), N-Acetylcysteine (Sigma) and/or N2 serum free supplement (Invitrogen), or Neurobasal (Gibco), TeSR (StemGent) may also be added to the medium.

In certain embodiments, the medium may contain one or more antibiotics to prevent contamination (such as Penicillin/streptomycin). In certain embodiments, the medium may have an endotoxin content of less that 0.1 endotoxin units per mL, or may have an endotoxin content less than 0.05 endotoxin units per mL. Methods for determining the endotoxin content of culture media are known in the art.

A cell culture medium according to the invention allows the survival and/or proliferation and/or differentiation of epithelial stem cells on an extracellular matrix. The term “cell culture medium” as used herein is synonymous with “medium,” “culture medium,” or “cell medium.”

The modified (growth) medium of the invention comprises, in a base medium, (a) a Notch agonist; (b) a ROCK (Rho Kinase) inhibitor; (c) a Bone Morphogenetic Protein (BMP) antagonist; (d) a Wnt agonist; (e) a mitogenic growth factor; and, (f) insulin or IGF; and the medium optionally further comprising at least one of: (g) a TGFβ signaling pathway inhibitor, such as TGFβ inhibitor, or a TGFβ receptor inhibitor); and, (h) nicotinamide or an analog, precursor, or mimic thereof.

Alternatively, the modified (growth) medium of the invention comprises, in a base medium, (a) a Notch agonist; (b) a ROCK (Rho Kinase) inhibitor; (c) a TGFβ signaling pathway inhibitor, such as TGFβ inhibitor, or a TGFβ receptor inhibitor); (d) a Wnt agonist; (e) nicotinamide or an analog, precursor, or mimic thereof, (f) a mitogenic growth factor; and, (g) insulin or IGF; the medium optionally further comprising (h) a Bone Morphogenetic Protein (BMP) antagonist.

The media of the invention may be prepared by adding one or more factors described above to a Base Medium.

Thus in one aspect, the invention provides a base medium (Base Medium) comprising: insulin or an insulin-like growth factor; T3 (3,3′,5-Triiodo-L-Thyronine);

hydrocortisone; adenine; EGF; and 10% fetal bovine serum (without heat inactivation), in DMEM:F12 3:1 medium supplemented with L-glutamine.

In certain embodiments, the Base Medium comprises about: 5 μg/mL insulin; 2×10⁻⁹ M T3 (3,3′,5-Triiodo-L-Thyronine); 400 ng/mL hydrocortisone; 24.3 μg/mL adenine; 10 ng/mL EGF; and 10% fetal bovine serum (without heat inactivation), in DMEM:F12 3:1 medium supplemented with 1.35 mM L-glutamine.

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value. For example, in an illustrative medium, insulin concentration may be 6 μg/mL (20% higher than the recited 5 μg/mL), EGF concentration may be 5 ng/mL (50% lower than the recited 10 ng/mL), while the remaining components each has the same concentration recited above.

In a related aspect, the invention provides a base medium containing in addition 1×10⁻¹⁰ M cholera enterotoxin. In other embodiments, the base medium does not contain cholera enterotoxin.

The Base Medium may further comprise one or more antibiotics, such as Pen/Strep, and/or gentamicin.

The base media may be used to produce Modified Growth Medium (or simply Modified Medium) by adding one or more of the factors above.

Several specific Modified Growth Media are described in detail below as Modified Growth Medium 1-5, or simply Modified Medium 1-5.

Thus, in one aspect, the invention provides a first modified medium (Modified Medium 1), comprising, in a Base Medium: Jagged-1 as a Notch agonist, Y-27632 as a ROCK inhibitor, Noggin as a BMP antagonist, R-spondin 1 as a Wnt agonist, EGF as a mitogenic growth factor, and insulin.

In certain embodiments, the Modified Medium 1 comprises, in a Base Medium: 1 μM Jagged-1 (188-204); 100 ng/mL noggin; 125 ng/mL R-spondin 1; and 2.5 μM rock inhibitor (R)-(+)-trans-N-(4-Pyridyl)-4-(1-aminoethyl)-cyclohexanecarboxamide (Y-27632).

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value.

In a related aspect, the invention provides a second modified medium (Modified Medium 2), comprising, in a Base Medium: Jagged-1 as a Notch agonist, Y-27632 as a ROCK inhibitor, Noggin as a BMP antagonist, R-spondin 1 as a Wnt agonist, SB431542 as TGF-β receptor inhibitor, EGF as a mitogenic growth factor, nicotinamide, and insulin. In certain embodiments, the Modified Medium 2 comprises, in a Base Medium: 1 μM Jagged-1 (188-204); 100 ng/mL noggin; 125 ng/mL R-spondin 1; 2.5 μM rock inhibitor (R)-(+)-trans-N-(4-Pyridyl)-4-(1-aminoethyl)-cyclohexanecarboxamide (Y-27632); 2 μM SB431542: 4-(4-(benzo[d][1,3]dioxol-5-yl)-5-(pyridin-2-yl)-1H-imidazol-2-yl)benzamide; and 10 mM nicotinamide.

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value.

In another related aspect, the invention provides a third modified medium (Modified Medium 3), comprising, in a Base Medium: Jagged-1 as a Notch agonist, Y-27632 as a ROCK inhibitor, Noggin as a BMP antagonist, R-spondin 1 as a Wnt agonist, SB431542 as TGF-β receptor inhibitor, EGF as a mitogenic growth factor, and insulin.

In certain embodiments, the Modified Medium 3 comprises, in a Base Medium: 1 μM Jagged-1 (188-204); 100 ng/mL noggin; 125 ng/mL R-spondin 1; 2.5 μM rock inhibitor (R)-(+)-trans-N-(4-Pyridyl)-4-(1-aminoethyl)-cyclohexanecarboxamide (Y-27632); and 2 μM SB431542: 4-(4-(benzo[d][1,3]dioxol-5-yl)-5-(pyridin-2-yl)-1H-imidazol-2-yl)benzamide.

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value.

In yet another related aspect, the invention provides a fourth modified medium (Modified Medium 4), comprising, in a Base Medium: Jagged-1 as a Notch agonist, Y-27632 as a ROCK inhibitor, Noggin as a BMP antagonist, R-spondin 1 as a Wnt agonist, EGF as a mitogenic growth factor, nicotinamide, and insulin.

In certain embodiments, the Modified Medium 4 comprises, in a Base Medium: 1 μM Jagged-1 (188-204); 100 ng/mL noggin; 125 ng/mL R-spondin 1; 2.5 μM rock inhibitor (R)-(+)-trans-N-(4-Pyridyl)-4-(1-aminoethyl)-cyclohexanecarboxamide (Y-27632); and 10 mM nicotinamide.

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value.

In a related aspect, the invention provides a fifth modified medium (Modified Medium 5), comprising, in a Base Medium: Jagged-1 as a Notch agonist, Y-27632 as a ROCK inhibitor, R-spondin 1 as a Wnt agonist, SB431542 as TGF-β receptor inhibitor, EGF as a mitogenic growth factor, nicotinamide, and insulin.

In certain embodiments, the Modified Medium 2 comprises, in a Base Medium: 1 μM Jagged-1 (188-204); 125 ng/mL R-spondin 1; 2.5 μM rock inhibitor (R)-(+)-trans-N-(4-Pyridyl)-4-(1-aminoethyl)-cyclohexanecarboxamide (Y-27632); 2 μM SB431542: 4-(4-(benzo[d][1,3]dioxol-5-yl)-5-(pyridin-2-yl)-1H-imidazol-2-yl)benzamide; and 10 mM nicotinamide.

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value.

The media of the invention (e.g., Modified Medium 1-5), when used according to the methods of the invention, are capable of expanding a population of isolated stem cells as single cell clones for at least 3, 4, 5, 6, 7, 8, 9, 10, 20, 30, 40, 50, 60, 70, 80, 90, 100, 150, 200, 250, 300, 350, 400, 450 or more passages under appropriate conditions.

In certain embodiments, stem cells may be isolated and cultured from fetal or adult small intestine tissues using any of the following media and culture conditions. Specifically, the modified medium Modified Medium 1 as described above may include in addition one or more of the following factors: an FGF receptor inhibitor, N-Acetyl-L-cysteine, a p38 inhibitor, Gastrin, PGE2, or TGFβ. The modified medium Modified Medium 4 as described above may include in addition one or more of the following factors: an FGF receptor inhibitor, a hedgehog protein (e.g., Shh), TGFβ, Wnt3a, or GSK3 inhibitor. Such culture conditions together with Modified Medium 2 are preferably used to isolate small intestine stem cells from fetal small intestine tissues.

In certain embodiments, the modified medium Modified Medium 3 as described above may include in addition one or more of the following factors: Gastrin, PGE2, or Wnt3a. The modified medium Modified Medium 1 as described above may include nicotinamide and a GSK3 inhibitor. Such culture conditions together with Modified Medium 3 are preferably used to isolate small intestine stem cells from adult small intestine tissues.

In certain embodiments, the modified medium Modified Medium 3 as described above may include in addition one or more of the following factors: Gastrin, PGE2, or Wnt3a. The modified medium MM1 as described above may include nicodinomide and a GSK3 inhibitor. Such culture conditions together with MM3 are preferably used to isolate small intestine stem cells from adult small intestine tissues.

As used here, “good” conditions means those under which at least about 40% of the cells have the morphology of immature stem cells in culture, and can be passaged while retaining self-renewal and differentiation capabilities; “better” conditions means those under which at least about 70% of the cells have the morphology of immature stem cells in culture, and can be passaged while retaining self-renewal and differentiation capabilities; “best” conditions means those under which about 90% of the cells in culture have the morphology of immature stem cells in culture, and can be passaged while retaining self-renewal and differentiation capabilities indefinitely in vitro.

In certain embodiments better conditions for fetal small intestine stem cells can be achieved when using Modified Medium 4, good conditions can be achieved when using Modified Medium 1, Modified Medium 1 supplemented with a FGF receptor inhibitor, or a p38 inhibitor, or PGE2, or N-Acetyl-L-cysteine, or Gastrin, or TGFβ, or supplementing Modified Medium 4 with TGFβ, or sonic hedgehog (shh), or Wnt3a, or GSK3 inhibitor, or using Modified Medium 2.

In certain embodiments better conditions for adult small intestine stem cells can be achieved when using Modified Medium 2, good conditions can be achieved when using Modified Medium 3, Modified Medium 3 supplemented with PGE2, or Gastrin, or Wnt3a, or using Modified Medium 4.

In certain embodiments, the media of the invention does not include the following conditions or combination of factors, which has been experimentally tested to show that the conditions or combination of factors do not support stem cell isolation and culturing (e.g., cannot achieve at least a “good” rating).

For fetal small intestine stem cells: Modified Medium 1 supplemented with FGF1; Modified Medium 1 supplemented with FGF1 and Wnt3a; Modified Medium 1 supplemented with Wnt5a; Modified Medium 3 supplemented with Wnt3a; Modified Medium 1 supplemented with Notch inhibitor; Modified Medium 1 supplemented with Wnt inhibitor (DKK1); Modified Medium 1 deficient of R-spondin 1; Modified Medium 1 without R-spondin 1 but supplemented with Wnt3a; Modified Medium 1 lacking R-spondin 1 but supplemented with Wnt5a; Modified Medium 4 supplemented with GDC-0449 (Vismodegib; 2-Chloro-N-(4-chloro-3-pyridin-2-ylphenyl)-4-methylsulfonylbenzamide; hedgehog signaling pathway inhibitor); Modified Medium 4 supplemented with XAV939 (2-(4-(trifluoromethyl)phenyl)-7,8-dihydro-5H-thiopyrano[4,3-d]pyrimidin-4-ol; Wnt inhibitor).

For adult small intestine stem cells: Modified Medium 1; Modified Medium 1 containing FGF1; Modified Medium 1 containing a FGF receptor inhibitor; Modified Medium 1 containing a FGF1 and Wnt3a; Modified Medium 1 containing Wnt3a; Modified Medium 1 containing Wnt5a; Modified Medium 1 containing a p38 inhibitor (e.g., SB202190); Modified Medium 1 containing PGE2; Modified Medium 1 containing N-Acetyl-L-Cys; Modified Medium 1 containing Gastrin; Modified Medium 1 without R-spondin 1; Modified Medium 3 without R-spondin 1; Modified Medium 1 without R-spondin 1 but plus Wnt3a; Modified Medium 1 without R-spondin 1 but containing Wnt5a.

### 4. Protein Sequences of the Representative Medium Factors

Several representative (non-limiting) protein factors used in the media and methods of the invention are provided below. For each listed factor, numerous homologs or functional equivalents are known in the art, and can be readily retrieved from public databases such as GenBank, EMBL, and/or NCBI RefSeq, just to name a few. Additional proteins or peptide fragments thereof, or polynucleotides encoding the same, including functional homologs from human or non-human mammals, can be readily retrieved from public sources through, for example, sequence-based searches such as NCBI BLASTp or BLASTn or both.

BMP inhibitors
Noggin: (GenBank: AAA83259.1), Homo sapiens:
(SEQ ID NO: 1)
MERCPSLGVT LYALVVVLGL RATPAGGQHY LHIRPAPSDN LPLVDLIEHP DPIFDPKEKD
LNETLLRSLL GGHYDPGFMA TSPPEDRPGG GGGAAGGAED LAELDQLLRQ RPSGAMPSEI
KGLEFSEGLA QGKKQRLSKK LRRKLQMWLW SQTFCPVLYA WNDLGSRFWP RYVKVGSCFS
KRSCSVPEGM VCKPSKSVHL TVLRWRCQRR GGQRCGWIPI QYPIISECKC SC
Chordin (GenBank: AAG35767.1), Homo sapiens:
(SEQ ID NO: 2)
MPSLPAPPAP LLLLGLLLLG SRPARGAGPE PPVLPIRSEK EPLPVRGAAG CTFGGKVYAL
DETWHPDLGE PFGVMRCVLC ACEAPQWGRR TRGPGRVSCK NIKPECPTPA CGQPRQLPGH
CCQTCPQERS SSERQPSGLS FEYPRDPEHR SYSDRGEPGA EERARGDGHT DFVALLTGPR
SQAVARARVS LLRSSLRFSI SYRRLDRPTR IRFSDSNGSV LFEHPAAPTQ DGLVCGVWRA
VPRLSLRLLR AEQLHVALVT LTHPSGEVWG PLIRHRALAA ETFSAILTLE GPPQQGVGGI
TLLTLSDTED SLHFLLLFRG LLEPRSGGLT QVPLRLQILH QGQLLRELQA NVSAQEPGFA
EVLPNLTVQE MDWLVLGELQ MALEWAGRPG LRISGHIAAR KSCDVLQSVL CGADALIPVQ
TGAAGSASLT LLGNGSLIYQ VQVVGTSSEV VAMTLETKPQ RRDQRTVLCH MAGLQPGGHT
AVGICPGLGA RGAHMLLQNE LFLNVGTKDF PDGELRGHVA ALPYCGHSAR HDTLPVPLAG
ALVLPPVKSQ AAGHAWLSLD THCHLHYEVL LAGLGGSEQG TVTAHLLGPP GTPGPRRLLK
GFYGSEAQGV VKDLEPELLR HLAKGMASLL ITTKGSPRGE LRGQVHIANQ CEVGGLRLEA
AGAEGVRALG APDTASAAPP VVPGLPALAP AKPGGPGRPR DPNTCFFEGQ QRPHGARWAP
NYDPLCSLCT CQRRTVICDP VVCPPPSCPH PVQAPDQCCP VCPEKQDVRD LPGLPRSRDP
GEGCYFDGDR SWRAAGTRWH PVVPPFGLIK CAVCTCKGGT GEVHCEKVQC PRLACAQPVR
VNPTDCCKQC PVGSGAHPQL GDPMQADGPR GCRFAGQWFP ESQSWHPSVP PFGEMSCITC
RCGAGVPHCE RDDCSLPLSC GSGKESRCCS RCTAHRRPAP ETRTDPELEK EAEGS
Follistatin (GenBank: AAH04107.1) Homo sapiens:
(SEQ ID NO: 3)
MVRARHQPGG LCLLLLLLCQ FMEDRSAQAG NCWLRQAKNG RCQVLYKTEL SKEECCSTGR
LSTSWTEEDV NDNTLFKWMI FNGGAPNCIP CKETCENVDC GPGKKCRMNK KNKPRCVCAP
DCSNITWKGP VCGLDGKTYR NECALLKARC KEQPELEVQY QGRCKKTCRD VFCPGSSTCV
VDQTNNAYCV TCNRICPEPA SSEQYLCGND GVTYSSACHL RKATCLLGRS IGLAYEGKCI
KAKSCEDIQC TGGKKCLWDF KVGRGRCSLC DELCPDSKSD EPVCASDNAT YASECAMKEA
ACSSGVLLEV KHSGSCNSIS EDTEEEEEDE DQDYSFPISS ILEW
DAN (GenBank: BAA92265.1) Homo sapiens:
(SEQ ID NO: 4)
MLRVLVGAVL PAMLLAAPPP INKLALFPDK SAWCEAKNIT QIVGHSGCEA KSIQNRACLG
QCFSYSVPNT FPQSTESLVH CDSCMPAQSM WEIVTLECPG HEEVPRVDKL VEKILHCSCQ
ACGKEPSHEG LSVYVQGEDG PGSQPGTHPH PHPHPHPGGQ TPEPEDPPGA PHTEEEGAED
Cerberus (NCBI Reference Sequence: NP_005445.1) Homo sapiens:
(SEQ ID NO: 5)
MHLLLFQLLV LLPLGKTTRH QDGRQNQSSL SPVLLPRNQR ELPTGNHEEA EEKPDLFVAV
PHLVATSPAG EGQRQREKML SRFGRFWKKP EREMHPSRDS DSEPFPPGTQ SLIQPIDGMK
MEKSPLREEA KKFWHHFMFR KTPASQGVIL PIKSHEVHWE TCRTVPFSQT ITHEGCEKVV
VQNNLCFGKC GSVHFPGAAQ HSHTSCSHCL PAKFTTMHLP LNCTELSSVI KVVMLVEECQ
CKVKTEHEDG HILHAGSQDS FIPGVSA
Gremlin (GenBank: AAF06677.1) Homo sapiens:
(SEQ ID NO: 6)
MSRTAYTVGA LLLLLGTLLP AAEGKKKGSQ GAIPPPDKAQ HNDSEQTQSP QQPGSRNRGR
GQGRGTAMPG EEVLESSQEA LHVTERKYLK RDWCKTQPLK QTIHEEGCNS RTIINRFCYG
QCNSFYIPRH IRKEEGSFQS CSFCKPKKFT TMMVTLNCPE LQPPTKKKRV TRVKQCRCIS
IDLD
Sclerostin/SOST (GenBank: AAK13451.1) Homo sapiens:
(SEQ ID NO: 7)
MQLPLALCLV CLLVHTAFRV VEGQGWQAFK NDATEIIPEL GEYPEPPPEL ENNKTMNRAE
NGGRPPHHPF ETKDVSEYSC RELHFTRYVT DGPCRSAKPV TELVCSGQCG PARLLPNAIG
RGKWWRPSGP DFRCIPDRYR AQRVQLLCPG GEAPRARKVR LVASCKCKRL TRFHNQSELK
DFGTEAARPQ KGRKPRPRAR SAKANQAELE NAY
Decorin (GenBank: AAB60901.1) Homo sapiens:
(SEQ ID NO: 8)
MKATIILLLL AQVSWAGPFQ QRGLFDFMLE DEASGIGPEV PDDRDFEPSL GPVCPFRCQC
HLRVVQCSDL
alpha-2 macroglobulin (GenBank: EAW88590.1) Homo sapiens:
(SEQ ID NO: 9)
MGKNKLLHPS LVLLLLVLLP TDASVSGKPQ YMVLVPSLLH TETTEKGCVL LSYLNETVTV
SASLESVRGN RSLFTDLEAE NDVLHCVAFA VPKSSSNEEV MFLTVQVKGP TQEFKKRTTV
MVKNEDSLVF VQTDKSIYKP GQTVKFRVVS MDENFHPLNE LIPLVYIQDP KGNRIAQWQS
FQLEGGLKQF SFPLSSEPFQ GSYKVVVQKK SGGRTEHPFT VEEFVLPKFE VQVTVPKIIT
ILEEEMNVSV CGLYTYGKPV PGHVTVSICR KYSDASDCHG EDSQAFCEKF SGQLNSHGCF
YQQVKTKVFQ LKRKEYEMKL HTEAQIQEEG TVVELTGRQS SEITRTITKL SFVKVDSHFR
QGIPFFGQVR LVDGKGVPIP NKVIFIRGNE ANYYSNATTD EHGLVQFSIN TTNVMGTSLT
VRVNYKDRSP CYGYQWVSEE HEEAHHTAYL VFSPSKSFVH LEPMSHELPC GHTQTVQAHY
ILNGGTLLGL KKLSFYYLIM AKGGIVRTGT HGLLVKQEDM KGHFSISIPV KSDIAPVARL
LIYAVLPTGD VIGDSAKYDV ENCLANKVDL SFSPSQSLPA SHAHLRVTAA PQSVCALRAV
DQSVLLMKPD AELSASSVYN LLPEKDLTGF PGPLNDQDDE DCINRHNVYI NGITYTPVSS
TNEKDMYSFL EDMGLKAFTN SKIRKPKMCP QLQQYEMHGP EGLRVGFYES DVMGRGHARL
VHVEEPHTET VRKYFPETWI WDLVVVNSAG VAEVGVTVPD TITEWKAGAF CLSEDAGLGI
SSTASLRAFQ PFFVELTMPY SVIRGEAFTL KATVLNYLPK CIRVSVQLEA SPAFLAVPVE
KEQAPHCICA NGRQTVSWAV TPKSLGNVNF TVSAEALESQ ELCGTEVPSV PEHGRKDTVI
KPLLVEPEGL EKETTFNSLL CPSGGEVSEE LSLKLPPNVV EESARASVSV LGDILGSAMQ
NTQNLLQMPY GCGEQNMVLF APNIYVLDYL NETQQLTPEI KSKAIGYLNT GYQRQLNYKH
YDGSYSTFGE RYGRNQGNTW LTAFVLKTFA QARAYIFIDE AHITQALIWL SQRQKDNGCF
RSSGSLLNNA IKGGVEDEVT LSAYITIALL EIPLTVTHPV VRNALFCLES AWKTAQEGDH
GSHVYTKALL AYAFALAGNQ DKRKEVLKSL NEEAVKKDNS VHWERPQKPK APVGHFYEPQ
APSAEVEMTS YVLLAYLTAQ PAPTSEDLTS ATNIVKWITK QQNAQGGFSS TQDTVVALHA
LSKYGAATFT RTGKAAQVTI QSSGTFSSKF QVDNNNRLLL QQVSLPELPG EYSMKVTGEG
CVYLQTSLKY NILPEKEEFP FALGVQTLPQ TCDEPKAHTS FQISLSVSYT GSRSASNMAI
VDVKMVSGFI PLKPTVKMLE RSNHVSRTEV SSNHVLIYLD KVSNQTLSLF FTVLQDVPVR
DLKPAIVKVY DYYETDEFAI AEYNAPCSKD LGNA
Wnt Agonists
R-spondin 1 (GenBank: ABC54570.1) Homo sapiens:
(SEQ ID NO: 10)
MRLGLCVVAL VLSWTHLTIS SRGIKGKRQR RISAEGSQAC AKGCELCSEV NGCLKCSPKL
FILLERNDIR QVGVCLPSCP PGYFDARNPD MNKCIKCKIE HCEACFSHNF CTKCKEGLYL
HKGRCYPACP EGSSAANGTM ECSSPAQCEM SEWSPWGPCS KKQQLCGFRR GSEERTRRVL
HAPVGDHAAC SDTKETRRCT VRRVPCPEGQ KRRKGGQGRR ENANRNLARK ESKEAGAGSR
RRKGQQQQQQ QGTVGPLTSA GPA
R-spondin 2 (NCBI Reference Sequence: NP_848660.3) Homo sapiens:
(SEQ ID NO: 11)
MQFRLFSFAL IILNCMDYSH CQGNRWRRSK RASYVSNPIC KGCLSCSKDN GCSRCQQKLF
FFLRREGMRQ YGECLHSCPS GYYGHRAPDM NRCARCRIEN CDSCFSKDFC TKCKVGFYLH
RGRCFDECPD GFAPLEETME CVEGCEVGHW SEWGTCSRNN RTCGFKWGLE TRTRQIVKKP
VKDTILCPTI AESRRCKMTM RHCPGGKRTP KAKEKRNKKK KRKLIERAQE QHSVFLATDR
ANQ
R-spondin 3 (NCBI Reference Sequence: NP_116173.2) Homo sapiens:
(SEQ ID NO: 12)
MHLRLISWLF IILNFMEYIG SQNASRGRRQ RRMHPNVSQG CQGGCATCSD YNGCLSCKPR
LFFALERIGM KQIGVCLSSC PSGYYGTRYP DINKCTKCKA DCDTCFNKNF CTKCKSGFYL
HLGKCLDNCP EGLEANNHTM ECVSIVHCEV SEWNPWSPCT KKGKTCGFKR GTETRVREII
QHPSAKGNLC PPTNETRKCT VQRKKCQKGE RGKKGRERKR KKPNKGESKE AIPDSKSLES
SKEIPEQREN KQQQKKRKVQ DKQKSVSVST VH
R-spondin 4 (NCBI Reference Sequence: NP_001025042.2)
Homo sapiens: isoform 1
(SEQ ID NO: 13)
MRAPLCLLLL VAHAVDMLAL NRRKKQVGTG LGGNCTGCII CSEENGCSTC QQRLFLFIRR
EGIRQYGKCL HDCPPGYFGI RGQEVNRCKK CGATCESCFS QDFCIRCKRQ FYLYKGKCLP
TCPPGTLAHQ NTRECQGECE LGPWGGWSPC THNGKTCGSA WGLESRVREA GRAGHEEAAT
CQVLSESRKC PIQRPCPGER SPGQKKGRKD RRPRKDRKLD RRLDVRPRQP GLQP
R-spondin 4 (NCBI Reference Sequence: NP_001035096.1)
Homo sapiens: isoform 2
(SEQ ID NO: 14)
MRAPLCLLLL VAHAVDMLAL NRRKKQVGTG LGGNCTGCII CSEENGCSTC QQRLFLFIRR
EGIRQYGKCL HDCPPGYFGI RGQEVNRCKK CGATCESCFS QDFCIRCKRQ FYLYKGKCLP
TCPPGTLAHQ NTRECQERSP GQKKGRKDRR PRKDRKLDRR LDVRPRQPGL QP
Norrin
norrin precursor [Homo sapiens]
NCBI Reference Sequence: NP_000257.1
(SEQ ID NO: 15)
MRKHVLAASF SMLSLLVIMG DTDSKTDSSF IMDSDPRRCM RHHYVDSISH PLYKCSSKMV
LLARCEGHCS QASRSEPLVS FSTVLKQPFR SSCHCCRPQT SKLKALRLRC SGGMRLTATY
RYILSCHCEE CNS
WNT3A [Homo sapiens]
GenBank: BAB61052.1
(SEQ ID NO: 16)
MAPLGYFLLL CSLKQALGSY PIWWSLAVGP QYSSLGSQPI LCASIPGLVP KQLRFCRNYV
EIMPSVAEGI KIGIQECQHQ FRGRRWNCTT VHDSLAIFGP VLDKATRESA FVHAIASAGV
AFAVTRSCAE GTAAICGCSS RHQGSPGKGW KWGGCSEDIE FGGMVSREFA DARENRPDAR
SAMNRHNNEA GRQAIASHMH LKCKCHGLSG SCEVKTCWWS QPDFRAIGDF LKDKYDSASE
MVVEKHRESR GWVETLRPRY TYFKVPTERD LVYYEASPNF CEPNPETGSF GTRDRTCNVS
SHGIDGCDLL CCGRGHNARA ERRREKCRCV FHWCCYVSCQ ECTRVYDVHT CK
WNT6 [Homo sapiens]
GenBank: AAG45154.1
(SEQ ID NO: 17)
AVGSPLVMDP TSICRKARRL AGRQAELCQA EPEVVAELAR GARLGVRECQ FQFRFRRWNC
SSHSKAFGRI LQQDIRETAF VFAITAAGAS HAVTQACSMG ELLQCGCQAP RGRAPPRPSG
LPGTPGPPGP AGSPEGSAAW EWGGCGDDVD FGDEKSRLFM DARHKRGRGD IRALVQLHNN
EAGRLAVRSH TRTECKCHGL SGSCALRTCW QKLPPFREVG ARLLERFHGA SRVMGTNDGK
ALLPAVRTLK PPGRADLLYA ADSPDFCAPN RRTGSPGTRG RACNSSAPDL SGCDLLCCGR
GHRQESVQLE ENCLCRFHWC CVVQCHRCRV RKELSLCL
Mitogenic Factors
FGF-2 = bFGF (niProtKB/Swiss-Prot: P09038.3) Homo sapiens:
(SEQ ID NO: 18)
MVGVGGGDVE DVTPRPGGCQ ISGRGARGCN GIPGAAAWEA ALPRRRPRRH PSVNPRSRAA
GSPRTRGRRT EERPSGSRLG DRGRGRALPG GRLGGRGRGR APERVGGRGR GRGTAAPRAA
PAARGSRPGP AGTMAAGSIT TLPALPEDGG SGAFPPGHFK DPKRLYCKNG GFFLRIHPDG
RVDGVREKSD PHIKLQLQAE ERGVVSIKGV CANRYLAMKE DGRLLASKCV TDECFFFERL
ESNNYNTYRS RKYTSWYVAL KRTGQYKLGS KTGPGQKAIL FLPMSAKS
FGF7 (GenBank: CAG46799.1) Homo sapiens:
(SEQ ID NO: 19)
MHKWILTWIL PTLLYRSCFH IICLVGTISL ACNDMTPEQM ATNVNCSSPE RHTRSYDYME
GGDIRVRRLF CRTQWYLRID KRGKVKGTQE MKNNYNIMEI RTVAVGIVAI KGVESEFYLA
MNKEGKLYAK KECNEDCNFK ELILENHYNT YASAKWTHNG GEMFVALNQK GIPVRGKKTK
KEQKTAHFLP MAIT
FGF10 (GenBank: CAG46489.1) Homo sapiens:
(SEQ ID NO: 20)
MWKWILTHCA SAFPHLPGCC CCCFLLLFLV SSVPVTCQAL GQVMVSPEAT NSSSSSFSSP
SSAGRHVRSY NHLQGDVRWR KLFSFTKYFL KIEKNGKVSG TKKENCPYSI LEITSVEIGV
VAVKAINSNY YLAMNKKGKL YGSKEFNNDC KLKERIEENG YNTYASFNWQ HNGRQMYVAL
NGKGAPRRGQ KTRRKNTSAH FLPMVVHS
EGF (GenBank: EAX06257.1) Homo sapiens:
(SEQ ID NO: 21)
MLLTLIILLP VVSKFSFVSL SAPQHWSCPE GTLAGNGNST CVGPAPFLIF SHGNSIFRID
TEGTNYEQLV VDAGVSVIMD FHYNEKRIYW VDLERQLLQR VFLNGSRQER VCNIEKNVSG
MAINWINEEV IWSNQQEGII TVTDMKGNNS HILLSALKYP ANVAVDPVER FIFWSSEVAG
SLYRADLDGV GVKALLETSE KITAVSLDVL DKRLFWIQYN REGSNSLICS CDYDGGSVHI
SKHPTQHNLF AMSLFGDRIF YSTWKMKTIW IANKHTGKDM VRINLHSSFV PLGELKVVHP
LAQPKAEDDT WEPEQKLCKL RKGNCSSTVC GQDLQSHLCM CAEGYALSRD RKYCEDVNEC
AFWNHGCTLG CKNTPGSYYC TCPVGFVLLP DGKRCHQLVS CPRNVSECSH DCVLTSEGPL
CFCPEGSVLE RDGKTCSGCS SPDNGGCSQL CVPLSPVSWE CDCFPGYDLQ LDEKSCAASG
PQPFLLFANS QDIRHMHFDG TDYGTLLSQQ MGMVYALDHD PVENKIYFAH TALKWIERAN
MDGSQRERLI EEGVDVPEGL AVDWIGRRFY WTDRGKSLIG RSDLNGKRSK IITKENISQP
RGIAVHPMAK RLFWTDTGIN PRIESSSLQG LGRLVIASSD LIWPSGITID FLTDKLYWCD
AKQSVIEMAN LDGSKRRRLT QNDVGHPFAV AVFEDYVWFS DWAMPSVMRV NKRTGKDRVR
LQGSMLKPSS LVVVHPLAKP GADPCLYQNG GCEHICKKRL GTAWCSCREG FMKASDGKTC
LALDGHQLLA GGEVDLKNQV TPLDILSKTR VSEDNITESQ HMLVAEIMVS DQDDCAPVGC
SMYARCISEG EDATCQCLKG FAGDGKLCSD IDECEMGVPV CPPASSKCIN TEGGYVCRCS
EGYQGDGIHC LDIDECQLGE HSCGENASCT NTEGGYTCMC AGRLSEPGLI CPDSTPPPHL
REDDHHYSVR NSDSECPLSH DGYCLHDGVC MYIEALDKYA CNCVVGYIGE RCQYRDLKWW
ELRHAGHGQQ QKVIVVAVCV VVLVMLLLLS LWGAHYYRTQ KLLSKNPKNP YEESSRDVRS
RRPADTEDGM SSCPQPWFVV IKEHQDLKNG GQPVAGEDGQ AADGSMQPTS WRQEPQLCGM
GTEQGCWIPV SSDKGSCPQV MERSFHMPSY GTQTLEGGVE KPHSLLSANP LWQQRALDPP
HQMELTQ
TGFs Homo sapiens:
protransforming growth factor alpha isoform 1 preproprotein
[Homo sapiens] NCBI Reference Sequence:
NP_003227.1
(SEQ ID NO: 22)
MVPSAGQLAL FALGIVLAAC QALENSTSPL SADPPVAAAV VSHFNDCPDS HTQFCFHGTC
RFLVQEDKPA CVCHSGYVGA RCEHADLLAV VAASQKKQAI TALVVVSIVA LAVLIITCVL
IHCCQVRKHC EWCRALICRH EKPSALLKGR TACCHSETVV
protransforming growth factor alpha isoform 2 preproprotein
[Homo sapiens] NCBI Reference Sequence:
NP_001093161.1
(SEQ ID NO: 23)
MVPSAGQLAL FALGIVLAAC QALENSTSPL SDPPVAAAVV SHFNDCPDSH TQFCFHGTCR
FLVQEDKPAC VCHSGYVGAR CEHADLLAVV AASQKKQAIT ALVVVSIVAL AVLIITCVLI
HCCQVRKHCE WCRALICRHE KPSALLKGRT ACCHSETVV
Transforming growth factor alpha [synthetic construct]
GenBank: AAX43291.1
(SEQ ID NO: 24)
MVPLAGQLAL FALGIVLAAC QALENSTSPL SDPPVAAAVV SHFNDCPDSH TQFCFHGTCR
FLVQEDKPAC VCHSGYVGAR CEHADLLAVV AASQKKQAIT ALVVVSIVAL AVLIITCVLI
HCCQVRKHCE WCRALICRHE KPSALLKGRT ACCHSETVVL
TGF alpha containing:
(SEQ ID NO: 25)
VVSHFNDCPD SHTQFCFHGT CRFLVQEDKP ACVCHSGYVG ARCEHA DLLA
BDNF (UniProtKB/Swiss-Prot: P23560.1) Homo sapiens:
(SEQ ID NO: 26)
MTILFLTMVI SYFGCMKAAP MKEANIRGQG GLAYPGVRTH GTLESVNGPK AGSRGLTSLA
DTFEHVIEEL LDEDQKVRPN EENNKDADLY TSRVMLSSQV PLEPPLLFLL EEYKNYLDAA
NMSMRVRRHS DPARRGELSV CDSISEWVTA ADKKTAVDMS GGTVTVLEKV PVSKGQLKQY
FYETKCNPMG YTKEGCRGID KRHWNSQCRT TQSYVRALTM DSKKRIGWRF IRIDTSCVCT
LTIKRGR
KGF (GenBank: AAB21431.1) Homo sapiens:
(SEQ ID NO: 27)
MHKWILTWIL PTLLYRSCFH IICLVGTISL ACNDMTPEQM ATNVNCSSPE RHTRSYDYME
GGDIRVRRLF CRTQWYLRID KRGKVKGTQE MKNNYNIMEI RTVAVGIVAI KGVESEFYLA
MNKEGKLYAK KECNEDCNFK ELILENHYNT YASAKWTHNG GEMFVALNQK GIPVRGKKTK
KEQKTAHFLP MAIT
Notch Agonist
Jagged-1 (GenBank: ACJ68517.1) Homo sapiens:
(SEQ ID NO: 28)
MRSPRTRGRS GRPLSLLLAL LCALRAKVCG ASGQFELEIL SMQNVNGELQ NGNCCGGARN
PGDRKCTRDE CDTYFKVCLK EYQSRVTAGG PCSFGSGSTP VIGGNTFNLK ASRGNDRNRI
VLPFSFAWPR SYTLLVEAWD SSNDTVQPDS IIEKASHSGM INPSRQWQTL KQNTGVAHFE
YQIRVTCDDY YYGFGCNKFC RPRDDFFGHY ACDQNGNKTC MEGWMGPECN RAICRQGCSP
KHGSCKLPGD CRCQYGWQGL YCDKCIPHPG CVHGICNEPW QCLCETNWGG QLCDKDLNYC
GTHQPCLNGG TCSNTGPDKY QCSCPEGYSG PNCEIAEHAC LSDPCHNRGS CKETSLGFEC
ECSPGWTGPT CSTNIDDCSP NNCSHGGTCQ DLVNGFKCVC PPQWTGKTCQ LDANECEAKP
CVNAKSCKNL IASYYCDCLP GWMGQNCDIN INDCLGQCQN DASCRDLVNG YRCICPPGYA
GDHCERDIDE CASNPCLNGG HCQNEINRFQ CLCPTGFSGN LCQLDIDYCE PNPCQNGAQC
YNRASDYFCK CPEDYEGKNC SHLKDHCRTT PCEVIDSCTV AMASNDTPEG VRYISSNVCG
PHGKCKSQSG GKFTCDCNKG FTGTYCHENI NDCESNPCRN GGTCIDGVNS YKCICSDGWE
GAYCETNIND CSQNPCHNGG TCRDLVNDFY CDCKNGWKGK TCHSRDSQCD EATCNNGGTC
YDEGDAFKCM CPGGWEGTTC NIARNSSCLP NPCHNGGTCV VNGESFTCVC KEGWEGPICA
QNTNDCSPHP CYNSGTCVDG DNWYRCECAP GFAGPDCRIN INECQSSPCA FGATCVDEIN
GYRCVCPPGH SGAKCQEVSG RPCITMGSVI PDGAKWDDDC NTCQCLNGRI ACSKVWCGPR
PCLLHKGHSE CPSGQSCIPI LDDQCFVHPC TGVGECRSSS LQPVKTKCTS DSYYQDNCAN
ITFTFNKEMM SPGLTTEHIC SELRNLNILK NVSAEYSIYI ACEPSPSANN EIHVAISAED
IRDDGNPIKE ITDKIIDLVS KRDGNSSLIA AVAEVRVQRR PLKNRTDFLV PLLSSVLTVA
WICCLVTAFY WCLRKRRKPG SHTHSASEDN TTNNVREQLN QIKNPIEKHG ANTVPIKDYE
NKNSKMSKIR THNSEVEEDD MDKHQQKARF AKQPAYTLVD REEKPPNGTP TKHPNWTNKQ
DNRDLESAQS LNRMEYIV
Jagged-1 peptide
(SEQ ID NO: 29)
MRGSHHHHHH GSIEGRSAVT CDDYYYGFGC NKFCRPRDDF FGHYACDQNG NKTCMEGWMG
PECNRAICRQ GCSPKHGSCK LPGDCRCQYG WQGLYCDKCI PHPGCVHGIC NEPWQCLCET
NWGGQLCDKD LNYCGTHQPC LNGGTCSNTG PDKYQCSCPE GYSGPNCEI
Jagged-1 peptide
CDDYYYGFGCNKFCRPR
(SEQ ID NO: 30)
Jagged2 [Homo sapiens]
GenBank: AAD15562.1
(SEQ ID NO: 31)
MRAQGRGRLP RRLLLLLALW VQAARPMGYF ELQLSALRNV NGELLSGACC DGDGRTTRAG
GCGHDECDTY VRVCLKEYQA KVTPTGPCSY GHGATPVLGG NSFYLPPAGA AGDRARARAR
AGGDQDPGLV VIPFQFAWPR SFTLIVEAWD WDNDTTPNEE LLIERVSHAG MINPEDRWKS
LHFSGHVAHL ELQIRVRCDE NYYSATCNKF CRPRNDFFGH YTCDQYGNKA CMDGWMGKEC
KEAVCKQGCN LLHGGCTVPG ECRCSYGWQG RFCDECVPYP GCVHGSCVEP WQCNCETNWG
GLLCDKDLNY CGSHHPCTNG GTCINAEPDQ YRCTCPDGYS GRNCEKAEHA CTSNPCANGG
SCHEVPSGFE CHCPSGWSGP TCALDIDECA SNPCAAGGTC VDQVDGFECI CPEQWVGATC
QLDANECEGK PCLNAFSCKN LIGGYYCDCI PGWKGINCHI NVNDCRGQCQ HGGTCKDLVN
GYQCVCPRGF GGRHCELERD ECASSPCHSG GLCEDLADGF HCHCPQGFSG PLCEVDVDLC
EPSPCRNGAR CYNLEGDYYC ACPDDFGGKN CSVPREPCPG GACRVIDGCG SDAGPGMPGT
AASGVCGPHG RCVSQPGGNF SCICDSGFTG TYCHENIDDC LGQPCRNGGT CIDEVDAFRC
FCPSGWEGEL CDTNPNDCLP DPCHSRGRCY DLVNDFYCAC DDGWKGKTCH SREFQCDAYT
CSNGGTCYDS GDTFRCACPP GWKGSTCAVA KNSSCLPNPC VNGGTCVGSG ASFSCICRDG
WEGRTCTHNT NDCNPLPCYN GGICVDGVNW FRCECAPGFA GPDCRINIDE CQSSPCAYGA
TCVDEINGYR CSCPPGRAGP RCQEVIGFGR SCWSRGTPFP HGSSWVEDCN SCRCLDGRRD
CSKVWCGWKP CLLAGQPEAL SAQCPLGQRC LEKAPGQCLR PPCEAWGECG AEEPPSTPCL
PRSGHLDNNC ARLTLHFNRD HVPQGTTVGA ICSGIRSLPA TRAVARDRLL VLLCDRASSG
ASAVEVAVSF SPARDLPDSS LIQGAAHAIV AAITQRGNSS LLLAVTEVKV ETVVTGGSST
GLLVPVLCGA FSVLWLACVV LCVWWTRKRR KERERSRLPR EESANNQWAP LNPIRNPIER
PGGHKDVLYQ CKNFTPPPRR ADEALPGPAG HAAVREDEED EDLGRGEEDS LEAEKFLSHK
FTKDPGRSPG RPAHWASGPK VDNRAVRSIN EARYAGKE
Delta 1 = delta-like protein 1 (NCBI Reference Sequence:
NP_005609.3; GenBank: AF196571.1) Homo sapiens:
(SEQ ID NO: 32)
MGSRCALALA VLSALLCQVW SSGVFELKLQ EFVNKKGLLG NRNCCRGGAG PPPCACRTFF
RVCLKHYQAS VSPEPPCTYG SAVTPVLGVD SFSLPDGGGA DSAFSNPIRF PFGFTWPGTF
SLIIEALHTD SPDDLATENP ERLISRLATQ RHLTVGEEWS QDLHSSGRTD LKYSYRFVCD
EHYYGEGCSV FCRPRDDAFG HFTCGERGEK VCNPGWKGPY CTEPICLPGC DEQHGFCDKP
GECKCRVGWQ GRYCDECIRY PGCLHGTCQQ PWQCNCQEGW GGLFCNQDLN YCTHHKPCKN
GATCTNTGQG SYTCSCRPGY TGATCELGID ECDPSPCKNG GSCTDLENSY SCTCPPGFYG
KICELSAMTC ADGPCFNGGR CSDSPDGGYS CRCPVGYSGF NCEKKIDYCS SSPCSNGAKC
VDLGDAYLCR CQAGFSGRHC DDNVDDCASS PCANGGTCRD GVNDFSCTCP PGYTGRNCSA
PVSRCEHAPC HNGATCHERG HRYVCECARG YGGPNCQFLL PELPPGPAVV DLTEKLEGQG
GPFPWVAVCA GVILVLMLLL GCAAVVVCVR LRLQKHRPPA DPCRGETETM NNLANCQREK
DISVSIIGAT QIKNTNKKAD FHGDHSADKN GFKARYPAVD YNLVQDLKGD DTAVRDAHSK
RDTKCQPQGS SGEEKGTPTT LRGGEASERK RPDSGCSTSK DTKYQSVYVI SEEKDECVIA
TEV
Delta-4 = delta-like protein 4 precursor [Homo sapiens]
NCBI Reference Sequence: NP_061947.1
(SEQ ID NO: 33)
MAAASRSASG WALLLLVALW QQRAAGSGVF QLQLQEFINE RGVLASGRPC EPGCRTFFRV
CLKHFQAVVS PGPCTFGTVS TPVLGTNSFA VRDDSSGGGR NPLQLPFNFT WPGTFSLIIE
AWHAPGDDLR PEALPPDALI SKIAIQGSLA VGQNWLLDEQ TSTLTRLRYS YRVICSDNYY
GDNCSRLCKK RNDHFGHYVC QPDGNLSCLP GWTGEYCQQP ICLSGCHEQN GYCSKPAECL
CRPGWQGRLC NECIPHNGCR HGTCSTPWQC TCDEGWGGLF CDQDLNYCTH HSPCKNGATC
SNSGQRSYTC TCRPGYTGVD CELELSECDS NPCRNGGSCK DQEDGYHCLC PPGYYGLHCE
HSTLSCADSP CFNGGSCRER NQGANYACEC PPNFTGSNCE KKVDRCTSNP CANGGQCLNR
GPSRMCRCRP GFTGTYCELH VSDCARNPCA HGGTCHDLEN GLMCTCPAGF SGRRCEVRTS
IDACASSPCF NRATCYTDLS TDTFVCNCPY GFVGSRCEFP VGLPPSFPWV AVSLGVGLAV
LLVLLGMVAV AVRQLRLRRP DDGSREAMNN LSDFQKDNLI PAAQLKNTNQ KKELEVDCGL
DKSNCGKQQN HTLDYNLAPG PLGRGTMPGK FPHSDKSLGE KAPLRLHSEK PECRISAICS
PRDSMYQSVC LISEERNECV IATEV
delta-like protein 3 isoform 1 precursor [Homo sapiens]
NCBI Reference Sequence: NP_058637.1
(SEQ ID NO: 34)
MVSPRMSGLL SQTVILALIF LPQTRPAGVF ELQIHSFGPG PGPGAPRSPC SARLPCRLFF
RVCLKPGLSE EAAESPCALG AALSARGPVY TEQPGAPAPD LPLPDGLLQV PFRDAWPGTF
SFIIETWREE LGDQIGGPAW SLLARVAGRR RLAAGGPWAR DIQRAGAWEL RFSYRARCEP
PAVGTACTRL CRPRSAPSRC GPGLRPCAPL EDECEAPLVC RAGCSPEHGF CEQPGECRCL
EGWTGPLCTV PVSTSSCLSP RGPSSATTGC LVPGPGPCDG NPCANGGSCS ETPRSFECTC
PRGFYGLRCE VSGVTCADGP CFNGGLCVGG ADPDSAYICH CPPGFQGSNC EKRVDRCSLQ
PCRNGGLCLD LGHALRCRCR AGFAGPRCEH DLDDCAGRAC ANGGTCVEGG GAHRCSCALG
FGGRDCRERA DPCAARPCAH GGRCYAHFSG LVCACAPGYM GARCEFPVHP DGASALPAAP
PGLRPGDPQR YLLPPALGLL VAAGVAGAAL LLVHVRRRGH SQDAGSRLLA GTPEPSVHAL
PDALNNLRTQ EGSGDGPSSS VDWNRPEDVD PQGIYVISAP SIYAREVATP LFPPLHTGRA
GQRQHLLFPY PSSILSVK
Delta-like protein 3 isoform 2 precursor [Homo sapiens]
NCBI Reference Sequence: NP_982353.1
(SEQ ID NO: 35)
MVSPRMSGLL SQTVILALIF LPQTRPAGVF ELQIHSFGPG PGPGAPRSPC SARLPCRLFF
RVCLKPGLSE EAAESPCALG AALSARGPVY TEQPGAPAPD LPLPDGLLQV PFRDAWPGTF
SFIIETWREE LGDQIGGPAW SLLARVAGRR RLAAGGPWAR DIQRAGAWEL RFSYRARCEP
PAVGTACTRL CRPRSAPSRC GPGLRPCAPL EDECEAPLVC RAGCSPEHGF CEQPGECRCL
EGWTGPLCTV PVSTSSCLSP RGPSSATTGC LVPGPGPCDG NPCANGGSCS ETPRSFECTC
PRGFYGLRCE VSGVTCADGP CFNGGLCVGG ADPDSAYICH CPPGFQGSNC EKRVDRCSLQ
PCRNGGLCLD LGHALRCRCR AGFAGPRCEH DLDDCAGRAC ANGGTCVEGG GAHRCSCALG
FGGRDCRERA DPCAARPCAH GGRCYAHFSG LVCACAPGYM GARCEFPVHP DGASALPAAP
PGLRPGDPQR YLLPPALGLL VAAGVAGAAL LLVHVRRRGH SQDAGSRLLA GTPEPSVHAL
PDALNNLRTQ EGSGDGPSSS VDWNRPEDVD PQGIYVISAP SIYAREA

### 5. Methods for Differentiating the Stem Cells

The isolated stem cells (e.g., adult stem cells) may be induced to differentiate into differentiated cells that normally reside in the tissue or organ from which the stem cells originates or are isolated. The differentiated cells may express markers characteristic of the differentiated cells, and can be readily distinguished from the stem cells which do not express such differentiated cell markers.

A list of representative markers expressed in adult stem cells include: SOX9, KRT19, KRT7, LGR5, CA9, FXYD2, CDH6, CLDN18, TSPAN8, BPIFB1, OLFM4, CDH17, and PPARGC1A.

In certain embodiments, the adult stem cells do not or negligibly express any of the differentiated markers described here.

A list of representative markers expressed in adult small intestinal stem cells include: OLFM4, SOX9, LGR5, CLDN18, CA9, BPIFB1, KRT19, CDH17, and TSPAN8.

A list of representative markers expressed in differentiated small intestinal cells include: MUC or PAS (goblet cell markers), CHGA (neuroendocrine cell marker), LYZ (Paneth cell marker), MUC7, MUC13, and KRT20.

A list of representative markers expressed in adult liver stem cells include: SOX9, KRT19, KRT7, FXYD2, and TSPAN8.

A list of representative markers expressed in differentiated liver cells include: albumin, HNF1α, HNF4α, and AFP.

A list of representative markers expressed in adult pancreatic stem cells include: SOX9, KRT19, KRT7, FXYD2, CA9, and CDH6.

A list of representative markers expressed in adult stomach stem cells include: SOX9, SOX2, CLDN18, TSPAN8, KRT7, KRT19, BPIFB1, and PPARGC1A.

A list of representative markers expressed in adult colon stem cells include: SOX9, OLFM4, LGR5, CLDN18, CA9, BPIFB1, KRT19, and PPARGC1A.

A list of representative markers expressed in adult intestinal metaplasia stem cells include: SOX9, CDH17, HEPH and RAB3B.

The intestinal metaplasia stem cells can differentiate into columnar epithelium that mimic the mature intestinal metaplasia, expressing the markers such as Cdx2 and Villin, but do not express gastric epithelium markers such as GKN1.

A list of representative markers expressed in adult kidney stem cells include: KRT19, KRT7, FXYD2, and CDH6.

A list of representative markers expressed in adult upper airway stem cells include: KRT14, KRT5, P63, KRT15 and SOX2.

A list of representative markers expressed in Fallopian tube stem cells include: ZFPM2, CLDN10, and PAX8.

A list of representative markers expressed in differentiated Fallopian tube cells include: FOXJ1 and PAX2.

Any of the markers described above are well known in the art, and the expression of which can be verified by any of many art-recognized methods, such as Western blot, Northern blot, immunohistochemistry, immunofluorescent staining, in situ RNA hybridization, etc.

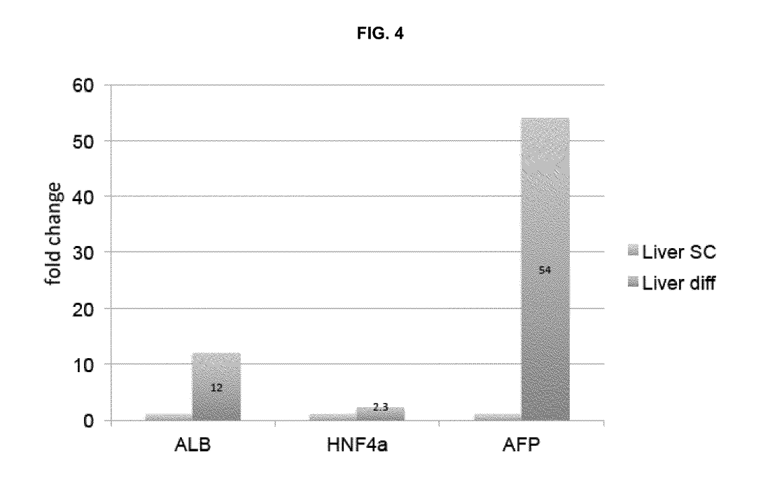

In certain embodiments, the level of expression of any specific marker genes can be assessed, and compared between the stem cells and differentiated cells, using a quantitative method such as real time PCR. See FIG. 4 and Example 7.

In certain embodiments, differentiation may be assessed by detecting a function of a differentiated cell, such as secretion of insulin by a pancreatic cell differentiated from a pancreatic stem cell that does not secret insulin.

Conditions for induced differentiation of the isolated stem cells are well known in the art.

For example, a differentiation medium that is designed to promote or induce the differentiation of pancreatic stem cells is capable of inducing the expression of at least one pancreatic differentiation marker after culturing the pancreatic stem cell in the medium for about 2, 3, 4, 5, 6, 7, 8, 9, 10 or more days.

The pancreatic differentiation marker Neurogenin-3 can be used to assess the commencement and/or extent of differentiation. The marker expression level can be detected by RT-PCR or by immunohistochemistry.

A representative pancreatic differentiation medium (e.g., minimal differentiation medium) comprises Epidermal Growth Factor, R-spondin 1 as Wnt agonist, supplemented with B27, N2, and N-Acetylcystein, and does not contain FGF or KGF or FGF10.

Another representative pancreatic differentiation medium (e.g., improved differentiation medium) comprises Noggin as BMP inhibitor, both Epidermal Growth Factor and Keratinocyte Growth Factor as mitogenic growth factors, and R-spondin 1 as Wnt agonist, supplemented with B27, N2, and N-Acetylcystein (KGF may be replaced by a FGF, or by FGF10), and is supplemented with [Leu15]-Gastrin I and/or Exendin.

An additional differentiation medium is designed to differentiate cells towards a gastric lineage, and comprises Epidermal Growth Factor as mitogenic growth factor, R-spondin 1 as Wnt agonist, Wnt-3a as Wnt agonist, Noggin as BMP inhibitor, and FGF10, supplemented with B27, N2, N-Acetylcystein and Gastrin. Gastrin is preferably used at a concentration of 1 nM.

The medium induces or promotes a specific differentiation of cells during at least 2, 3, 4, 5, 6, 7, 8, 9, 10 days of culture or longer to a gastric lineage. Differentiation may be measured by detecting the presence of a specific marker associated with the gastric lineage, such as MUC5AC (a pit cell marker), GASTRIN and/or SOMATOSTATIN (both, endocrine cell markers). The presence of at least one of said markers can be carried out using RT-PCR and/or immunohistochemistry or immunofluorescence. The presence of at least one of these markers may be detectable after at least 6 days in the differentiation conditions, or at least 10 days.

Yet another differentiation medium comprise Advanced-DMEM/F12 supplemented with Glutamax, Penicillin/Streptomycin, 10 mM Hepes, B27, N2, 200ng/ml N-Acetylcystein, 10 nM [Leu15]-Gastrin I, 100 nM Exendin4, 50 ng/ml EGF, 1 μg/ml R-spondin 1, 100 ng/ml Noggin.

Further differentiation media are described in WO 2010/090513, WO 2012/014076, WO 2012/168930, and WO 2012/044992, all incorporated herein by reference.

Additional differentiation media are described in detail in the Examples below (see Examples 7-10, 13, and 14), which conditions and variations thereof constitute part of this section.

### 6. Markers

This section describes representative marker genes that may be used to identify isolated stem cells from different tissues or organs, or cells differentiated therefrom. In general, gene expression may be measured at RNA level for all of the markers described below. In addition, the expression of certain markers can also be detected by protein expression using, for example, antibody specific for proteins encoded by the marker genes.

### Adult Small Intestinal Stem Cells

In their undifferentiated state, adult human small intestinal stem cells express one or more of the following biomarkers: OLFM4, SOX9, LGR5, CLDN18, CA9, BPIFB1, KRT19, CDH17, TSPAN8. Gene expression may be measured at RNA level for all of these markers, or at the protein level for SOX9, CLDN18, CA9, KRT19, CDH17, and TSPAN8.

### Adult Colon Stem Cells

In their undifferentiated state, adult human colon stem cells express at least on of the following biomarkers: OLFM4, SOX9, LGR5, CLDN18, CA9, BPIFB1, KRT19 and PPARGC1A. Gene expression may be measured at RNA level for all of these markers, or at the protein level for SOX9, CLDN18, CA9, and KRT19.

### Adult Gastric Stem Cells

In their undifferentiated state, adult human gastric stem cells express at least on of the following biomarkers: SOX9, SOX2, CLDN18, TSPAN8, KRT7, KRT19, BPIFB1, PPARGC1A. Gene expression may be measured at RNA level for all of these markers, or at the protein level for SOX9, SOX2, CLDN18, TSPAN8, KRT7, and KRT19.

### Adult Liver Stem Cells

In their undifferentiated state, adult human liver stem cells express at least on of the following biomarkers: SOX9, KRT7, KRT19, FXYD2 and TSPAN8. Gene expression may be measured at RNA level for all of these markers, or at the protein level for SOX9, KRT7, KRT19, and TSPAN8.

### Adult Pancreatic Stem Cells

In their undifferentiated state, adult human pancreatic stem cells express at least on of the following biomarkers: SOX9, KRT7, KRT19, FXYD2, CA9 and CDH6. Gene expression may be measured at RNA level for all of these markers, or at the protein level for SOX9, KRT7, KRT19 and CA9.

### Adult Renal Stem Cells

In their undifferentiated state, adult human renal stem cells express at least on of the following biomarkers: KRT7, KRT19, FXYD2, and CDH6. Gene expression may be measured at RNA level for all of these markers, or at the protein level for KRT7 and KRT19.

### Fallopian Tube Stem Cells

In their undifferentiated state, adult human renal stem cells express at least on of the following biomarkers: ZFPM2, CLDN10 and PAX8. Gene expression may be measured at RNA level for all of these markers.

### Adult Intestinal Metaplasia Stem Cells

In their undifferentiated state, adult human intestinal metaplasia stem cells express at least on of the following biomarkers: SOX9, CDH17, HEPH and RAB3B. Gene expression may be measured at RNA or protein level for all of these markers.

Specific marker genes and their sequences are provided herewith.

### BPIFB1

BPI fold containing family B, member 1 (BPIFB1) s a member of the BPI/LBP/PLUNC protein superfamily. BPIFB1 is also known as LPLUNC1 or C20orf114. BPIFB1 expression has been detected in small intestinal stem cells, colon stem cells, and gastric stem cells. RNA expression can be measure for example by RT-PCR, RT-qPCR, RNA-Seq, microarray approaches or RNA in situ hybridization.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art.

The human cDNA sequence is listed below (NCBI Reference Sequence: NM_{—}033197.2)

| (SEQ ID NO: 36) | |
|---|---|
| 1 | ggtctgaggc ctctgcctaa agacaaagcc tgtgctgggg tgtgcaggat ataaggttgg |
| 61 | acttccagac ccactgcccg ggagaggaga ggagcgggcc gaggactcca gcgtgcccag |
| 121 | gtctggcatc ctgcacttgc tgccctctga cacctgggaa gatggccggc ccgtggacct |
| 181 | tcacccttct ctgtggtttg ctggcagcca ccttgatcca agccaccctc agtcccactg |
| 241 | cagttctcat cctcggccca aaagtcatca aagaaaagct gacacaggag ctgaaggacc |
| 301 | acaacgccac cagcatcctg cagcagctgc cgctgctcag tgccatgcgg gaaaagccag |
| 361 | ccggaggcat ccctgtgctg ggcagcctgg tgaacaccgt cctgaagcac atcatctggc |
| 421 | tgaaggtcat cacagctaac atcctccagc tgcaggtgaa gccctcggcc aatgaccagg |
| 481 | agctgctagt caagatcccc ctggacatgg tggctggatt caacacgccc ctggtcaaga |
| 541 | ccatcgtgga gttccacatg acgactgagg cccaagccac catccgcatg gacaccagtg |
| 601 | caagtggccc cacccgcctg gtcctcagtg actgtgccac cagccatggg agcctgcgca |
| 661 | tccaactgct gcataagctc tccttcctgg tgaacgcctt agctaagcag gtcatgaacc |
| 721 | tcctagtgcc atccctgccc aatctagtga aaaaccagct gtgtcccgtg atcgaggctt |
| 781 | ccttcaatgg catgtatgca gacctcctgc agctggtgaa ggtgcccatt tccctcagca |
| 841 | ttgaccgtct ggagtttgac cttctgtatc ctgccatcaa gggtgacacc attcagctct |
| 901 | acctgggggc caagttgttg gactcacagg gaaaggtgac caagtggttc aataactctg |
| 961 | cagcttccct gacaatgccc accctggaca acatcccgtt cagcctcatc gtgagtcagg |
| 1021 | acgtggtgaa agctgcagtg gctgctgtgc tctctccaga agaattcatg gtcctgttgg |
| 1081 | actctgtgct tcctgagagt gcccatcggc tgaagtcaag catcgggctg atcaatgaaa |
| 1141 | aggctgcaga taagctggga tctacccaga tcgtgaagat cctaactcag gacactcccg |
| 1201 | agttttttat agaccaaggc catgccaagg tggcccaact gatcgtgctg gaagtgtttc |
| 1261 | cctccagtga agccctccgc cctttgttca ccctgggcat cgaagccagc tcggaagctc |
| 1321 | agttttacac caaaggtgac caacttatac tcaacttgaa taacatcagc tctgatcgga |
| 1381 | tccagctgat gaactctggg attggctggt tccaacctga tgttctgaaa aacatcatca |
| 1441 | ctgagatcat ccactccatc ctgctgccga accagaatgg caaattaaga tctggggtcc |
| 1501 | cagtgtcatt ggtgaaggcc ttgggattcg aggcagctga gtcctcactg accaaggatg |
| 1561 | cccttgtgct tactccagcc tccttgtgga aacccagctc tcctgtctcc cagtgaagac |
| 1621 | ttggatggca gccatcaggg aaggctgggt cccagctggg agtatgggtg tgagctctat |
| 1681 | agaccatccc tctctgcaat caataaacac ttgcctgtga tgcctgcaaa aaaa |

### CA9

Carbonic anhydrase IX (CA9), also known as MN or CAIX, s a transmembrane protein and belongs to a large family of zinc metalloenzymes.

CA9 expression has been detected in small intestinal stem cells, colon stem cells, and pancreatic stem cells. RNA expression can be measure for example by RT-PCR, RT-qPCR, RNA-Seq, microarray approaches or RNA in situ hybridization. Protein expression, measurable for example by immunofluorescence, immunohistochemistry, FACS, flow cytometry, Western blot or ELISA of CA9 and can be used to characterize the stem cells.

In situ probes can be obtained for example from Advanced Cell Diagnostics RNAscope (cat no. 559341). qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA) or Abnova (Neihu District. Taipei City, Taiwan).

The human cDNA sequence is listed below (NCBI Reference Sequence: NM_{—}001216)

| (SEQ ID NO: 37) | |
|---|---|
| 1 | gcccgtacac accgtgtgct gggacacccc acagtcagcc gcatggctcc cctgtgcccc |
| 61 | agcccctggc tccctctgtt gatcccggcc cctgctccag gcctcactgt gcaactgctg |
| 121 | ctgtcactgc tgcttctggt gcctgtccat ccccagaggt tgccccggat gcaggaggat |
| 181 | tcccccttgg gaggaggctc ttctggggaa gatgacccac tgggcgagga ggatctgccc |
| 241 | agtgaagagg attcacccag agaggaggat ccacccggag aggaggatct acctggagag |
| 301 | gaggatctac ctggagagga ggatctacct gaagttaagc ctaaatcaga agaagagggc |
| 361 | tccctgaagt tagaggatct acctactgtt gaggctcctg gagatcctca agaaccccag |
| 421 | aataatgccc acagggacaa agaaggggat gaccagagtc attggcgcta tggaggcgac |
| 481 | ccgccctggc cccgggtgtc cccagcctgc gcgggccgct tccagtcccc ggtggatatc |
| 541 | cgcccccagc tcgccgcctt ctgcccggcc ctgcgccccc tggaactcct gggcttccag |
| 601 | ctcccgccgc tcccagaact gcgcctgcgc aacaatggcc acagtgtgca actgaccctg |
| 661 | cctcctgggc tagagatggc tctgggtccc gggcgggagt accgggctct gcagctgcat |
| 721 | ctgcactggg gggctgcagg tcgtccgggc tcggagcaca ctgtggaagg ccaccgtttc |
| 781 | cctgccgaga tccacgtggt tcacctcagc accgcctttg ccagagttga cgaggccttg |
| 841 | gggcgcccgg gaggcctggc cgtgttggcc gcctttctgg aggagggccc ggaagaaaac |
| 901 | agtgcctatg agcagttgct gtctcgcttg gaagaaatcg ctgaggaagg ctcagagact |
| 961 | caggtcccag gactggacat atctgcactc ctgccctctg acttcagccg ctacttccaa |
| 1021 | tatgaggggt ctctgactac accgccctgt gcccagggtg tcatctggac tgtgtttaac |
| 1081 | cagacagtga tgctgagtgc taagcagctc cacaccctct ctgacaccct gtggggacct |
| 1141 | ggtgactctc ggctacagct gaacttccga gcgacgcagc ctttgaatgg gcgagtgatt |
| 1201 | gaggcctcct tccctgctgg agtggacagc agtcctcggg ctgctgagcc agtccagctg |
| 1261 | aattcctgcc tggctgctgg tgacatccta gccctggttt ttggcctcct ttttgctgtc |
| 1321 | accagcgtcg cgttccttgt gcagatgaga aggcagcaca gaaggggaac caaagggggt |
| 1381 | gtgagctacc gcccagcaga ggtagccgag actggagcct agaggctgga tcttggagaa |
| 1441 | tgtgagaagc cagccagagg catctgaggg ggagccggta actgtcctgt cctgctcatt |
| 1501 | atgccacttc cttttaactg ccaagaaatt ttttaaaata aatatttata ataaaaaaaa |
| 1561 | a |

### CDH17

Cadherin 17 (CDH17), also known as LI cadherin (liver-intestine), human peptide transporter 1 (HPT1 or HPT-1), or CDH16 is a member of the cadherin superfamily. CDH17 expression has been detected in small intestinal stem cells, and intestinal metaplasia stem cells. RNA expression can be measure for example by RT-PCR, RT-qPCR, RNA-Seq, microarray approaches or RNA in situ hybridization. Protein expression, measurable for example by immunofluorescence, immunohistochemistry, FACS, flow cytometry, Western blot or ELISA of CDH17 can be used to characterize the stem cells.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA) or Abnova (Neihu District. Taipei City, Taiwan).

The human cDNA sequences is listed below (NCBI Reference Sequence: NM_{—}004063.3; transcript variant 1 and NM_{—}001144663.1; transcript variant 2).

NCBI Reference Sequence: NM_{—}004063.3; Transcript Variant 1

| (SEQ ID NO: 38) | |
|---|---|
| 1 | ggaagaggga gtgttcccgg gggagatact ccagtcgtag caagagtctc gaccactgaa |
| 61 | tggaagaaaa ggacttttaa ccaccatttt gtgacttaca gaaaggaatt tgaataaaga |
| 121 | aaactatgat acttcaggcc catcttcact ccctgtgtct tcttatgctt tatttggcaa |
| 181 | ctggatatgg ccaagagggg aagtttagtg gacccctgaa acccatgaca ttttctattt |
| 241 | atgaaggcca agaaccgagt caaattatat tccagtttaa ggccaatcct cctgctgtga |
| 301 | cttttgaact aactggggag acagacaaca tatttgtgat agaacgggag ggacttctgt |
| 361 | attacaacag agccttggac agggaaacaa gatctactca caatctccag gttgcagccc |
| 421 | tggacgctaa tggaattata gtggagggtc cagtccctat caccataaaa gtgaaggaca |
| 481 | tcaacgacaa tcgacccacg tttctccagt caaagtacga aggctcagta aggcagaact |
| 541 | ctcgcccagg aaagcccttc ttgtatgtca atgccacaga cctggatgat ccggccactc |
| 601 | ccaatggcca gctttattac cagattgtca tccagcttcc catgatcaac aatgtcatgt |
| 661 | actttcagat caacaacaaa acgggagcca tctctcttac ccgagaggga tctcaggaat |
| 721 | tgaatcctgc taagaatcct tcctataatc tggtgatctc agtgaaggac atgggaggcc |
| 781 | agagtgagaa ttccttcagt gataccacat ctgtggatat catagtgaca gagaatattt |
| 841 | ggaaagcacc aaaacctgtg gagatggtgg aaaactcaac tgatcctcac cccatcaaaa |
| 901 | tcactcaggt gcggtggaat gatcccggtg cacaatattc cttagttgac aaagagaagc |
| 961 | tgccaagatt cccattttca attgaccagg aaggagatat ttacgtgact cagcccttgg |
| 1021 | accgagaaga aaaggatgca tatgtttttt atgcagttgc aaaggatgag tacggaaaac |
| 1081 | cactttcata tccgctggaa attcatgtaa aagttaaaga tattaatgat aatccaccta |
| 1141 | catgtccgtc accagtaacc gtatttgagg tccaggagaa tgaacgactg ggtaacagta |
| 1201 | tcgggaccct tactgcacat gacagggatg aagaaaatac tgccaacagt tttctaaact |
| 1261 | acaggattgt ggagcaaact cccaaacttc ccatggatgg actcttccta atccaaacct |
| 1321 | atgctggaat gttacagtta gctaaacagt ccttgaagaa gcaagatact cctcagtaca |
| 1381 | acttaacgat agaggtgtct gacaaagatt tcaagaccct ttgttttgtg caaatcaacg |
| 1441 | ttattgatat caatgatcag atccccatct ttgaaaaatc agattatgga aacctgactc |
| 1501 | ttgctgaaga cacaaacatt gggtccacca tcttaaccat ccaggccact gatgctgatg |
| 1561 | agccatttac tgggagttct aaaattctgt atcatatcat aaagggagac agtgagggac |
| 1621 | gcctgggggt tgacacagat ccccatacca acaccggata tgtcataatt aaaaagcctc |
| 1681 | ttgattttga aacagcagct gtttccaaca ttgtgttcaa agcagaaaat cctgagcctc |
| 1741 | tagtgtttgg tgtgaagtac aatgcaagtt cttttgccaa gttcacgctt attgtgacag |
| 1801 | atgtgaatga agcacctcaa ttttcccaac acgtattcca agcgaaagtc agtgaggatg |
| 1861 | tagctatagg cactaaagtg ggcaatgtga ctgccaagga tccagaaggt ctggacataa |
| 1921 | gctattcact gaggggagac acaagaggtt ggcttaaaat tgaccacgtg actggtgaga |
| 1981 | tctttagtgt ggctccattg gacagagaag ccggaagtcc atatcgggta caagtggtgg |
| 2041 | ccacagaagt aggggggtct tccttgagct ctgtgtcaga gttccacctg atccttatgg |
| 2101 | atgtgaatga caaccctccc aggctagcca aggactacac gggcttgttc ttctgccatc |
| 2161 | ccctcagtgc acctggaagt ctcattttcg aggctactga tgatgatcag cacttatttc |
| 2221 | ggggtcccca ttttacattt tccctcggca gtggaagctt acaaaacgac tgggaagttt |
| 2281 | ccaaaatcaa tggtactcat gcccgactgt ctaccaggca cacagagttt gaggagaggg |
| 2341 | agtatgtcgt cttgatccgc atcaatgatg ggggtcggcc acccttggaa ggcattgttt |
| 2401 | ctttaccagt tacattctgc agttgtgtgg aaggaagttg tttccggcca gcaggtcacc |
| 2461 | agactgggat acccactgtg ggcatggcag ttggtatact gctgaccacc cttctggtga |
| 2521 | ttggtataat tttagcagtt gtgtttatcc gcataaagaa ggataaaggc aaagataatg |
| 2581 | ttgaaagtgc tcaagcatct gaagtcaaac ctctgagaag ctgaatttga aaaggaatgt |
| 2641 | ttgaatttat atagcaagtg ctatttcagc aacaaccatc tcatcctatt acttttcatc |
| 2701 | taacgtgcat tataattttt taaacagata ttccctcttg tcctttaata tttgctaaat |
| 2761 | atttcttttt tgaggtggag tcttgctctg tcgcccaggc tggagtacag tggtgtgatc |
| 2821 | ccagctcact gcaacctccg cctcctgggt tcacatgatt ctcctgcctc agcttcctaa |
| 2881 | gtagctgggt ttacaggcac ccaccaccat gcccagctaa tttttgtatt tttaatagag |
| 2941 | acggggtttc gccatttggc caggctggtc ttgaactcct gacgtcaagt gatctgcctg |
| 3001 | ccttggtctc ccaatacagg catgaaccac tgcacccacc tacttagata tttcatgtgc |
| 3061 | tatagacatt agagagattt ttcatttttc catgacattt ttcctctctg caaatggctt |
| 3121 | agctacttgt gtttttccct tttggggcaa gacagactca ttaaatattc tgtacatttt |
| 3181 | ttctttatca aggagatata tcagtgttgt ctcatagaac tgcctggatt ccatttatgt |
| 3241 | tttttctgat tccatcctgt gtccccttca tccttgactc ctttggtatt tcactgaatt |
| 3301 | tcaaacattt gtcagagaag aaaaacgtga ggactcagga aaaataaata aataaaagaa |
| 3361 | cagccttttc ccttagtatt aacagaaatg tttctgtgtc attaaccatc tttaatcaat |
| 3421 | gtgacatgtt gctctttggc tgaaattctt caacttggaa atgacacaga cccacagaag |
| 3481 | gtgttcaaac acaacctact ctgcaaacct tggtaaagga accagtcagc tggccagatt |
| 3541 | tcctcactac ctgccatgca tacatgctgc gcatgttttc ttcattcgta tgttagtaaa |
| 3601 | gttttggtta ttatatattt aacatgtgga agaaaacaag acatgaaaag agtggtgaca |
| 3661 | aatcaagaat aaacactggt tgtagtcagt tttgtttg |

NCBI Reference Sequence: NM_{—}001144663.1; Transcript Variant 2

| (SEQ ID NO: 39) | |
|---|---|
| 1 | aatcacggtg gaagtatgat attttggctg tggatctgag ttgatcaatc tgcttagtgg |
| 61 | acttgagtcc ccccaccccc gcttgtctga ttggggctcc tgggaggaat ttgaataaag |
| 121 | aaaactatga tacttcaggc ccatcttcac tccctgtgtc ttcttatgct ttatttggca |
| 181 | actggatatg gccaagaggg gaagtttagt ggacccctga aacccatgac attttctatt |
| 241 | tatgaaggcc aagaaccgag tcaaattata ttccagttta aggccaatcc tcctgctgtg |
| 301 | acttttgaac taactgggga gacagacaac atatttgtga tagaacggga gggacttctg |
| 361 | tattacaaca gagccttgga cagggaaaca agatctactc acaatctcca ggttgcagcc |
| 421 | ctggacgcta atggaattat agtggagggt ccagtcccta tcaccataaa agtgaaggac |
| 481 | atcaacgaca atcgacccac gtttctccag tcaaagtacg aaggctcagt aaggcagaac |
| 541 | tctcgcccag gaaagccctt cttgtatgtc aatgccacag acctggatga tccggccact |
| 601 | cccaatggcc agctttatta ccagattgtc atccagcttc ccatgatcaa caatgtcatg |
| 661 | tactttcaga tcaacaacaa aacgggagcc atctctctta cccgagaggg atctcaggaa |
| 721 | ttgaatcctg ctaagaatcc ttcctataat ctggtgatct cagtgaagga catgggaggc |
| 781 | cagagtgaga attccttcag tgataccaca tctgtggata tcatagtgac agagaatatt |
| 841 | tggaaagcac caaaacctgt ggagatggtg gaaaactcaa ctgatcctca ccccatcaaa |
| 901 | atcactcagg tgcggtggaa tgatcccggt gcacaatatt ccttagttga caaagagaag |
| 961 | ctgccaagat tcccattttc aattgaccag gaaggagata tttacgtgac tcagcccttg |
| 1021 | gaccgagaag aaaaggatgc atatgttttt tatgcagttg caaaggatga gtacggaaaa |
| 1081 | ccactttcat atccgctgga aattcatgta aaagttaaag atattaatga taatccacct |
| 1141 | acatgtccgt caccagtaac cgtatttgag gtccaggaga atgaacgact gggtaacagt |
| 1201 | atcgggaccc ttactgcaca tgacagggat gaagaaaata ctgccaacag ttttctaaac |
| 1261 | tacaggattg tggagcaaac tcccaaactt cccatggatg gactcttcct aatccaaacc |
| 1321 | tatgctggaa tgttacagtt agctaaacag tccttgaaga agcaagatac tcctcagtac |
| 1381 | aacttaacga tagaggtgtc tgacaaagat ttcaagaccc tttgttttgt gcaaatcaac |
| 1441 | gttattgata tcaatgatca gatccccatc tttgaaaaat cagattatgg aaacctgact |
| 1501 | cttgctgaag acacaaacat tgggtccacc atcttaacca tccaggccac tgatgctgat |
| 1561 | gagccattta ctgggagttc taaaattctg tatcatatca taaagggaga cagtgaggga |
| 1621 | cgcctggggg ttgacacaga tccccatacc aacaccggat atgtcataat taaaaagcct |
| 1681 | cttgattttg aaacagcagc tgtttccaac attgtgttca aagcagaaaa tcctgagcct |
| 1741 | ctagtgtttg gtgtgaagta caatgcaagt tcttttgcca agttcacgct tattgtgaca |
| 1801 | gatgtgaatg aagcacctca attttcccaa cacgtattcc aagcgaaagt cagtgaggat |
| 1861 | gtagctatag gcactaaagt gggcaatgtg actgccaagg atccagaagg tctggacata |
| 1921 | agctattcac tgaggggaga cacaagaggt tggcttaaaa ttgaccacgt gactggtgag |
| 1981 | atctttagtg tggctccatt ggacagagaa gccggaagtc catatcgggt acaagtggtg |
| 2041 | gccacagaag taggggggtc ttccttgagc tctgtgtcag agttccacct gatccttatg |
| 2101 | gatgtgaatg acaaccctcc caggctagcc aaggactaca cgggcttgtt cttctgccat |
| 2161 | cccctcagtg cacctggaag tctcattttc gaggctactg atgatgatca gcacttattt |
| 2221 | cggggtcccc attttacatt ttccctcggc agtggaagct tacaaaacga ctgggaagtt |
| 2281 | tccaaaatca atggtactca tgcccgactg tctaccaggc acacagagtt tgaggagagg |
| 2341 | gagtatgtcg tcttgatccg catcaatgat gggggtcggc cacccttgga aggcattgtt |
| 2401 | tctttaccag ttacattctg cagttgtgtg gaaggaagtt gtttccggcc agcaggtcac |
| 2461 | cagactggga tacccactgt gggcatggca gttggtatac tgctgaccac ccttctggtg |
| 2521 | attggtataa ttttagcagt tgtgtttatc cgcataaaga aggataaagg caaagataat |
| 2581 | gttgaaagtg ctcaagcatc tgaagtcaaa cctctgagaa gctgaatttg aaaaggaatg |
| 2641 | tttgaattta tatagcaagt gctatttcag caacaaccat ctcatcctat tacttttcat |
| 2701 | ctaacgtgca ttataatttt ttaaacagat attccctctt gtcctttaat atttgctaaa |
| 2761 | tatttctttt ttgaggtgga gtcttgctct gtcgcccagg ctggagtaca gtggtgtgat |
| 2821 | cccagctcac tgcaacctcc gcctcctggg ttcacatgat tctcctgcct cagcttccta |
| 2881 | agtagctggg tttacaggca cccaccacca tgcccagcta atttttgtat ttttaataga |
| 2941 | gacggggttt cgccatttgg ccaggctggt cttgaactcc tgacgtcaag tgatctgcct |
| 3001 | gccttggtct cccaatacag gcatgaacca ctgcacccac ctacttagat atttcatgtg |
| 3061 | ctatagacat tagagagatt tttcattttt ccatgacatt tttcctctct gcaaatggct |
| 3121 | tagctacttg tgtttttccc ttttggggca agacagactc attaaatatt ctgtacattt |
| 3181 | tttctttatc aaggagatat atcagtgttg tctcatagaa ctgcctggat tccatttatg |
| 3241 | ttttttctga ttccatcctg tgtccccttc atccttgact cctttggtat ttcactgaat |
| 3301 | ttcaaacatt tgtcagagaa gaaaaacgtg aggactcagg aaaaataaat aaataaaaga |
| 3361 | acagcctttt cccttagtat taacagaaat gtttctgtgt cattaaccat ctttaatcaa |
| 3421 | tgtgacatgt tgctctttgg ctgaaattct tcaacttgga aatgacacag acccacagaa |
| 3481 | ggtgttcaaa cacaacctac tctgcaaacc ttggtaaagg aaccagtcag ctggccagat |
| 3541 | ttcctcacta cctgccatgc atacatgctg cgcatgtttt cttcattcgt atgttagtaa |
| 3601 | agttttggtt attatatatt taacatgtgg aagaaaacaa gacatgaaaa gagtggtgac |
| 3661 | aaatcaagaa taaacactgg ttgtagtcag ttttgtttg |

### CDH6

Cadherin 6, type 2, K-cadherin (fetal kidney) (CDH6), also known as CAD6 pr KCAD is a member of the cadherin superfamily calcium-dependent cell-cell adhesion molecules that mediate cell-cell binding in a hemophilic manner. The full-length CDH6 cDNA was cloned by, Shimoyama et al. 1995 (Cancer Res. 55:2206-2211). CDH6 expression has been detected in pancreatic stem cells, and renal stem cells. RNA expression can be measure for example by RT-PCR, RT-qPCR, RNA-Seq, microarray approaches or RNA in situ hybridization.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art.

The human cDNA sequence is listed below (NCBI Reference Sequence: NM_{—}004932.3):

| (SEQ ID NO: 40) | |
|---|---|
| 1 | ctttaacaaa gtcctcctct ctttgctccc tcccacttca ttcacttgca aatcagtgtg |
| 61 | tgcccacaag agccagctct cccgagcccg taaccttcgc atcccaagag ctgcagtttc |
| 121 | agccgcgaca gcaagaacgg cagagccggc gaccgcggcg gcggcggcgg cggaggcagg |
| 181 | agcagcctgg gcgggtcgca gggtctccgc gggcgcagga aggcgagcag agatatcctc |
| 241 | tgagagccaa gcaaagaaca ttaaggaagg aaggaggaat gaggctggat acggtgcagt |
| 301 | gaaaaaggca cttccaagag tggggcactc actacgcaca gactcgacgg tgccatcagc |
| 361 | atgagaactt accgctactt cttgctgctc ttttgggtgg gccagcccta cccaactctc |
| 421 | tcaactccac tatcaaagag gactagtggt ttcccagcaa agaaaagggc cctggagctc |
| 481 | tctggaaaca gcaaaaatga gctgaaccgt tcaaaaagga gctggatgtg gaatcagttc |
| 541 | tttctcctgg aggaatacac aggatccgat tatcagtatg tgggcaagtt acattcagac |
| 601 | caggatagag gagatggatc acttaaatat atcctttcag gagatggagc aggagatctc |
| 661 | ttcattatta atgaaaacac aggcgacata caggccacca agaggctgga cagggaagaa |
| 721 | aaacccgttt acatccttcg agctcaagct ataaacagaa ggacagggag acccgtggag |
| 781 | cccgagtctg aattcatcat caagatccat gacatcaatg acaatgaacc aatattcacc |
| 841 | aaggaggttt acacagccac tgtccctgaa atgtctgatg tcggtacatt tgttgtccaa |
| 901 | gtcactgcga cggatgcaga tgatccaaca tatgggaaca gtgctaaagt tgtctacagt |
| 961 | attctacagg gacagcccta tttttcagtt gaatcagaaa caggtattat caagacagct |
| 1021 | ttgctcaaca tggatcgaga aaacagggag cagtaccaag tggtgattca agccaaggat |
| 1081 | atgggcggcc agatgggagg attatctggg accaccaccg tgaacatcac actgactgat |
| 1141 | gtcaacgaca accctccccg attcccccag agtacatacc agtttaaaac tcctgaatct |
| 1201 | tctccaccgg ggacaccaat tggcagaatc aaagccagcg acgctgatgt gggagaaaat |
| 1261 | gctgaaattg agtacagcat cacagacggt gaggggctgg atatgtttga tgtcatcacc |
| 1321 | gaccaggaaa cccaggaagg gattataact gtcaaaaagc tcttggactt tgaaaagaag |
| 1381 | aaagtgtata cccttaaagt ggaagcctcc aatccttatg ttgagccacg atttctctac |
| 1441 | ttggggcctt tcaaagattc agccacggtt agaattgtgg tggaggatgt agatgagcca |
| 1501 | cctgtcttca gcaaactggc ctacatctta caaataagag aagatgctca gataaacacc |
| 1561 | acaataggct ccgtcacagc ccaagatcca gatgctgcca ggaatcctgt caagtactct |
| 1621 | gtagatcgac acacagatat ggacagaata ttcaacattg attctggaaa tggttcgatt |
| 1681 | tttacatcga aacttcttga ccgagaaaca ctgctatggc acaacattac agtgatagca |
| 1741 | acagagatca ataatccaaa gcaaagtagt cgagtacctc tatatattaa agttctagat |
| 1801 | gtcaatgaca acgccccaga atttgctgag ttctatgaaa cttttgtctg tgaaaaagca |
| 1861 | aaggcagatc agttgattca gaccctgcat gctgttgaca aggatgaccc ttatagtgga |
| 1921 | caccaatttt cgttttcctt ggcccctgaa gcagccagtg gctcaaactt taccattcaa |
| 1981 | gacaacaaag acaacacggc gggaatctta actcggaaaa atggctataa tagacacgag |
| 2041 | atgagcacct atctcttgcc tgtggtcatt tcagacaacg actacccagt tcaaagcagc |
| 2101 | actgggacag tgactgtccg ggtctgtgca tgtgaccacc acgggaacat gcaatcctgc |
| 2161 | catgcggagg cgctcatcca ccccacggga ctgagcacgg gggctctggt tgccatcctt |
| 2221 | ctgtgcatcg tgatcctact agtgacagtg gtgctgtttg cagctctgag gcggcagcga |
| 2281 | aaaaaagagc ctttgatcat ttccaaagag gacatcagag ataacattgt cagttacaac |
| 2341 | gacgaaggtg gtggagagga ggacacccag gcttttgata tcggcaccct gaggaatcct |
| 2401 | gaagccatag aggacaacaa attacgaagg gacattgtgc ccgaagccct tttcctaccc |
| 2461 | cgacggactc caacagctcg cgacaacacc gatgtcagag atttcattaa ccaaaggtta |
| 2521 | aaggaaaatg acacggaccc cactgccccg ccatacgact ccttggccac ttacgcctat |
| 2581 | gaaggcactg gctccgtggc ggattccctg agctcgctgg agtcagtgac cacggatgca |
| 2641 | gatcaagact atgattacct tagtgactgg ggacctcgat tcaaaaagct tgcagatatg |
| 2701 | tatggaggag tggacagtga caaagactcc taatctgttg cctttttcat tttccaatac |
| 2761 | gacactgaaa tatgtgaagt ggctatttct ttatatttat ccactactcc gtgaaggctt |
| 2821 | ctctgttcta cccgttccaa aagccaatgg ctgcagtccg tgtggatcca atgttagaga |
| 2881 | cttttttcta gtacactttt atgagcttcc aaggggcaaa tttttatttt ttagtgcatc |
| 2941 | cagttaacca agtcagccca acaggcaggt gccggagggg aggacaggga acagtatttc |
| 3001 | cacttgttct cagggcagcg tgcccgcttc cgctgtcctg gtgttttact acactccatg |
| 3061 | tcaggtcagc caactgccct aactgtacat ttcacaggct aatgggataa aggactgtgc |
| 3121 | tttaaagata aaaatatcat catagtaaaa gaaatgaggg catatcggct cacaaagaga |
| 3181 | taaactacat aggggtgttt atttgtgtca caaagaattt aaaataacac ttgcccatgc |
| 3241 | tatttgttct tcaagaactt tctctgccat caactactat tcaaaacctc aaatccaccc |
| 3301 | atatgttaaa attctcatta ctcttaagga atagaagcaa attaaacggt aacatccaaa |
| 3361 | agcaaccaca aacctagtac gacttcattc cttccactaa ctcatagttt gttatatcct |
| 3421 | agactagaca tgcgaaagtt tgcctttgta ccatataaag ggggagggaa atagctaata |
| 3481 | atgttaacca aggaaatata ttttaccata catttaaagt tttggccacc acatgtatca |
| 3541 | cgggtcactt gaaattcttt cagctatcag taggctaatg tcaaaattgt ttaaaaattc |
| 3601 | ttgaaagaat tttcctgaga caaattttaa cttcttgtct atagttgtca gtattattct |
| 3661 | actatactgt acatgaaagt agcagtgtga agtacaataa ttcatattct tcatatcctt |
| 3721 | cttacacgac taagttgaat tagtaaagtt agattaaata aaacttaaat ctcactctag |
| 3781 | gagttcagtg gagaggttag agccagccac acttgaacct aataccctgc ccttgacatc |
| 3841 | tggaaacctc tacatattta tataacgtga tacatttgga taaacaacat tgagattatg |
| 3901 | atgaaaacct acatattcca tgtttggaag acccttggaa gaggaaaatt ggattccctt |
| 3961 | aaacaaaagt gtttaagatt gtaattaaaa tgatagttga ttttcaaaag cattaatttt |
| 4021 | ttttcattgt ttttaacttt gctttcatga ccatcctgcc atccttgact ttgaactaat |
| 4081 | gataaagtaa tgatctcaaa ctatgacaga aaagtaatgt aaaatccatc caatctatta |
| 4141 | tttctctaat tatgcaatta gcctcatagt tattatccag aggacccaac tgaactgaac |
| 4201 | taatccttct ggcagattca aatcgtttat ttcacacgct gttctaatgg cacttatcat |
| 4261 | tagaatctta ccttgtgcag tcatcagaaa ttccagcgta ctataatgaa aacatccttg |
| 4321 | ttttgaaaac ctaaaagaca ggctctgtat atatatatac ttaagaatat gctgacttca |
| 4381 | cttattagtc ttagggattt attttcaatt aatattaatt ttctacaaat aattttagtg |
| 4441 | tcatttccat ttggggatat tgtcatatca gcacatattt tctgtttgga aacacactgt |
| 4501 | tgtttagtta agttttaaat aggtgtatta cccaagaagt aaagatggaa acgttaaaag |
| 4561 | aagagaaatg tagtattttg ggttacctga ttagagtgaa aattttttac aatcatatta |
| 4621 | ttccttgtgt cttctgaatg gtttccgatt ttataatgga ctgccctata tagtaacaag |
| 4681 | tatttcatgc ttgagctatt tcctgctttc agggtttctt ttttctagtt cttcatacac |
| 4741 | acacatacac acacacacac acacacacac acacacacac gaatgcaaac aaaaggctat |
| 4801 | atgaggtctt cactctaatg aattgatatg tatcatagtc acaggtaagt gttgaaaaaa |
| 4861 | gcttagtaaa gttagaagct acttactcat agcaatagaa cagcacctta atcacacgat |
| 4921 | ttactgtaaa attaaagagg tctctatctg tatgtttcat gtcacgtaac aaattgaatc |
| 4981 | aaggaagata gtcctgtaaa aagaaaggta tcatctgaag ttgaggattg acactagcag |
| 5041 | tttccaatgt ttaaaggtaa gatctgagtt ctcctaataa gtaaaagtaa gtagttctat |
| 5101 | agcagaatat ctgagatgta attggcaagg tattttatcc ctccctgcag atgacacagc |
| 5161 | ataccaagaa caggttaata tgattactta tggaaataac tttaatctct tatcataaaa |
| 5221 | gctgatgatg aagtaaattt ataggaaatt ggataatttg agactggggc taaatattta |
| 5281 | gtaccagggt actgtaagta tcaagttgga gtgacgtttt cctataattc agactctttg |
| 5341 | acatcgtgga accaataaga gtcatagttc catcattctc cagcttcgtc tcacttcctt |
| 5401 | cccaccccac ctgagtatca ggtcaaacat cattgcatgc gcaggttttt tttttaattg |
| 5461 | ctaggtccca ggcaacatga aagattattg gagaaaaaaa taattttcag cccagttttt |
| 5521 | tcattgtctg tttcctaatt ttagatgttg gtgatgggaa agatggaagg agagtgggaa |
| 5581 | gaagtaaaat tttaatattt gtttcaatca ctttgaaact aaaattcatt aagcataacc |
| 5641 | agattgcttt tgtgggttgt ttcaaggaca ttgagagctt tctgatgata tgtttttgcc |
| 5701 | ctctattcaa aagcaagagt tcctttaaac tactaagata ttccctagaa taagctgaat |
| 5761 | ttaaaaaaac attaagccat tgtttaaagc cccttcactt cctggccact tacttctgaa |
| 5821 | aggcctaaaa aacatttgtg cccaaataag taaataaacc aaatgggaaa gaagcaaaga |
| 5881 | ttattccata gaaccacaag agagggaatg tgggcacagt aaatagatgt ttctttcaga |
| 5941 | actttcctgc ctttacagtt tgtgtccata aagggatgtt cagcaatgaa attactccct |
| 6001 | tttcagatgg aacaaaacct gcccatttaa ttttaacgca gtataaaaaa cgtgtggttt |
| 6061 | agtttttatt ttcagctccc aaagagttgt gcagaaaatc ttaaaatttt tttttttttt |
| 6121 | tttttttttt ttttgagaca gaatctcgct ctgtcgccca ggctggaatg cagtggcgcg |
| 6181 | atctctgctc actacaagct ccgcctcccg ggttcacgcc attctcctgc ctcagcgccc |
| 6241 | ccagtagctg ggactacagg cacacaccac cacgcccggc taatttttgt tgtatttttt |
| 6301 | agtggagaca gggtttcacc atgttagcca ggatgatctc catctcctga cctcgtggtc |
| 6361 | cgcccgcctc ggcctcccaa agtgctggga ttacaggcgt gagccaccgc gcccggcctt |
| 6421 | aaaaattgaa tctgtagctt aggccatcca aattttataa atccaaatta actttagaat |
| 6481 | gtttctatta ctttcacttt tacatatata aattttaagt gtcctgattg gctgaacaat |
| 6541 | atctcacatc aaatgctttg cctggaaata gatatcccac tggggatagt ggtgtgtaaa |
| 6601 | ctatgacttg gacaattcta tatactcaag caccataaaa agtatgcagt tgaaaagaaa |
| 6661 | atcaaagttg attcctgggt gccaactaaa tattcaaatc aggtactcat ccttatcagc |
| 6721 | taaattcatt ttcaccagga acagaccacc aaataaatta ttttatccta ataactagtt |
| 6781 | ttgaagcagt gtaattactc tggaagaagg ctctaaaaag tcatgattcc cccactattt |
| 6841 | tgaaatgtat cctctaacaa ggatcattat agtgtaatct taatttttat gttttatcaa |
| 6901 | gatgaaatct tgtttgaatt gtgatattat aaaaggggac tcaaaaatcc aagcagtcta |
| 6961 | ctgtgtttaa attaacacca caaccttcct tatcagatta taagagtaga aaaattaaca |
| 7021 | cttggtgtgt gaatcttcag gaaaatgagc tatttcataa gctcaaacaa gcagcttcct |
| 7081 | tttccagaga atatagaatt atattatggt ctccttaaat gtttagtagc tcttatggtc |
| 7141 | acagcatttt taatctccct atggcatctt tatggaataa ttttctaaag ggtaattctc |
| 7201 | tactaaaaat atcagacccc gaccatattt aatgtggaga gcaataccct cttagaaaga |
| 7261 | aaatacattg actcatacac ttgttaaaag ttaataaaga aatagctcat ttttaaagcc |
| 7321 | ggaagtttat ggtctctgca tcgtcaattt aatttaagca ttgctgagac aatctttaat |
| 7381 | ctactcccct ttttgtaata ccttatttat ggtgcatttt catttttatt tgggggaaac |
| 7441 | gttagcccaa cagagccggc agatgaaagt gttgaaaaga ggtcaaatgg aaacaaaggc |
| 7501 | tcttacccgc tgtatttcag acaggactga ggcacttagc cgaggagcca ctgggttatt |
| 7561 | agattaattt caaaagagct tttacaagtt gcttaattcc tttttttttt tttttttttt |
| 7621 | tcaaaaaccc atgaaccaca aactcaaatt tctcctcaaa tggggttaat ctgacaaacg |
| 7681 | aggcatggac ccagccttgt ggaaaaagca ttccacgcta atgagatctt ggtctttctt |
| 7741 | gtgaggctac gttatttatg taaatatgtc tggaggcacc ttctctaagc ttttagtttt |
| 7801 | ctatgatcta ttagtttagt gtttattaaa gaatcaaatg tatagaatta ccaggcattc |
| 7861 | gtggggaatg ctgtgtagca aatgtaaaac tgacctgctc ggaagaaacg taggaacgct |
| 7921 | tcaaacccac tgtaatgttt ggtttgagat tattttcatt gctttgagag tgaactgcct |
| 7981 | aagagtaggc cttataataa atgctatgtg cgtcttcagt agttccaagc taaagcaatt |
| 8041 | tggcattctc ccactgtgat ttgtgacttt taaacccaca aaataaaagc tttttggtat |
| 8101 | tgattgtttt taattaaaaa tacttccaag tataaattga aacggatgcc acccttgaag |
| 8161 | atttactggc gggaatgctc actcttgtcg ttttcctcag tatcgttcat gtctttggca |
| 8221 | acaagaacac ctgatgaaag caagcaatgc tcagttccca tcaacatttc tagttagggg |
| 8281 | gattctcata accccacagt ttacctgaga aagttttctg tgttagaaga atggggtcga |
| 8341 | gagtattacc ttttagctca gtgtggccgg gccttttgtt gcagtcaaat ggcaaatacg |
| 8401 | cactccttga aatggcttct tttatttggt tttgttttct tagacttata aatttgaaaa |
| 8461 | gaatgcaatt taaaaagtga tttctcacaa agagtaaata tgccttttgc aaatcaattt |
| 8521 | ttgtaacaag ttatttatat gatattactt aataaactgg tttttttcta a |

### CLDN18

Claudin 18 (CLDN18), also known as surfactant associated 5 (SFTA5), surfactant associated protein J or SFTPJ is a member of the claudin family. Claudins are integral membrane proteins and components of tight junction strands. CLDN18 expression has been detected in small intestinal stem cells, colon stem cells, and gastric stem cells. RNA expression can be measure for example by RT-PCR, RT-qPCR, RNA-Seq, microarray approaches or RNA in situ hybridization. Protein expression, measurable for example by immunofluorescence, immunohistochemistry, FACS, flow cytometry, Western blot or ELISA of CLDN18 can be used to characterize the stem cells.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA) or Abnova (Neihu District. Taipei City, Taiwan). For example, Niimi et al. (Mol. Cell Biol. 2001, 21(21):7380-90) describes RT-PCR primers and the generation of CLDN18 specific antibodies, and the differences between the two isoforms with isoform 2 being prevalent in stomach.

The human cDNA sequences are listed below (NM_{—}016369.3 claudin-18 isoform 1 precursor and NM_{—}001002026. claudin-18 isoform 2):

NCBI Reference Sequence: NM_{—}016369.3 Claudin-18 Isoform 1 Precursor

| (SEQ ID NO: 41) | |
|---|---|
| 1 | cacaccttcg gcagcaggag ggcggcagct tctcgcaggc ggcagggcgg gcggccagga |
| 61 | tcatgtccac caccacatgc caagtggtgg cgttcctcct gtccatcctg gggctggccg |
| 121 | gctgcatcgc ggccaccggg atggacatgt ggagcaccca ggacctgtac gacaaccccg |
| 181 | tcacctccgt gttccagtac gaagggctct ggaggagctg cgtgaggcag agttcaggct |
| 241 | tcaccgaatg caggccctat ttcaccatcc tgggacttcc agccatgctg caggcagtgc |
| 301 | gagccctgat gatcgtaggc atcgtcctgg gtgccattgg cctcctggta tccatctttg |
| 361 | ccctgaaatg catccgcatt ggcagcatgg aggactctgc caaagccaac atgacactga |
| 421 | cctccgggat catgttcatt gtctcaggtc tttgtgcaat tgctggagtg tctgtgtttg |
| 481 | ccaacatgct ggtgactaac ttctggatgt ccacagctaa catgtacacc ggcatgggtg |
| 541 | ggatggtgca gactgttcag accaggtaca catttggtgc ggctctgttc gtgggctggg |
| 601 | tcgctggagg cctcacacta attgggggtg tgatgatgtg catcgcctgc cggggcctgg |
| 661 | caccagaaga aaccaactac aaagccgttt cttatcatgc ctcaggccac agtgttgcct |
| 721 | acaagcctgg aggcttcaag gccagcactg gctttgggtc caacaccaaa aacaagaaga |
| 781 | tatacgatgg aggtgcccgc acagaggacg aggtacaatc ttatccttcc aagcacgact |
| 841 | atgtgtaatg ctctaagacc tctcagcacg ggcggaagaa actcccggag agctcaccca |
| 901 | aaaaacaagg agatcccatc tagatttctt cttgcttttg actcacagct ggaagttaga |
| 961 | aaagcctcga tttcatcttt ggagaggcca aatggtctta gcctcagtct ctgtctctaa |
| 1021 | atattccacc ataaaacagc tgagttattt atgaattaga ggctatagct cacattttca |
| 1081 | atcctctatt tcttttttta aatataactt tctactctga tgagagaatg tggttttaat |
| 1141 | ctctctctca cattttgatg atttagacag actccccctc ttcctcctag tcaataaacc |
| 1201 | cattgatgat ctatttccca gcttatcccc aagaaaactt ttgaaaggaa agagtagacc |
| 1261 | caaagatgtt attttctgct gtttgaattt tgtctcccca cccccaactt ggctagtaat |
| 1321 | aaacacttac tgaagaagaa gcaataagag aaagatattt gtaatctctc cagcccatga |
| 1381 | tctcggtttt cttacactgt gatcttaaaa gttaccaaac caaagtcatt ttcagtttga |
| 1441 | ggcaaccaaa cctttctact gctgttgaca tcttcttatt acagcaacac cattctagga |
| 1501 | gtttcctgag ctctccactg gagtcctctt tctgtcgcgg gtcagaaatt gtccctagat |
| 1561 | gaatgagaaa attatttttt ttaatttaag tcctaaatat agttaaaata aataatgttt |
| 1621 | tagtaaaatg atacactatc tctgtgaaat agcctcaccc ctacatgtgg atagaaggaa |
| 1681 | atgaaaaaat aattgctttg acattgtcta tatggtactt tgtaaagtca tgcttaagta |
| 1741 | caaattccat gaaaagctca ctgatcctaa ttctttccct ttgaggtctc tatggctctg |
| 1801 | attgtacatg atagtaagtg taagccatgt aaaaagtaaa taatgtctgg gcacagtggc |
| 1861 | tcacgcctgt aatcctagca ctttgggagg ctgaggagga aggatcactt gagcccagaa |
| 1921 | gttcgagact agcctgggca acatggagaa gccctgtctc tacaaaatac agagagaaaa |
| 1981 | aatcagccag tcatggtggc ctacacctgt agtcccagca ttccgggagg ctgaggtggg |
| 2041 | aggatcactt gagcccaggg aggttggggc tgcagtgagc catgatcaca ccactgcact |
| 2101 | ccagccaggt gacatagcga gatcctgtct aaaaaaataa aaaataaata atggaacaca |
| 2161 | gcaagtccta ggaagtaggt taaaactaat tctttaaaaa aaaaaaaaag ttgagcctga |
| 2221 | attaaatgta atgtttccaa gtgacaggta tccacatttg catggttaca agccactgcc |
| 2281 | agttagcagt agcactttcc tggcactgtg gtcggttttg ttttgttttg ctttgtttag |
| 2341 | agacggggtc tcactttcca ggctggcctc aaactcctgc actcaagcaa ttcttctacc |
| 2401 | ctggcctccc aagtagctgg aattacaggt gtgcgccatc acaactagct ggtggtcagt |
| 2461 | tttgttactc tgagagctgt tcacttctct gaattcacct agagtggttg gaccatcaga |
| 2521 | tgtttgggca aaactgaaag ctctttgcaa ccacacacct tccctgagct tacatcactg |
| 2581 | cccttttgag cagaaagtct aaattccttc caagacagta gaattccatc ccagtaccaa |
| 2641 | agccagatag gccccctagg aaactgaggt aagagcagtc tctaaaaact acccacagca |
| 2701 | gcattggtgc aggggaactt ggccattagg ttattatttg agaggaaagt cctcacatca |
| 2761 | atagtacata tgaaagtgac ctccaagggg attggtgaat actcataagg atcttcaggc |
| 2821 | tgaacagact atgtctgggg aaagaacgga ttatgcccca ttaaataaca agttgtgttc |
| 2881 | aagagtcaga gcagtgagct cagaggccct tctcactgag acagcaacat ttaaaccaaa |
| 2941 | ccagaggaag tatttgtgga actcactgcc tcagtttggg taaaggatga gcagacaagt |
| 3001 | caactaaaga aaaaagaaaa gcaaggagga gggttgagca atctagagca tggagtttgt |
| 3061 | taagtgctct ctggatttga gttgaagagc atccatttga gttgaaggcc acagggcaca |
| 3121 | atgagctctc ccttctacca ccagaaagtc cctggtcagg tctcaggtag tgcggtgtgg |
| 3181 | ctcagctggg tttttaatta gcgcattctc tatccaacat ttaattgttt gaaagcctcc |
| 3241 | atatagttag attgtgcttt gtaattttgt tgttgttgct ctatcttatt gtatatgcat |
| 3301 | tgagtattaa cctgaatgtt ttgttactta aatattaaaa acactgttat cctacagtt |

NCBI Reference Sequence: NM_{—}001002026.2 Claudin-18 Isoform 2

| (SEQ ID NO: 42) | |
|---|---|
| 1 | agaattgcgc tgtccacttg tcgtgtggct ctgtgtcgac actgtgcgcc accatggccg |
| 61 | tgactgcctg tcagggcttg gggttcgtgg tttcactgat tgggattgcg ggcatcattg |
| 121 | ctgccacctg catggaccag tggagcaccc aagacttgta caacaacccc gtaacagctg |
| 181 | ttttcaacta ccaggggctg tggcgctcct gtgtccgaga gagctctggc ttcaccgagt |
| 241 | gccggggcta cttcaccctg ctggggctgc cagccatgct gcaggcagtg cgagccctga |
| 301 | tgatcgtagg catcgtcctg ggtgccattg gcctcctggt atccatcttt gccctgaaat |
| 361 | gcatccgcat tggcagcatg gaggactctg ccaaagccaa catgacactg acctccggga |
| 421 | tcatgttcat tgtctcaggt ctttgtgcaa ttgctggagt gtctgtgttt gccaacatgc |
| 481 | tggtgactaa cttctggatg tccacagcta acatgtacac cggcatgggt gggatggtgc |
| 541 | agactgttca gaccaggtac acatttggtg cggctctgtt cgtgggctgg gtcgctggag |
| 601 | gcctcacact aattgggggt gtgatgatgt gcatcgcctg ccggggcctg gcaccagaag |
| 661 | aaaccaacta caaagccgtt tcttatcatg cctcaggcca cagtgttgcc tacaagcctg |
| 721 | gaggcttcaa ggccagcact ggctttgggt ccaacaccaa aaacaagaag atatacgatg |
| 781 | gaggtgcccg cacagaggac gaggtacaat cttatccttc caagcacgac tatgtgtaat |
| 841 | gctctaagac ctctcagcac gggcggaaga aactcccgga gagctcaccc aaaaaacaag |
| 901 | gagatcccat ctagatttct tcttgctttt gactcacagc tggaagttag aaaagcctcg |
| 961 | atttcatctt tggagaggcc aaatggtctt agcctcagtc tctgtctcta aatattccac |
| 1021 | cataaaacag ctgagttatt tatgaattag aggctatagc tcacattttc aatcctctat |
| 1081 | ttcttttttt aaatataact ttctactctg atgagagaat gtggttttaa tctctctctc |
| 1141 | acattttgat gatttagaca gactccccct cttcctccta gtcaataaac ccattgatga |
| 1201 | tctatttccc agcttatccc caagaaaact tttgaaagga aagagtagac ccaaagatgt |
| 1261 | tattttctgc tgtttgaatt ttgtctcccc acccccaact tggctagtaa taaacactta |
| 1321 | ctgaagaaga agcaataaga gaaagatatt tgtaatctct ccagcccatg atctcggttt |
| 1381 | tcttacactg tgatcttaaa agttaccaaa ccaaagtcat tttcagtttg aggcaaccaa |
| 1441 | acctttctac tgctgttgac atcttcttat tacagcaaca ccattctagg agtttcctga |
| 1501 | gctctccact ggagtcctct ttctgtcgcg ggtcagaaat tgtccctaga tgaatgagaa |
| 1561 | aattattttt tttaatttaa gtcctaaata tagttaaaat aaataatgtt ttagtaaaat |
| 1621 | gatacactat ctctgtgaaa tagcctcacc cctacatgtg gatagaagga aatgaaaaaa |
| 1681 | taattgcttt gacattgtct atatggtact ttgtaaagtc atgcttaagt acaaattcca |
| 1741 | tgaaaagctc actgatccta attctttccc tttgaggtct ctatggctct gattgtacat |
| 1801 | gatagtaagt gtaagccatg taaaaagtaa ataatgtctg ggcacagtgg ctcacgcctg |
| 1861 | taatcctagc actttgggag gctgaggagg aaggatcact tgagcccaga agttcgagac |
| 1921 | tagcctgggc aacatggaga agccctgtct ctacaaaata cagagagaaa aaatcagcca |
| 1981 | gtcatggtgg cctacacctg tagtcccagc attccgggag gctgaggtgg gaggatcact |
| 2041 | tgagcccagg gaggttgggg ctgcagtgag ccatgatcac accactgcac tccagccagg |
| 2101 | tgacatagcg agatcctgtc taaaaaaata aaaaataaat aatggaacac agcaagtcct |
| 2161 | aggaagtagg ttaaaactaa ttctttaaaa aaaaaaaaaa gttgagcctg aattaaatgt |
| 2221 | aatgtttcca agtgacaggt atccacattt gcatggttac aagccactgc cagttagcag |
| 2281 | tagcactttc ctggcactgt ggtcggtttt gttttgtttt gctttgttta gagacggggt |
| 2341 | ctcactttcc aggctggcct caaactcctg cactcaagca attcttctac cctggcctcc |
| 2401 | caagtagctg gaattacagg tgtgcgccat cacaactagc tggtggtcag ttttgttact |
| 2461 | ctgagagctg ttcacttctc tgaattcacc tagagtggtt ggaccatcag atgtttgggc |
| 2521 | aaaactgaaa gctctttgca accacacacc ttccctgagc ttacatcact gcccttttga |
| 2581 | gcagaaagtc taaattcctt ccaagacagt agaattccat cccagtacca aagccagata |
| 2641 | ggccccctag gaaactgagg taagagcagt ctctaaaaac tacccacagc agcattggtg |
| 2701 | caggggaact tggccattag gttattattt gagaggaaag tcctcacatc aatagtacat |
| 2761 | atgaaagtga cctccaaggg gattggtgaa tactcataag gatcttcagg ctgaacagac |
| 2821 | tatgtctggg gaaagaacgg attatgcccc attaaataac aagttgtgtt caagagtcag |
| 2881 | agcagtgagc tcagaggccc ttctcactga gacagcaaca tttaaaccaa accagaggaa |
| 2941 | gtatttgtgg aactcactgc ctcagtttgg gtaaaggatg agcagacaag tcaactaaag |
| 3001 | aaaaaagaaa agcaaggagg agggttgagc aatctagagc atggagtttg ttaagtgctc |
| 3061 | tctggatttg agttgaagag catccatttg agttgaaggc cacagggcac aatgagctct |
| 3121 | cccttctacc accagaaagt ccctggtcag gtctcaggta gtgcggtgtg gctcagctgg |
| 3181 | gtttttaatt agcgcattct ctatccaaca tttaattgtt tgaaagcctc catatagtta |
| 3241 | gattgtgctt tgtaattttg ttgttgttgc tctatcttat tgtatatgca ttgagtatta |
| 3301 | acctgaatgt tttgttactt aaatattaaa aacactgtta tcctacagtt |

### FXYD2

FXYD domain containing ion transport regulator 2 (FXYD2), also known as HOMG2 or ATP1G1, is member of the FXYD family of transmembrane proteins. This particular protein encodes the sodium/potassium-transporting ATPase subunit gamma.

FXYD2 expression has been detected in liver stem cells, pancreatic stem cells, and renal stem cells. RNA expression can be measure for example by RT-PCR, RT-qPCR, RNA-Seq, microarray approaches or RNA in situ hybridization. In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art.

The human cDNA sequences are listed below (NM_{—}001680.4 sodium/potassium-transporting ATPase subunit gamma isoform 1 and NM_{—}021603.3 sodium/potassium-transporting ATPase subunit gamma isoform 2):

NCBI Reference Sequence: NM_{—}001680.4 Sodium/Potassium-Transporting ATPase Subunit Gamma Isoform 1

| (SEQ ID NO: 43) | |
|---|---|
| 1 | agacactctc caaaaagcag agacagcagg aagaggggag tggaggcagc ccattcacct |
| 61 | ggggaaatga ctgggttgtc gatggacggt ggcggcagcc ccaaggggga cgtggacccg |
| 121 | ttctactatg actatgagac cgttcgcaat gggggcctga tcttcgctgg actggccttc |
| 181 | atcgtggggc tcctcatcct cctcagcaga agattccgct gtgggggcaa taagaagcgc |
| 241 | aggcaaatca atgaagatga gccgtaacag cagcctcggc ggtgccaccc actgcactgg |
| 301 | ggccagctgg gaagccaagc atggccctgc ctctggcgcc tccccttctt ccctgggctt |
| 361 | tagacctttg tccccgtcac tgccagcgct tgggctgaag gaagctccag actcaatgtg |
| 421 | acccccaggt ggcatcgcca actcctgcct cgtgccacct catgcttata ataaagccgg |
| 481 | cgtcagagac cgctgcttcc ctcacctgcc tgcctgtctc cctcctctgt caccaccagc |
| 541 | ctctccaagc tcaagtacaa atacagccgg gaaaaaaaaa aaaa |

NCBI Reference Sequence: NM_{—}021603.3 Sodium/Potassium-Transporting ATPase Subunit Gamma Isoform 2

| (SEQ ID NO: 44) | |
|---|---|
| 1 | gccactctcc atccaggccc caggcaagca gcacctccct gctctcctgc actcctggac |
| 61 | acaaccagca gctcctgcca tggacaggtg gtacctgggc ggcagcccca agggggacgt |
| 121 | ggacccgttc tactatgact atgagaccgt tcgcaatggg ggcctgatct tcgctggact |
| 181 | ggccttcatc gtggggctcc tcatcctcct cagcagaaga ttccgctgtg ggggcaataa |
| 241 | gaagcgcagg caaatcaatg aagatgagcc gtaacagcag cctcggcggt gccacccact |
| 301 | gcactggggc cagctgggaa gccaagcatg gccctgcctc tggcgcctcc ccttcttccc |
| 361 | tgggctttag acctttgtcc ccgtcactgc cagcgcttgg gctgaaggaa gctccagact |
| 421 | caatgtgacc cccaggtggc atcgccaact cctgcctcgt gccacctcat gcttataata |
| 481 | aagccggcgt cagagaccgc tgcttccctc acctgcctgc ctgtctccct cctctgtcac |
| 541 | caccagcctc tccaagctca agtacaaata cagccgggaa aaaaaaaaaa a |

### HEPH

Hephaestin (HEPH), also known as CPL, is s similar to an iron transport protein. Three transcript variants encoding different isoforms have been described.

HEPH expression has been detected in intestinal metaplasia stem cells. RNA expression can be measure for example by RT-PCR, RT-qPCR, RNA-Seq, microarray approaches or RNA in situ hybridization. Protein expression can be detected for example by immunofluorescence, immunohistochemistry, FACS, flow cytometry, Western blot or ELISA. In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA).

The human cDNA sequences are listed below (NM_{—}138737.3 hephaestin isoform; NM_{—}014799.2 hephaestin isoform b and NM_{—}001130860.2 hephaestin isoform c precursor):

NCBI Reference Sequence: NM_{—}138737.3 Hephaestin Isoform a

| (SEQ ID NO: 45) | |
|---|---|
| 1 | actgagcatt tctaagggag ttgaggctgg tggctcctcc ttccttccta ctggtgcttc |
| 61 | cacctgcctt ggtctgagtt gcagtccatg gggcagcgcc taagtgtctg agcacactta |
| 121 | agaatctcta gtggtttatg acccagactt tgccctacca cctcagtctt ctgaatgttc |
| 181 | tcttccctgg accctgctcc agacacttta aattcagaag aggaaaatgt gcccagcctg |
| 241 | cctggagaaa agtgtctgct cctagccaag atctcctcat cacaaaagta atgtgggcca |
| 301 | tggagtcagg ccacctcctc tgggctctgc tgttcatgca gtccttgtgg cctcaactga |
| 361 | ctgatggagc cactcgagtc tactacctgg gcatccggga tgtgcagtgg aactatgctc |
| 421 | ccaagggaag aaatgtcatc acgaaccagc ctctggacag tgacatagtg gcttccagct |
| 481 | tcttaaagtc tgacaagaac cggatagggg gaacctacaa gaagaccatc tataaagaat |
| 541 | acaaggatga ctcatacaca gatgaagtgg cccagcctgc ctggttgggc ttcctggggc |
| 601 | cagtgttgca ggctgaagtg ggggatgtca ttcttattca cctgaagaat tttgccactc |
| 661 | gtccctatac catccaccct catggtgtct tctacgagaa ggactctgaa ggttccctat |
| 721 | acccagatgg ctcctctggg ccactgaaag ctgatgactc tgttcccccg gggggcagcc |
| 781 | atatctacaa ctggaccatt ccagaaggcc atgcacccac cgatgctgac ccagcgtgcc |
| 841 | tcacctggat ctaccattct catgtagatg ctccacgaga cattgcaact ggcctaattg |
| 901 | ggcctctcat cacctgtaaa agaggagccc tggatgggaa ctcccctcct caacgccagg |
| 961 | atgtagacca tgatttcttc ctcctcttca gtgtggtaga tgagaacctc agctggcatc |
| 1021 | tcaatgagaa cattgccact tactgctcag atcctgcttc agtggacaaa gaagatgaga |
| 1081 | catttcagga gagcaatagg atgcatgcaa tcaatggctt tgtttttggg aatttacctg |
| 1141 | agctgaacat gtgtgcacag aaacgtgtgg cctggcactt gtttggcatg ggcaatgaaa |
| 1201 | ttgatgtcca cacagcattt ttccatggac agatgctgac tacccgtgga caccacactg |
| 1261 | atgtggctaa catctttcca gccacctttg tgactgctga gatggtgccc tgggaacctg |
| 1321 | gtacctggtt aattagctgc caagtgaaca gtcactttcg agatggcatg caggcactct |
| 1381 | acaaggtcaa gtcttgctcc atggcccctc ctgtggacct gctcacaggc aaagttcgac |
| 1441 | agtacttcat tgaggcccat gagattcaat gggactatgg cccgatgggg catgatggga |
| 1501 | gtactgggaa gaatttgaga gagccaggca gtatctcaga taagtttttc cagaagagct |
| 1561 | ccagccgaat tgggggcact tactggaaag tgcgatatga agcctttcaa gatgagacat |
| 1621 | tccaagagaa gatgcatttg gaggaagata ggcatcttgg aatcctgggg ccagtgatcc |
| 1681 | gggctgaggt gggtgacacc attcaggtgg tcttctacaa ccgtgcctcc cagccattca |
| 1741 | gcatgcagcc ccatggggtc ttttatgaga aagactatga aggcactgtg tacaatgatg |
| 1801 | gctcatctta ccctggcttg gttgccaagc cctttgagaa agtaacatac cgctggacag |
| 1861 | tcccccctca tgccggtccc actgctcagg atcctgcttg tctcacttgg atgtacttct |
| 1921 | ctgctgcaga tcccataaga gacacaaatt ctggcctggt gggcccgctg ctggtgtgca |
| 1981 | gggctggtgc cttgggtgca gatggcaagc agaaaggggt ggataaagaa ttctttcttc |
| 2041 | tcttcactgt gttggatgag aacaagagct ggtacagcaa tgccaatcaa gcagctgcta |
| 2101 | tgttggattt ccgactgctt tcagaggata ttgagggctt ccaagactcc aatcggatgc |
| 2161 | atgccattaa tgggtttctg ttctctaacc tgcccaggct ggacatgtgc aagggtgaca |
| 2221 | cagtggcctg gcacctgctc ggcctgggca cagagactga tgtgcatgga gtcatgttcc |
| 2281 | agggcaacac tgtgcagctt cagggcatga ggaagggtgc agctatgctc tttcctcata |
| 2341 | cctttgtcat ggccatcatg cagcctgaca accttgggac atttgagatt tattgccagg |
| 2401 | caggcagcca tcgagaagca gggatgaggg caatctataa tgtctcccag tgtcctggcc |
| 2461 | accaagccac ccctcgccaa cgctaccaag ctgcaagaat ctactatatc atggcagaag |
| 2521 | aagtagagtg ggactattgc cctgaccgga gctgggaacg ggaatggcac aaccagtctg |
| 2581 | agaaggacag ttatggttac attttcctga gcaacaagga tgggctcctg ggttccagat |
| 2641 | acaagaaagc tgtattcagg gaatacactg atggtacatt caggatccct cggccaagga |
| 2701 | ctggaccaga agaacacttg ggaatcttgg gtccacttat caaaggtgaa gttggtgata |
| 2761 | tcctgactgt ggtattcaag aataatgcca gccgccccta ctctgtgcat gctcatggag |
| 2821 | tgctagaatc tactactgtc tggccactgg ctgctgagcc tggtgaggtg gtcacttatc |
| 2881 | agtggaacat cccagagagg tctggccctg ggcccaatga ctctgcttgt gtttcctgga |
| 2941 | tctattattc tgcagtggat cccatcaagg acatgtatag tggcctggtg gggcccttgg |
| 3001 | ctatctgcca aaagggcatc ctggagcccc atggaggacg gagtgacatg gatcgggaat |
| 3061 | ttgcattgtt gttcttgatt tttgatgaaa ataagtcttg gtatttggag gaaaatgtgg |
| 3121 | caacccatgg gtcccaggat ccaggcagta ttaacctaca ggatgaaact ttcttggaga |
| 3181 | gcaataaaat gcatgcaatc aatgggaaac tctatgccaa ccttaggggt cttaccatgt |
| 3241 | accaaggaga acgagtggcc tggtacatgc tggccatggg ccaagatgtg gatctacaca |
| 3301 | ccatccactt tcatgcagag agcttcctct atcggaatgg cgagaactac cgggcagatg |
| 3361 | tggtggatct gttcccaggg acttttgagg ttgtggagat ggtggccagc aaccctggga |
| 3421 | catggctgat gcactgccat gtgactgacc atgtccatgc tggcatggag accctcttca |
| 3481 | ctgttttttc tcgaacagaa cacttaagcc ctctcaccgt catcaccaaa gagactgaaa |
| 3541 | aagcagtgcc ccccagagac attgaagaag gcaatgtgaa gatgctgggc atgcagatcc |
| 3601 | ccataaagaa tgttgagatg ctggcctctg ttttggttgc cattagtgtc acccttctgc |
| 3661 | tcgttgttct ggctcttggt ggagtggttt ggtaccaaca tcgacagaga aagctacgac |
| 3721 | gcaataggag gtccatcctg gatgacagct tcaagcttct gtctttcaaa cagtaacatc |
| 3781 | tggagcctgg agatatcctc aggaagcaca tctgtagtgc actcccagca ggccatggac |
| 3841 | tagtcactaa ccccacactc aaaggggcat gggtggtgga gaagcagaag gagcaatcaa |
| 3901 | gcttatctgg atatttcttt ctttatttat tttacatgga aataatatga tttcactttt |
| 3961 | tctttagttt ctttgctcta cgtgggcacc tggcactaag ggagtacctt attatcctac |
| 4021 | atcgcaaatt tcaacagcta cattatattt ccttctgaca cttggaaggt attgaaattt |
| 4081 | ctagaaatgt atccttctca caaagtagag accaagagaa aaactcattg attgggtttc |
| 4141 | tacttctttc aaggactcag gaaatttcac tttgaactga ggccaagtga gctgttaaga |
| 4201 | taacccacac ttaaactaaa ggctaagaat ataggcttga tgggaaattg aaggtaggct |
| 4261 | gagtattggg aatccaaatt gaattttgat tctccttggc agtgaactac tttgaagaag |
| 4321 | tggtcaatgg gttgttgctg ccatgagcat gtacaacctc tggagctaga agctcctcag |
| 4381 | gaaagccagt tctccaagtt cttaacctgt ggcactgaaa ggaatgttga gttacctctt |
| 4441 | catgttttag acagcaaacc ctatccatta aagtacttgt tagaacactg aaaaaaaaaa |
| 4501 | aaaaaaaaa |

NCBI Reference Sequence: NM_{—}014799.2 Hephaestin Isoform b:

| (SEQ ID NO: 46) | |
|---|---|
| 1 | ggaaaagagg gcacccagcc cttccccctc cctcatcctc ccatcccagt aaaccctgcc |
| 61 | aaattggaat cctggactta atttaggaga aaggccctgt aaccaagata ctgactgaac |
| 121 | atggctggcg gactcaggct ggggtctgca gtgcagcatt aatgggccgc tgacatgaat |
| 181 | atggagtagt tttctctagc aaagagtggc ttccagcttc ttaaagtctg acaagaaccg |
| 241 | gataggggga acctacaaga agaccatcta taaagaatac aaggatgact catacacaga |
| 301 | tgaagtggcc cagcctgcct ggttgggctt cctggggcca gtgttgcagg ctgaagtggg |
| 361 | ggatgtcatt cttattcacc tgaagaattt tgccactcgt ccctatacca tccaccctca |
| 421 | tggtgtcttc tacgagaagg actctgaagg ttccctatac ccagatggct cctctgggcc |
| 481 | actgaaagct gatgactctg ttcccccggg gggcagccat atctacaact ggaccattcc |
| 541 | agaaggccat gcacccaccg atgctgaccc agcgtgcctc acctggatct accattctca |
| 601 | tgtagatgct ccacgagaca ttgcaactgg cctaattggg cctctcatca cctgtaaaag |
| 661 | aggagccctg gatgggaact cccctcctca acgccaggat gtagaccatg atttcttcct |
| 721 | cctcttcagt gtggtagatg agaacctcag ctggcatctc aatgagaaca ttgccactta |
| 781 | ctgctcagat cctgcttcag tggacaaaga agatgagaca tttcaggaga gcaataggat |
| 841 | gcatgcaatc aatggctttg tttttgggaa tttacctgag ctgaacatgt gtgcacagaa |
| 901 | acgtgtggcc tggcacttgt ttggcatggg caatgaaatt gatgtccaca cagcattttt |
| 961 | ccatggacag atgctgacta cccgtggaca ccacactgat gtggctaaca tctttccagc |
| 1021 | cacctttgtg actgctgaga tggtgccctg ggaacctggt acctggttaa ttagctgcca |
| 1081 | agtgaacagt cactttcgag atggcatgca ggcactctac aaggtcaagt cttgctccat |
| 1141 | ggcccctcct gtggacctgc tcacaggcaa agttcgacag tacttcattg aggcccatga |
| 1201 | gattcaatgg gactatggcc cgatggggca tgatgggagt actgggaaga atttgagaga |
| 1261 | gccaggcagt atctcagata agtttttcca gaagagctcc agccgaattg ggggcactta |
| 1321 | ctggaaagtg cgatatgaag cctttcaaga tgagacattc caagagaaga tgcatttgga |
| 1381 | ggaagatagg catcttggaa tcctggggcc agtgatccgg gctgaggtgg gtgacaccat |
| 1441 | tcaggtggtc ttctacaacc gtgcctccca gccattcagc atgcagcccc atggggtctt |
| 1501 | ttatgagaaa gactatgaag gcactgtgta caatgatggc tcatcttacc ctggcttggt |
| 1561 | tgccaagccc tttgagaaag taacataccg ctggacagtc ccccctcatg ccggtcccac |
| 1621 | tgctcaggat cctgcttgtc tcacttggat gtacttctct gctgcagatc ccataagaga |
| 1681 | cacaaattct ggcctggtgg gcccgctgct ggtgtgcagg gctggtgcct tgggtgcaga |
| 1741 | tggcaagcag aaaggggtgg ataaagaatt ctttcttctc ttcactgtgt tggatgagaa |
| 1801 | caagagctgg tacagcaatg ccaatcaagc agctgctatg ttggatttcc gactgctttc |
| 1861 | agaggatatt gagggcttcc aagactccaa tcggatgcat gccattaatg ggtttctgtt |
| 1921 | ctctaacctg cccaggctgg acatgtgcaa gggtgacaca gtggcctggc acctgctcgg |
| 1981 | cctgggcaca gagactgatg tgcatggagt catgttccag ggcaacactg tgcagcttca |
| 2041 | gggcatgagg aagggtgcag ctatgctctt tcctcatacc tttgtcatgg ccatcatgca |
| 2101 | gcctgacaac cttgggacat ttgagattta ttgccaggca ggcagccatc gagaagcagg |
| 2161 | gatgagggca atctataatg tctcccagtg tcctggccac caagccaccc ctcgccaacg |
| 2221 | ctaccaagct gcaagaatct actatatcat ggcagaagaa gtagagtggg actattgccc |
| 2281 | tgaccggagc tgggaacggg aatggcacaa ccagtctgag aaggacagtt atggttacat |
| 2341 | tttcctgagc aacaaggatg ggctcctggg ttccagatac aagaaagctg tattcaggga |
| 2401 | atacactgat ggtacattca ggatccctcg gccaaggact ggaccagaag aacacttggg |
| 2461 | aatcttgggt ccacttatca aaggtgaagt tggtgatatc ctgactgtgg tattcaagaa |
| 2521 | taatgccagc cgcccctact ctgtgcatgc tcatggagtg ctagaatcta ctactgtctg |
| 2581 | gccactggct gctgagcctg gtgaggtggt cacttatcag tggaacatcc cagagaggtc |
| 2641 | tggccctggg cccaatgact ctgcttgtgt ttcctggatc tattattctg cagtggatcc |
| 2701 | catcaaggac atgtatagtg gcctggtggg gcccttggct atctgccaaa agggcatcct |
| 2761 | ggagccccat ggaggacgga gtgacatgga tcgggaattt gcattgttgt tcttgatttt |
| 2821 | tgatgaaaat aagtcttggt atttggagga aaatgtggca acccatgggt cccaggatcc |
| 2881 | aggcagtatt aacctacagg atgaaacttt cttggagagc aataaaatgc atgcaatcaa |
| 2941 | tgggaaactc tatgccaacc ttaggggtct taccatgtac caaggagaac gagtggcctg |
| 3001 | gtacatgctg gccatgggcc aagatgtgga tctacacacc atccactttc atgcagagag |
| 3061 | cttcctctat cggaatggcg agaactaccg ggcagatgtg gtggatctgt tcccagggac |
| 3121 | ttttgaggtt gtggagatgg tggccagcaa ccctgggaca tggctgatgc actgccatgt |
| 3181 | gactgaccat gtccatgctg gcatggagac cctcttcact gttttttctc gaacagaaca |
| 3241 | cttaagccct ctcaccgtca tcaccaaaga gactgaaaaa gcagtgcccc ccagagacat |
| 3301 | tgaagaaggc aatgtgaaga tgctgggcat gcagatcccc ataaagaatg ttgagatgct |
| 3361 | ggcctctgtt ttggttgcca ttagtgtcac ccttctgctc gttgttctgg ctcttggtgg |
| 3421 | agtggtttgg taccaacatc gacagagaaa gctacgacgc aataggaggt ccatcctgga |
| 3481 | tgacagcttc aagcttctgt ctttcaaaca gtaacatctg gagcctggag atatcctcag |
| 3541 | gaagcacatc tgtagtgcac tcccagcagg ccatggacta gtcactaacc ccacactcaa |
| 3601 | aggggcatgg gtggtggaga agcagaagga gcaatcaagc ttatctggat atttctttct |
| 3661 | ttatttattt tacatggaaa taatatgatt tcactttttc tttagtttct ttgctctacg |
| 3721 | tgggcacctg gcactaaggg agtaccttat tatcctacat cgcaaatttc aacagctaca |
| 3781 | ttatatttcc ttctgacact tggaaggtat tgaaatttct agaaatgtat ccttctcaca |
| 3841 | aagtagagac caagagaaaa actcattgat tgggtttcta cttctttcaa ggactcagga |
| 3901 | aatttcactt tgaactgagg ccaagtgagc tgttaagata acccacactt aaactaaagg |
| 3961 | ctaagaatat aggcttgatg ggaaattgaa ggtaggctga gtattgggaa tccaaattga |
| 4021 | attttgattc tccttggcag tgaactactt tgaagaagtg gtcaatgggt tgttgctgcc |
| 4081 | atgagcatgt acaacctctg gagctagaag ctcctcagga aagccagttc tccaagttct |
| 4141 | taacctgtgg cactgaaagg aatgttgagt tacctcttca tgttttagac agcaaaccct |
| 4201 | atccattaaa gtacttgtta gaacactgaa a |

NCBI Reference Sequence: NM_{—}001130860.2 Hephaestin Isoform c Precursor

| (SEQ ID NO: 47) | |
|---|---|
| 1 | ggaacaggac attccagtag ttttgtttct ggaaaagagg gcacccagcc cttccccctc |
| 61 | cctcatcctc ccatcccagt aaaccctgcc aaattggaat cctggactta atttaggaga |
| 121 | aaggccctgt aaccaagata ctgactgaac atggctggcg gactcaggct ggggtctgca |
| 181 | gtgcagcatt aatgggccgc tgacatgaat atggagtagt tttctctagc aaagagtaat |
| 241 | gtgggccatg gagtcaggcc acctcctctg ggctctgctg ttcatgcagt ccttgtggcc |
| 301 | tcaactgact gatggagcca ctcgagtcta ctacctgggc atccgggatg tgcagtggaa |
| 361 | ctatgctccc aagggaagaa atgtcatcac gaaccagcct ctggacagtg acatagtggc |
| 421 | ttccagcttc ttaaagtctg acaagaaccg gataggggga acctacaaga agaccatcta |
| 481 | taaagaatac aaggatgact catacacaga tgaagtggcc cagcctgcct ggttgggctt |
| 541 | cctggggcca gtgttgcagg ctgaagtggg ggatgtcatt cttattcacc tgaagaattt |
| 601 | tgccactcgt ccctatacca tccaccctca tggtgtcttc tacgagaagg actctgaagg |
| 661 | ttccctatac ccagatggct cctctgggcc actgaaagct gatgactctg ttcccccggg |
| 721 | gggcagccat atctacaact ggaccattcc agaaggccat gcacccaccg atgctgaccc |
| 781 | agcgtgcctc acctggatct accattctca tgtagatgct ccacgagaca ttgcaactgg |
| 841 | cctaattggg cctctcatca cctgtaaaag aggagccctg gatgggaact cccctcctca |
| 901 | acgccaggat gtagaccatg atttcttcct cctcttcagt gtggtagatg agaacctcag |
| 961 | ctggcatctc aatgagaaca ttgccactta ctgctcagat cctgcttcag tggacaaaga |
| 1021 | agatgagaca tttcaggaga gcaataggat gcatgcaatc aatggctttg tttttgggaa |
| 1081 | tttacctgag ctgaacatgt gtgcacagaa acgtgtggcc tggcacttgt ttggcatggg |
| 1141 | caatgaaatt gatgtccaca cagcattttt ccatggacag atgctgacta cccgtggaca |
| 1201 | ccacactgat gtggctaaca tctttccagc cacctttgtg actgctgaga tggtgccctg |
| 1261 | ggaacctggt acctggttaa ttagctgcca agtgaacagt cactttcgag atggcatgca |
| 1321 | ggcactctac aaggtcaagt cttgctccat ggcccctcct gtggacctgc tcacaggcaa |
| 1381 | agttcgacag tacttcattg aggcccatga gattcaatgg gactatggcc cgatggggca |
| 1441 | tgatgggagt actgggaaga atttgagaga gccaggcagt atctcagata agtttttcca |
| 1501 | gaagagctcc agccgaattg ggggcactta ctggaaagtg cgatatgaag cctttcaaga |
| 1561 | tgagacattc caagagaaga tgcatttgga ggaagatagg catcttggaa tcctggggcc |
| 1621 | agtgatccgg gctgaggtgg gtgacaccat tcaggtggtc ttctacaacc gtgcctccca |
| 1681 | gccattcagc atgcagcccc atggggtctt ttatgagaaa gactatgaag gcactgtgta |
| 1741 | caatgatggc tcatcttacc ctggcttggt tgccaagccc tttgagaaag taacataccg |
| 1801 | ctggacagtc ccccctcatg ccggtcccac tgctcaggat cctgcttgtc tcacttggat |
| 1861 | gtacttctct gctgcagatc ccataagaga cacaaattct ggcctggtgg gcccgctgct |
| 1921 | ggtgtgcagg gctggtgcct tgggtgcaga tggcaagcag aaaggggtgg ataaagaatt |
| 1981 | ctttcttctc ttcactgtgt tggatgagaa caagagctgg tacagcaatg ccaatcaagc |
| 2041 | agctgctatg ttggatttcc gactgctttc agaggatatt gagggcttcc aagactccaa |
| 2101 | tcggatgcat gccattaatg ggtttctgtt ctctaacctg cccaggctgg acatgtgcaa |
| 2161 | gggtgacaca gtggcctggc acctgctcgg cctgggcaca gagactgatg tgcatggagt |
| 2221 | catgttccag ggcaacactg tgcagcttca gggcatgagg aagggtgcag ctatgctctt |
| 2281 | tcctcatacc tttgtcatgg ccatcatgca gcctgacaac cttgggacat ttgagattta |
| 2341 | ttgccaggca ggcagccatc gagaagcagg gatgagggca atctataatg tctcccagtg |
| 2401 | tcctggccac caagccaccc ctcgccaacg ctaccaagct gcaagaatct actatatcat |
| 2461 | ggcagaagaa gtagagtggg actattgccc tgaccggagc tgggaacggg aatggcacaa |
| 2521 | ccagtctgag aaggacagtt atggttacat tttcctgagc aacaaggatg ggctcctggg |
| 2581 | ttccagatac aagaaagctg tattcaggga atacactgat ggtacattca ggatccctcg |
| 2641 | gccaaggact ggaccagaag aacacttggg aatcttgggt ccacttatca aaggtgaagt |
| 2701 | tggtgatatc ctgactgtgg tattcaagaa taatgccagc cgcccctact ctgtgcatgc |
| 2761 | tcatggagtg ctagaatcta ctactgtctg gccactggct gctgagcctg gtgaggtggt |
| 2821 | cacttatcag tggaacatcc cagagaggtc tggccctggg cccaatgact ctgcttgtgt |
| 2881 | ttcctggatc tattattctg cagtggatcc catcaaggac atgtatagtg gcctggtggg |
| 2941 | gcccttggct atctgccaaa agggcatcct ggagccccat ggaggacgga gtgacatgga |
| 3001 | tcgggaattt gcattgttgt tcttgatttt tgatgaaaat aagtcttggt atttggagga |
| 3061 | aaatgtggca acccatgggt cccaggatcc aggcagtatt aacctacagg atgaaacttt |
| 3121 | cttggagagc aataaaatgc atgcaatcaa tgggaaactc tatgccaacc ttaggggtct |
| 3181 | taccatgtac caaggagaac gagtggcctg gtacatgctg gccatgggcc aagatgtgga |
| 3241 | tctacacacc atccactttc atgcagagag cttcctctat cggaatggcg agaactaccg |
| 3301 | ggcagatgtg gtggatctgt tcccagggac ttttgaggtt gtggagatgg tggccagcaa |
| 3361 | ccctgggaca tggctgatgc actgccatgt gactgaccat gtccatgctg gcatggagac |
| 3421 | cctcttcact gttttttctc gaacagaaca cttaagccct ctcaccgtca tcaccaaaga |
| 3481 | gactgaaaaa gtgcccccca gagacattga agaaggcaat gtgaagatgc tgggcatgca |
| 3541 | gatccccata aagaatgttg agatgctggc ctctgttttg gttgccatta gtgtcaccct |
| 3601 | tctgctcgtt gttctggctc ttggtggagt ggtttggtac caacatcgac agagaaagct |
| 3661 | acgacgcaat aggaggtcca tcctggatga cagcttcaag cttctgtctt tcaaacagta |
| 3721 | acatctggag cctggagata tcctcaggaa gcacatctgt agtgcactcc cagcaggcca |
| 3781 | tggactagtc actaacccca cactcaaagg ggcatgggtg gtggagaagc agaaggagca |
| 3841 | atcaagctta tctggatatt tctttcttta tttattttac atggaaataa tatgatttca |
| 3901 | ctttttcttt agtttctttg ctctacgtgg gcacctggca ctaagggagt accttattat |
| 3961 | cctacatcgc aaatttcaac agctacatta tatttccttc tgacacttgg aaggtattga |
| 4021 | aatttctaga aatgtatcct tctcacaaag tagagaccaa gagaaaaact cattgattgg |
| 4081 | gtttctactt ctttcaagga ctcaggaaat ttcactttga actgaggcca agtgagctgt |
| 4141 | taagataacc cacacttaaa ctaaaggcta agaatatagg cttgatggga aattgaaggt |
| 4201 | aggctgagta ttgggaatcc aaattgaatt ttgattctcc ttggcagtga actactttga |
| 4261 | agaagtggtc aatgggttgt tgctgccatg agcatgtaca acctctggag ctagaagctc |
| 4321 | ctcaggaaag ccagttctcc aagttcttaa cctgtggcac tgaaaggaat gttgagttac |
| 4381 | ctcttcatgt tttagacagc aaaccctatc cattaaagta cttgttagaa cactgaaaaa |
| 4441 | aaaaaaaaaa aaaa |

### KRT19

Keratin 19 (KRT19), also known as K19; CK19; K1CS is a member of the keratin family. KRT19 is the smallest known (40 kD) acidic keratin and has been shown to be expressed in epithelial cells in culture (Savtchenko et al. 1988, Am. J. Hum. Genet. 43:630-637; Bader et al. 1988, Europ. J. Cell Biol. 47:300-319). KRT19 expression has been detected in small intestinal stem cells, colon stem cells, gastric stem cells, liver stem cells, pancreatic stem cells and renal stem cells. RNA expression can be measure for example by RT-PCR, RT-qPCR, RNA-Seq, microarray approaches or RNA in situ hybridization. Protein expression can be detected for example by immunofluorescence, immunohistochemistry, FACS, flow cytometry, Western blot or ELISA. In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA).

The Human cDNA Sequence is Listed Below (NCBI Reference Sequence: NM_{—}002276.4):

| (SEQ ID NO: 48) | |
|---|---|
| 1 | agatatccgc ccctgacacc attcctccct tcccccctcc accggccgcg ggcataaaag |
| 61 | gcgccaggtg agggcctcgc cgctcctccc gcgaatcgca gcttctgaga ccagggttgc |
| 121 | tccgtccgtg ctccgcctcg ccatgacttc ctacagctat cgccagtcgt cggccacgtc |
| 181 | gtccttcgga ggcctgggcg gcggctccgt gcgttttggg ccgggggtcg cctttcgcgc |
| 241 | gcccagcatt cacgggggct ccggcggccg cggcgtatcc gtgtcctccg cccgctttgt |
| 301 | gtcctcgtcc tcctcggggg cctacggcgg cggctacggc ggcgtcctga ccgcgtccga |
| 361 | cgggctgctg gcgggcaacg agaagctaac catgcagaac ctcaacgacc gcctggcctc |
| 421 | ctacctggac aaggtgcgcg ccctggaggc ggccaacggc gagctagagg tgaagatccg |
| 481 | cgactggtac cagaagcagg ggcctgggcc ctcccgcgac tacagccact actacacgac |
| 541 | catccaggac ctgcgggaca agattcttgg tgccaccatt gagaactcca ggattgtcct |
| 601 | gcagatcgac aatgcccgtc tggctgcaga tgacttccga accaagtttg agacggaaca |
| 661 | ggctctgcgc atgagcgtgg aggccgacat caacggcctg cgcagggtgc tggatgagct |
| 721 | gaccctggcc aggaccgacc tggagatgca gatcgaaggc ctgaaggaag agctggccta |
| 781 | cctgaagaag aaccatgagg aggaaatcag tacgctgagg ggccaagtgg gaggccaggt |
| 841 | cagtgtggag gtggattccg ctccgggcac cgatctcgcc aagatcctga gtgacatgcg |
| 901 | aagccaatat gaggtcatgg ccgagcagaa ccggaaggat gctgaagcct ggttcaccag |
| 961 | ccggactgaa gaattgaacc gggaggtcgc tggccacacg gagcagctcc agatgagcag |
| 1021 | gtccgaggtt actgacctgc ggcgcaccct tcagggtctt gagattgagc tgcagtcaca |
| 1081 | gctgagcatg aaagctgcct tggaagacac actggcagaa acggaggcgc gctttggagc |
| 1141 | ccagctggcg catatccagg cgctgatcag cggtattgaa gcccagctgg gcgatgtgcg |
| 1201 | agctgatagt gagcggcaga atcaggagta ccagcggctc atggacatca agtcgcggct |
| 1261 | ggagcaggag attgccacct accgcagcct gctcgaggga caggaagatc actacaacaa |
| 1321 | tttgtctgcc tccaaggtcc tctgaggcag caggctctgg ggcttctgct gtcctttgga |
| 1381 | gggtgtcttc tgggtagagg gatgggaagg aagggaccct tacccccggc tcttctcctg |
| 1441 | acctgccaat aaaaatttat ggtccaaggg aaaaaaaaaa aaaaaaaaaa |

### KRT7

Keratin 7 (KRT7), also known as K7; CK7; SCL; or K2C7 is a member of the keratin family. KRT7 is a type II keratin of simple nonkeratinizing epithelia (Glass et al., 1985, J. Cell Biol. 101:2366-237). KRT7 expression has been detected in gastric stem cells, liver stem cells, pancreatic stem cells and renal stem cells. Expression may be detected either at the RNA level or protein level. RNA expression can be measure for example by RT-PCR, RT-qPCR, RNA-Seq, microarray approaches or RNA in situ hybridization. Protein expression can be detected for example by immunofluorescence, immunohistochemistry, FACS, flow cytometry, Western blot or ELISA. In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA).

The Human cDNA Sequence is Listed Below (NCBI Reference Sequence: NM_{—}005556.3 Keratin, Type II Cytoskeletal 7):

| (SEQ ID NO: 49) | |
|---|---|
| 1 | cagccccgcc cctacctgtg gaagcccagc cgcccgctcc cgcggataaa aggcgcggag |
| 61 | tgtccccgag gtcagcgagt gcgcgctcct cctcgcccgc cgctaggtcc atcccggccc |
| 121 | agccaccatg tccatccact tcagctcccc ggtattcacc tcgcgctcag ccgccttctc |
| 181 | gggccgcggc gcccaggtgc gcctgagctc cgctcgcccc ggcggccttg gcagcagcag |
| 241 | cctctacggc ctcggcgcct cacggccgcg cgtggccgtg cgctctgcct atgggggccc |
| 301 | ggtgggcgcc ggcatccgcg aggtcaccat taaccagagc ctgctggccc cgctgcggct |
| 361 | ggacgccgac ccctccctcc agcgggtgcg ccaggaggag agcgagcaga tcaagaccct |
| 421 | caacaacaag tttgcctcct tcatcgacaa ggtgcggttt ctggagcagc agaacaagct |
| 481 | gctggagacc aagtggacgc tgctgcagga gcagaagtcg gccaagagca gccgcctccc |
| 541 | agacatcttt gaggcccaga ttgctggcct tcggggtcag cttgaggcac tgcaggtgga |
| 601 | tgggggccgc ctggaggcgg agctgcggag catgcaggat gtggtggagg acttcaagaa |
| 661 | taagtacgaa gatgaaatta accaccgcac agctgctgag aatgagtttg tggtgctgaa |
| 721 | gaaggatgtg gatgctgcct acatgagcaa ggtggagctg gaggccaagg tggatgccct |
| 781 | gaatgatgag atcaacttcc tcaggaccct caatgagacg gagttgacag agctgcagtc |
| 841 | ccagatctcc gacacatctg tggtgctgtc catggacaac agtcgctccc tggacctgga |
| 901 | cggcatcatc gctgaggtca aggcgcagta tgaggagatg gccaaatgca gccgggctga |
| 961 | ggctgaagcc tggtaccaga ccaagtttga gaccctccag gcccaggctg ggaagcatgg |
| 1021 | ggacgacctc cggaataccc ggaatgagat ttcagagatg aaccgggcca tccagaggct |
| 1081 | gcaggctgag atcgacaaca tcaagaacca gcgtgccaag ttggaggccg ccattgccga |
| 1141 | ggctgaggag cgtggggagc tggcgctcaa ggatgctcgt gccaagcagg aggagctgga |
| 1201 | agccgccctg cagcggggca agcaggatat ggcacggcag ctgcgtgagt accaggaact |
| 1261 | catgagcgtg aagctggccc tggacatcga gatcgccacc taccgcaagc tgctggaggg |
| 1321 | cgaggagagc cggttggctg gagatggagt gggagccgtg aatatctctg tgatgaattc |
| 1381 | cactggtggc agtagcagtg gcggtggcat tgggctgacc ctcgggggaa ccatgggcag |
| 1441 | caatgccctg agcttctcca gcagtgcggg tcctgggctc ctgaaggctt attccatccg |
| 1501 | gaccgcatcc gccagtcgca ggagtgcccg cgactgagcc gcctcccacc actccactcc |
| 1561 | tccagccacc acccacaatc acaagaagat tcccacccct gcctcccatg cctggtccca |
| 1621 | agacagtgag acagtctgga aagtgatgtc agaatagctt ccaataaagc agcctcattc |
| 1681 | tgaggcctga gtgatccacg tgaaaaaaaa aaaaaaaaaa aaaaaaaaaa aaaaaaaaaa |
| 1741 | aaaaaaaaaa aaa |

### LGR5

LGR5 (leucine-rich-repeat-containing G-protein-coupled receptor 5), also known as GRP49, FEX, HG38, or GPR67 is a marker for stem cells in small intestine and colon (Barker, N. et al. 2007; Nature 449:1003-1007). LGR5 RNA expression has been detected in small intestinal stem cells, and colon stem cells. RNA expression can be measure for example by RT-PCR, RT-qPCR, RNA-Seq, microarray approaches or RNA in situ hybridization. For example, in situ probes comprising a 1 kb N-terminal fragment of mouse Lgr5 can be generated from Image Clone 30873333. In situ probes can be obtained for example from Advanced Cell Diagnostics RNAscope (cat no. 311021). qPCR primers can be obtained from OriGene Technologies (Rockville, Md.) and QIAGEN (Germantown, Md.), and other suppliers. RT-PCR primers and in situ probes can be designed using methods known in the art.

The Human cDNA Sequence is Listed Below (NCBI Reference Sequence: NM_{—}003667.2)

| (SEQ ID NO: 50) | |
|---|---|
| 1 | tgctgctctc cgcccgcgtc cggctcgtgg ccccctactt cgggcaccat ggacacctcc |
| 61 | cggctcggtg tgctcctgtc cttgcctgtg ctgctgcagc tggcgaccgg gggcagctct |
| 121 | cccaggtctg gtgtgttgct gaggggctgc cccacacact gtcattgcga gcccgacggc |
| 181 | aggatgttgc tcagggtgga ctgctccgac ctggggctct cggagctgcc ttccaacctc |
| 241 | agcgtcttca cctcctacct agacctcagt atgaacaaca tcagtcagct gctcccgaat |
| 301 | cccctgccca gtctccgctt cctggaggag ttacgtcttg cgggaaacgc tctgacatac |
| 361 | attcccaagg gagcattcac tggcctttac agtcttaaag ttcttatgct gcagaataat |
| 421 | cagctaagac acgtacccac agaagctctg cagaatttgc gaagccttca atccctgcgt |
| 481 | ctggatgcta accacatcag ctatgtgccc ccaagctgtt tcagtggcct gcattccctg |
| 541 | aggcacctgt ggctggatga caatgcgtta acagaaatcc ccgtccaggc ttttagaagt |
| 601 | ttatcggcat tgcaagccat gaccttggcc ctgaacaaaa tacaccacat accagactat |
| 661 | gcctttggaa acctctccag cttggtagtt ctacatctcc ataacaatag aatccactcc |
| 721 | ctgggaaaga aatgctttga tgggctccac agcctagaga ctttagattt aaattacaat |
| 781 | aaccttgatg aattccccac tgcaattagg acactctcca accttaaaga actaggattt |
| 841 | catagcaaca atatcaggtc gatacctgag aaagcatttg taggcaaccc ttctcttatt |
| 901 | acaatacatt tctatgacaa tcccatccaa tttgttggga gatctgcttt tcaacattta |
| 961 | cctgaactaa gaacactgac tctgaatggt gcctcacaaa taactgaatt tcctgattta |
| 1021 | actggaactg caaacctgga gagtctgact ttaactggag cacagatctc atctcttcct |
| 1081 | caaaccgtct gcaatcagtt acctaatctc caagtgctag atctgtctta caacctatta |
| 1141 | gaagatttac ccagtttttc agtctgccaa aagcttcaga aaattgacct aagacataat |
| 1201 | gaaatctacg aaattaaagt tgacactttc cagcagttgc ttagcctccg atcgctgaat |
| 1261 | ttggcttgga acaaaattgc tattattcac cccaatgcat tttccacttt gccatcccta |
| 1321 | ataaagctgg acctatcgtc caacctcctg tcgtcttttc ctataactgg gttacatggt |
| 1381 | ttaactcact taaaattaac aggaaatcat gccttacaga gcttgatatc atctgaaaac |
| 1441 | tttccagaac tcaaggttat agaaatgcct tatgcttacc agtgctgtgc atttggagtg |
| 1501 | tgtgagaatg cctataagat ttctaatcaa tggaataaag gtgacaacag cagtatggac |
| 1561 | gaccttcata agaaagatgc tggaatgttt caggctcaag atgaacgtga ccttgaagat |
| 1621 | ttcctgcttg actttgagga agacctgaaa gcccttcatt cagtgcagtg ttcaccttcc |
| 1681 | ccaggcccct tcaaaccctg tgaacacctg cttgatggct ggctgatcag aattggagtg |
| 1741 | tggaccatag cagttctggc acttacttgt aatgctttgg tgacttcaac agttttcaga |
| 1801 | tcccctctgt acatttcccc cattaaactg ttaattgggg tcatcgcagc agtgaacatg |
| 1861 | ctcacgggag tctccagtgc cgtgctggct ggtgtggatg cgttcacttt tggcagcttt |
| 1921 | gcacgacatg gtgcctggtg ggagaatggg gttggttgcc atgtcattgg ttttttgtcc |
| 1981 | atttttgctt cagaatcatc tgttttcctg cttactctgg cagccctgga gcgtgggttc |
| 2041 | tctgtgaaat attctgcaaa atttgaaacg aaagctccat tttctagcct gaaagtaatc |
| 2101 | attttgctct gtgccctgct ggccttgacc atggccgcag ttcccctgct gggtggcagc |
| 2161 | aagtatggcg cctcccctct ctgcctgcct ttgccttttg gggagcccag caccatgggc |
| 2221 | tacatggtcg ctctcatctt gctcaattcc ctttgcttcc tcatgatgac cattgcctac |
| 2281 | accaagctct actgcaattt ggacaaggga gacctggaga atatttggga ctgctctatg |
| 2341 | gtaaaacaca ttgccctgtt gctcttcacc aactgcatcc taaactgccc tgtggctttc |
| 2401 | ttgtccttct cctctttaat aaaccttaca tttatcagtc ctgaagtaat taagtttatc |
| 2461 | cttctggtgg tagtcccact tcctgcatgt ctcaatcccc ttctctacat cttgttcaat |
| 2521 | cctcacttta aggaggatct ggtgagcctg agaaagcaaa cctacgtctg gacaagatca |
| 2581 | aaacacccaa gcttgatgtc aattaactct gatgatgtcg aaaaacagtc ctgtgactca |
| 2641 | actcaagcct tggtaacctt taccagctcc agcatcactt atgacctgcc tcccagttcc |
| 2701 | gtgccatcac cagcttatcc agtgactgag agctgccatc tttcctctgt ggcatttgtc |
| 2761 | ccatgtctct aattaatatg tgaaggaaaa tgttttcaaa ggttgagaac ctgaaaatgt |
| 2821 | gagattgagt atatcagagc agtaattaat aagaagagct gaggtgaaac tcggtttaaa |

### OLFM4

OLFM4 (olfactomedin 4) also known as antiapoptotic protein GW112; G-CSF-stimulated clone 1 protein; GC1; OLM4; OlfD; hGC-1; hOLfD; UNQ362; bA209J19.1 was originally cloned from human myeloblasts and found to be selectively expressed in inflamed colonic epithelium (Shinozaki et al. (2001, Gut 48: 623-239). OLFM4 has been described as robust stem cell marker by van der Flier et al., 2009 (Gastroenterology 137(1):15-7). BPIFB1 RNA expression has been detected in small intestinal stem cells, and colon stem cells. RNA expression can be measure for example by RT-PCR, RT-qPCR, RNA-Seq, microarray approaches or RNA in situ hybridization. In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art.

The Human cDNA (NCBI Reference Sequence: NM_{—}006418.4) is Listed Below:

| (SEQ ID NO: 51) | |
|---|---|
| 1 | ttttcctaca tgctggccat ggggaaatca ccactgggca ctataagaag cccctgggct |
| 61 | ctctgcagag ccagcggctc cagctaagag gacaagatga ggcccggcct ctcatttctc |
| 121 | ctagcccttc tgttcttcct tggccaagct gcaggggatt tgggggatgt gggacctcca |
| 181 | attcccagcc ccggcttcag ctctttccca ggtgttgact ccagctccag cttcagctcc |
| 241 | agctccaggt cgggctccag ctccagccgc agcttaggca gcggaggttc tgtgtcccag |
| 301 | ttgttttcca atttcaccgg ctccgtggat gaccgtggga cctgccagtg ctctgtttcc |
| 361 | ctgccagaca ccacctttcc cgtggacaga gtggaacgct tggaattcac agctcatgtt |
| 421 | ctttctcaga agtttgagaa agaactttcc aaagtgaggg aatatgtcca attaattagt |
| 481 | gtgtatgaaa agaaactgtt aaacctaact gtccgaattg acatcatgga gaaggatacc |
| 541 | atttcttaca ctgaactgga cttcgagctg atcaaggtag aagtgaagga gatggaaaaa |
| 601 | ctggtcatac agctgaagga gagttttggt ggaagctcag aaattgttga ccagctggag |
| 661 | gtggagataa gaaatatgac tctcttggta gagaagcttg agacactaga caaaaacaat |
| 721 | gtccttgcca ttcgccgaga aatcgtggct ctgaagacca agctgaaaga gtgtgaggcc |
| 781 | tctaaagatc aaaacacccc tgtcgtccac cctcctccca ctccagggag ctgtggtcat |
| 841 | ggtggtgtgg tgaacatcag caaaccgtct gtggttcagc tcaactggag agggttttct |
| 901 | tatctatatg gtgcttgggg tagggattac tctccccagc atccaaacaa aggactgtat |
| 961 | tgggtggcgc cattgaatac agatgggaga ctgttggagt attatagact gtacaacaca |
| 1021 | ctggatgatt tgctattgta tataaatgct cgagagttgc ggatcaccta tggccaaggt |
| 1081 | agtggtacag cagtttacaa caacaacatg tacgtcaaca tgtacaacac cgggaatatt |
| 1141 | gccagagtta acctgaccac caacacgatt gctgtgactc aaactctccc taatgctgcc |
| 1201 | tataataacc gcttttcata tgctaatgtt gcttggcaag atattgactt tgctgtggat |
| 1261 | gagaatggat tgtgggttat ttattcaact gaagccagca ctggtaacat ggtgattagt |
| 1321 | aaactcaatg acaccacact tcaggtgcta aacacttggt ataccaagca gtataaacca |
| 1381 | tctgcttcta acgccttcat ggtatgtggg gttctgtatg ccacccgtac tatgaacacc |
| 1441 | agaacagaag agatttttta ctattatgac acaaacacag ggaaagaggg caaactagac |
| 1501 | attgtaatgc ataagatgca ggaaaaagtg cagagcatta actataaccc ttttgaccag |
| 1561 | aaactttatg tctataacga tggttacctt ctgaattatg atctttctgt cttgcagaag |
| 1621 | ccccagtaag ctgtttagga gttagggtga aagagaaaat gtttgttgaa aaaatagtct |
| 1681 | tctccactta cttagatatc tgcaggggtg tctaaaagtg tgttcatttt gcagcaatgt |
| 1741 | ttaggtgcat agttctacca cactagagat ctaggacatt tgtcttgatt tggtgagttc |
| 1801 | tcttgggaat catctgcctc ttcaggcgca ttttgcaata aagtctgtct agggtgggat |
| 1861 | tgtcagaggt ctaggggcac tgtgggccta gtgaagccta ctgtgaggag gcttcactag |
| 1921 | aagccttaaa ttaggaatta aggaacttaa aactcagtat ggcgtctagg gattctttgt |
| 1981 | acaggaaata ttgcccaatg actagtcctc atccatgtag caccactaat tcttccatgc |
| 2041 | ctggaagaaa cctggggact tagttaggta gattaatatc tggagctcct cgagggacca |
| 2101 | aatctccaac ttttttttcc cctcactagc acctggaatg atgctttgta tgtggcagat |
| 2161 | aagtaaattt ggcatgctta tatattctac atctgtaaag tgctgagttt tatggagaga |
| 2221 | ggccttttta tgcattaaat tgtacatggc aaataaatcc cagaaggatc tgtagatgag |
| 2281 | gcacctgctt tttcttttct ctcattgtcc accttactaa aagtcagtag aatcttctac |
| 2341 | ctcataactt ccttccaaag gcagctcaga agattagaac cagacttact aaccaattcc |
| 2401 | accccccacc aacccccttc tactgcctac tttaaaaaaa ttaatagttt tctatggaac |
| 2461 | tgatctaaga ttagaaaaat taattttctt taatttcatt atgaactttt atttacatga |
| 2521 | ctctaagact ataagaaaat ctgatggcag tgacaaagtg ctagcattta ttgttatcta |
| 2581 | ataaagacct tggagcatat gtgcaactta tgagtgtatc agttgttgca tgtaattttt |
| 2641 | gcctttgttt aagcctggaa cttgtaagaa aatgaaaatt taattttttt ttctaggacg |
| 2701 | agctatagaa aagctattga gagtatctag ttaatcagtg cagtagttgg aaaccttgct |
| 2761 | ggtgtatgtg atgtgcttct gtgcttttga atgactttat catctagtct ttgtctattt |
| 2821 | ttcctttgat gttcaagtcc tagtctatag gattggcagt ttaaatgctt tactccccct |
| 2881 | tttaaaataa atgattaaaa tgtgctttga aaaaagtcaa aaaaaaaaaa aaaaa |

### PPARGC1A

Peroxisome proliferator-activated receptor gamma, coactivator 1 alpha (PPARGC1A), also known as LEM6; PGC1; PGC1A; PGC-1v; PPARGC1; or PGC-1(alpha), is a transcriptional coactivator that regulates the genes involved in energy metabolism. This protein interacts with PPARgamma, which permits the interaction of this protein with multiple transcription factors. PPARGC1A RNA expression has been detected in colon stem cells, and gastric stem cells. RNA expression can be measure for example by RT-PCR, RT-qPCR, RNA-Seq, microarray approaches or RNA in situ hybridization. In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art.

The Human cDNA (NCBI Reference Sequence: NM_{—}013261.3) is Listed Below:

| (SEQ ID NO: 52) | |
|---|---|
| 1 | tagtaagaca ggtgccttca gttcactctc agtaaggggc tggttgcctg catgagtgtg |
| 61 | tgctctgtgt cactgtggat tggagttgaa aaagcttgac tggcgtcatt caggagctgg |
| 121 | atggcgtggg acatgtgcaa ccaggactct gagtctgtat ggagtgacat cgagtgtgct |
| 181 | gctctggttg gtgaagacca gcctctttgc ccagatcttc ctgaacttga tctttctgaa |
| 241 | ctagatgtga acgacttgga tacagacagc tttctgggtg gactcaagtg gtgcagtgac |
| 301 | caatcagaaa taatatccaa tcagtacaac aatgagcctt caaacatatt tgagaagata |
| 361 | gatgaagaga atgaggcaaa cttgctagca gtcctcacag agacactaga cagtctccct |
| 421 | gtggatgaag acggattgcc ctcatttgat gcgctgacag atggagacgt gaccactgac |
| 481 | aatgaggcta gtccttcctc catgcctgac ggcacccctc caccccagga ggcagaagag |
| 541 | ccgtctctac ttaagaagct cttactggca ccagccaaca ctcagctaag ttataatgaa |
| 601 | tgcagtggtc tcagtaccca gaaccatgca aatcacaatc acaggatcag aacaaaccct |
| 661 | gcaattgtta agactgagaa ttcatggagc aataaagcga agagtatttg tcaacagcaa |
| 721 | aagccacaaa gacgtccctg ctcggagctt ctcaaatatc tgaccacaaa cgatgaccct |
| 781 | cctcacacca aacccacaga gaacagaaac agcagcagag acaaatgcac ctccaaaaag |
| 841 | aagtcccaca cacagtcgca gtcacaacac ttacaagcca aaccaacaac tttatctctt |
| 901 | cctctgaccc cagagtcacc aaatgacccc aagggttccc catttgagaa caagactatt |
| 961 | gaacgcacct taagtgtgga actctctgga actgcaggcc taactccacc caccactcct |
| 1021 | cctcataaag ccaaccaaga taaccctttt agggcttctc caaagctgaa gtcctcttgc |
| 1081 | aagactgtgg tgccaccacc atcaaagaag cccaggtaca gtgagtcttc tggtacacaa |
| 1141 | ggcaataact ccaccaagaa agggccggag caatccgagt tgtatgcaca actcagcaag |
| 1201 | tcctcagtcc tcactggtgg acacgaggaa aggaagacca agcggcccag tctgcggctg |
| 1261 | tttggtgacc atgactattg ccagtcaatt aattccaaaa cagaaatact cattaatata |
| 1321 | tcacaggagc tccaagactc tagacaacta gaaaataaag atgtctcctc tgattggcag |
| 1381 | gggcagattt gttcttccac agattcagac cagtgctacc tgagagagac tttggaggca |
| 1441 | agcaagcagg tctctccttg cagcacaaga aaacagctcc aagaccagga aatccgagcc |
| 1501 | gagctgaaca agcacttcgg tcatcccagt caagctgttt ttgacgacga agcagacaag |
| 1561 | accggtgaac tgagggacag tgatttcagt aatgaacaat tctccaaact acctatgttt |
| 1621 | ataaattcag gactagccat ggatggcctg tttgatgaca gcgaagatga aagtgataaa |
| 1681 | ctgagctacc cttgggatgg cacgcaatcc tattcattgt tcaatgtgtc tccttcttgt |
| 1741 | tcttctttta actctccatg tagagattct gtgtcaccac ccaaatcctt attttctcaa |
| 1801 | agaccccaaa ggatgcgctc tcgttcaagg tccttttctc gacacaggtc gtgttcccga |
| 1861 | tcaccatatt ccaggtcaag atcaaggtct ccaggcagta gatcctcttc aagatcctgc |
| 1921 | tattactatg agtcaagcca ctacagacac cgcacgcacc gaaattctcc cttgtatgtg |
| 1981 | agatcacgtt caagatcgcc ctacagccgt cggcccaggt atgacagcta cgaggaatat |
| 2041 | cagcacgaga ggctgaagag ggaagaatat cgcagagagt atgagaagcg agagtctgag |
| 2101 | agggccaagc aaagggagag gcagaggcag aaggcaattg aagagcgccg tgtgatttat |
| 2161 | gtcggtaaaa tcagacctga cacaacacgg acagaactga gggaccgttt tgaagttttt |
| 2221 | ggtgaaattg aggagtgcac agtaaatctg cgggatgatg gagacagcta tggtttcatt |
| 2281 | acctaccgtt atacctgtga tgcttttgct gctcttgaaa atggatacac tttgcgcagg |
| 2341 | tcaaacgaaa ctgactttga gctgtacttt tgtggacgca agcaattttt caagtctaac |
| 2401 | tatgcagacc tagattcaaa ctcagatgac tttgaccctg cttccaccaa gagcaagtat |
| 2461 | gactctctgg attttgatag tttactgaaa gaagctcaga gaagcttgcg caggtaacat |
| 2521 | gttccctagc tgaggatgac agagggatgg cgaatacctc atgggacagc gcgtccttcc |
| 2581 | ctaaagacta ttgcaagtca tacttaggaa tttctcctac tttacactct ctgtacaaaa |
| 2641 | acaaaacaaa acaacaacaa tacaacaaga acaacaacaa caataacaac aatggtttac |
| 2701 | atgaacacag ctgctgaaga ggcaagagac agaatgatat ccagtaagca catgtttatt |
| 2761 | catgggtgtc agctttgctt ttcctggagt ctcttggtga tggagtgtgc gtgtgtgcat |
| 2821 | gtatgtgtgt gtgtatgtat gtgtgtggtg tgtgtgcttg gtttagggga agtatgtgtg |
| 2881 | ggtacatgtg aggactgggg gcacctgacc agaatgcgca agggcaaacc atttcaaatg |
| 2941 | gcagcagttc catgaagaca cgcttaaaac ctagaacttc aaaatgttcg tattctattc |
| 3001 | aaaaggaaat atatatatat atatatatat atatatatat atatataaat taaaaaggaa |
| 3061 | agaaaactaa caaccaacca accaaccaac caaccacaaa ccaccctaaa atgacagccg |
| 3121 | ctgatgtctg ggcatcagcc tttgtactct gtttttttaa gaaagtgcag aatcaacttg |
| 3181 | aagcaagctt tctctcataa cgtaatgatt atatgacaat cctgaagaaa ccacaggttc |
| 3241 | catagaacta atatcctgtc tctctctctc tctctctctc tctctttttt ttttcttttt |
| 3301 | ccttttgcca tggaatctgg gtgggagagg atactgcggg caccagaatg ctaaagtttc |
| 3361 | ctaacatttt gaagtttctg tagttcatcc ttaatcctga cacccatgta aatgtccaaa |
| 3421 | atgttgatct tccactgcaa atttcaaaag ccttgtcaat ggtcaagcgt gcagcttgtt |
| 3481 | cagcggttct ttctgaggag cggacaccgg gttacattac taatgagagt tgggtagaac |
| 3541 | tctctgagat gtgttcagat agtgtaattg ctacattctc tgatgtagtt aagtatttac |
| 3601 | agatgttaaa tggagtattt ttattttatg tatatactat acaacaatgt tcttttttgt |
| 3661 | tacagctatg cactgtaaat gcagccttct tttcaaaact gctaaatttt tcttaatcaa |
| 3721 | gaatattcaa atgtaattat gaggtgaaac aattattgta cactaacata tttagaagct |
| 3781 | gaacttactg cttatatata tttgattgta aaaacaaaaa gacagtgtgt gtgtctgttg |
| 3841 | agtgcaacaa gagcaaaatg atgctttccg cacatccatc ccttaggtga gcttcaatct |
| 3901 | aagcatcttg tcaagaaata tcctagtccc ctaaaggtat taaccacttc tgcgatattt |
| 3961 | ttccacattt tcttgtcgct tgtttttctt tgaagtttta tacactggat ttgttagggg |
| 4021 | aatgaaattt tctcatctaa aatttttcta gaagatatca tgattttatg taaagtctct |
| 4081 | caatgggtaa ccattaagaa atgtttttat tttctctatc aacagtagtt ttgaaactag |
| 4141 | aagtcaaaaa tctttttaaa atgctgtttt gttttaattt ttgtgatttt aatttgatac |
| 4201 | aaaatgctga ggtaataatt atagtatgat ttttacaata attaatgtgt gtctgaagac |
| 4261 | tatctttgaa gccagtattt ctttcccttg gcagagtatg acgatggtat ttatctgtat |
| 4321 | tttttacagt tatgcatcct gtataaatac tgatatttca ttcctttgtt tactaaagag |
| 4381 | acatatttat cagttgcaga tagcctattt attataaatt atgagatgat gaaaataata |
| 4441 | aagccagtgg aaattttcta cctaggatgc atgacaattg tcaggttgga gtgtaagtgc |
| 4501 | ttcatttggg aaattcagct tttgcagaag cagtgtttct acttgcacta gcatggcctc |
| 4561 | tgacgtgacc atggtgttgt tcttgatgac attgcttctg ctaaatttaa taaaaacttc |
| 4621 | agaaaaacct ccattttgat catcaggatt tcatctgagt gtggagtccc tggaatggaa |
| 4681 | ttcagtaaca tttggagtgt gtattcaagt ttctaaattg agattcgatt actgtttggc |
| 4741 | tgacatgact tttctggaag acatgataca cctactactc aattgttctt ttcctttctc |
| 4801 | tcgcccaaca cgatcttgta agatggattt cacccccagg ccaatgcagc taattttgat |
| 4861 | agctgcattc atttatcacc agcatattgt gttctgagtg aatccactgt ttgtcctgtc |
| 4921 | ggatgcttgc ttgatttttt ggcttcttat ttctaagtag atagaaagca ataaaaatac |
| 4981 | tatgaaatga aagaacttgt tcacaggttc tgcgttacaa cagtaacaca tctttaatcc |
| 5041 | gcctaattct tgttgttctg taggttaaat gcaggtattt taactgtgtg aacgccaaac |
| 5101 | taaagtttac agtctttctt tctgaatttt gagtatcttc tgttgtagaa taataataaa |
| 5161 | aagactatta agagcaataa attattttta agaaatcgag atttagtaaa tcctattatg |
| 5221 | tgttcaagga ccacatgtgt tctctatttt gcctttaaat ttttgtgaac caattttaaa |
| 5281 | tacattctcc tttttgccct ggattgttga catgagtgga atacttggtt tcttttctta |
| 5341 | cttatcaaaa gacagcacta cagatatcat attgaggatt aatttatccc ccctaccccc |
| 5401 | agcctgacaa atattgttac catgaagata gttttcctca atggacttca aattgcatct |
| 5461 | agaattagtg gagcttttgt atcttctgca gacactgtgg gtagcccatc aaaatgtaag |
| 5521 | ctgtgctcct ctcattttta tttttatttt tttgggagag aatatttcaa atgaacacgt |
| 5581 | gcaccccatc atcactggag gcaaatttca gcatagatct gtaggatttt tagaagaccg |
| 5641 | tgggccattg ccttcatgcc gtggtaagta ccacatctac aattttggta accgaactgg |
| 5701 | tgctttagta atgtggattt ttttcttttt taaaagagat gtagcagaat aattcttcca |
| 5761 | gtgcaacaaa atcaattttt tgctaaacga ctccgagaac aacagttggg ctgtcaacat |
| 5821 | tcaaagcagc agagagggaa ctttgcacta ttggggtatg atgtttgggt cagttgataa |
| 5881 | aaggaaacct tttcatgcct ttagatgtga gcttccagta ggtaatgatt atgtgtcctt |
| 5941 | tcttgatggc tgtaatgaga acttcaatca ctgtagtcta agacctgatc tatagatgac |
| 6001 | ctagaatagc catgtactat aatgtgatga ttctaaattt gtacctatgt gacagacatt |
| 6061 | ttcaataatg tgaactgctg atttgatgga gctactttaa gatttgtagg tgaaagtgta |
| 6121 | atactgttgg ttgaactatg ctgaagaggg aaagtgagcg attagttgag cccttgccgg |
| 6181 | gccttttttc cacctgccaa ttctacatgt attgttgtgg ttttattcat tgtatgaaaa |
| 6241 | ttcctgtgat tttttttaaa tgtgcagtac acatcagcct cactgagcta ataaagggaa |
| 6301 | acgaatgttt caaatcta |

### RAB3B

RAB3B, member RAS oncogene family (RAB3B) is a polymeric immunoglobulin receptor, expressed in epithelial cells (Van Uzendoorn et al. 2002, Dev. Cell 2:219-228). We detect RAB3B protein expression in intestinal metaplasia stem cells by immunostaining. Expression may be detected either at the RNA level or protein level. RNA expression can be measured for example by RT-PCR, RNA in situ hybridization or RNA-Seq or microarrays. Protein expression can be detected for example by immunofluorescence, immunohistochemistry, FACS, flow cytometry, Western blot or ELISA.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA); Abcam (e.g. anti-RAB3B antibody; c dat. no. ab55655) (Cambridge, Mass., USA).

The Human cDNA (NCBI Reference Sequence: NM_{—}002867.3) is Listed Below:

| SEQ ID NO: 53) | |
|---|---|
| 1 | agactccgcc cttgggcggg gcctggatgc ggccggagcg gagcagtgct ggagcgggag |
| 61 | cctcagccct caggcgccac tgtgaggacc tgaccggacc agaccatccc gcagcgcccc |
| 121 | gccccggccc cctccgcgcc ctcccgacgc caggtcctgc cgtcccgccg accgtccggg |
| 181 | agcgaacccg tcgtcccgca ctcggagtcc gcgatggctt cagtgacaga tggtaaaact |
| 241 | ggagtcaaag atgcctctga ccagaatttt gactacatgt ttaaactgct tatcattggc |
| 301 | aacagcagtg ttggcaagac ctccttcctc ttccgctatg ctgatgacac gttcacccca |
| 361 | gccttcgtta gcaccgtggg catcgacttc aaggtgaaga cagtctaccg tcacgagaag |
| 421 | cgggtgaaac tgcagatctg ggacacagct gggcaggagc ggtaccggac catcacaaca |
| 481 | gcctattacc gtggggccat gggcttcatt ctgatgtatg acatcaccaa tgaagagtcc |
| 541 | ttcaatgctg tccaagactg ggctactcag atcaagacct actcctggga caatgcacaa |
| 601 | gttattctgg tggggaacaa gtgtgacatg gaggaagaga gggttgttcc cactgagaag |
| 661 | ggccagctcc ttgcagagca gcttgggttt gatttctttg aagccagtgc aaaggagaac |
| 721 | atcagtgtaa ggcaggcctt tgagcgcctg gtggatgcca tttgtgacaa gatgtctgat |
| 781 | tcgctggaca cagacccgtc gatgctgggc tcctccaaga acacgcgtct ctcggacacc |
| 841 | ccaccgctgc tgcagcagaa ctgctcatgc taggcaaggc ccaccttcct gacctcccct |
| 901 | cattgtggcc ccacacccag tctgcttctc cctgttacac actgtccgct ctcagcccac |
| 961 | tctccctgtt acacactgcc cacactcaga gcaagatgag ttgctgctat tctttgcctg |
| 1021 | cccctggggt tctctgcaga tggtcccagt aatagatact cagcactaga ctaacataac |
| 1081 | aggtcactac acgggtgcag aatcacttta caaaagaaga ctctgtttta cgaaggggat |
| 1141 | tcactacagg gacttagaga acagtctctt ttctgccttt aaaatgagag ttcctccatt |
| 1201 | taccaaaatt tgacacgcac acattcttca ggggcatgcc aattgcgtaa agtgaggctc |
| 1261 | gcctgcatag ctaatcctgt taaagacaac ttctcaaagc acaacgtgct tgtttcctat |
| 1321 | cgggctccct gcggggcttt ctctcactac aagtcaagct tgggctctca aagccctgcg |
| 1381 | cctgttacca cggatgccca cagggcctgg gcagttgctg tggcgacagg aagagctaat |
| 1441 | cttcagagag ctcagactct ctaatgatgc tgaaggagca aaggctgagt cagaaacaca |
| 1501 | cttaagagaa aaggattggc cgggcgcggt ggctcacgcc tgtaatccca gcactttggg |
| 1561 | aggccgaggc gggtggatca tgaggtcagg agatcgagac catcctggct aacaaggtga |
| 1621 | aaccccgtct ctactaaaaa tacaaaaaat tagccgggcg cggtggcggg cgcctgtagt |
| 1681 | cccagctact cgggaggctg aggcaggaga atggcgtgaa cccgggaagc ggagcttgca |
| 1741 | gtgagccgag attgcgccac tgcagtccgc agtccggcct gggcgacaga gcgagactcc |
| 1801 | gtctcaaaaa aaaaaaaaaa aaaaaaaaga gaaaaggatt atcccctaca aaatgtcaga |
| 1861 | ggttcctgct atatgaaaga gcaagtaggt atgctcaaga aagacaaaca gagaaaaaga |
| 1921 | gaaacaggca agatcaagaa acagatcatg agtttctgat tttgctgctt tccagttggt |
| 1981 | tcttaactgt gggaacttag tgaaattggt tattagttct tagactccta gaacctgagg |
| 2041 | attttagatt tgacgggatg cccaaattta cctagtctga ctagtcagtt ctaaccttcc |
| 2101 | tttttctgac aagtgactgt caagcctaac aatcaaatct ctttctttta aagcacacct |
| 2161 | tctaggcagg gacaggagct cattttccac accatctttg tcaactctca tagaaagttt |
| 2221 | tccttgtatc gagctcaaat ctgcctcctg gaaattcttc ttcttcttcc ctccctgttg |
| 2281 | gtaccagctc tgctgtcaga gacttcacag tctgtgctcc ctctgccctg tgacgtcttc |
| 2341 | agactatttg agaacaggaa tcatgactcc tgggacttgc cttttctcta ggtcaaatac |
| 2401 | ctctataatt ccatctgctg ttcttcatag ggtcttctcc ctatcctgcc cttttcctcc |
| 2461 | aatccatctt ttaactgctc ttgagcagtc taactgagaa gtatgattca aagcaaaata |
| 2521 | aatcttaagg tggcatgact ctgaaaaaat tgagaaaatt gaactcagag atcccgatcc |
| 2581 | caaccccttt ctcctgggag tgaaacctta gtttctacca gagagtgtgg gaaaccactt |
| 2641 | ctggtggaag ccccttaatt aaatacctga ggaaaaaaat aaaagaaact cagagaccag |
| 2701 | aataaattag ctcattattc tagcttgctt ggccacaggg acatattttg ttttggctga |
| 2761 | aataatgaca tggaactggc agtgattcca gaaaaccttt cttctctatc atggcctgaa |
| 2821 | tcctcagcca cctcaaaagt cagcgggcag gaggagtctc tcgccagttt tcttttcatt |
| 2881 | tcaaatgagg ctcattgtcc tagaaaagta attaactagc aaccagtcca atgactaaat |
| 2941 | aaaaggacca tccagctgtg gctcacacct gtaatcccag cactttggaa agccaaggca |
| 3001 | ggaggaacac ttgaggccag gagtttgaga ccagcctggg caatgtggtc aaattctatc |
| 3061 | cctacaaaaa aaaaaattag ccagatgtga tggtgcatgc ctgtagtccc agctacttgg |
| 3121 | gaggctgagg caggaggatt gcttgagccc aagaatttga ggttgcagtg agctgtaatt |
| 3181 | atgccactgc attccagtct gggtgacaga gtaagaataa gaccctgtct ctctgtctct |
| 3241 | ctttctctct tttttttttt aaaggagtca gctctacaaa gatgttgctt tctttgatgc |
| 3301 | aatgcagaga gcagagcttt ggacttggaa tcaggagacc cggactctgt cattaaatca |
| 3361 | actgtgactc tgggccagtt actttccact tttgagtctt gatttcctac ttataaaatg |
| 3421 | agggagctta tttggatgat ctttaaggtc tcttttggca ctaataactc ggtgtctctt |
| 3481 | ttttttcacc ttcaccattt cagttgatcc accaaacaaa cctgagagat caggattggc |
| 3541 | atccaagagt tgtctcggcc aactctgatg tcatgcttac tctgtactag acattgttcc |
| 3601 | aagcatttta cgtgcattaa ctcatttatc ttcccaacat cttgtgaggg aggcactata |
| 3661 | gtgagcctca tttgaagatg aggaaacaaa ggtacaaaga ggttctagct ggacctctaa |
| 3721 | agtcacataa taagtaagtg gtagagctgg agttcacatc caggcagtag gctccaaggt |
| 3781 | ctgtgctctt aaccacattc tgggctgcat cttttataga caaactatga tccagagaga |
| 3841 | ttacgagact tggatcacat accaagagag tgttaaagcc acattaggat tcaattccag |
| 3901 | ggccatcaga ttccaagtcc actggagaaa agatgtatat ctctaatctg ttaacaaatt |
| 3961 | gctcaactac tcagactaat cccaggtgat ggatgtctaa tgctcaggaa aggcgagtca |
| 4021 | gtctctgagg caacagatcc catgggcctg ggtagaaaat gcccagtgct tcccagtccc |
| 4081 | aagtgctggc tttccctgta tctgcctctg ccaggcaaca cttatcaggc tcccaatcag |
| 4141 | caggagcctc catgctccac tttgaacagc ctctatgctc cagcaatggg gcatttgtga |
| 4201 | agagtgactt gattaacttt tctgaccatg ggtataatac agttgcttca gagggcagtg |
| 4261 | gttctgggtg tgatttttac actgtaacat tgtatacagt gtcatggata attactattt |
| 4321 | ttttctggtc attaacactc acctactcta gtactaggat ttcagaccaa ggtcctcatg |
| 4381 | acgcctggat attttagtat ctatatccaa taatcttttc tctcctactg aatatccagg |
| 4441 | caaagatgaa atcgttttct ttaaaactgt caaattctgt aaaactcagg agccagttca |
| 4501 | agggaacaag catcttcaca atagatggaa tcaagagtta aatgttatag tggcaagctt |
| 4561 | gtctactggg caacagacaa ccagacctgc ttgtgagatg gcagctcccc agccctgctc |
| 4621 | tgtgacctca tttctgtcaa atgaaaggca gcagcttcca gctgattgca gcatagtgtt |
| 4681 | catcaatcac agtaatagcg caattagcca ccaaggttca agctgtgtaa tatgtgttag |
| 4741 | tggcaacttg tcctggattt aatcttcctc aacaatccaa ataaaatatt taaaaactct |
| 4801 | tgacttctgg ctgggcgcag cagctcatgc ctgtaatccc agcactttgg gaggccgagg |
| 4861 | tgggcagatc acctgaggtc aggagttcaa gaccagcctg ggcaacatgg tgaaaccccc |
| 4921 | atctctacta aaaatacaaa aattagccag gcgtggtggt gggcacctga aatccctact |
| 4981 | caggaggctg aggcagagaa tcgcttgaac ctgggaggca gaggttgcag tgagccgaga |
| 5041 | tcgtgccact gcacttcagc ctgggtgaca gagcgagact ccatctcaaa acaaaacaag |
| 5101 | caaacaaaca acaacaacaa aaaacacctc ttgacttcta aagacgcaaa agtggccaaa |
| 5161 | agtgcaatac agtattgtgt ttatttacat ctattttaaa tgcatgtgta tctgtaaata |
| 5221 | caaagtgatt cgtgactcat tgtctcctca gtctatagca ttattaactt ctaggagcag |
| 5281 | cagtggagta gagtgtactg aatggtcaca gactcatcga ttatcagatc tggaaaggag |
| 5341 | cttagagaag atctgttcca ggctcctatt ttatagaagg gaaggttgac atccaaagaa |
| 5401 | tggaaggaaa tctcctaatt attctgagag tatcacagtg atggagccag gactaggtcc |
| 5461 | tggatcacct ctaagaagac acttagctat ttgactatcg actagggcct agcattatta |
| 5521 | agcactagat aaatacagat gaaaaaaaaa atgatccctg cctgcaaggt cctatgatct |
| 5581 | aatggagatg ctgtttctaa aatattatta tcccaatttg gcagtcaagg aaacagccct |
| 5641 | ggaaaagtta acatgctcaa gtcacccact agcatcattt gaaccctcct ctgtctgact |
| 5701 | catgctcttt caaatttttt tcttcagatt gtcttagcag aagggtagat gggatatacc |
| 5761 | ctctggtagt accaggctcc caaggattct tagagttaaa taacctcagt taattaaata |
| 5821 | gccacaattg cttggtgacc gaagccttat aacatccaca gaataagacc attctccaga |
| 5881 | cctgactccc caactcatat cacctgctcc tgccggccac taagctcctt gcttggatat |
| 5941 | cgagttttct ggagtatcct gaggaatgtt tgtttgactt tgtttgccaa cagtttaggg |
| 6001 | gaaggggaaa gaactacaat aaccagtgtc ctgggatctc attgatttca gattccctgc |
| 6061 | cccaagccta cacccaatta cctgccatag ttggggaatc aagtagcatc ctgtggctgg |
| 6121 | aagtaaatgc aaaacactag tccgtgagat ataaatactg ttaaatgatg gttttttaag |
| 6181 | gtcctgatcc attatatgaa gtagacaaaa ttcaaattta tttattcatt tattttctca |
| 6241 | acaaatgaat atatattatg tgccaggata caagtagtgg caaattagac acagttcttg |
| 6301 | ctttcatgaa acgtatagct tcatgattta gtatagacat tgtcaaatca tcacccaaat |
| 6361 | ataattacaa agtactctaa aggaaaggca cgtgatgctg tgagaacact caactgggaa |
| 6421 | accggaatca cctttgagaa actgtttcag gggctcttgg aagagtctac tgctcccaaa |
| 6481 | tatctctgct acccactggc cattgcttta cattcctcaa ctaagctttc accttttagt |
| 6541 | actaaccttt gatgactgat caaatacaaa tgccccaaga agactgagga taggagaaag |
| 6601 | aatatctcta cctgtgaaac attgttagac tgcctggcta ggagttcatt gttgttttct |
| 6661 | gaaggacgta accaaccact ccaaaactta caggcttaaa acaacaaaca tgtatcattt |
| 6721 | cttatgattc tgtgggttgg ctgggtggtt cttctggctg aggcaggatg gtctaggata |
| 6781 | gctacatcca catgtctggg gtcccagctg agatgactgg ggctgttgag gcctttctcc |
| 6841 | ctgtggtgtc atcctccaga aggctgccca gatttgtcca tatggtagca ggagtttcct |
| 6901 | cgaagcaaga gagggcaaga tccaacacag aagcactttt caagctctgt ttccatcaca |
| 6961 | tttgccaatg tctcactgat gaacacaagt tccatggcca agtccagttt taagaaatgg |
| 7021 | agaaataggg cttggctcag tggctcatgt ctgtaatccc agcactttgg gaggccaagg |
| 7081 | catgcggatc atttgaggtc aggagttcca gaccagcctg gccaacatgg tgaaaaccca |
| 7141 | tctctactaa aaatacaaaa attagctggg tgtggtggcg ggcatctgta atcccagcta |
| 7201 | tttgggaggc tgaagcacaa gaattgcttg aacccaggag gaggaggttg caatgagcct |
| 7261 | aaatcgcacc actgcacttc agcctgggcg atagagccag actcagtctc aaaaaaaaaa |
| 7321 | aaggggaggg ggaaatagat gccatctctt tatgggagga gctacaaaat atggtgacca |
| 7381 | atttttcaat ctaccacagg aagcaccctc agtcctctga aactaagtct ggtagatgtc |
| 7441 | ctggggtctt aaaacatggc tccgatgata tcaccaaaga caagtggcaa aactgtatag |
| 7501 | ggcagggcag tcttatcatt tgtttaatag tgatccaaag gatttacttt ggaggaatca |
| 7561 | agacactcga gatgaagaag ttttgatgct tgttaaacag tccatttgga tacctcttag |
| 7621 | ctatccccga gggatgaatc tgacttctca tttcacagga ttcaccgtag ataatggttg |
| 7681 | taattcctac cggaagttcc tggccagaag cccagcagaa agattcagta tatatagaaa |
| 7741 | agatggctcc aagaacagtt gggccttctg ttctaactgt acttccttct ttgatgtact |
| 7801 | cgtctagtcc cgaggcttta gatgccaagt ctttgataat aacgtgtatc taagtgccta |
| 7861 | ctggacattt tcatgtctca aacttaacat gtccaaattg aaactcttga ttctgccccc |
| 7921 | aaacttgttt gaaccccagt cttcacagaa aactcatcct taattctttg atttttctct |
| 7981 | ttttctcagc ctccttgtct aatctagcag cagatcctag ggttttactt ctaaatatat |
| 8041 | ctcaaatctg atcatttttc tccattttca ttggcatgac cttggtccag gccaccattg |
| 8101 | ttttctgccc tagagagcta ccacagagtt cctaacattt ccctacttac gtaattactc |
| 8161 | cactctagtc cattctgtct cacaggagta acatttttta tatatatata tatatatata |
| 8221 | tatatatata tatatatata tatatttttt tttttttaat agagacggtc ttgctatgtt |
| 8281 | gcccaagctg gtttcaaatt ctggcctcaa gcgatcctct tgccttggcc tcctgagtca |
| 8341 | ctaggattgt acgtatgagc caccgcatcc agcctcaatg gcaatctctt aaaaatctaa |
| 8401 | ataaatgaac ggctcagtaa cactgaggtt tacttcacac aaaaacaatc caaaccttgg |
| 8461 | caagacggtg aaaccctgtc tctacaaaaa atacaaaaaa ttaactgggc aaagtagcct |
| 8521 | gcacctatag tcccagctac ttgggaggct gaggtgggag gatcgattga gccctggagg |
| 8581 | tcaagaatac agtgagccat agccatgatt gtacactgtg ccactccagc ctgggtgaca |
| 8641 | gagcaagacc ctgtccccct ctcaaaaaaa aaaaaaaaag aaagaaagaa agaaaaaaaa |
| 8701 | gaagaaaagg aaagaaatga agagaattca gagacttcca ttattattaa tacctatttt |
| 8761 | attgattctg tttctagccc tgagtccgct cctaacttgc tataggatct ctggtaaatc |
| 8821 | atttcctgta ataagcagct gtcacctctc tccttgtttc ttccagaaat agtaatctct |
| 8881 | tctttagtag tactactact ccctaaccca aaccaggtga ttctagtgaa gactgtcaat |
| 8941 | aaacggagca tgtgatcaag cagggcccat cagaatcctt ccctaagatt tttataaaaa |
| 9001 | gctggaccta ttctttttcc atttgagtgg caaatatttg aagatatgag gtctaaagct |
| 9061 | gtgatggctt attctccatc cctgtgtaaa ttctggtcta tagtaagcga aaacaaggcc |
| 9121 | attaggcaga gggcagcaga gacataaggt gagaaagagt gtggtctctg gttttctaga |
| 9181 | ccctgattct ggtttggagg cttggctgat cacctcttcc tttgattctg atagaaagct |
| 9241 | caatgtatct ttctaataaa accccccttt gctttgcttg ttggagttag gttcttatcc |
| 9301 | cttgcaacca aaaatatatt gtctcttctt ttgttctcag ttttctcatt tatatatcct |
| 9361 | tctagctcca aagcacagaa attctaaaac aaacaaacaa acaaacaaaa acaaacaaaa |
| 9421 | aaaacctggg tcattcagaa aatcccactg atatagactt tctgatccag aatgtataat |
| 9481 | ctgaaaagaa gcctaccctc gtctccatcc tctcttcttg tacctgaagg aacgaagaag |
| 9541 | agggatttct caaggtgaga agcagttctc catggacact gatgacagca caggcaaagt |
| 9601 | ttcctatgac tagggatcac tgtccacaca gagtctggct tcccaggtat ccagcaggta |
| 9661 | gacaaaacag ctaactccac tgccactcct ttctccacat ccgttcctat ttctcagcca |
| 9721 | tctcagtgac atccgccatc ttgagagtca actactgact ggactgagtt gtgtggtata |
| 9781 | tgcttctgtt tacttctctt ctgtcttttt taagtggcca aatagcaaac gcttaaatag |
| 9841 | gaaatctctg ggagacttga ataaaagact ttgcttggta gaaaatcatg tcacagaaag |
| 9901 | gctaatagac agcaaagtaa atcagcaagt ccctgagcag taggattagg attcctgtct |
| 9961 | cctttcagat tcaaatgcat ctgtttctgg ggttaacagt ggactgttaa gaggctgtgc |
| 10021 | agcttgggtt aagtcattct tatctctggg cttcaggagc ttagaccaga tagtttctac |
| 10081 | aggctctctt ggtgctgatg ccttgggatt ctgtggctgt tttctgtaag atctgcaagg |
| 10141 | gggaaacagg attttggcag caatcctttc attactaaag cttcctttct tttcgggtac |
| 10201 | agtgaaaaga gccaaggctg tgtgaccccc tcatcactta gccaggcgta tggtcctggt |
| 10261 | ttctgaggct gccagaaagc atcttagcaa tttgtgtttg gatggtccat gcctgactat |
| 10321 | tctaggctgg aggttcctaa agagtaacaa gaggaagaga aacaagaatc tctgacactt |
| 10381 | gttgagaata gagcacagtc ccatttgttt gaaaagagac accaggcagc catgtttatg |
| 10441 | tgccagaaat gcattccacc tcaaggagga cttaatttat ggacccgtgt gtgccaggct |
| 10501 | gagctgggca agatctttct caggacaaac tctgccatgc agctaaaagc ctggaaacta |
| 10561 | aaggatttca tgtagtaaac tatcttccaa cccctgtaga catcagacca caggatgagg |
| 10621 | tttcagaagg tcataaggca gaatagttaa gcctacaggg cttacagtct gacagacctg |
| 10681 | ggttcagttc ttgggtcttc atcactagtt ttgtgacttc gggaagatga ctcccggagc |
| 10741 | ctcagtgagc ctcagttact tcatatgtaa atgaagtaat actatctact tcacaaggct |
| 10801 | gttgaaagga ttaaatggag aatgggtgta aaacccttag tgcagtgccg tgcacacaca |
| 10861 | gtagatgccg aacgtgtgat gttggcacta cacaatgtgt aatcccaatc aggcagagct |
| 10921 | aggcaggcaa atctaatcca ggatctttgt aaggggactg agaaccagag actggagaaa |
| 10981 | gccagtgtaa acaccatgag caaaggagca agagaagggg cattgtgtaa gtaggagatg |
| 11041 | gagcttgaac ttactaagtg gatcagggta gaagaatcca gtcaggacca agggaggaga |
| 11101 | gtccaggaaa atgccatgag cagctctgta gcatgacctt gttgggctgg gttaaagtag |
| 11161 | ggtctgccac cagtcatgtg acagaaaggt acctcatgca cttcctcctt cccccagaaa |
| 11221 | tcagcctcca ggagtgagga atgagcccag aatgagagtt tagagtgctc cagagccttt |
| 11281 | gttagaggtg ccctccgaca ttcagaaaac caggattcca gagacctggg tttgagtcct |
| 11341 | gactttgcag catactaact gtgtgatctt gaaccaacat attttcacct aatgaggctg |
| 11401 | acaatcttcc ctacttcaca aaatagttat gagagtcaaa taaaagtaca ttttagaaag |
| 11461 | tgaaatgctg tggacattta aggtggagcc actgtgagag tctaggggga tagatggtat |
| 11521 | tcgtctcaga atgaaacgaa tacacccctc tcagagccct ttccaaggat cccctccttc |
| 11581 | tttcagctcc ttccctccac ctcaatacac actcctgtcc caggaaccta acctcatcta |
| 11641 | gaaataccag ggccagcatg ccttacacct agaggtttgg ttggcttcag agaaacttct |
| 11701 | ggaggctaaa agcagccaag aagaatcagc cactacatgc tgggcctgga tgaacagagc |
| 11761 | agtgagctgt gatggggctg gggctggggc ccaggaggag caggcaggag agtttgtatg |
| 11821 | caccgtgatt caaatattat aacaaaaatc atcgatcatg tgttaggcac tttacagttc |
| 11881 | ccaaagcact ttcccatcca tgccctgatg atctttgaca caacactgtg atgtgggttt |
| 11941 | tattatttcc agtacagatg aggaagactg aggcctgcat cagtgaagca acctatccaa |
| 12001 | gactacatag agaaggcagt aaatggcagg gttagtctca gaacagggga gggtctgttc |
| 12061 | cccccgcagt gggcagtcct aattctgaac ttcacctatc tgggggtgat agaggggaac |
| 12121 | aagaggaagc ctgctgaaga gaaaacctaa acatctgttt tgtctacgta tgacttcctc |
| 12181 | tgcttgtggg agagaaggaa ggaaaggaac acattgttgt cagccccaca accccaacag |
| 12241 | aattaaaccc tggagcaggt tgaacagcag aggcttccct cagatcaagg agccaggagc |
| 12301 | agatgatcta tctctgtggc cacacagaga gatgtcacct tatgcaattt gcatatcata |
| 12361 | ttcaattccc ccaactgctc tttctaattt attcaactgg ggaccaggct ggtctcatgc |
| 12421 | caacctagga gatgtaccat agcagtatga gcagaattcc tcaggaggaa caattagcaa |
| 12481 | aaactgcagt tgcctctcga taggcctgag cagagagagg aacaatagct ctcacgtctc |
| 12541 | tcctcatcag attctaacta agcagatgtt ctcatgcttt tttcttcttc ctatgttctg |
| 12601 | tatactgaca cctcttctca gtggcatatg aaatatgaaa tgtcatgtgt tgtgagtttg |
| 12661 | tataaatata aaggaatata tatacacagt agcaaaagag aagatctcat ttacaaatat |
| 12721 | ctatggtgtt tccttgttct gtgttgatct gttttattga tacaaactga attttcttaa |
| 12781 | tgtatcttct atctctatta tagtggcaat gatggtatat gcattaaagt tcttctgaat |
| 12841 | tgtg |

### SOX2

SRY (sex determining region Y)-box 2 (SOX2), also known as ANOP3; MCOPS3, is a member of the SRY-related HMG-box (SOX) family of transcription factors. It has been shown that SOX2 is critical for embryonic stem cell pluripotency and plays a role in re-programming (Takahashi and Yamanaka, 2006, Cell 126:663-676). Detection of SOX2 expression has been observed in gastric stem cells. Expression may be detected either at the RNA level or protein level. RNA expression can be measured for example by RT-PCR, RNA in situ hybridization or RNA-Seq or microarrays. Protein expression can be detected for example by immunofluorescence, immunohistochemistry, FACS, flow cytometry, Western blot or ELISA.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA).

The Human cDNA (NCBI Reference Sequence: NM_{—}003106.3) is Listed Below:

| (SEQ ID NO: 54) | |
|---|---|
| 1 | ggatggttgt ctattaactt gttcaaaaaa gtatcaggag ttgtcaaggc agagaagaga |
| 61 | gtgtttgcaa aagggggaaa gtagtttgct gcctctttaa gactaggact gagagaaaga |
| 121 | agaggagaga gaaagaaagg gagagaagtt tgagccccag gcttaagcct ttccaaaaaa |
| 181 | taataataac aatcatcggc ggcggcagga tcggccagag gaggagggaa gcgctttttt |
| 241 | tgatcctgat tccagtttgc ctctctcttt ttttccccca aattattctt cgcctgattt |
| 301 | tcctcgcgga gccctgcgct cccgacaccc ccgcccgcct cccctcctcc tctccccccg |
| 361 | cccgcgggcc ccccaaagtc ccggccgggc cgagggtcgg cggccgccgg cgggccgggc |
| 421 | ccgcgcacag cgcccgcatg tacaacatga tggagacgga gctgaagccg ccgggcccgc |
| 481 | agcaaacttc ggggggcggc ggcggcaact ccaccgcggc ggcggccggc ggcaaccaga |
| 541 | aaaacagccc ggaccgcgtc aagcggccca tgaatgcctt catggtgtgg tcccgcgggc |
| 601 | agcggcgcaa gatggcccag gagaacccca agatgcacaa ctcggagatc agcaagcgcc |
| 661 | tgggcgccga gtggaaactt ttgtcggaga cggagaagcg gccgttcatc gacgaggcta |
| 721 | agcggctgcg agcgctgcac atgaaggagc acccggatta taaataccgg ccccggcgga |
| 781 | aaaccaagac gctcatgaag aaggataagt acacgctgcc cggcgggctg ctggcccccg |
| 841 | gcggcaatag catggcgagc ggggtcgggg tgggcgccgg cctgggcgcg ggcgtgaacc |
| 901 | agcgcatgga cagttacgcg cacatgaacg gctggagcaa cggcagctac agcatgatgc |
| 961 | aggaccagct gggctacccg cagcacccgg gcctcaatgc gcacggcgca gcgcagatgc |
| 1021 | agcccatgca ccgctacgac gtgagcgccc tgcagtacaa ctccatgacc agctcgcaga |
| 1081 | cctacatgaa cggctcgccc acctacagca tgtcctactc gcagcagggc acccctggca |
| 1141 | tggctcttgg ctccatgggt tcggtggtca agtccgaggc cagctccagc ccccctgtgg |
| 1201 | ttacctcttc ctcccactcc agggcgccct gccaggccgg ggacctccgg gacatgatca |
| 1261 | gcatgtatct ccccggcgcc gaggtgccgg aacccgccgc ccccagcaga cttcacatgt |
| 1321 | cccagcacta ccagagcggc ccggtgcccg gcacggccat taacggcaca ctgcccctct |
| 1381 | cacacatgtg agggccggac agcgaactgg aggggggaga aattttcaaa gaaaaacgag |
| 1441 | ggaaatggga ggggtgcaaa agaggagagt aagaaacagc atggagaaaa cccggtacgc |
| 1501 | tcaaaaagaa aaaggaaaaa aaaaaatccc atcacccaca gcaaatgaca gctgcaaaag |
| 1561 | agaacaccaa tcccatccac actcacgcaa aaaccgcgat gccgacaaga aaacttttat |
| 1621 | gagagagatc ctggacttct ttttggggga ctatttttgt acagagaaaa cctggggagg |
| 1681 | gtggggaggg cgggggaatg gaccttgtat agatctggag gaaagaaagc tacgaaaaac |
| 1741 | tttttaaaag ttctagtggt acggtaggag ctttgcagga agtttgcaaa agtctttacc |
| 1801 | aataatattt agagctagtc tccaagcgac gaaaaaaatg ttttaatatt tgcaagcaac |
| 1861 | ttttgtacag tatttatcga gataaacatg gcaatcaaaa tgtccattgt ttataagctg |
| 1921 | agaatttgcc aatatttttc aaggagaggc ttcttgctga attttgattc tgcagctgaa |
| 1981 | atttaggaca gttgcaaacg tgaaaagaag aaaattattc aaatttggac attttaattg |
| 2041 | tttaaaaatt gtacaaaagg aaaaaattag aataagtact ggcgaaccat ctctgtggtc |
| 2101 | ttgtttaaaa agggcaaaag ttttagactg tactaaattt tataacttac tgttaaaagc |
| 2161 | aaaaatggcc atgcaggttg acaccgttgg taatttataa tagcttttgt tcgatcccaa |
| 2221 | ctttccattt tgttcagata aaaaaaacca tgaaattact gtgtttgaaa tattttctta |
| 2281 | tggtttgtaa tatttctgta aatttattgt gatattttaa ggttttcccc cctttatttt |
| 2341 | ccgtagttgt attttaaaag attcggctct gtattatttg aatcagtctg ccgagaatcc |
| 2401 | atgtatatat ttgaactaat atcatcctta taacaggtac attttcaact taagttttta |
| 2461 | ctccattatg cacagtttga gataaataaa tttttgaaat atggacactg aaaaaaaaaa |

### SOX9

SRY (sex determining region Y)-box 9 (SOX9), also known as CMD1; SRA1; CMPD1, is a member of the SRY-related HMG-box (SOX) family of transcription factors. SOX9 was first described for its functions in chondrogenesis and sex determination, but more recently its role in epithelial cells is under investigation (Furuyama et al. 2011, Nature Genet. 43:34-41). Detection of SOX9 expression has been observed in intestinal stem cells, gastric stem cells, colon stem cells, liver stem cells, pancreatic stem cells and intestinal metaplasia stem cell. Expression may be detected either at the RNA level or protein level. RNA expression can be measured for example by RT-PCR, RNA in situ hybridization or RNA-Seq or microarrays. Protein expression can be detected for example by immunofluorescence, immunohistochemistry, FACS, flow cytometry, Western blot or ELISA.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA).

The Human cDNA (NCBI Reference Sequence: NM_{—}000346.3) is Listed Below:

| (SEQ ID NO: 55) | |
|---|---|
| 1 | ggagagccga aagcggagct cgaaactgac tggaaacttc agtggcgcgg agactcgcca |
| 61 | gtttcaaccc cggaaacttt tctttgcagg aggagaagag aaggggtgca agcgccccca |
| 121 | cttttgctct ttttcctccc ctcctcctcc tctccaattc gcctcccccc acttggagcg |
| 181 | ggcagctgtg aactggccac cccgcgcctt cctaagtgct cgccgcggta gccggccgac |
| 241 | gcgccagctt ccccgggagc cgcttgctcc gcatccgggc agccgagggg agaggagccc |
| 301 | gcgcctcgag tccccgagcc gccgcggctt ctcgcctttc ccggccacca gccccctgcc |
| 361 | ccgggcccgc gtatgaatct cctggacccc ttcatgaaga tgaccgacga gcaggagaag |
| 421 | ggcctgtccg gcgcccccag ccccaccatg tccgaggact ccgcgggctc gccctgcccg |
| 481 | tcgggctccg gctcggacac cgagaacacg cggccccagg agaacacgtt ccccaagggc |
| 541 | gagcccgatc tgaagaagga gagcgaggag gacaagttcc ccgtgtgcat ccgcgaggcg |
| 601 | gtcagccagg tgctcaaagg ctacgactgg acgctggtgc ccatgccggt gcgcgtcaac |
| 661 | ggctccagca agaacaagcc gcacgtcaag cggcccatga acgccttcat ggtgtgggcg |
| 721 | caggcggcgc gcaggaagct cgcggaccag tacccgcact tgcacaacgc cgagctcagc |
| 781 | aagacgctgg gcaagctctg gagacttctg aacgagagcg agaagcggcc cttcgtggag |
| 841 | gaggcggagc ggctgcgcgt gcagcacaag aaggaccacc cggattacaa gtaccagccg |
| 901 | cggcggagga agtcggtgaa gaacgggcag gcggaggcag aggaggccac ggagcagacg |
| 961 | cacatctccc ccaacgccat cttcaaggcg ctgcaggccg actcgccaca ctcctcctcc |
| 1021 | ggcatgagcg aggtgcactc ccccggcgag cactcggggc aatcccaggg cccaccgacc |
| 1081 | ccacccacca cccccaaaac cgacgtgcag ccgggcaagg ctgacctgaa gcgagagggg |
| 1141 | cgccccttgc cagagggggg cagacagccc cctatcgact tccgcgacgt ggacatcggc |
| 1201 | gagctgagca gcgacgtcat ctccaacatc gagaccttcg atgtcaacga gtttgaccag |
| 1261 | tacctgccgc ccaacggcca cccgggggtg ccggccacgc acggccaggt cacctacacg |
| 1321 | ggcagctacg gcatcagcag caccgcggcc accccggcga gcgcgggcca cgtgtggatg |
| 1381 | tccaagcagc aggcgccgcc gccacccccg cagcagcccc cacaggcccc gccggccccg |
| 1441 | caggcgcccc cgcagccgca ggcggcgccc ccacagcagc cggcggcacc cccgcagcag |
| 1501 | ccacaggcgc acacgctgac cacgctgagc agcgagccgg gccagtccca gcgaacgcac |
| 1561 | atcaagacgg agcagctgag ccccagccac tacagcgagc agcagcagca ctcgccccaa |
| 1621 | cagatcgcct acagcccctt caacctccca cactacagcc cctcctaccc gcccatcacc |
| 1681 | cgctcacagt acgactacac cgaccaccag aactccagct cctactacag ccacgcggca |
| 1741 | ggccagggca ccggcctcta ctccaccttc acctacatga accccgctca gcgccccatg |
| 1801 | tacaccccca tcgccgacac ctctggggtc ccttccatcc cgcagaccca cagcccccag |
| 1861 | cactgggaac aacccgtcta cacacagctc actcgacctt gaggaggcct cccacgaagg |
| 1921 | gcgaagatgg ccgagatgat cctaaaaata accgaagaaa gagaggacca accagaattc |
| 1981 | cctttggaca tttgtgtttt tttgtttttt tattttgttt tgttttttct tcttcttctt |
| 2041 | cttccttaaa gacatttaag ctaaaggcaa ctcgtaccca aatttccaag acacaaacat |
| 2101 | gacctatcca agcgcattac ccacttgtgg ccaatcagtg gccaggccaa ccttggctaa |
| 2161 | atggagcagc gaaatcaacg agaaactgga ctttttaaac cctcttcaga gcaagcgtgg |
| 2221 | aggatgatgg agaatcgtgt gatcagtgtg ctaaatctct ctgcctgttt ggactttgta |
| 2281 | attatttttt tagcagtaat taaagaaaaa agtcctctgt gaggaatatt ctctatttta |
| 2341 | aatattttta gtatgtactg tgtatgattc attaccattt tgaggggatt tatacatatt |
| 2401 | tttagataaa attaaatgct cttatttttc caacagctaa actactctta gttgaacagt |
| 2461 | gtgccctagc ttttcttgca accagagtat ttttgtacag atttgctttc tcttacaaaa |
| 2521 | agaaaaaaaa aatcctgttg tattaacatt taaaaacaga attgtgttat gtgatcagtt |
| 2581 | ttgggggtta actttgctta attcctcagg ctttgcgatt taaggaggag ctgccttaaa |
| 2641 | aaaaaataaa ggccttattt tgcaattatg ggagtaaaca atagtctaga gaagcatttg |
| 2701 | gtaagcttta tcatatatat attttttaaa gaagagaaaa acaccttgag ccttaaaacg |
| 2761 | gtgctgctgg gaaacatttg cactctttta gtgcatttcc tcctgccttt gcttgttcac |
| 2821 | tgcagtctta agaaagaggt aaaaggcaag caaaggagat gaaatctgtt ctgggaatgt |
| 2881 | ttcagcagcc aataagtgcc cgagcacact gcccccggtt gcctgcctgg gccccatgtg |
| 2941 | gaaggcagat gcctgctcgc tctgtcacct gtgcctctca gaacaccagc agttaacctt |
| 3001 | caagacattc cacttgctaa aattatttat tttgtaagga gaggttttaa ttaaaacaaa |
| 3061 | aaaaaattct tttttttttt tttttccaat tttaccttct ttaaaatagg ttgttggagc |
| 3121 | tttcctcaaa gggtatggtc atctgttgtt aaattatgtt cttaactgta accagttttt |
| 3181 | ttttatttat ctctttaatc tttttttatt attaaaagca agtttctttg tattcctcac |
| 3241 | cctagatttg tataaatgcc tttttgtcca tccctttttt ctttgttgtt tttgttgaaa |
| 3301 | acaaactgga aacttgtttc tttttttgta taaatgagag attgcaaatg tagtgtatca |
| 3361 | ctgagtcatt tgcagtgttt tctgccacag acctttgggc tgccttatat tgtgtgtgtg |
| 3421 | tgtgggtgtg tgtgtgtttt gacacaaaaa caatgcaagc atgtgtcatc catatttctc |
| 3481 | tgcatcttct cttggagtga gggaggctac ctggagggga tcagcccact gacagacctt |
| 3541 | aatcttaatt actgctgtgg ctagagagtt tgaggattgc tttttaaaaa agacagcaaa |
| 3601 | cttttttttt tatttaaaaa aagatatatt aacagtttta gaagtcagta gaataaaatc |
| 3661 | ttaaagcact cataatatgg catccttcaa tttctgtata aaagcagatc tttttaaaaa |
| 3721 | gatacttctg taacttaaga aacctggcat ttaaatcata ttttgtcttt aggtaaaagc |
| 3781 | tttggtttgt gttcgtgttt tgtttgtttc acttgtttcc ctcccagccc caaacctttt |
| 3841 | gttctctccg tgaaacttac ctttcccttt ttctttctct tttttttttt tgtatattat |
| 3901 | tgtttacaat aaatatacat tgcattaaaa agaaaaaaaa aaaaaaaaaa aaaaaaaaaa |
| 3961 | aaa |

### TSPAN8

Tetraspanin 8 (TSPAN8), also known as CO-029; TM4SF3, is a member of the transmembrane 4 superfamily, also known as the tetraspanin family. TSPAN8 expression has been detected in small intestinal stem cells, gastric stem cells, and liver stem cells. Expression may be detected either at the RNA level or protein level. RNA expression can be measured for example by RT-PCR, RNA in situ hybridization or RNA-Seq or microarrays. Protein expression can be detected for example by immunofluorescence, immunohistochemistry, FACS, flow cytometry, Western blot or ELISA.

In situ probes can be obtained for example from Advanced Cell Diagnostics RNAscope. qPCR primers can be obtained from OriGene Technologies (Rockville, Md., USA) and QIAGEN (Germantown, Md.), and other suppliers. RT-PCR primers and in situ probes can be designed using methods known in the art. Antibodies can be obtained for example from R&D Systems (Minneapolis, Minn.), EMD Millipore (Billerica, Mass., USA), Novus Biologicals (Littleton, Colo., USA); OriGene Technologies, Inc., Rockville, Md., USA); Abnova (Neihu District. Taipei City, Taiwan); or Santa Cruz Biotechnology, Inc. (Dallas, Tex., USA).

The Human cDNA (NCBI Reference Sequence: NM_{—}004616.2) is Listed Below:

| (SEQ ID NO: 56) | |
|---|---|
| 1 | agtgccccag gagctatgac aagcaaagga acatacttgc ctggagatag cctttgcgat |
| 61 | atttaaatgt ccgtggatac agaaatctct gcaggcaagt tgctccagag catattgcag |
| 121 | gacaagcctg taacgaatag ttaaattcac ggcatctgga ttcctaatcc ttttccgaaa |
| 181 | tggcaggtgt gagtgcctgt ataaaatatt ctatgtttac cttcaacttc ttgttctggc |
| 241 | tatgtggtat cttgatccta gcattagcaa tatgggtacg agtaagcaat gactctcaag |
| 301 | caatttttgg ttctgaagat gtaggctcta gctcctacgt tgctgtggac atattgattg |
| 361 | ctgtaggtgc catcatcatg attctgggct tcctgggatg ctgcggtgct ataaaagaaa |
| 421 | gtcgctgcat gcttctgttg tttttcatag gcttgcttct gatcctgctc ctgcaggtgg |
| 481 | cgacaggtat cctaggagct gttttcaaat ctaagtctga tcgcattgtg aatgaaactc |
| 541 | tctatgaaaa cacaaagctt ttgagcgcca caggggaaag tgaaaaacaa ttccaggaag |
| 601 | ccataattgt gtttcaagaa gagtttaaat gctgcggttt ggtcaatgga gctgctgatt |
| 661 | ggggaaataa ttttcaacac tatcctgaat tatgtgcctg tctagataag cagagaccat |
| 721 | gccaaagcta taatggaaaa caagtttaca aagagacctg tatttctttc ataaaagact |
| 781 | tcttggcaaa aaatttgatt atagttattg gaatatcatt tggactggca gttattgaga |
| 841 | tactgggttt ggtgttttct atggtcctgt attgccagat cgggaacaaa tgaatctgtg |
| 901 | gatgcatcaa cctatcgtca gtcaaacccc tttaaaatgt tgctttggct ttgtaaattt |
| 961 | aaatatgtaa gtgctatata agtcaggagc agctgtcttt ttaaaatgtc tcggctagct |
| 1021 | agaccacaga tatcttctag acatattgaa cacatttaag atttgaggga tataagggaa |
| 1081 | aatgatatga atgtgtattt ttactcaaaa taaaagtaac tgtttacgtt aaaaaaaaaa |
| 1141 | aaaaaaaaaa aaaaaaaaa |

### 7. Methods of Use

In a further aspect, the invention provides the use of the subject stem cells isolated from the various non-embryonic cultures in a drug discovery screen, toxicity assay, animal-based disease model, or in medicine, such as regenerative medicine.

### Genetic Manipulation of Cloned Stem Cells

For instance, stem cells isolated by the methods of the invention are suitable for numerous types of genetic manipulation, including introduction of exogenous genetic materials that may modulate the expression of one or more target genes of interest. Such kind of gene therapy can be used, for example, in a method directed at repairing damaged or diseased tissue. In brief, any suitable vectors, including an adenoviral, elntiviral, or retroviral gene delivery vehicle (see below), may be used to deliver genetic information, like DNA and/or RNA to any of the subject stem cells. A skilled person can replace or repair particular genes targeted in gene therapy. For example, a normal gene may be inserted into a nonspecific location within the genome of a diseased cell to replace a nonfunctional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the stem cells are ex vivo treated by a gene therapy approach and are subsequently transferred to the mammal, preferably a human being in need of treatment.

Any art recognized methods for genetic manipulation may be applied to the stem cells so isolated, including transfection and infection (e.g., by a viral vector) by various types of nucleic acid constructs.

For example, heterologous nucleic acids (e.g., DNA) can be introduced into the subject stem cells by way of physical treatment (e.g., electroporation, sonoporation, optical transfection, protoplast fusion, impalefection, hydrodynamic delivery, nanoparticles, magnetofection), using chemical materials or biological vectors (viruses). Chemical-based transfection can be based on calcium phosphate, cyclodextrin, polymers (e.g., cationic polymers such as DEAE-dextran or polyethylenimine), highly branched organic compounds such as dendrimers, liposomes (such as cationic liposomes, lipofection such as lipofection using Lipofectamine, etc.), or nanoparticles (with or without chemical or viral functionalization).

A nucleic acid construct comprises a nucleic acid molecule of interest, and is generally capable of directing the expression of the nucleic acid molecule of interest in the cells into which it has been introduced.

In certain embodiments, the nucleic acid construct is an expression vector wherein a nucleic acid molecule encoding a gene product, such as a polypeptide or a nucleic acid that antagonizes the expression of a polypeptide (e.g., an siRNA, miRNA, shRNA, antisense sequence, aptamer, rybozyme etc.) is operably linked to a promoter capable of directing expression of the nucleic acid molecule in the target cells (e.g., the isolated stem cell).

The term “expression vector” generally refers to a nucleic acid molecule that is capable of effecting expression of a gene/nucleic acid molecule it contains in a cell compatible with such sequences. These expression vectors typically include at least suitable promoter sequences and optionally, transcription termination signals. A nucleic acid or DNA or nucleotide sequence encoding a polypeptide is incorporated into a DNA/nucleic acid construct capable of introduction into and expression in an in vitro cell culture as identified in a method of the invention.

A DNA construct prepared for introduction into a particular cell typically include a replication system recognized by the cell, an intended DNA segment encoding a desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading phase. However, enhancers need not be contiguous with a coding sequence whose transcription they control. Linking is accomplished by ligation at convenient restriction sites or at adapters or linkers inserted in lieu thereof.

The selection of an appropriate promoter sequence generally depends upon the host cell selected for the expression of a DNA segment. Examples of suitable promoter sequences include eukaryotic promoters well known in the art (see, e.g., Sambrook and Russell, Molecular Cloning: A Laboratory Manual, Third Edition, 2001). A transcriptional regulatory sequence typically includes a heterologous enhancer or promoter that is recognized by the cell. Suitable promoters include the CMV promoter. An expression vector includes the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36.

Some aspects of the invention concern the use of a nucleic acid construct or expression vector comprising a nucleotide sequence as defined above, wherein the vector is a vector that is suitable for gene therapy. Vectors that are suitable for gene therapy are known in the art, such as those described in Anderson (Nature 392: 25-30, 1998); Walther and Stein (Drugs 60: 249-71, 2000); Kay et al. (Nat. Med. 7: 33-40, 2001); Russell (J. Gen. Virol. 81:2573-604, 2000); Amado and Chen (Science 285:674-6, 1999); Federico (Curr. Opin. Biotechnol. 10:448-53, 1999); Vigna and Naldini (J. Gene Med. 2:308-16, 2000); Marin et al. (Mol. Med. Today 3:396-403, 1997); Peng and Russell (Curr. Opin. Biotechnol. 10:454-7, 1999); Sommerfelt (J. Gen. Virol. 80:3049-64, 1999); Reiser (Gene Ther. 7: 910-3, 2000); and references cited therein (all incorporated by reference). Examples include integrative and non-integrative vectors such as those based on retroviruses, adenoviruses (AdV), adeno-associated viruses (AAV), lentiviruses, pox viruses, alphaviruses, and herpes viruses.

A particularly suitable gene therapy vector includes an Adenoviral (Ad) and Adeno-associated virus (AAV) vector. These vectors infect a wide number of dividing and non-dividing cell types. In addition, adenoviral vectors are capable of high levels of transgene expression. However, because of the episomal nature of the adenoviral and AAV vectors after cell entry, these viral vectors are most suited for therapeutic applications requiring only transient expression of the transgene (Russell, J. Gen. Virol. 81:2573-2604, 2000; Goncalves, Virol J. 2(1):43, 2005) as indicated above. Preferred adenoviral vectors are modified to reduce the host response as reviewed by Russell (2000, supra). Safety and efficacy of AAV gene transfer has been extensively studied in humans with encouraging results in the liver, muscle, CNS, and retina (Manno et al., Nat. Medicine 2006; Stroes et al., ATVB 2008; Kaplitt, Feigin, Lancet 2009; Maguire, Simonelli et al. NEJM 2008; Bainbridge et al., NEJM 2008).

AAV2 is the best characterized serotype for gene transfer studies both in humans and experimental models. AAV2 presents natural tropism towards skeletal muscles, neurons, vascular smooth muscle cells and hepatocytes. Other examples of adeno-associated virus-based non-integrative vectors include AAV1, AAV3, AAV4, AAV5, AAV 6, AAV7, AAV8, AAV9, AAV 10, AAV11 and pseudotyped AAV. The use of non-human serotypes, like AAV8 and AAV9, might be useful to overcome these immunological responses in subjects, and clinical trials have just commenced (ClinicalTrials dot gov Identifier: NCT00979238). For gene transfer into a liver cell, an adenovirus serotype 5 or an AAV serotype 2, 7 or 8 have been shown to be effective vectors and therefore a preferred Ad or AAV serotype (Gao, Molecular Therapy 13:77-87, 2006).

An exemplary retroviral vector for application in the present invention is a lentiviral based expression construct. Lentiviral vectors have the unique ability to infect non-dividing cells (Amado and Chen, Science 285:674-676, 1999). Methods for the construction and use of lentiviral based expression constructs are described in U.S. Pat. Nos. 6,165,782, 6,207,455, 6,218,181, 6,277,633, and 6,323,031, and in Federico (Curr. Opin. Biotechnol. 10:448-53, 1999) and Vigna et al. (J. Gene Med. 2:308-16, 2000).

Generally, gene therapy vectors will be as the expression vectors described above in the sense that they comprise a nucleotide sequence encoding a gene product (e.g., a polypeptide) of the invention to be expressed, whereby a nucleotide sequence is operably linked to the appropriate regulatory sequences as indicated above. Such regulatory sequence will at least comprise a promoter sequence. Suitable promoters for expression of a nucleotide sequence encoding a polypeptide from gene therapy vectors include, e.g., cytomegalovirus (CMV) intermediate early promoter, viral long terminal repeat promoters (LTRs), such as those from murine Moloney leukaemia virus (MMLV) rous sarcoma virus, or HTLV-1, the simian virus 40 (SV 40) early promoter and the herpes simplex virus thymidine kinase promoter. Additional suitable promoters are described below.

Several inducible promoter systems have been described that may be induced by the administration of small organic or inorganic compounds. Such inducible promoters include those controlled by heavy metals, such as the metallothionine promoter (Brinster et al., Nature 296:39-42, 1982; Mayo et al., Cell 29:99-108, 1982), RU-486 (a progesterone antagonist) (Wang et al., Proc. Natl. Acad. Sci. USA 91:8180-8184, 1994), steroids (Mader and White, Proc. Natl. Acad. Sci. USA 90:5603-5607, 1993), tetracycline (Gossen and Bujard, Proc. Natl. Acad. Sci. USA 89:5547-5551, 1992; U.S. Pat. No. 5,464,758; Furth et al., Proc. Natl. Acad. Sci. USA 91:9302-9306, 1994; Howe et al., J. Biol. Chem. 270:14168-14174, 1995; Resnitzky et al., Mol. Cell. Biol. 14:1669-1679, 1994; Shockett et al., Proc. Natl. Acad. Sci. USA 92:6522-6526, 1995) and the tTAER system that is based on the multi-chimeric transactivator composed of a tetR polypeptide, as activation domain of VP 16, and a ligand binding domain of an estrogen receptor (Yee et al., 2002, U.S. Pat. No. 6,432,705).

Suitable promoters for nucleotide sequences encoding small RNAs for knock down of specific genes by RNA interference (see below) include, in addition to the above mentioned polymerase II promoters, polymerase III promoters. The RNA polymerase III (pol III) is responsible for the synthesis of a large variety of small nuclear and cytoplasmic non-coding RNAs including 5S, U6, adenovirus VA1, Vault, telomerase RNA, and tRNAs. The promoter structures of a large number of genes encoding these RNAs have been determined and it has been found that RNA pol III promoters fall into three types of structures (for a review see Geiduschek and Tocchini-Valentini, Annu. Rev. Biochem. 57: 873-914, 1988; Willis, Eur. J. Biochem. 212:1-11, 1993; Hernandez, J. Biol. Chem. 276:26733-36, 2001). Particularly suitable for expression of siRNAs are the type 3 of the RNA pol III promoters, whereby transcription is driven by cis-acting elements found only in the 5′-flanking region, i.e., upstream of the transcription start site. Upstream sequence elements include a traditional TATA box (Mattaj et al., Cell 55:435-442, 1988), proximal sequence element and a distal sequence element (DSE; Gupta and Reddy, Nucleic Acids Res. 19:2073-2075, 1991). Examples of genes under the control of the type 3 pol III promoter are U6 small nuclear RNA (U6 snRNA), 7SK, Y, MRP, HI and telomerase RNA genes (see, e.g., Myslinski et al., Nucl. Acids Res. 21:2502-09, 2001).

A gene therapy vector may optionally comprise a second or one or more further nucleotide sequence coding for a second or further polypeptide. A second or further polypeptide may be a (selectable) marker polypeptide that allows for the identification, selection and/or screening for cells containing the expression construct. Suitable marker proteins for this purpose are, e.g., the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russell, Molecular Cloning: A Laboratory Manual (3rd edition), Cold Spring Harbor Laboratory, Cold Spring Harbor Laboratory Press, New York, 2001.

Alternatively, a second or further nucleotide sequence may encode a polypeptide that provides for fail-safe mechanism that allows a subject from the transgenic cells to be cured, if deemed necessary. Such a nucleotide sequence, often referred to as a suicide gene, encodes a polypeptide that is capable of converting a prodrug into a toxic substance that is capable of killing the transgenic cells in which the polypeptide is expressed. Suitable examples of such suicide genes include, e.g., the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see, e.g., Clair et al., Antimicrob. Agents Chemother. 31:844-849, 1987).

For knock down of expression of a specific polypeptide, a gene therapy vector or other expression construct is used for the expression of a desired nucleotide sequence that preferably encodes an RNAi agent, i.e., an RNA molecule that is capable of RNA interference or that is part of an RNA molecule that is capable of RNA interference. Such RNA molecules are referred to as siRNA (short interfering RNA, including, e.g., a short hairpin RNA).

A desired nucleotide sequence comprises an antisense code DNA coding for the antisense RNA directed against a region of the target gene mRNA, and/or a sense code DNA coding for the sense RNA directed against the same region of the target gene mRNA. In a DNA construct of the invention, an antisense and sense code DNAs are operably linked to one or more promoters as herein defined above that are capable of expressing an antisense and sense RNAs, respectively. “siRNA” includes a small interfering RNA that is a short-length double-stranded RNA that is not toxic in mammalian cells (Elbashir et al., Nature 411:494-98, 2001; Caplen et al., Proc. Natl. Acad. Sci. USA 98:9742-47, 2001). The length is not necessarily limited to 21 to 23 nucleotides. There is no particular limitation in the length of siRNA as long as it does not show toxicity. “siRNAs” can be, e.g., at least about 15, 18 or 21 nucleotides and up to 25, 30, 35 or 49 nucleotides long. Alternatively, the double-stranded RNA portion of a final transcription product of siRNA to be expressed can be, e.g., at least about 15, 18 or 21 nucleotides, and up to 25, 30, 35 or 49 nucleotides long.

“Antisense RNA” is preferably an RNA strand having a sequence complementary to a target gene mRNA, and thought to induce RNAi by binding to the target gene mRNA.

“Sense RNA” has a sequence complementary to the antisense RNA, and annealed to its complementary antisense RNA to form siRNA.

The term “target gene” in this context includes a gene whose expression is to be silenced due to siRNA to be expressed by the present system, and can be arbitrarily selected. As this target gene, for example, genes whose sequences are known but whose functions remain to be elucidated, and genes whose expressions are thought to be causative of diseases are preferably selected. A target gene may be one whose genome sequence has not been fully elucidated, as long as a partial sequence of mRNA of the gene having at least 15 nucleotides or more, which is a length capable of binding to one of the strands (antisense RNA strand) of siRNA, has been determined. Therefore, genes, expressed sequence tags (ESTs) and portions of mRNA, of which some sequence (preferably at least 15 nucleotides) has been elucidated, may be selected as the “target gene” even if their full length sequences have not been determined.

The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. A non-pairing portions can be contained to the extent that they do not interfere with siRNA formation.

The “bulge” used herein may comprise 1 to 2 non-pairing nucleotides, and the double-stranded RNA region of siRNAs in which two RNA strands pair up contains preferably 1 to 7, more preferably 1 to 5 bulges.

The term “mismatch” as used herein may be contained in the double-stranded RNA region of siRNAs in which two RNA strands pair up, preferably 1 to 7, more preferably 1 to 5, in number. In certain mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, a double-stranded RNA region of siRNAs in which two RNA strands pair up may contain both bulge and mismatched, which sum up to, preferably 1 to 7, more preferably 1 to 5 in number. Such non-pairing portions (mismatches or bulges, etc.) can suppress the below-described recombination between antisense and sense code DNAs and make the siRNA expression system as described below stable. Furthermore, although it is difficult to sequence stem loop DNA containing no non-pairing portion in the double-stranded RNA region of siRNAs in which two RNA strands pair up, the sequencing is enabled by introducing mismatches or bulges as described above. Moreover, siRNAs containing mismatches or bulges in the pairing double-stranded RNA region have the advantage of being stable in E. coli or animal cells.

The terminal structure of siRNA may be either blunt or cohesive (overhanging) as long as siRNA enables to silence the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 by double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is necessarily the cut off structure at both ends as described above, and may have a stem-loop structure in which ends of one side of double-stranded RNA are connected by a linker RNA (a “shRNA”). The length of the double-stranded RNA region (stem-loop portion) can be, e.g., at least 15, 18 or 21 nucleotides and up to 25, 30, 35 or 49 nucleotides long. Alternatively, the length of the double-stranded RNA region that is a final transcription product of siRNAs to be expressed is, e.g., at least 15, 18 or 21 nucleotides and up to 25, 30, 35 or 49 nucleotides long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA, snRNA or viral RNA, or an artificial RNA molecule.

To express antisense and sense RNAs from the antisense and sense code DNAs respectively, a DNA construct of the present invention comprise a promoter as defined above. The number and the location of the promoter in the construct can in principle be arbitrarily selected as long as it is capable of expressing antisense and sense code DNAs. As a simple example of a DNA construct of the invention, a tandem expression system can be formed, in which a promoter is located upstream of both antisense and sense code DNAs. This tandem expression system is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. Herein, there is no particular limitation in the length and sequence of the linker DNA, which may have any length and sequence as long as its sequence is not the termination sequence, and its length and sequence do not hinder the stem portion pairing during the mature RNA production as described above. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA is not hindered. If this state hinders the gene silencing, it is preferable to perform trimming of the transcript using a trimming means (for example, ribozyme as are known in the art). It will be clear to the skilled person that an antisense and sense RNAs may be expressed in the same vector or in different vectors. To avoid the addition of excess sequences downstream of the sense and antisense RNAs, it is preferred to place a terminator of transcription at the 3′ ends of the respective strands (strands coding for antisense and sense RNAs). The terminator may be a sequence of four or more consecutive adenine (A) nucleotides.

### Genome Editing

Genome editing may be used to change the genomic sequence of the subject cloned stem cells, including cloned cancer (or other disease) stem cells, by introducing heterologous transgene or by inhibiting expression of a target endogenous gene. Such genetically engineered stem cells can be used, for regenerative medicine (see below) or wound healing. Thus in certain embodiments, the subject methods of regenerative medicine (see below) comprise using a subject stem cell the genome sequence of which has been modified by genomic editing.

Genome editing may be performed using any art-recognized technology, such as ZFN/TALEN or CRISPR technologies (see review by Gaj et al., Trends in Biotech. 31(7): 397-405, 2013, the entire text and all cited references therein are incorporated herein by reference). Such technologies enable one to manipulate virtually any gene in a diverse range of cell types and organisms, thus enabling a broad range of genetic modifications by inducing DNA double-strand (DSB) breaks that stimulate error-prone nonhomologous end joining (NHEJ) or homology-directed repair (HDR) at specific genomic locations.

Zinc-finger nucleases (ZFNs) and Transcription activator-like effector nucleases (TALENs) are chimeric nucleases composed of programmable, sequence-specific DNA-binding modules linked to a nonspecific DNA cleavage domain. They are artificial restriction enzymes (REs) generated by fusing a zinc-finger or TAL effector DNA binding domain to a DNA cleavage domain. A zinc-finger (ZF) or transcription activator-like effector (TALE) can be engineered to bind any desired target DNA sequence, and be fused to a DNA cleavage domain of an RE, thus creating an engineer restriction enzyme (ZFN or TALEN) that is specific for the desired target DNA sequence. When ZFN/TALEN is introduced into cells, it can be used for genome editing in situ. Indeed, the versatility of the ZFNs and TALENs can be expanded to effector domains other than nucleases, such as transcription activators and repressors, recombinases, transposases, DNA and histone methyl transferases, and histone acetyltransferases, to affect genomic structure and function.

The Cys₂-His₂ zinc-finger domain is among the most common types of DNA-binding motifs found in eukaryotes and represents the second most frequently encoded protein domain in the human genome. An individual zinc-finger has about 30 amino acids in a conserved ββα configuration. Key to the application of zinc-finger proteins for specific DNA recognition was the development of unnatural arrays that contain more than three zinc-finger domains. This advance was facilitated by the structure-based discovery of a highly conserved linker sequence that enabled construction of synthetic zinc-finger proteins that recognized DNA sequences 9-18 bp in length. This design has proven to be the optimal strategy for constructing zinc-finger proteins that recognize contiguous DNA sequences that are specific in complex genomes. Suitable zinc-fingers may be obtained by modular assembly approach (e.g., using a preselected library of zinc-finger modules generated by selection of large combinatorial libraries or by rational design). Zinc-finger domains have been developed that recognize nearly all of the 64 possible nucleotide triplets, preselected zinc-finger modules can be linked together in tandem to target DNA sequences that contain a series of these DNA triplets. Alternatively, selection-based approaches, such as oligomerized pool engineering (OPEN) can be used to select for new zinc-finger arrays from randomized libraries that take into consideration context-dependent interactions between neighboring fingers. A combination of the two approaches is also used.

Engineered zinc fingers are commercially available. Sangamo Biosciences (Richmond, Calif., USA) has developed a propriety platform (CompoZr) for zinc-finger construction in partnership with Sigma-Aldrich (St. Louis, Mo., USA), which platform allows investigators to bypass zinc-finger construction and validation altogether, and many thousands of proteins are already available. Broadly, zinc-finger protein technology enables targeting of virtually any sequence.

TAL effectors are proteins secreted by the plant pathogenic Xanthomonas bacteria, with DNA binding domain containing a repeated highly conserved 33-34 amino acid sequence, with the exception of the 12th and 13th amino acids. These two locations are highly variable (Repeat Variable Diresidue, or RVD) and show a strong correlation with specific nucleotide recognition. This simple relationship between amino acid sequence and DNA recognition has allowed for the engineering of specific DNA binding domains by selecting a combination of repeat segments containing the appropriate RVDs. Like zinc fingers, modular TALE repeats are linked together to recognize contiguous DNA sequences. Numerous effector domains have been made available to fuse to TALE repeats for targeted genetic modifications, including nucleases, transcriptional activators, and site-specific recombinases. Rapid assembly of custom TALE arrays can be achieved by using strategies include “Golden Gate” molecular cloning, high-throughput solid-phase assembly, and ligation-independent cloning techniques, all can be used in the instant invention for genome editing of the cloned stem cells.

TALE repeats can be easily assembled using numerous tools available in the art, such as a library of TALENs targeting 18,740 human protein-coding genes (Kim et al., Nat. Biotechnol. 31, 251-258, 2013). Custom-designed TALE arrays are also commercially available through, for example, Cellectis Bioresearch (Paris, France), Transposagen Biopharmaceuticals (Lexington, Ky., USA), and Life Technologies (Grand Island, N.Y., USA).

The non-specific DNA cleavage domain from the end of a RE, such as the FokI endonuclease (or FokI cleavage domain variants, such as Sharkey, with mutations designed to improve cleavage specificity and/or cleavage activity), can be used to construct hybrid nucleases that are active in a yeast assay (also active in plant cells and in animal cells). To improve ZFN activity, transient hypothermic culture conditions can be used to increase nuclease expression levels; co-delivery of site-specific nucleases with DNA end-processing enzymes, and the use of fluorescent surrogate reporter vectors that allow for the enrichment of ZFN- and TALEN-modified cells, may also be used. The specificity of ZFN-mediated genome editing can also be refined by using zinc-finger nickases (ZFNickases), which take advantage of the finding that induction of nicked DNA stimulates HDR without activating the error-prone NHEJ repair pathway.

The simple relationship between amino acid sequence and DNA recognition of the TALE binding domain allows for designable proteins. A publicly available software program (DNAWorks) can be used to calculate oligonucleotides suitable for assembly in a two step PCR. A number of modular assembly schemes for generating engineered TALE constructs have also been reported and known in the art. Both methods offer a systematic approach to engineering DNA binding domains that is conceptually similar to the modular assembly method for generating zinc finger DNA recognition domains.

Once the TALEN genes have been assembled, they are introduced into the target cell on a vector using any art recognized methods (such as electroporation or transfection using cationic lipid-based reagents, using plasmid vectors, various viral vectors such as adenoviral, AAV, and Integrase-deficient lentiviral vectors (IDLVs)). Alternatively, TALENs can be delivered to the cell as mRNA, which removes the possibility of genomic integration of the TALEN-expressing protein. It can also dramatically increase the level of homology directed repair (HDR) and the success of introgression during gene editing. Finally, direct delivery of purified ZFN/TALEN proteins into cells may also be used. This approach does not carry the risk of insertional mutagenesis, and leads to fewer off-target effects than delivery systems that rely on expression from nucleic acids, and thus may be optimally used for studies that require precise genome engineering in cells, such as the instant stem cells.

TALENs can be used to edit genomes by inducing double-strand breaks (DSB), which cells respond to with repair mechanisms. Non-homologous end joining (NHEJ) reconnects DNA from either side of a double-strand break where there is very little or no sequence overlap for annealing. A simple heteroduplex cleavage assay can be run which detects any difference between two alleles amplified by PCR. Cleavage products can be visualized on simple agarose gels or slab gel systems. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes. TALENs have been used to generate stably modified human embryonic stem cell and induced pluripotent stem cell (iPSCs) clones to generate knockout C. elegans, rats, and zebrafish.

For stem cell based therapy, ZFNs and TALENs are capable of correcting the underlying cause of the disease, therefore permanently eliminating the symptoms with precise genome modifications. For example, ZFN-induced HDR has been used to directly correct the disease-causing mutations associated with X-linked severe combined immune deficiency (SCID), hemophilia B, sickle-cell disease, α1-antitrypsin deficiency and numerous other genetic diseases, either by repair defective target genes, or by knocking out a target gene. In addition, these site-specific nucleases can also be used to safely insert a therapeutic transgenes into the subject stem cell, at a specific “safe harbor” locations in the human genome. Such techniques, in combination with the stem cells of the invention, can be used in gene therapy, including treatments based on autologous stem cell transplantation, where one or more genes of the cloned (diseased or normal) stem cells are manipulated to increase or decrease/eliminate a target gene expression.

Alternatively, CRISPR/Cas system can also be used to efficiently induce targeted genetic alterations into the subject stem cells. CRISPR/Cas (CRISPR associated) systems or “Clustered Regulatory Interspaced Short Palindromic Repeats” are loci that contain multiple short direct repeats, and provide acquired immunity to bacteria and archaea. CRISPR systems rely on crRNA and tracrRNA for sequence-specific silencing of invading foreign DNA. The term “tracrRNA” stands for trans-activating chimeric RNA, which is noncoding RNA that promotes crRNA processing, and is required for activating RNA-guided cleavage by Cas9. CRISPR RNA or crRNA base pairs with tracrRNA to form a two-RNA structure that guides the Cas9 endonuclease to complementary DNA sites for cleavage.

Three types of CRISPR/Cas systems exist: in type II systems, Cas9 serves as an RNA-guided DNA endonuclease that cleaves DNA upon crRNA-tracrRNA target recognition. In bacteria, the CRISPR system provides acquired immunity against invading foreign DNA via RNA-guided DNA cleavage. The CRISPR/Cas system can be retargeted to cleave virtually any DNA sequence by redesigning the crRNA. Indeed, the CRISPR/Cas system has been shown to be directly portable to human cells by co-delivery of plasmids expressing the Cas9 endonuclease and the necessary crRNA components. These programmable RNA-guided DNA endonucleases have demonstrated multiplexed gene disruption capabilities and targeted integration in iPS cells, and can thus be used similarly in the subject stem cells.

### Cancer Stem Cells

The methods and reagents of the invention also enable culturing and isolating cancer-derived cancer stem cells (CSCs), which in turn may be used in numerous applications previously impossible or impractical to carry out, partly due to the inability to obtaining such CSCs in large quantity and as single cell clones.

For example, the libraries of CSCs established from a single patient using the methods of the invention enable comparison between patient-matched sensitive and resistant clones for directed drug discovery efforts. Certain genes may be up-regulated or down-regulated in the resistant clones compared to the sensitive clones. Inhibitors for the up-regulated genes may be further validated as a drug target gene, by testing, for example, the ability of down-regulation of the target gene in the resistant clones, and determining its effect on drug resistance. Conversely, restoring or overexpressing the down-regulated genes in the resistant clones may also overcome drug resistance.

Thus in one aspect, the invention provides a drug discovery method using CSCs isolated using the subject methods and media, for identifying genes up- or down-regulated in drug resistant CSC clones, the method comprising: (1) using the method of the invention, obtaining a plurality of cell clones from a cancerous tissue (such as one from a cancer patient); (2) contacting the plurality of cell clones with one or more chemical compound (e.g., cancer drug), under conditions in which a small percentage (e.g., no more than 1%, 0.5%, 0.2%, 0.1%, 0.05%, 0.01% or fewer) of drug-resistant clones survive; (3) comparing gene expression profiles of the drug-resistant clones with that of the sensitive clones (e.g., one or more randomly picked plurality of cell clones before step (2), which are presumably sensitive to drug treatment), thus identifying genes up- or down-regulated in the surviving drug-resistant clones.

In certain embodiments, the method further comprises inhibiting the expression of an up-regulated gene in the surviving drug-resistant clone. For example, the up-regulated gene may be commonly up-regulated in two or more surviving drug-resistant clones, either from the same type of tumors or different types of tumors, either from the same patient, or from different patients. In certain embodiments, the up-regulated gene may be specific for the patient from whom the CSCs are isolated. This can be helpful in designing personalized medicine or treatment regimens for the patient.

In certain embodiments, the method further comprises restoring or increasing the expression of a down-regulated gene in the surviving drug-resistant clone. For example, the down-regulated gene may be commonly down-regulated in two or more surviving drug-resistant clones, either from the same type of tumors or different types of tumors, either from the same patient, or from different patients. In certain embodiments, the down-regulated gene may be specific for the patient from whom the CSCs are isolated. This can also be helpful in designing personalized medicine or treatment regimens for the patient.

In a related aspect, the invention provides a drug discovery method using CSCs isolated using the subject methods and media, for identifying a candidate compound that inhibit the growth or promote the killing of a drug-resistant CSC, the method comprising: (1) using the method of the invention, obtaining a plurality of cell clones from a cancerous tissue (such as one from a cancer patient); (2) contacting the plurality of cell clones with one or more chemical compound (e.g., cancer drug), under conditions in which a small percentage (e.g., no more than 1%, 0.5%, 0.2%, 0.1%, 0.05%, 0.01% or fewer) of drug-resistant clones survive; (3) contacting the surviving drug-resistant clones with a plurality of candidate compounds, and (4) identifying one or more candidate compounds that inhibit the growth or promote the killing of the surviving drug-resistant clones. In certain embodiments, the method is performed using high-throughput screens format, for candidate drugs that target resistant cells.

In certain embodiments, the method further comprises testing general toxicity of the identified candidate compounds on the matching sensitive clones (e.g., one or more randomly picked plurality of cell clones before step (2), which are presumably sensitive to drug treatment), and/or the matching healthy cells from the same patient from whom the CSCs are isolated. Preferably, any identified candidate compounds specifically or preferentially inhibit the growth or promote the killing of the drug-resistant CSC, compared to the matching sensitive clones and/or the matching healthy cells.

In certain embodiments, the healthy cells are patient-matched normal stem cells similarly isolated using the methods and reagents of the invention.

The above embodiment is partly based on the discovery that, in many cases, drug-resistant CSCs grow more slowly compared to drug-sensitive clones. While not wishing to be bound by any particular theory, Applicant believes that the slow growth is likely a consequence of gene expression alterations in the drug-resistant CSCs for evading chemotherapy. Thus, it is expected that certain agents may inhibit the growth or kill drug resistant cells preferentially while being less toxic than standard chemotherapy drugs (such as cisplatin or paclitaxel) used to treat the cancer in the first place.

In another aspect, the invention provides a method for identifying a suitable or effective treatment for a patient in need of treating a disease, the method comprising: (1) using the method of the invention, obtaining a plurality of stem cell clones from a disease tissue (such as a cancerous tissue) from the patient; (2) subjecting the plurality of cell clones to one or more candidate treatments; (3) determining the effectiveness of each of said one or more candidate treatments; thereby identifying a suitable or effective treatment for the patient in need of treating the disease. This can be useful, for example, when the patient has several possible treatment options, each may or may not be suitable or effective for the patient.

In a related aspect, the invention provides a method for screening for the most suitable or effective treatment among a plurality of candidate treatments, for treating a patient in need of treating a disease, the method comprising: (1) using the method of the invention, obtaining a plurality of stem cell clones from a disease tissue (such as a cancerous tissue) from the patient; (2) subjecting the plurality of cell clones to said candidate treatments; (3) comparing the relative effectiveness of said one or more candidate treatments; thereby identifying the most suitable or effective treatment for the patient. This can be useful, for example, when the patient has several alternative treatment options that may each be effective against a specific patient population but not necessarily effective for others.

In certain embodiments, the disease is a cancer, such as any of the cancers from which a cancer stem cell can be isolated.

In certain embodiments, the treatment is a chemotherapy regimen, such as one utilizing one or more chemotherapeutic agents. In certain embodiments, the treatment is radiotherapy. In certain embodiments, the treatment is immunotherapy, such as one using a cell-binding agent (e.g., antibody) that specifically binds to a surface ligand (e.g., surface antigen) of a cancer cell. In certain embodiments, the treatment is a combination therapy of surgery, chemotherapy, radiotherapy, and/or immunotherapy.

In certain embodiments, the disease is an inflammatory disease, a disease from which a disease-associated stem cell can be isolated, or any disease referenced herein.

In certain embodiments, the method further comprises treating the patient using one or more identified suitable or effective treatment for the disease.

In certain embodiments, the method further comprises producing a report that provides the effectiveness of each of said candidate treatments, such as the effectiveness of each of the candidate chemotherapeutic agents tested, either individually or in combination (including sequentially or simultaneously).

In certain embodiments, the method further comprises providing a recommendation for the most effective treatment.

In a related aspect, the invention provides kits and reagents for carrying out the methods of the invention.

In certain embodiments, the general screening method of the invention (not necessarily limited to cancer stem cells) is carried out in high-throughput/automatic fashion. For high-throughput purposes, the expanded stem cell population can be cultured in multiwell plates such as, for example, 96-well plates or 384-well plates. Libraries of molecules are used to identify a molecule that affects the plated stem cells. Preferred libraries include (without limitation) antibody fragment libraries, peptide phage display libraries, peptide libraries (e.g., LOPAP™, Sigma Aldrich), lipid libraries (BioMol), synthetic compound libraries (e.g., LOP AC™, Sigma Aldrich) or natural compound libraries (Specs, TimTec). Furthermore, genetic libraries can be used that induce or repress the expression of one of more genes in the progeny of the stem cells. These genetic libraries comprise cDNA libraries, antisense libraries, and siRNA or other non-coding RNA libraries.

The stem cells are preferably exposed to multiple concentrations of a test/candidate agent for a certain period of time. At the end of the exposure period, the cultures are evaluated for a pre-determined effect, such as any changes in a cell, including, but not limited to, a reduction in, or loss of, proliferation, a morphological change, and cell death.

The expanded stem cell population can also be used to identify drugs that specifically target epithelial carcinoma cells or stem cells isolated therefrom, but not the expanded stem cell population itself.

The ready cloning of cancer stem cells also enables immunological approaches to tumor destruction. The technology described herein enables the high-efficiency cloning of CSCs and therefore potentially provides information that would aid approaches to eradicating these cells via immune activation.

For example, upon isolating the CSCs (either drug-sensitive or drug-resistant), one or more epitopes of such CSCs, preferably CSC-specific epitopes compared to healthy control (e.g., epitopes on the cell surface or secretome of CSCs), may be used to vaccinate antigen-presenting cells (APCs) to direct lymphocytes to target these CSCs. The immunological approaches might include, as was done to melanoma, the identification and targeting of molecules on the cell surface or secretome of CSCs that suppress immune surveillance.

### Regenerative Medicine

The subject stem cells isolated from the various sources of tissues, including non-embryonic human tissues, are useful in regenerative medicine, for example in post-trauma, post-radiation, and/or post-surgery repair of the various damaged tissues or organs. For example, the isolated intestinal stem cells, such as those isolated from the healthy tissues of a patient or from a healthy donor, can be used to generate intestinal epithelium in the repair of intestinal epithelium in patients suffering from inflammatory bowel disease (IBD), such as Crohn's disease and ulcerative colitis (UC), and in the repair of the intestinal epithelium in patients suffering from short bowel syndrome.

Further use can be found in the repair of the intestinal epithelium in patients with hereditary diseases of the small intestine/colon. Cultures comprising pancreatic stem cells may be used in regenerative medicine, for example as implants after resection of the pancreas or part thereof, and for treatment of diabetes such as diabetes I and diabetes II.

In an alternative embodiment, the expanded epithelial stem cells (e.g., pancreatic stem cells) are differentiated into pancreatic beta-cells. For example, human pancreatic stem cells of the invention may be transplanted under the peri-renal capsule in mice, to allow these cells differentiate to form mature beta cells that secrete insulin. Thus, even if the population of stem cells of the invention does not secrete insulin at a detectable level, the stem cells may be cultured in vitro for differentiation into pancreatic beta-cells, and these cells may be useful for transplantation into a patient for the treatment of an insulin-deficiency disorder such as diabetes.

In yet another embodiment, a small biopsy or tissue sample can be taken from adult donors, and stem cells therein can be isolated and expanded, and optionally differentiated, to generate transplantable epithelium for regenerative purposes. The fact that the subject stem cells can be frozen and thawed and put back into culture without losing the stem cell character and without significant cell death further adds to the applicability of the subject stem cells for transplantation purposes.

Thus the invention provides a stem cell or expanded clone thereof or differentiation product thereof (or collectively “stem cell” in the context of regenerative medicinal use) for use in transplantation into a mammal, preferably into a human. Also provided is a method of treating a patient in need of a transplant comprising transplanting a population of the stem cell of the invention into the patient, wherein the patient is a mammal, preferably a human.

In another embodiment, the expanded epithelial stem cells are differentiated into related tissue fates such as, for example, pancreatic cells including pancreatic β-cells, and liver cells.

Thus, another aspect of the invention provides a method of treating a human or non-human animal patient through cellular therapy. Such cellular therapy encompasses the application or administration of the stem cells of the invention (such as tissue matched stem cells of the invention) to the patient through any appropriate means. Specifically, such methods of treatment involve the regeneration of damaged tissue or wound healing.

In accordance with the invention, a patient can be treated with allogeneic or autologous stem cells or clonal expansion thereof. “Autologous” cells are cells which originated from the same organism into which they are being re-introduced for cellular therapy, for example in order to permit tissue regeneration. However, the cells have not necessarily been isolated from the same tissue as the tissue they are being introduced into. An autologous cell does not require matching to the patient in order to overcome the problems of rejection. “Allogeneic” cells are cells which originated from an individual which is different from the individual into which the cells are being introduced for cellular therapy, for example in order to permit tissue regeneration, although of the same species. Some degree of patient matching may still be required to prevent the problems of rejection.

Generally the stem cells of the invention are introduced into the body of the patient by injection or implantation. Generally the cells will be directly injected into the tissue in which they are intended to act. Alternatively, the cells will be injected through the portal vein. A syringe containing cells of the invention and a pharmaceutically acceptable carrier is included within the scope of the invention. A catheter attached to a syringe containing cells of the invention and a pharmaceutically acceptable carrier is also included within the scope of the invention.

Stem cells of the invention can also be used in the regeneration of tissue. In order to achieve this function, cells may be injected or implanted directly into the damaged tissue, where they may multiply and eventually differentiate into the required cell type, in accordance with their location in the body, and/or after homing to their tissue of origin. Alternatively, the subject stem cells can be injected or implanted directly into the damaged tissue. Tissues that are susceptible to treatment include all damaged tissues, particularly including those which may have been damaged by disease, injury, trauma, an autoimmune reaction, or by a viral or bacterial infection. In some embodiments of the invention, the stem cells of the invention are used to regenerate the lung, esophagus, stomach, small intestine, colon, intestinal metaplasia, fallopian tube, kidney, pancreas, bladder, liver, or gastric system, or a portion/section thereof.

In certain embodiments, the patient is a human, but may alternatively be a non-human mammal, such as a cat, dog, horse, cow, pig, sheep, rabbit or mouse.

In certain embodiments, the stem cells of the invention are injected into a patient using a syringe, such as a Hamilton syringe.

The skilled person will be aware what the appropriate dosage of stem cells of the invention will be for a particular condition to be treated.

In certain embodiments, the stem cells of the invention, either in solution, in microspheres, or in microparticles of a variety of compositions, are administered into the artery irrigating the tissue or the part of the damaged organ in need of regeneration. Generally such administration will be performed using a catheter. The catheter may be one of the large variety of balloon catheters used for angioplasty and/or cell delivery or a catheter designed for the specific purpose of delivering the cells to a particular local of the body.

For certain uses, the stem cells may be encapsulated into microspheres made of a number of different biodegradable compounds, and with a diameter of about 15 μm. This method may allow intravascularly administered stem cells to remain at the site of damage, and not to go through the capillary network and into the systemic circulation in the first passage. The retention at the arterial side of the capillary network may also facilitate their translocation into the extravascular space.

In certain embodiments, the stem cells may be retrograde injected into the vascular tree, either through a vein to deliver them to the whole body or locally into the particular vein that drains into the tissue or body part to which the stem cells are directed.

In another embodiment, the stem cells of the invention may be implanted into the damaged tissue adhered to a biocompatible implant. Within this embodiment, the cells may be adhered to the biocompatible implant in vitro, prior to implantation into the patient. As will be clear to a person skilled in the art, any one of a number of adherents may be used to adhere the cells to the implant, prior to implantation. By way of example only, such adherents may include fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

In another embodiment, the stem cells of the invention may be embedded in a matrix, prior to implantation of the matrix into the patient. Generally, the matrix will be implanted into the damaged tissue of the patient. Examples of matrices include collagen based matrices, fibrin based matrices, laminin based matrices, fibronectin based matrices and artificial matrices. This list is provided by way of illustration only, and is not intended to be limiting.

In a further embodiment, the stem cells of the invention may be implanted or injected into the patient together with a matrix forming component. This may allow the cells to form a matrix following injection or implantation, ensuring that the stem cells remain at the appropriate location within the patient. Examples of matrix forming components include fibrin glue liquid alkyl, cyanoacrylate monomers, plasticizers, polysaccharides such as dextran, ethylene oxide-containing oligomers, block co-polymers such as poloxamer and Pluronics, non-ionic surfactants such as Tween and Triton 8, and artificial matrix forming components. This list is provided by way of illustration only, and is not intended to be limiting. It will be clear to a person skilled in the art, that any combination of one or more matrix forming components may be used.

In a further embodiment, the stem cells of the invention may be contained within a microsphere. Within this embodiment, the cells may be encapsulated within the center of the microsphere. Also within this embodiment, the cells may be embedded into the matrix material of the microsphere. The matrix material may include any suitable biodegradable polymer, including but not limited to alginates, Poly ethylene glycol (PLGA), and polyurethanes. This list is provided by way of example only, and is not intended to be limiting.

In a further embodiment, the stem cells of the invention may be adhered to a medical device intended for implantation. Examples of such medical devices include stents, pins, stitches, splits, pacemakers, prosthetic joints, artificial skin, and rods. This list is provided by way of illustration only, and is not intended to be limiting. It will be clear to a person skilled in the art, that the cells may be adhered to the medical device by a variety of methods. For example, the stem cells may be adhered to the medical device using fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

Numerous diseases or tissue damages can be treated using the subject stem cells as regenerative medicine. Non-limiting examples include: wound healing, diabetic ulcer, skin graft or regeneration, type 1 diabetes mellitus, cardiovascular repair (such as that after myocardial infarction and cardiac failure), CNS injury repair (such as one after stroke, brain trauma, cerebral palsy and other forms of brain injury), spinal-cord injury, Parkinson's disease, Huntington's disease, Alzheimer's disease, Celiac disease, Graft-versus-host disease, Crohn's disease and ulcerative colitis, blindness and vision impairment (e.g., due to macular degeneration), ALS, infertility, etc. Various veterinary uses of the subject stem cells are also within the scope of the invention, including without limitation, myocardial infarction, stroke, tendon and ligament damage, osteoarthritis, osteochondrosis and muscular dystrophy both in large animals.

For example, the subject liver stem cells may be useful in regenerative medicine in post-radiation and/or post-surgery repair of the liver epithelium, or in the repair of the epithelium in patients suffering from chronic or acute liver failure or disease. Treatable liver diseases include, but are not limited to Hepatocellular Carcinoma, Alagille Syndrome, Alpha-1-Antitrypsin Deficiency, Autoimmune Hepatitis, Biliary Atresia, Chronic Hepatitis, Cancer of the Liver, Cirrhosis Liver Cysts Fatty Liver, Galactosemia Gilbert's Syndrome, Primary Biliary Cirrhosis, Hepatitis A, Hepatitis B, Hepatitis C, Primary Sclerosing Cholangitis, Reye's Syndrome, Sarcoidosis, Tyrosinemia, Type I Glycogen Storage Disease, Wilson's Disease, Neonatal Hepatitis, Non-alchoholic SteatoHepatitis, Porphyria, and Hemochromatosis.

Genetic conditions that lead to liver failure could also benefit from cell-based therapy in the form of partial or full cell replacement using stem cells cultured according to the media and/or methods of the invention. A non-limiting list of genetic conditions that lead to liver failure includes: Progressive familial intrahepatic cholestasis, Glycogen storage disease type III, Tyrosinemia, Deoxyguanosine kinase deficiency, Pyruvate carboxylase deficiency, Congenital dyserythropoietic anemia, Polycystic Liver Disease Polycystic Kidney Disease, Alpha-1 antitrypsine deficiency, Ureum cycle defects, Organic acidemiea, lysosomal storage diseases, and Fatty Acid Oxydation Disorders. Other conditions that may also benefit from cell-based therapy include Wilson's Disease and Hereditary Amyloidosis (FAP).

Other non-hepatocyte related causes of liver failure that would require a full liver transplant to reach full therapeutic effect, may still benefit from some temporary restoration of function using cell-based therapy using cells cultured according to the media and/or methods of the invention. A non-limiting list of examples of such conditions includes: Primary Biliary Cirrhosis, Primary Sclerosing Cholangitis, Aglagille syndrome, Homozygous Familial hypercholesterolemia, Hepatitis B with cirrhosis, Hepatitis C with cirrhosis, Budd-Chiari syndrome, Primary hyperoxaluria, Autoimmune Hepatitis, and Alcoholic liver disease.

The liver stem cells of the invention may be used in a method of treating a hereditary disease that involves malfunctioning hepatocytes. Such diseases may be early onset or late onset. Early onset disease include metabolite related organ failure (e.g., alpha-1-antitrypsin deficiency), glycogen storage diseases (e.g., GSD II, Pompe disease), tyrosinemia, mild DGUOK, CDA type I, Ureum cycle defects (e.g., OTC deficiency), organic academia and fatty acid oxidation disorders. Late onset diseases include primary hyperoxaluria, familial hypercholesterolemia, Wilson's disease, Hereditary Amyloidosis and Polycystic liver disease. Partial or full replacement with healthy hepatocytes arising from liver stem cells of the invention may be used to restore liver function or to postpone liver failure.

The liver stem cells of the invention may also be used in a method of treating chronic liver failure arising due to hereditary metabolic disease or as a result of hepatocyte infection. Treatment of a hereditary metabolic disease may involve administration of genetically modified autologous liver stem cells of the invention. Treatment of hepatocyte infections may involve administration of allogeneic liver stem cells of the invention. In some embodiments, the liver stem cells are administered over a period of 2-3 months.

The liver stem cells of the invention may be used to treat acute liver failure, for example, as a result of liver intoxication which may result from use of paracetamol, medication or alcohol. In some embodiments, the therapy to restore liver function will comprise injecting hepatocyte suspension from frozen, ready to use allogenic hepatocytes obtained from stem cells of the invention. The ability to freeze suitable stem cells of the invention means that the stem cells can be available for immediate delivery and so it is not necessary to wait for a blood transfusion.

In the case of replacement or correction of deficient liver function, it may be possible to construct a cell-matrix structure from one or more liver stem cells generated according to the present invention. It is thought that only about 10% of hepatic cell mass is necessary for adequate function. This makes implantation of stem cells compositions into children especially preferable to whole organ transplantation, due to the relatively limited availability of donors and smaller size of juvenile organs. For example, an 8-month-old child has a normal liver that weighs approximately 250 g. That child would therefore need about 25 g of tissue. An adult liver weighs-approximately 1500 g; therefore, the required implant would only be about 1.5% of the adult liver. When liver stem cells according to the invention are implanted, optionally attached to a polymer scaffold, proliferation in the new host will occur, and the resulting hepatic cell mass replaces the deficient host function. Hence, the invention provides a new source of hepatocytes for liver regeneration, replacement or correction of deficient liver function.

The inventors have also demonstrated successful transplantation of the genetically manipulated stem cells of the invention, grown by methods of the present invention, into immunodeficient mice (see Example 15), with transplanted stem cell-derived cells homing to the liver and generating hepatocytes in vivo. Therefore, in one embodiment the invention provides stem cells of the invention for transplanting into human or animals.

Accordingly, included within the scope of the invention are methods of treatment of a human or animal patient through cellular therapy. The term “animal” here denotes all mammalian animals, preferably human patients. It also includes an individual animal in all stages of development, including embryonic and foetal stages. For example, the patient may be an adult, or the therapy may be for pediatric use (e.g., newborn, child or adolescent). Such cellular therapy encompasses the administration of stem cells generated according to the invention to a patient through any appropriate means. Specifically, such methods of treatment involve the regeneration of damaged tissue or wound healing. The term “administration” as used herein refers to well recognized forms of administration, such as intravenous or injection, as well as to administration by transplantation, for example transplantation by surgery, grafting or transplantation of tissue engineered liver derived from the stem cells according to the present invention. In the case of cells, systemic administration to an individual may be possible, for example, by infusion into the superior mesenteric artery, the celiac artery, the subclavian vein via the thoracic duct, infusion into the heart via the superior vena cava, or infusion into the peritoneal cavity with subsequent migration of cells via subdiaphragmatic lymphatics, or directly into liver sites via infusion into the hepatic arterial blood supply or into the portal vein.

Between 10⁴ and 10¹³ cells per 100 kg person may be administered per infusion. Preferably, between about 1-5×10⁴ and 1-5×10⁷ cells may be infused intravenously per 100 kg person. More preferably, between about 1×10⁴ and 10×10⁶ cells may be infused intravenously per 100 kg person. In some embodiments, a single administration of the subject stem cells is provided. In other embodiments, multiple administrations are used. Multiple administrations can be provided over an initial treatment regime, for example, of 3-7 consecutive days, and then repeated at other times.

In some embodiments it is desirable to repopulate/replace 10-20% of a patient's liver with healthy hepatocytes arising from a liver stem cell of the invention.

In certain embodiments, the liver stem cell used in the regenerative medicinal use is a clonal expansion of a single liver stem cell. This single cell may have been modified by introduction of a nucleic acid construct as defined herein, for example, to correct a genetic deficiency or mutation. It would also be possible to specifically ablate expression, as desired, for example, using siRNA. Potential polypeptides to be expressed could be any of those that are deficient in metabolic liver diseases, including, for example, AAT (alpha antitrypsin). For elucidating liver physiology, it may also be desirable to express or inactivate genes implicated in the Wnt, EGF, FGF, BMP or notch pathway. Also, for screening of drug toxicity, the expression or inactivation of genes responsible for liver drug metabolism (for example, genes in the CYP family) would be of high interest.

It will be clear to a skilled person that gene therapy can additionally be used in a method directed at repairing damaged or diseased tissue. Use can, for example, be made of an adenoviral or retroviral gene delivery vehicle to deliver genetic information, like DNA and/or RNA to stem cells. A skilled person can replace or repair particular genes targeted in gene therapy. For example, a normal gene may be inserted into a nonspecific location within the genome to replace a non functional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the stem cells are ex vivo treated by a gene therapy approach and are subsequently transferred to the mammal, preferably a human being in need of treatment. For example, stem cell-derived cells may be genetically modified in culture before transplantation into patients.

### Toxicity Assay

The expanded stem cell population can further replace the use of cell lines such as Caco-2 cells in toxicity assays of potential novel drugs or of known or novel food supplements. Such toxicity assay may be conducted using patient matched or tissue/organ matched stem cells, which may be useful in personalized medicine.

Such toxicity assays may be in vitro assays using a cell derived from, e.g., a liver stem cell or a differentiated structure thereof (such as a structure differentiated from a liver stem cell on the ALI, collectively “(liver) stem cell” in the context of toxicity assay). Such liver stem cells and differentiated progeny thereof are easy to culture, and more closely resemble primary epithelial cells than, for example, epithelial cell lines such as Caco-2 (ATCC HTB-37), 1-407 (ATCC CCL6), and XBF (ATCC CRL 8808) which are currently used in toxicity assays. Toxicity results obtained with such liver stem cells, especially patient matched liver stem cells, more closely resemble results obtained in patients.

A cell-based toxicity test is used for determining organ specific cytotoxicity. Compounds that may be tested comprise cancer chemopreventive agents, environmental chemicals, food supplements, and potential toxicants. The cells are exposed to multiple concentrations of a test agent for certain period of time. The concentration ranges for test agents in the assay are determined in a preliminary assay using an exposure of five days and log dilutions from the highest soluble concentration. At the end of the exposure period, the cultures are evaluated for inhibition of growth. Data are analyzed to determine the concentration that inhibited end point by 50 percent (TC50).

For example, induction of cytochrome P450 enzymes in liver hepatocytes is a key factor that determines the efficacy and toxicity of drugs. In particular, induction of P450s is an important mechanism of troublesome drug-drug interactions, and it is also an important factor that limits drug efficacy and governs drug toxicity. Cytochrome P450 induction assays have been difficult to develop, because they require intact normal human hepatocytes. These cells have proven intractable to production in numbers sufficient to sustain mass production of high throughput assays.

For example, according to this aspect of the invention, a candidate compound may be contacted with the stem cell as described herein, and any change to the cells or in to activity of the cells may be monitored. Examples of other non-therapeutic uses of the stem cells of the present invention include research of liver embryology, liver cell lineages, and differentiation pathways; gene expression studies including recombinant gene expression; mechanisms involved in liver injury and repair; research of inflammatory and infectious diseases of the liver; studies of pathogenetic mechanisms; and studies of mechanisms of liver cell transformation and aetiology of liver cancer.

For high-throughput purposes, the liver stem cells are cultured in multiwell plates such as, for example, 96-well plates or 384-well plates. Libraries of molecules are used to identify a molecule that affects the stem cells. Preferred libraries comprise antibody fragment libraries, peptide phage display libraries, peptide libraries (e.g., LOPAP™, Sigma Aldrich), lipid libraries (BioMol), synthetic compound libraries (e.g., LOP AC™, Sigma Aldrich) or natural compound libraries (Specs, TimTec). Furthermore, genetic libraries can be used that induce or repress the expression of one of more genes in the progeny of the adenoma cells. These genetic libraries comprise cDNA libraries, antisense libraries, and siRNA or other non-coding RNA libraries. The cells are preferably exposed to multiple concentrations of a test agent for certain period of time. At the end of the exposure period, the cultures are evaluated. The term “affecting” is used to cover any change in a cell, including, but not limited to, a reduction in, or loss of, proliferation, a morphological change, and cell death The liver stem cells can also be used to identify drugs that specifically target epithelial carcinoma cells, but not the liver stem cells.

### Animal Model

Furthermore, the expanded stem cell population can also be used for culturing of a pathogen such as a norovirus which presently lacks a suitable tissue culture, or animal model.

Thus one aspect of the invention provides an animal model comprising a subject stem cell, such as a subject cancer stem cell.

In certain embodiments, the animal is an immunodeficient non-human animal (such as a rodent, e.g., a mouse or a rat), since such animal is less likely to cause rejection reaction. As an immunodeficient animal, it is preferred to use a non-human animal deficient in functional T cells, such as a nude mouse and rat, and a non-human animal deficient in functional T and B cells, such as a SCID mouse and a NOD-SCID mouse. Particularly, a mouse deficient in T, B, and NK cells (for example, a severely immunodeficient mouse obtained by crossing a SCID, RAG2KO, or RAG1KO mouse with an IL-2Rg^{null} mouse, which includes NOD/SCID/gammac^{null} mouse, NOD-scid, IL-2Rg^{null} mouse, and BALB/c-Rag2^{null}, IL-2Re^{null} mouse), which shows excellent transplantability, is preferably used.

Regarding the age of non-human animals, when athymic nude mice, SCID mice, NOD/SCID mice, or NOG mice are used, those of 4-100 weeks old are preferably used.

NOG mice can be produced, for example, by the method described in WO 2002/043477 (incorporated by reference), or can be obtained from the Central Institute for Experimental Animals or the Jackson Laboratory (NSG mice).

Cells to be transplanted may be any types of cells, including a stem cell mass/clone, a tissue section differentiated from the subject stem cell, singly dispersed stem cells, stem cells cultured after isolation or freeze/thaw, and stem cells transplanted to another animal and again isolated from the animal. The number of cells to be transplanted may be 10⁶ or less, but a greater number of cells may be transplanted.

In certain embodiments, subcutaneous transplantation is preferable because of its simple transplantation techniques. However, the site of transplantation is not particularly limited and preferably appropriately selected depending on the animal used. The procedure for transplanting NOG established cancer cell lines is not particularly limited, and any conventional transplantation procedures can be used.

Such animal models can be used to, for example, search for drug target molecules and to assess drugs. Assessment methods for drugs include screening for drugs and screening for anticancer agents. Methods of searching for target molecules include, but are not limited to, methods for identifying genes such as DNAs and RNAs highly expressed in cancer stem cells (e.g., cancer stem cell markers) using Gene-chip analysis, and methods for identifying proteins, peptides, or metabolites highly expressed in cancer stem cells using proteomics.

Screening methods for searching for target molecules include methods in which substances that inhibit the growth of cancer stem cells are screened from a small molecule library, antibody library, micro RNA library, or RNAi library, etc., using cell growth inhibition assay. After an inhibitor is obtained, its target can be revealed.

Thus the invention also provides a method of identifying a target molecule of a drug, the method comprising: (1) producing a non-human animal model by transplanting a cancer stem cell of the invention to a non-human animal (e.g., an immuno-compromised mouse or rat); (2) before and after administering the drug, collecting a tissue section showing a tissue structure characteristic of a cancer development process of said cancer stem cell population or showing a biological property thereof; (3) examining/comparing the tissue sections (before vs. after) collected in (2) for the expression of a DNA, RNA, protein, peptide, or metabolite; and (4) identifying a DNA, RNA, protein, peptide or metabolite that varies depending on a structure formed from the cancer stem cells, a cancer development process originating from the cancer stem cells, or a biological property of the cancer stem cells, in the tissue section.

The invention also provides a method of assessing a drug, the method comprising: (1) producing a non-human animal model by transplanting a cancer stem cell of the invention to a non-human animal (e.g., an immuno-compromised mouse or rat); (2) administering a test substance to the non-human animal model of (1); (3) collecting a tissue section showing a tissue structure characteristic of a cancer development process originating from cancer stem cells or showing a biological property thereof; (4) observing a change in the cancer stem cells over time, cancer development process, or a biological property thereof, in the tissue section; and (5) identifying formation of a structure formed from the cancer stem cells, a cancer development process originating from the cancer stem cells, or a biological property of the cancer stem cells, that is inhibited by the test substance.

The invention also provides a method of screening for a drug, the method comprising: (1) producing a non-human animal model by transplanting a cancer stem cell of the invention to a non-human animal (e.g., an immuno-compromised mouse or rat); (2) administering a test substance to the non-human animal model of (1); (3) collecting a tissue section that shows a tissue structure characteristic of a cancer development process originating from cancer stem cells, or shows a biological property thereof; (4) observing a change in the cancer stem cells over time, cancer development process, or a biological property thereof, in the tissue section; and (5) identifying a test substance that inhibits formation of a structure formed from specific cancer stem cells, a cancer development process originating from cancer stem cells, or a biological property of cancer stem cells.

All patent and literature references cited in the present specification are hereby incorporated by reference in their entirety.

The following examples are offered for illustrative purposes only, and are not intended to limit the scope of the present invention in any way.

### EXAMPLES

### Example 1

### Isolation, Cloning and Culturing of Human Intestinal Epithelial Stem Cells

In brief, a human adult or fetal intestinal biopsy was enzymatically digested and seeded on the irradiated 3T3-J2 feeder (originally obtained from Prof. Howard Green's laboratory at the Harvard Medical School, Boston, Mass., USA) in the presence of a modified growth medium. The stem cells selectively grow under these conditions and can be passaged indefinitely in vitro.

The day prior to receiving the human tissues, irradiated 3T3-J2 cells were seeded on Matrigel coated plates (BD Matrigel™, Basement Membrane Matrix, Growth Factor Reduced (GFR), cat. no. 354230). For this the Matrigel was thawed on ice and diluted in cold 3T3-J2 medium at the concentration of 10%. The 3T3-J2 growth medium contains DMEM (Invitrogen cat. no. 11960; high glucose (4.5 g/L), no L-glutamine, no sodium pyruvate), 10% bovine calf serum (not heat inactivated), 1% penicillin-streptomycin and 1% L-glutamine. The tissue culture plates were pre-cooled at −20° C. for 15 min, then diluted Matrigel was added on the cold plates, and the plates were swirled to evenly distribute the diluted Matrigel, then superfluous Matrigel was removed. Subsequently the plates were incubated for 15 min in a 37° C. incubator to allow the Matrigel layer to solidify.

Frozen irradiated 3T3-J2 cells were thawed and plated on the top of the Matrigel in the presence of 3T3-J2 growth medium. The next morning the 3T3-J2 medium was replaced by basic growth medium before being used as feeder layer for human cells. 1 L of basic growth medium contains 675 ml DMEM (Invitrogen cat. no. 11960; high glucose (4.5 g/L), no L-glutamine, no sodium pyruvate), 225 ml F12 (F-12 nutrient mixture (HAM), Invitrogen cat. no. 11765; containing L-glutamine), 100 ml FBS (Hyclone cat. no. SV30014.03; not heat inactivated), 6.75 ml of 200 mM L-glutamine (GIBCO cat. no. 25030), 10 ml adenine (Calbiochem cat. no. 1152; for the stock solution 243 mg of adenine were added to 100 ml of 0.05 M HCl and stirred for about one hour at RT until the solution was dissolved before filter sterilization. The solution can be stored at −20° C. until use), 1 ml of a 5 mg/ml stock solution of insulin (Sigma cat. no. 1-5500), 1 ml of 2×10⁻⁶ M T3 (3,3′,5-Triiodo-L-Thyronine) solution (Sigma cat. no. T-2752; for the stock solution 13.6 mg T3 were dissolved in 15 ml of 0.02N NaOH, and adjusted to 100 ml with phosphate buffered saline (PBS), resulting in a concentrated stock of 2×10⁻⁴ M, that can be stored at −20° C. 0.1 ml of the concentrated stock were diluted to 10 ml with PBS to create a working stock of 2×10⁻⁶ M), 2 ml of 200 μg/ml hydrocortisone (Sigma cat. no. H-0888), 1 ml of 10 μg/ml EGF (Upstate Biotechnology cat. no. 01-107), and 10 ml Penicillin-Streptomycin containing 10,000 units of penicillin and 10,000 μg of streptomycin per ml (GIBCO cat. no. 15140).

Human intestinal biopsies (transferred from hospital in cold wash buffer on ice) were washed vigorously using 30 ml cold wash buffer (F12: DMEM 1:1; 1.0% penicillin-streptomycin; 0.1% fungizone and 2.5 ml of 100 μg/ml gentamycin) for three times and followed once by cold PBS. The biopsy was minced and soaked in digestion medium (BD Cell Recovery Solution cat. no. 354253) and incubated at 4° C. for 8-12 h with gentle shaking. Alternatively, the tissue can be digested using 2 mg/mL collagenase type IV (Gibco, cat. no. 17104-109) and incubated at 37° C. for 1-2 h while gently shaking. The digested tissues were pelleted and washed five times with 30 mL cold wash buffer each. After the final wash, the samples were spun down and resuspended in modified growth medium and seeded on the feeder. The modified growth medium for human adult intestine epithelial stem cells consisted of basic growth medium and the following factors: rock inhibitor (R)-(+)-trans-N-(4-Pyridyl)-4-(1-aminoethyl)-cyclohexanecarboxamide (Y-27632, Rho Kinase Inhibitor VI, Calbiochem, cat. no. 688000) at a working concentration of 2.5 μM; recombinant R-spondin 1 protein (R&D, cat. no. 4645-RS) at a working concentration of 125 ng/ml; recombinant noggin protein (Peprotech, cat. no. 120-10c) at a working concentration of 100 ng/ml; Jagged-1 peptide (188-204) (AnaSpec Inc., cat. no. 61298) at a working concentration of 1 μM; SB431542: 4-(4-(benzo[d][1,3]dioxol-5-yl)-5-(pyridin-2-yl)-1H-imidazol-2-yl)benzamide (Cayman chemical company, cat. no. 13031) at a working concentration of 2 μM; nicotinamide (Sigma, cat. no. N0636-100G) at a working concentration of 10 mM. The modified growth medium for human fetal intestine epithelial stem cells consisted of basic growth medium and the following factors: rock inhibitor (R)-(+)-trans-N-(4-Pyridyl)-4-(1-aminoethyl)-cyclohexanecarboxamide (Y-27632, Rho Kinase Inhibitor VI, Calbiochem, cat. no. 688000) at a working concentration of 2.5 μM; recombinant R-spondin 1 protein (R&D, cat. no. 4645-RS) at a working concentration of 125 ng/ml; recombinant noggin protein (Peprotech, cat. no. 120-10c) at a working concentration of 100 ng/ml; Jagged-1 peptide (188-204) (AnaSpec Inc., cat. no. 61298) at a working concentration of 1 μM; nicotinamide (Sigma, cat. no. N0636-100G) at a working concentration of 10 mM. After three to four days the first epithelial cell colonies were detectable. Then cells were trypsinized with warm 0.25% trypsin (Invitrogen, cat. no 25200056) for 10 min, neutralized, resuspended in the modified growth medium, passed through 40 micron cell strainer and seeded as single cells onto a new plate containing a 3T3-J2 feeder layer. The medium was changed every two days. 3 days later, individual clones of adult human epithelial stem cells were observed. For fetal intestine epithelial stem cells, the SCM medium in Example 16 can also be used in this example.

A single colony was be picked using a cloning ring and expanded to develop a pedigree cell line, i.e. a cell line that has been derived from a single cell.

Alternatively, single cells from the dissociated single cell suspension derived from these colonies can be selected using a glass pipette under a microscope and individually transferred to 96 well plates previously coated with 10% Matrigel and seeded with the feeder cells. Once the single cell forms colony in the 96 well plates, the colony can be expanded to develop a pedigree cell line.

More than 70% of the intestine epithelial cells in culture maintain the clonogenic ability indicating that they are stem cells. This evidence supports the culture system presented here is capable of maintaining self-renewal ability of human intestine epithelial stem cells. Furthermore, after more than 400 cell divisions, these intestine epithelial stem cells maintain their ability for multipotent differentiation and form intestine-like structures in the air-liquid interface assay.

### Example 2

### Isolation, Cloning and Culturing of Human Intestinal Metaplasia Stem Cells

In brief, a human intestinal metaplasia biopsy was enzymatically digested and seeded on the irradiated 3T3-J2 feeder (originally obtained from Prof. Howard Green's laboratory at the Harvard Medical School) in the presence of a modified growth medium. The stem cells selectively grow under these conditions and can be passaged indefinitely in vitro.

The day prior to receiving the human tissues, irradiated 3T3-J2 cells were seeded on Matrigel coated plates (BD Matrigel™, Basement Membrane Matrix, Growth Factor Reduced (GFR), cat. no. 354230). For this the Matrigel was thawed on ice and diluted in cold 3T3-J2 medium at the concentration of 10%. The 3T3-J2 growth medium contains DMEM (Invitrogen cat. no. 11960; high glucose (4.5 g/L), no L-glutamine, no sodium pyruvate), 10% bovine calf serum (not heat inactivated), 1% penicillin-streptomycin and 1% L-glutamine. The tissue culture plates were pre-cooled at −20° C. for 15 min, then diluted Matrigel was added on the cold plates, and the plates were swirled to evenly distribute the diluted Matrigel, then superfluous Matrigel was removed. Subsequently the plates were incubated for 15 min in a 37° C. incubator to allow the Matrigel layer to solidify.

Frozen irradiated 3T3-J2 cells were thawed and plated on the top of the Matrigel in the presence of 3T3-J2 growth medium. The next morning the 3T3-J2 medium was replaced by basic growth medium before being used as feeder layer for human cells. 1 L of basic growth medium contains 675 ml DMEM (Invitrogen cat. no. 11960; high glucose (4.5 g/L), no L-glutamine, no sodium pyruvate), 225 ml F12 (F-12 nutrient mixture (HAM), Invitrogen cat. no. 11765; containing L-glutamine), 100 ml FBS (Hyclone cat. no. SV30014.03; not heat inactivated), 6.75 ml of 200 mM L-glutamine (GIBCO cat. no. 25030), 10 ml adenine (Calbiochem cat. no. 1152; 2.43 mg/ml), 1 ml of a 5 mg/ml stock solution of insulin (Sigma cat. no. 1-5500), 1 ml of 2×10⁻⁶ M T3 (3,3′,5-Triiodo-L-Thyronine) solution (Sigma cat. no. T-2752; for the stock solution 13.6 mg T3 were dissolved in 15 ml of 0.02N NaOH, and adjusted to 100 ml with phosphate buffered saline (PBS), resulting in a concentrated stock of 2×10⁻⁴ M, that can be stored at −20° C. 0.1 ml of the concentrated stock were diluted to 10 ml with PBS to create a working stock of 2×10⁻⁶ M), 2 ml of 200 μg/ml hydrocortisone (Sigma cat. no. H-0888), 1 ml of 1 mg/ml EGF (Upstate Biotechnology cat. no. 01-107) in 0.1% bovine serum albumin (Sigma cat. no. A-2058), and 10 ml Penicillin-Streptomycin containing 10,000 units of penicillin and 10,000 μg of streptomycin per ml (GIBCO cat. no. 15140).

Human intestinal metaplasia biopsies (transferred from hospital in cold wash buffer on ice) were washed vigorously using 30 ml cold wash buffer (F12: DMEM 1:1; 1.0% penicillin-streptomycin; 0.1% fungizone and 2.5 ml of 100 μg/ml gentamycin) for three times and one time followed by cold PBS. The biopsy was minced and soaked in digestion medium (DMEM:F12 1:1; 1.0% penicillin-streptomycin; 100 μg/ml gentamicin; 2 mg/ml collagenase (Roche, cat. no. 11088793001)) and incubated at 37° C. for 1-2 h while gently shaking. The digested tissues were pelleted and washed five times with 30 ml cold wash buffer each. After the final wash, the samples were spun down and resuspended in modified growth medium and seeded on the feeder. The modified growth medium consisted of basic growth medium and the following factors: 2.5 μM rock inhibitor (R)-(+)-trans-N-(4-Pyridyl)-4-(1-aminoethyl)-cyclohexanecarboxamide (Y-27632, Rho Kinase Inhibitor VI, Calbiochem, cat. no. 688000); 125 ng/ml recombinant R-spondin 1 protein (R&D, cat. no. 4645-RS); 100 ng/ml recombinant noggin protein (Peprotech, cat. no. 120-10c); 1 μM Jagged-1 peptide (188-204) (AnaSpec Inc., cat. no. 61298); and 2 μM SB431542: 4-(4-(benzo[d][1,3]dioxol-5-yl)-5-(pyridin-2-yl)-1H-imidazol-2-yl)benzamide (Cayman chemical company, cat. no. 13031); nicotinamide (Sigma, cat. no. N0636-100G) at a working concentration of 10 mM.

After three to four days the first epithelial cell colonies were detectable. Then cells were trypsinized with warm 0.25% trypsin (Invitrogen, cat. no 25200056) for 10 min, neutralized, resuspended in the modified growth medium, passed through 40 micron cell strainer and seeded as single cells onto a new plate containing a 3T3-J2 feeder layer. The medium was changed every two days. 4-5 days later, individual clones of adult human epithelial stem cells were observed.

A single colony can be picked using a cloning ring and expanded to develop a pedigree cell line, i.e., a cell line that has been derived from a single cell. Alternatively, single cells from the dissociated single cell suspension derived from these colonies can be selected using a glass pipette under a microscope and individually transferred to 96 well plates previously coated with 10% Matrigel and seeded with the feeder cells. Once the single cell forms colony in the 96 well plates, the colony can be expanded to develop a pedigree cell line.

### Example 3

### Isolation, Cloning and Culturing of Human Stomach Epithelial Stem Cells

In brief, a human stomach epithelial biopsy was enzymatically digested and seeded on the irradiated 3T3-J2 feeder in the presence of a modified growth medium. The stem cells selectively grow under these conditions and can be passaged indefinitely in vitro.

The day prior to receiving the human tissues, irradiated 3T3-J2 cells were seeded on Matrigel coated plates (BD Matrigel™, Basement Membrane Matrix, Growth Factor Reduced (GFR), cat. no. 354230). For this the Matrigel was thawed on ice and diluted in cold 3T3-J2 medium at the concentration of 10%. The 3T3-J2 growth medium contains DMEM (Invitrogen cat. no. 11960; high glucose (4.5 g/L), no L-glutamine, no sodium pyruvate), 10% bovine calf serum (not heat inactivated), 1% penicillin-streptomycin and 1% L-glutamine. The tissue culture plates were pre-cooled at −20° C. for 15 min, then diluted Matrigel was added on the cold plates, and the plates were swirled to evenly distribute the diluted Matrigel, then superfluous Matrigel was removed. Subsequently the plates were incubated for 15 min in a 37° C. incubator to allow the Matrigel layer to solidify.

Frozen irradiated 3T3-J2 cells were thawed and plated on the top of the Matrigel in the presence of 3T3-J2 growth medium. The next morning the 3T3-J2 medium was replaced by basic growth medium before being used as feeder layer for human cells. 1 L of basic growth medium contains 675 ml DMEM (Invitrogen cat. no. 11960; high glucose (4.5 g/L), no L-glutamine, no sodium pyruvate), 225 ml F12 (F-12 nutrient mixture (HAM), Invitrogen cat. no. 11765; containing L-glutamine), 100 ml FBS (Hyclone cat. no. SV30014.03; not heat inactivated), 6.75 ml of 200 mM L-glutamine (GIBCO cat. no. 25030), 10 ml adenine (Calbiochem cat. no. 1152; 2.43 mg/ml), 1 ml of a 5 mg/ml stock solution of insulin (Sigma cat. no. 1-5500), 1 ml of 2×10⁻⁶ M T3 (3,3′,5-Triiodo-L-Thyronine) solution (Sigma cat. no. T-2752; for the stock solution 13.6 mg T3 were dissolved in 15 ml of 0.02N NaOH, and adjusted to 100 ml with phosphate buffered saline (PBS), resulting in a concentrated stock of 2×10⁻⁴ M, that can be stored at −20° C. 0.1 ml of the concentrated stock were diluted to 10 ml with PBS to create a working stock of 2×10⁻⁶ M), 2 ml of 200 μg/ml hydrocortisone (Sigma cat. no. H-0888), 1 ml of 1 mg/ml EGF (Upstate Biotechnology cat. no. 01-107) in 0.1% bovine serum albumin (Sigma cat. no. A-2058), and 10 ml Penicillin-Streptomycin containing 10,000 units of penicillin and 10,000 μg of streptomycin per ml (GIBCO cat. no. 15140).

Human stomach epithelial tissue biopsies (transferred from hospital in cold wash buffer on ice) were washed vigorously using 30 ml cold wash buffer (F12: DMEM 1:1; 1.0% penicillin-streptomycin; 0.1% fungizone and 2.5 ml of 100 μg/ml gentamycin) for three times and one time followed by cold PBS. The biopsy was minced and soaked in digestion medium (DMEM:F12 1:1; 1.0% penicillin-streptomycin; 100 μg/ml gentamicin; 2 mg/ml collagenase (Roche, cat. no. 11088793001)) and incubated at 37° C. for 1-2 h while gently shaking. The digested tissues were pelleted and washed five times with 30 ml cold wash buffer each. After the final wash, the samples were spun down and resuspended in modified growth medium and seeded on the feeder. The modified growth medium consisted of basic growth medium and the following factors: 2.5 μM rock inhibitor (Y-27632, Rho Kinase Inhibitor VI, Calbiochem, cat. no. 688000); 125 ng/ml recombinant R-spondin 1 protein (R&D, cat. no. 4645-RS); 100 ng/ml recombinant noggin protein (Peprotech, cat. no. 120-10c); 1 μM Jagged-1 peptide (188-204) (AnaSpec Inc., cat. no. 61298); and 2 μM SB431542 (Cayman chemical company, cat. no. 13031) and 10 mM nicotinamide (Sigma, cat. no. N0636-100G).

After three to four days the first stomach epithelial cell colonies were detectable. Then cells were trypsinized with warm 0.25% trypsin (Invitrogen, cat. no 25200056) for 10 min, neutralized, resuspended in the modified growth medium, passed through 40 micron cell strainer and seeded as single cells onto a new plate containing a 3T3-J2 feeder layer. The medium was changed every two days. 3 to 4 days later, individual stomach epithelial stem cells were detectable.

A single colony can be picked using a cloning ring and expanded to develop a pedigree cell line, i.e. a cell line that has been derived from a single cell. Alternatively, single cells from the dissociated single cell suspension derived from these colonies can be selected using a glass pipette under a microscope and individually transferred to 96 well plates previously coated with 10% Matrigel and seeded with the feeder cells. Once the single cell forms colony in the 96 well plates, the colony can be expanded to develop a pedigree cell line.

More than 70% of the stomach epithelial cells in culture maintain the clonogenic ability indicating that they are stem cells. This evidence supports the culture system presented here is capable of maintaining self-renewal ability of human stomach epithelial stem cells. Furthermore, after 400 cell divisions, these stomach epithelial stem cells maintain their ability for multipotent differentiation and form stomach-like structures in the Matrigel assay.

Using substantially the same method, regiospecific stem cells (see Example 21) in stomach, including cardia, funds, body, antrum, etc., have also been cloned from those regions of the stomach, each representing distinct stem cells in the stomach.

### Example 4

### Isolation, Cloning and Culturing of Human Liver Epithelial Stem Cells

Chronic liver damage and resulting fibrosis kills 25,000 Americans each year, and results in more than 3 billion dollars in health costs. End-stage liver damage due to hepatitis A, B, and C viral infections, alcohol abuse, or nonalcoholic fatty liver disease (NAFLD) is fibrosis, and requires allogeneic transplantation, though complications of immunosuppression, viral superinfection, and recidivism limits the effectiveness of such therapy.

The field of human adult stem cells of the liver has been mired in controversy and variable progress (Koike and Taniguchi, J. Hepatobiliary Pancreat Sci. 19:587-593, 2012). A recent report shows that certain mouse liver “organoids” containing a certain (albeit small) number of stem cells can be used for ex-vivo differentiation of clusters of liver cells to repopulate mouse livers following acute liver damage (Huch et al., Nature 494:247-250, 2013). However, given the limited expansion of these organoids in vitro, and their small number of stem cells they contain, they do not lend themselves to genetic modification by any of the emerging technologies.

Induced pluripotent (iPS) stem cells may potentially enable the production of patient-specific hepatocytes, and may be modified by certain genetic engineering technologies (Yagi et al., Crit. Rev. Biomed. Eng. 37:377-398, 2009). However, it is less clear whether iPS cells can be induced to form adult stem cells for the liver, as iPS cells in general have not been shown to produce adult stem cells of other tissues.

Applicant has now developed technologies to clone human liver stem cells from adult and fetal tissues, in a manner that maintains their immature state, with high proliferative rates and unlimited expandability. This example provides an exemplary method for cloning stem cells of human hepatocytes, from both adult and fetal human tissues. The cloned liver stem cells can be induced to differentiate into hepatocyte-like cells highly expressing albumin in vitro; and can be genetically modified (e.g., through introducing heterologous genetic materials using any of the art-recognized methods, such as transfection, or infection by a viral vector, such as a retroviral or lentiviral vector, etc.). Such isolated, clonally expanded, and/or genetically modified liver stem cells can be used in a variety of uses, including (without limitation) tissue regeneration, wound healing, or gene therapy to correct genetic defects, such as the liver diseases referenced above.

In brief, a human liver biopsy was enzymatically digested and seeded on the irradiated 3T3-J2 feeder in the presence of a modified growth medium. The liver epithelial stem cells selectively grow under these conditions and can be passaged numerous times in vitro.

The day prior to receiving the human tissues, irradiated 3T3-J2 cells were seeded on Matrigel coated plates (BD Matrigel™, Basement Membrane Matrix, Growth Factor Reduced (GFR), cat. no. 354230). For this the Matrigel was thawed on ice and diluted in cold 3T3-J2 medium at the concentration of 10%. The 3T3-J2 growth medium contains DMEM (Invitrogen cat. no. 11960; high glucose (4.5 g/L), no L-glutamine, no sodium pyruvate), 10% bovine calf serum (not heat inactivated), 1% penicillin-streptomycin and 1% L-glutamine. The tissue culture plates were pre-cooled at −20° C. for 15 min, then diluted Matrigel was added on the cold plates, and the plates were swirled to evenly distribute the diluted Matrigel, then superfluous Matrigel was removed. Subsequently the plates were incubated for 15 min in a 37° C. incubator to allow the Matrigel layer to solidify.

Frozen irradiated 3T3-J2 cells were thawed and plated on the top of the Matrigel in the presence of 3T3-J2 growth medium. The next morning the 3T3-J2 medium was replaced by basic growth medium before being used as feeder layer for human cells. 1 L of basic growth medium contains 675 ml DMEM (Invitrogen cat. no. 11960; high glucose (4.5 g/L), no L-glutamine, no sodium pyruvate), 225 ml F12 (F-12 nutrient mixture (HAM), Invitrogen cat. no. 11765; containing L-glutamine), 100 ml FBS (Hyclone cat. no. SV30014.03; not heat inactivated), 6.75 ml of 200 mM L-glutamine (GIBCO cat. no. 25030), 10 ml adenine (Calbiochem cat. no. 1152; 2.43 mg/ml), 1 ml of a 5 mg/ml stock solution of insulin (Sigma cat. no. I-5500), 1 ml of 2×10⁻⁶ M T3 (3,3′,5-Triiodo-L-Thyronine) solution (Sigma cat. no. T-2752; for the stock solution 13.6 mg T3 were dissolved in 15 ml of 0.02N NaOH, and adjusted to 100 ml with phosphate buffered saline (PBS), resulting in a concentrated stock of 2×10⁻⁴ M, that can be stored at −20° C. 0.1 ml of the concentrated stock were diluted to 10 ml with PBS to create a working stock of 2×10⁻⁶ M), 2 ml of 200 μg/ml hydrocortisone (Sigma cat. no. H-0888), 1 ml of 1 mg/ml EGF (Upstate Biotechnology cat. no. 01-107) in 0.1% bovine serum albumin (Sigma cat. no. A-2058), and 10 ml Penicillin-Streptomycin containing 10,000 units of penicillin and 10,000 μg of streptomycin per ml (GIBCO cat. no. 15140).

Human liver biopsy (transferred from hospital in cold wash buffer on ice) was washed vigorously using 30 ml cold wash buffer (F12: DMEM 1:1; 1.0% penicillin-streptomycin; 0.1% fungizone and 2.5 ml of 100 μg/ml gentamycin) for three times and one time followed by cold PBS. The biopsy was minced and soaked in digestion medium (F12: DMEM 1:1; 1 u/ml penicillin-streptomycin; 1 μg/ml gentamycin and 2 mg/ml collagenase A) and incubated at 37° C. for 1-2 h while gently shaking. The digested tissues were pelleted and washed five times with 30 ml cold wash buffer each. After the final wash, the samples were spun down and resuspended in modified growth medium and seeded on the feeder. The modified growth medium consisted of basic growth medium and the following factors: 2.5 μM rock inhibitor (Y-27632, Rho Kinase Inhibitor VI, Calbiochem, cat. no. 688000); 125 ng/ml recombinant R-spondin 1 protein (R&D, cat. no. 4645-RS); 100 ng/ml recombinant noggin protein (Peprotech, cat. no. 120-10c); 1 μM Jagged-1 peptide (188-204) (AnaSpec Inc., cat. no. 61298); and 2 μM SB431542 (Cayman Chemical Company, cat. no. 13031) and 10 mM nicotinamide (Sigma, cat. no. N0636-100G).

After three to four days the first liver epithelial cell colonies were detectable. Then cells were trypsinized with warm 0.25% trypsin (Invitrogen, cat. no 25200056) for 10 min, neutralized, resuspended in the modified growth medium, passed through 40 micron cell strainer and seeded as single cells onto a new plate containing a 3T3-J2 feeder layer. The medium was changed every two days. 3 to 4 days later, individual liver epithelial stem cells were detectable.

A single colony can be picked using a cloning ring and expanded to develop a pedigree cell line, i.e. a cell line that has been derived from a single cell. Alternatively, single cells from the dissociated single cell suspension derived from these colonies can be selected using a glass pipette under a microscope and individually transferred to 96 well plates previously coated with 10% Matrigel and seeded with the feeder cells. Once the single cell forms colony in the 96 well plates, the colony can be expanded to develop a pedigree cell line.

Using substantially the same procedure described herein, liver stem cells and clonal expansion from single cloned liver stem cells have been obtained. These cells are highly proliferative and can be passaged indefinitely in vitro (data not shown).

Immature colonies from cloned liver stem cell pedigree in early passage exhibit substantially the same morphology and appearance in culture, even after about 400 cell divisions (results not shown), demonstrating that the cloned liver stem cells maintain their immature state, with high proliferative rates and unlimited expandability, after long term culture in vitro.

### Example 5

### Isolation, Cloning and Culturing of Human Pancreas Epithelial Stem Cells

In brief, a human pancreas tissue was enzymatically digested and seeded on the irradiated 3T3-J2 feeder in the presence of a modified growth medium. The pancreas epithelial stem cells selectively grow under these conditions and can be passaged numerous times in vitro.

The day prior to receiving the human tissues, irradiated 3T3-J2 cells were seeded on Matrigel coated plates (BD Matrigel™, Basement Membrane Matrix, Growth Factor Reduced (GFR), cat. no. 354230). For this the Matrigel was thawed on ice and diluted in cold 3T3-J2 medium at the concentration of 10%. The 3T3-J2 growth medium contains DMEM (Invitrogen cat. no. 11960; high glucose (4.5 g/L), no L-glutamine, no sodium pyruvate), 10% bovine calf serum (not heat inactivated), 1% penicillin-streptomycin and 1% L-glutamine. The tissue culture plates were pre-cooled at −20° C. for 15 min, then diluted Matrigel was added on the cold plates, and the plates were swirled to evenly distribute the diluted Matrigel, then superfluous Matrigel was removed. Subsequently the plates were incubated for 15 min in a 37° C. incubator to allow the Matrigel layer to solidify.

Frozen irradiated 3T3-J2 cells were thawed and plated on the top of the Matrigel in the presence of 3T3-J2 growth medium. The next morning the 3T3-J2 medium was replaced by basic growth medium before being used as feeder layer for human cells. 1 L of basic growth medium contains 675 ml DMEM (Invitrogen cat. no. 11960; high glucose (4.5 g/L), no L-glutamine, no sodium pyruvate), 225 ml F12 (F-12 nutrient mixture (HAM), Invitrogen cat. no. 11765; containing L-glutamine), 100 ml FBS (Hyclone cat. no. SV30014.03; not heat inactivated), 6.75 ml of 200 mM L-glutamine (GIBCO cat. no. 25030), 10 ml adenine (Calbiochem cat. no. 1152; 2.43 mg/ml), 1 ml of a 5 mg/ml stock solution of insulin (Sigma cat. no. 1-5500), 1 ml of 2×10⁻⁶ M T3 (3,3′,5-Triiodo-L-Thyronine) solution (Sigma cat. no. T-2752; for the stock solution 13.6 mg T3 were dissolved in 15 ml of 0.02N NaOH, and adjusted to 100 ml with phosphate buffered saline (PBS), resulting in a concentrated stock of 2×10⁻⁴ M, that can be stored at −20° C. 0.1 ml of the concentrated stock were diluted to 10 ml with PBS to create a working stock of 2×10⁻⁶ M), 2 ml of 200 μg/ml hydrocortisone (Sigma cat. no. H-0888), 1 ml of 1 mg/ml EGF (Upstate Biotechnology cat. no. 01-107) in 0.1% bovine serum albumin (Sigma cat. no. A-2058), and 10 ml Penicillin-Streptomycin containing 10,000 units of penicillin and 10,000 μg of streptomycin per ml (GIBCO cat. no. 15140).

Human pancreas tissue (transferred from hospital in cold wash buffer on ice) was washed vigorously using 30 ml cold wash buffer (F12: DMEM 1:1; 1.0% penicillin-streptomycin; 0.1% fungizone and 2.5 ml of 100 μg/ml gentamycin) for three times and one time followed by cold PBS. The biopsy was minced and soaked in digestion medium (F12: DMEM 1:1; 1 u/ml penicillin-streptomycin; 1 μg/ml gentamycin and 2 mg/ml collagenase A) and incubated at 37° C. for 1-2 h while gently shaking. The digested tissues were pelleted and washed five times with 30 ml cold wash buffer each. After the final wash, the samples were spun down and resuspended in modified growth medium and seeded on the feeder. The modified growth medium consisted of basic growth medium and the following factors: 2.5 μM rock inhibitor (Y-27632, Rho Kinase Inhibitor VI, Calbiochem, cat. no. 688000); 125 ng/ml recombinant R-spondin 1 protein (R&D, cat. no. 4645-RS); 100 ng/ml recombinant noggin protein (Peprotech, cat. no. 120-10c); 1 μM Jagged-1 peptide (188-204) (AnaSpec Inc., cat. no. 61298); and 2 μM SB431542 (Cayman chemical company, cat. no. 13031) and 10 mM nicotinamide (Sigma, cat. no. N0636-100G).

After three to four days the first pancreas epithelial cell colonies were detectable. Then cells were trypsinized with warm 0.25% trypsin (Invitrogen, cat. no 25200056) for 10 min, neutralized, resuspended in the modified growth medium, passed through 40 micron cell strainer and seeded as single cells onto a new plate containing a 3T3-J2 feeder layer. The medium was changed every two days. 3 to 4 days later, individual pancreas epithelial stem cells were detectable.

A single colony can be picked using a cloning ring and expanded to develop a pedigree cell line, i.e. a cell line that has been derived from a single cell. Alternatively, single cells from the dissociated single cell suspension derived from these colonies can be selected using a glass pipette under a microscope and individually transferred to 96 well plates previously coated with 10% Matrigel and seeded with the feeder cells. Once the single cell forms colony in the 96 well plates, the colony can be expanded to develop a pedigree cell line.

### Example 6

### Cloned Stem Cells Maintain Self-Renewal Capability in Culture

Pedigree cell lines were established by clonal expansion of a single cloned human liver stem cell according to the procedure substantially the same as described in Example 4. The procedure was repeatedly used to isolate single cells from the expanded pedigree cell line, in order to determine whether the repeatedly isolated cells maintains stem cell characteristics over multiple generations of cell division, e.g., the self-renewal capability while being propagated in vitro.

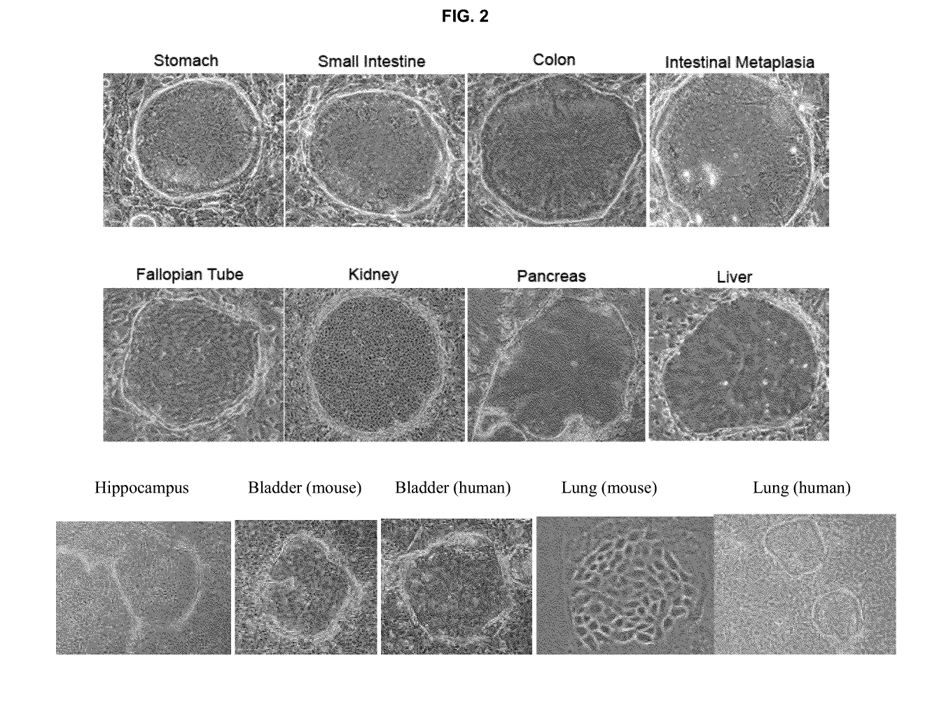

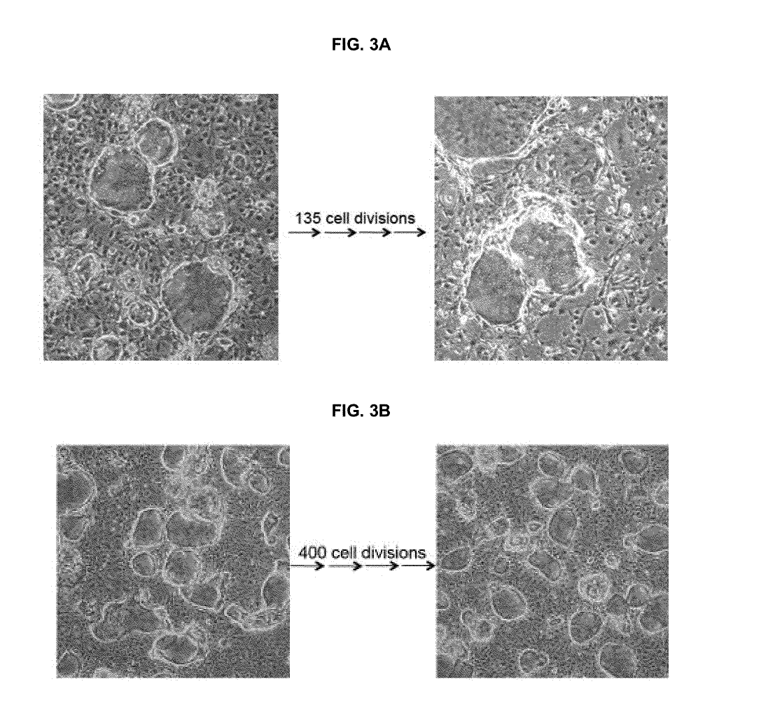

FIG. 3A shows that adult liver stem cells isolated using the methods of the invention can propagate in vitro for more than 100 (e.g., 135) divisions while still maintaining the immature cell morphology (cf. FIG. 2). Note the same small, round morphology of the cells within the clone, with relatively large nucleus and high nuclear/cytoplasm ratio. FIG. 3B shows that the immature cell morphology was maintained even after 400 cell divisions in in vitro culture.

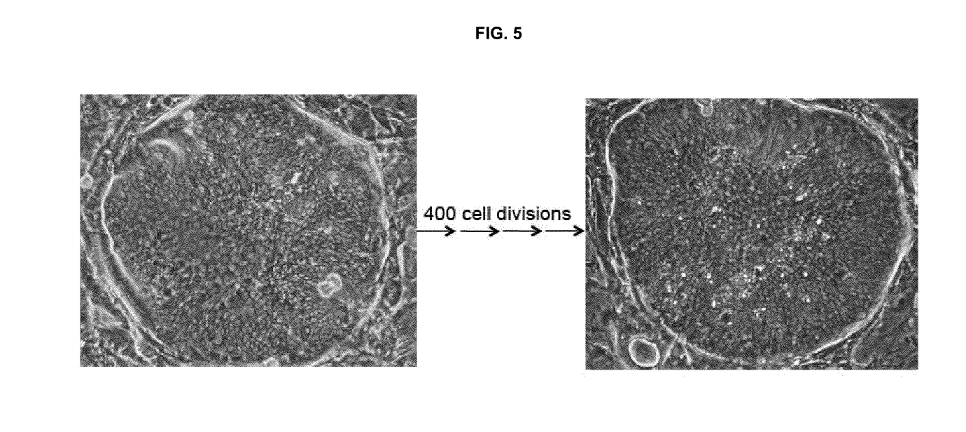

In a similar experiment, pedigree cell lines were established by clonal expansion of a single cloned human small intestine stem cell according to the procedure substantially the same as described in Example 2. FIG. 5 shows that the pedigree cell line repeated expanded from the cloned human small intestine stem cell can propagate in vitro for more than 400 generations while still maintaining the immature cell morphology (cf. FIG. 2).

In another experiment, equal number of cells from passage 4 and passage 40 of cloned intestinal stem cells were seeded on the feeder layer as previously described. The comparable number of observed colonies suggests that the clonogenic ability of the intestine stem cells is not affected by passaging, nor is level of differentiation ability.

### Example 7

### Differentiation of Liver Stem Cells in MATRIGEL™

Cloned immature liver stem cells (including those cloned from fetal tissue) express marker of proliferation, such as Ki67 (as detected by antibodies against such marker proteins, results not shown), as well as liver stem cell markers such as Sox9 and Krt7 (results not shown). Sox9 is a transcription factor believed to mark the putative stem cells in liver, see Huch & Clevers (Nature Genetics 43, 9-10, 2011).

Meanwhile, the cloned immature liver stem cells lack expression of albumin, alpha-fetoprotein (AFP), HNF4a, FOXA2, and other hepatocyte markers (see FIG. 4, and data not shown). However, expression of these markers can be readily induced upon activation in 2- and 3-D differentiation systems. This example demonstrates that the cloned immature liver stem cells can readily differentiate in the presence of MATRIGEL™ basement membrane matrix (BD), and express various hepatocyte markers upon differentiation.

Liver stem cells were digested by 0.05% trypsin for 30 to 60 seconds. The epithelial stem cells were separated from the irradiated 3T3-J2 fibroblast feeder, and the trypsin was neutralized by the serum containing medium.

The liver epithelial stem cells were then plated on the MATRIGEL™ basement membrane matrix (BD) coated tissue culture plates, and grown in the presence of the growth medium (CFAD+1 μM Jagged-1+100 ng/mL Noggin+125 ng/mL R-Spondin-1+2.5 μM Rock inhibitor+2 μM SB431542+10 mM Nicotinamide).

After 3 to 5 days, the growth medium was changed to differentiation medium (HBM Basal Medium (Lonza, cat. no. CC-3199) and Hepatocyte Culture Medium HCM™ SingleQuots™ Kit (Lonza, cat. no. CC-4182). The differentiation medium was changed every 2 days. After about 10 days, the differentiation structures were harvested for sectioning, IHC (immunohistochemistry), IF (immunofluorescent) staining, and/or RNA collection.

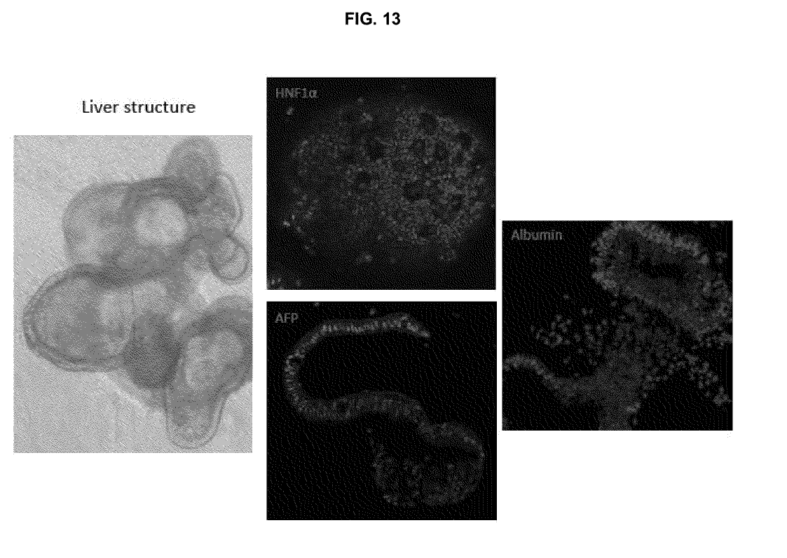

The isolated liver stem cell differentiated into organized structures in MATRIGEL™ basement membrane matrix (BD) under the conditions described (FIG. 13). The isolated liver stem cell was also differentiated into organized structures in air-liquid interface (ALI), using substantially the same condition as that in Example 14 below.

IF (immunofluorescent) staining of the differentiated structure shows that the differentiated cells expressed the hallmark liver marker genes such as albumin, HNF-1α (hepatocyte nuclear factor 1 alpha), FOXA2, and alpha-fetoprotein (AFP), demonstrating that the liver stem cells have differentiated into mature liver cells (see FIGS. 4 & 13, and results not shown). In another experiment, quantitative RT-PCR with specific primers of SOX9, AFP and Albumin, using RNA extracted from liver stem cells and in vitro differentiated stem cells, was performed to measure and compare expression level of the respective marker genes. The data was consistent with the observation in IF experiments.

Meanwhile, expression of liver stem cell marker Sox9 (as measured by qRT-PCR) was down-regulated by about 5-fold when comparing expression level in liver stem cells and hepatocyte differentiated on air-liquid interface (ALI) (results not shown).

Heatmap (data not shown) of gene expression of liver stem cells, in vitro differentiated stem cells, and mature hepatocyte cultures was generated in order to further investigate the gene expression differences in these cells. These in vitro differentiated stem cells yield whole genome expression patterns overlapping to some extent to that of mature hepatocyte. Moreover, gene expression microarray analysis revealed the enrichment of pathways regulating specific liver functions in the in vitro differentiated stem cells, including pathways specific for regulating liver functions, such as drug metabolism and metabolism of xenobiotic by cytochrome P450 (data not shown).

### Example 8

### Cloned Small Intestine Stem Cells can Differentiate In Vitro

A pedigree cell line was established based on a single isolated human small intestine stem cell according to a procedure substantially the same as that described in Example 2. Cells from the small intestine stem cell pedigree cell line were then differentiated into intestine-tissue-like structures in the air-liquid interface (ALI) cell culture system, substantially as described in Example 14.

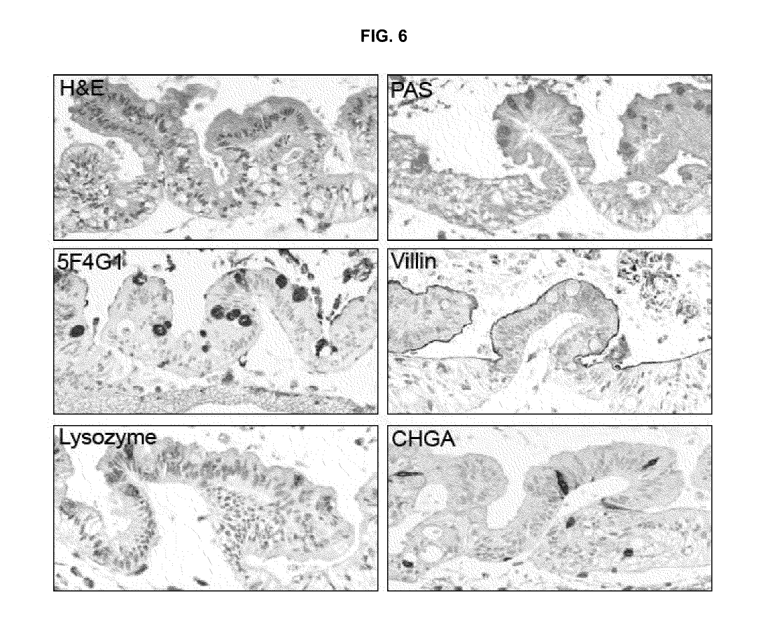

Immunofluorescent staining was performed on the differentiated cells, using antibodies specific for the various differentiated cell markers. FIG. 6 shows that cells clonally expanded from one single isolated intestinal stem cell can differentiate into the goblet cells based on PAS staining and 5F4G1 antibody staining (which is specific for differentiated goblet cells); the Paneth cells based on LYZ (lysozyme) staining; and the neuroendocrine cells based on CHGA staining. In addition, the intestine-tissue-like structure also expresses Villin that stains the microvilli-covered surface of small intestine tract where absorption takes place.

However, the intestinal stem cells used to generate these differentiated cells do not detectably express any of these differentiated cell markers based on similar immunofluorescent staining (data not shown). Specifically, the cloned intestine epithelial stem cells are positively stained with E-CAD (a marker for epithelial cell origin) and SOX9 (an intestinal stem cell marker), but do not express the differentiated cell markers such as MUC (goblet cell marker), CHGA (neuroendocrine cell marker) and LYZ (Paneth cell marker).

Furthermore, gene expression arrays of the isolated small intestine stem cells and differentiated structures show that the stem cell population highly expresses the stem cell markers such as Bmi1, LGR4, OLFM4 and LGR5 (data not shown). Meanwhile, the differentiated structures express markers such as MUC13, neuroendocrine cell markers (CHGA, CHGB), secretory cell marker (MUC7), other differentiation markers such as Krt 20, etc., that are typical markers for differentiated small intestine cells not expressed in the immature intestine stem cells. In addition, the PCA map shows the distinct separate of stem cells and differentiated structures based on gene expression pattern.

### Example 9

### Cloned Stomach Stem Cells can Differentiate In Vitro

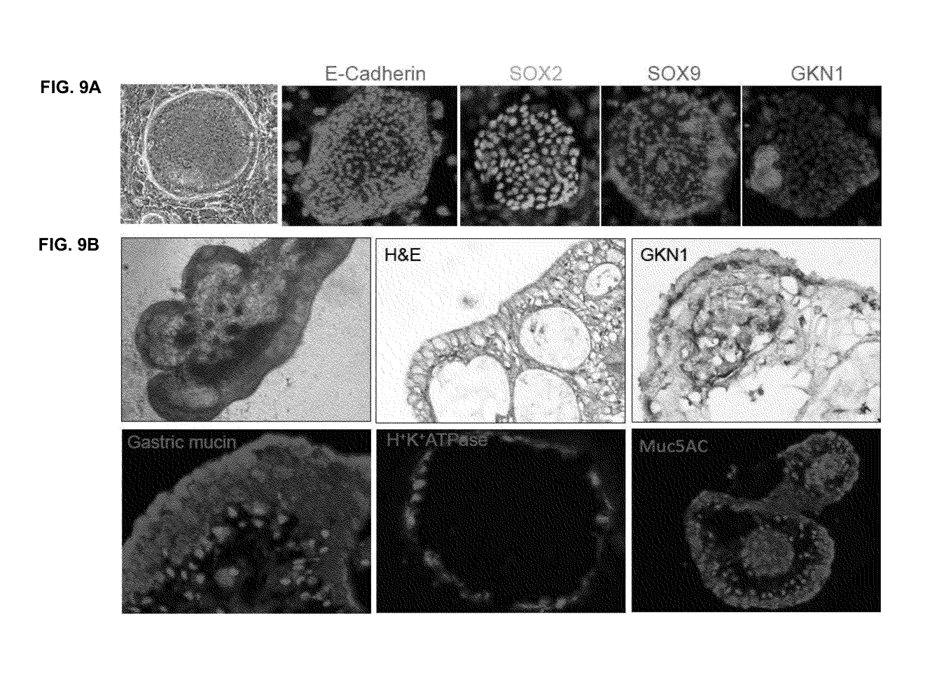

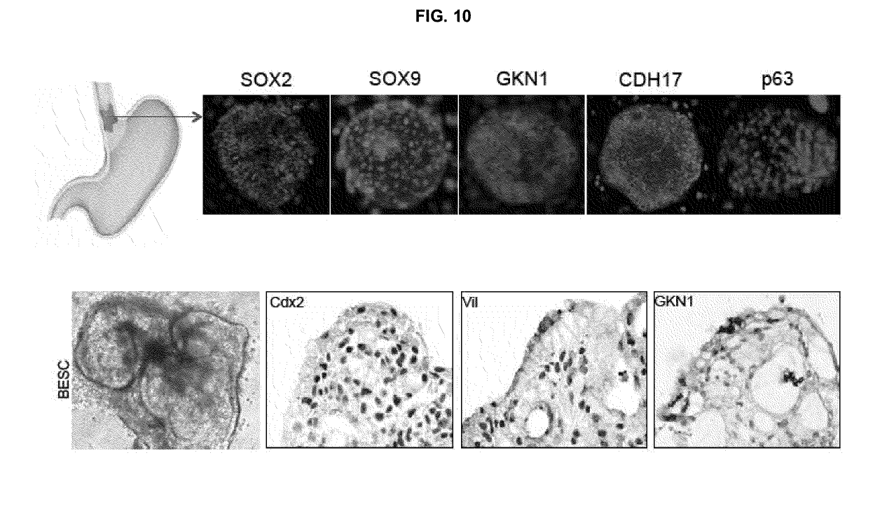

Human stomach stem cells were isolated according to a procedure substantially the same as described in Example 3. Immunofluorescent staining shows that the cloned human stomach epithelial stem cells display the typical immature morphology (small, round cells with relatively large nucleus and high nuclear/cytoplasm ratio (FIG. 9A). In addition, the cells are positively stained for E-Cadherin (epithelial cell origin), SOX2 and SOX9 (stem cells marker for gastric epithelial stem cells). FIG. 9A. Occasionally, a couple of cells in culture express GKN1 which is a typical gastric epithelium differentiation marker, suggesting the cells are derived from the stomach. FIG. 9A.

Pedigree cell line was established from a single cloned human stomach stem cell, and were differentiated in vitro to form columnar epithelium expressing mature gastric epithelium markers such as GKN1, Gastric mucin, H⁺K⁺ ATPase and Muc5Ac. The result demonstrates that the cloned stomach stem cells can be clonally expanded while maintaining the ability to differentiate in vitro to various differentiated gastric epithelium cell types.

### Example 10

### Different Adult Stem Cells Differentiate into Distinct Tissues/Structures

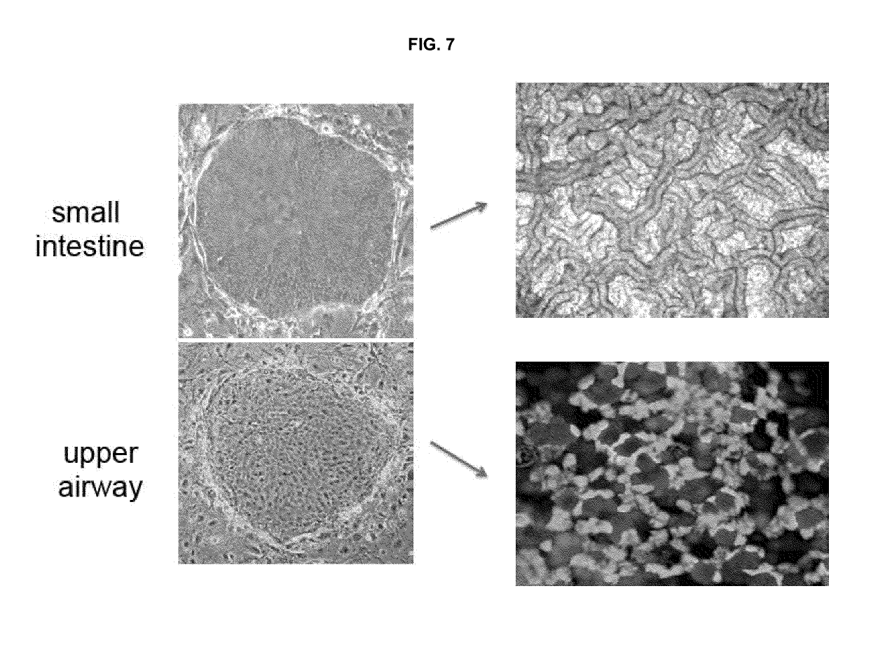

Stratified epithelial stem cells (from human upper airway) and the columnar epithelial stem cells (from small intestine) were isolated according to the methods of the invention (see Examples 1 and 2). These stem cells looked similar morphologically in culture (see FIG. 7, the two left panels), but they displayed distinct differentiation capacity in the air-liquid interface (ALI) culture system (see Example 14).

Specifically, the small intestine stem cells differentiated into mature intestine-like structures (FIG. 7, right side, upper panel), while the upper airway stem cells differentiated into mature upper airway epithelium in the same differentiation system (FIG. 7, right side, lower panel). Here, Mucin5AC stains differentiated upper airway goblet cells in an isolated pattern, while tubulin stains differentiated upper airway ciliated cells in a relatively continuous pattern surrounding the Mucin5AC stained goblet cells.

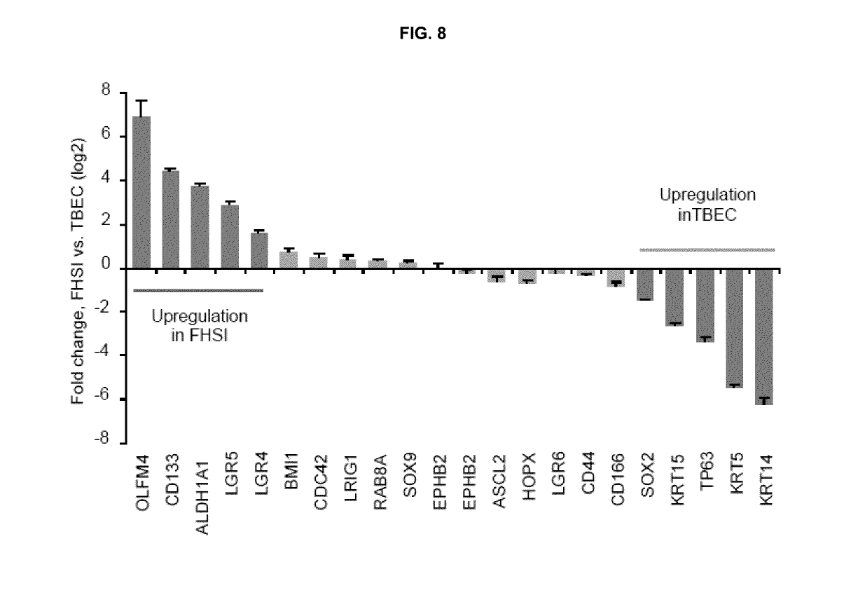

Gene expression comparison between the intestine epithelial stem cells and the upper airway epithelial stem cells (FIG. 8) showed that intestinal stem cells highly expressed markers such as OLFM4, CD133, ALDH1A1, LGR5 and LGR4, while upper airway stem cells highly expressed markers such as Krt14, Krt5, p63, Krt15 and SOX2.

Additional comparison of gene expression between intestine stem cells and upper airway stem cells (data not shown) showed that the intestine stem cells highly express a number of receptors that regulate important signal transduction pathways such as Wnt (FZD4, FZD3, LRP6, LGR4, LGR5, FZD7 and FZD5) and TGFbeta-BMP (TGFBR1, TGFBR2, TGFBR3, ACVR1B, ACVR2A, BMPR1A). However, in comparison with the upper airway stem cells, the intestine stem cells barely express the ligands for Hedgehog, Notch, Wnt and TGFbeta-BMP pathways. This might implicate the reason of Paneth cells function as the supporter for stem cells of intestine. However, the upper airway stem cells might have an autocrine signaling mechanism so their self-renew doesn't require the existence of Paneth-cell-like cell type.

Overall, such differences in gene expression pattern suggests that there are alternative mechanisms of maintaining immaturity in the isolated stem cells.

Similarly, cloned human colon stem cells displayed distinct gene expression pattern in comparison with cloned human small intestine stem cells (data not shown). Both cloned colon stem cells and small intestine stem cells are highly proliferative based on the positive Ki67 staining throughout the whole colony (data not shown). Interestingly, the small intestine stem cells differentiated into Paneth cells that are Lyozyme (LYZ) positive, but the colon stem cells did not differentiate into Paneth cells under the same condition. This observation is consistent with the fact that human colon tissue does not contain Paneth cells.

The cloned colon stem cells can be used to regenerate colonic epithelium in the patients that suffer extreme erosion due to inflammation.

### Example 11

### Cloned Adult Stem Cells from Fallopian Tube

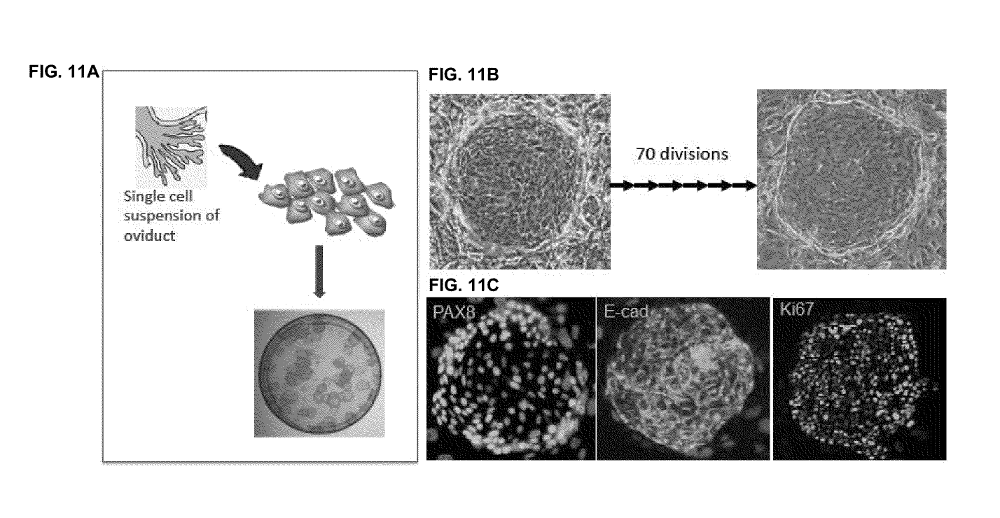

According to the method of the invention (see, for example, Example 1), human fallopian tube tissue was enzymatically digested and seeded on the feeder layer to form colonies consisting hundreds of epithelial stem cells (FIG. 11A), and adult stem cells were isolated from the Fallopian tubes.

The isolated stem cells can divide more than 70 times in vitro without differentiation or senescence (FIG. 11B). The cloned cells were stained by the PAX8 marker (a typical markers for fallopian tube epithelium), and were E-Cadherin positive (Epithelial cell marker) and Ki67 positive (proliferation marker).

### Example 12

### Cloned Adult Stem Cells from Pancreas

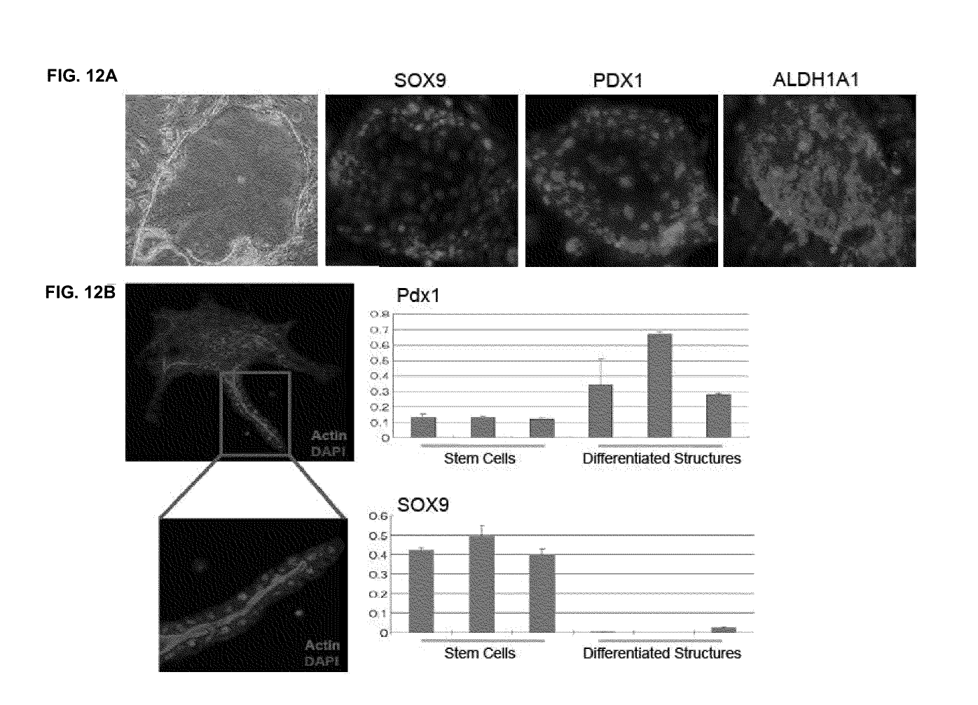

Human pancreatic stem cells were isolated according to the methods of the invention (see Examples 1 and 5). The cloned human pancreas stem cells express putative stem cell markers such as SOX9, Pdx1 and ALDH1A1 (FIG. 12A). The cells can also differentiate into tubal structures in vitro (FIG. 12B). Real time PCR results obtained by using gene specific primers showed that the Pdx1 and SOX9 marker gene expression was dramatically down-regulated when these cells differentiate.

### Example 13

### Differentiation of Barrett's Esophagus Stem Cell and Gastric Cardia Stem Cell

Barrett's esophagus and gastric cardia cells were digested by 0.05% trypsin for 30 to 60 seconds. The epithelial stem cells were separated from the irradiated (3T3-J2) fibroblast feeder by manual shaking, and the stem cell clones were removed by pipetting up and down several times. Trypsin was neutralized by the serum containing medium, and the cluster of stem cell clones were suspended in matrigel culture medium (advanced F12/DMEM reduced serum medium 1:1, Hepes 10 mM, Pen 100 Unit/mL/Strep 100 pg/ml, L-glutamine 2 mM, N-2 supplement 1×, B-27 supplement 1×, EGF 50 ng/mL, FGF10 100 ng/mL, Wnt3a 100 ng/mL, R-Spondin1 100 ng/mL, Noggin 100 ng/mL, SB431542 2 μM, SB203580 10 μM, Nicotinamide 10 mM, Y27632 2.5 μM) and plated on the MATRIGEL™ basement membrane matrix (BD) coated tissue culture plates.

After 3 to 5 days, the matrigel culture medium was changed to differentiation medium (advanced F12/DMEM reduced serum medium 1:1, Hepes 10 mM, Pen 100 Unit/mL/Strep 100 μg/mL, L-glutamine 2 mM, N-2 supplement 1×, B-27 supplement 1×, EGF 50 ng/mL, FGF10 100 ng/mL, Wnt3a 100 ng/mL, R-Spondin1 100 ng/mL, Noggin 100 ng/mL, Y27632 2.5 μM, DBZ 10 μM). The differentiation medium was changed every 2 days. After 2 weeks, the differentiation structures were harvested for sectioning, immunohistochemistry (IHC), immunofluorescence (IF) staining and RNA collection.

The components for the medium used in this experiment are listed below:

### Barrett's Esophagus Matrigel Culture Medium

| Name of product | Final conc. | | Company and cat no. |
|---|---|---|---|
| Advanced F12/DMEM | | | Gibco. 12643 |
| reduced serum | | | |
| medium (1:1) | | | |
| HEPEs | 10 | mM | homemade |
| Pen Strep | Pen 100 | unit/ml | Gibco. 15146 |
| | Strep 100 | μg/ml | |
| L-Glutamine | 2 | mM | Gibco. 25030-081 |
| N-2 supplement (100×) | 1× | | Gibco. 17502-048 |
| B-27 supplement (50×) | 1× | | Gibco. 10889-038 |
| EGF | 50 | ng/ml | Upstate |
| | | | Biotechnology 01-107 |
| FGF10 | 100 | ng/ml | R&D 345-FG |
| Wnt3a | 100 | ng/ml | R&D 5036-WN/CF |
| R-spondin1 | 100 | ng/ml | R&D 4645-RS |
| Noggin | 100 | ng/ml | Prospec cyt-475 |
| Y-27632 | 2 | μM | Calbiocam 688000 |
| SB431542 | 2 | μM | Tocris 1614 |
| SB203580 | 10 | μM | Promega V116A |
| Nicotinamide | 10 | mM | Sigma N0636 |

### Barrett's Esophagus Differentiation Medium

| Name of product | Final conc. | | Company and cat no. |
|---|---|---|---|
| Advanced F12/DMEM | | | Gibco. 12643 |
| reduced serum | | | |
| medium (1:1) | | | |
| HEPEs | 10 | mM | homemade |
| Pen Strep | Pen 100 | unit/ml | Gibco. 15146 |
| | Strep 100 | μg/ml | |
| L-Glutamine | 2 | mM | Gibco. 25030-081 |
| N-2 supplement (100×) | 1× | | Gibco. 17502-048 |
| B-27 supplement (50×) | 1× | | Gibco. 10889-038 |
| EGF | 50 | ng/ml | Upstate |
| | | | Biotechnology 01-107 |
| FGF10 | 100 | ng/ml | R&D 345-FG |
| Wnt3a | 100 | ng/ml | R&D 5036-WN/CF |
| R-spondin1 | 100 | ng/ml | R&D 4645-RS |
| Noggin | 100 | ng/ml | Prospec cyt-475 |
| Y-27632 | 2 | μM | Calbiocam 688000 |
| DBZ | 10 | μM | Sigma 143650 |

### Example 14

### Differentiation of Small Intestine Stem Cells on Air Liquid Interface

Isolated small intestine stem cells can be differentiated on air-liquid interface (ALI) with collagen and 3T3-J2 insert according to the method described in the example.

About 1×10⁵ 3 T3-J2 cells were first plated on each well of a Transwell-COL plate (Collagen coated transwell, 24 well plate, Cat. 3495, Corning Inc.). About 700 μL of 3T3 growth Medium was added to the outside chamber of each well, and about 200 μL of 3T3 growth medium (DMEM Invitrogen cat. no. 11960, high glucose (4.5 g/L), no L-glutamine, no sodium pyruvate; 10% bovine calf serum, not heat inactivated; 1% penicillin-streptomycin and 1% L-glutamine) was added to the inside chamber of each well.

The day after, 3T3 cells were washed once with the CFAD medium (or the Base Medium), then intestine stem cell clones were transferred onto the transwell. Each outside chamber of the transwell plate was filled by about 700 μL of stem cell growth medium (CFAD+1 μM Jagged-1+100 ng/mL Noggin+125 ng/mL R-Spondin-1+2.5 μM Rock inhibitor), and each inside chamber of the transwell was filled by 200 μL of stem cell growth medium.

The stem cell growth medium was changed about every 1-2 days, both inside and outside of each transwell insert. After confluence was reached (roughly 8-10 days for intestinal stem cells), the medium was change to differentiation medium (stem cell growth medium plus 2 μM GSK3 inhibitor), with about 700 μL of differentiation medium in the outside chamber of each transwell, but with no medium in the inside chambers. The differentiated structure was formed in about one month.

Using this method that is able to trigger differentiation in a wide range of epithelial cells, cloned intestinal stem cells were differentiated in air-liquid interface (ALI) to intestine crypt structures. Unlike upper airway stem cell pedigrees, which form upper airway epithelia complete with ciliated and goblet cells, the small intestine pedigrees formed, over a period of 10 days, a serpentine columnar epithelium similar in many respects to the villi of small intestine. Using markers for particular cell types specific to the small intestine, Applicant showed that the small intestine stem cells gave rise to goblet cells, Paneth cells, neuroendocrine cells, and a villin-containing brush border of enterocytes. Notably, these air-liquid interface cultures were characterized by high electrical resistance, suggesting that they formed a continuous array of tight junctions and thus have the potential to be functionalized for barrier function, transport, and even, conceivably, for microbiome containment assays. Assay using Beta-Ala-Lys (AMCA), a fluorescent dipeptide derivative, also suggests that the intestine structures differentiated in vitro has oligopeptide transport function like that of the human intestine.

Whole-genome transcriptome analysis showed expression of certain differentiation markers, such as brush border enzymes, that are upregulated in the ALI structures. Such analysis also showed that the intestine stem cell and upper airway epithelial stem cell differ only in expression of about 300-400 genes. However, they displayed thousands of gene expression differences after they were induced to differentiation in vitro. This data suggests that tissue specific stem cells are committed. This commitment is maintained by a small number of genes, and is niche independent because all the cells are cultured in the same condition and same medium.

The above data supports the notion that immature intestine stem cells can be cloned and cultured in vitro using the subject methods, and, upon induction, these stem cells can differentiate into the differentiated epithelium, including all the cell types existing in vivo. Since the differentiation assay is performed using pedigree cell line, the data supports the multipotent ability of stem cells. The data also illustrates that Paneath cell, Goblet cell, and Neuroendocrine cell in human small intestine are all derived from one single stem cell.

Similarly, human adult pedigree colon stem cells were also differentiated in air-liquid interface cell culture system. A single stem cell can differentiate into goblet cells (mucin 2 positive) and neuroendocrine cells (CHGA positive). The formed structure is polarized (e.g., Villin staining positively at the apical region). The structure also expressed other differentiation markers such as Krt20. Some of the cells in the structures were still proliferating and were labeled with Ki67. Differentiated colon stem cells displayed much higher ratio of goblet cells in comparison with differentiated small intestine stem cells. This distinct feature is consistent with the appearance of small intestine and colon in human body.

Human fetal pedigree colon stem cells were also differentiated in ALI, and the differentiated cells showed the same phenotype as human adult colon stem cells, with prominent number of goblet cells (mucin 2 positive) and some neuroendocrine cells (CHGA positive). Villin stained positively at the apical region, and Ki67 stained proliferating cells.

Colon stem cells derived from patient with Crohn's disease (see Example 21) can also differentiate into colon-like epithelium in the air-liquid interface cell culture system. One single stem cell can generate a pedigree cell line and differentiate into both goblet cells (mucin 2 positive) or neuroendocrine cells (CHGA positive).

### Example 15

### Liver Reconstitution in a Mouse Model with Differentiated Adult Stem Cells

Liver epithelial stem cells are differentiated towards liver cells using methods known in the art and are described for example in Cai et al. (Hepatology 2007, 45:1229-1239); Hay et al., 26:894-902, 2008; Kheolamai and Dickson, BMC Molecular Biology. 2009; Kajiwara et al. Proc. Natl. Acad. Sci. USA, 2012; 109(31):12538-12543, for example into liver progenitor cells.

The engraftment potential of the cells can be evaluated by transplanting such into a suitable animal model. Such model include immunodeficient mouse models, such as nude, severe combined immunodeficient (SCID), RAG-deficient, NOD-SCID mouse, the NOD-SCID/FAH mouse, NOD scid gamma (NSG; NOD.Cg-Prkdc<scid>Il2rg<tm1Wj1>/SzJ), NOD Rag gamma (NRG; NOD.Cg-Prkdc<tm1Mom>Il2rg<tm1Wj1>/SzJ); immunodeficient fumarylacetoacetate hydrolase (Fah) deficient mice, NSG/FAH (NSG, Fah−/−), NRG/FAH (NRG, Fah−/−), SCID-Alb-uPA (SCID mice expressing urokinase-type plasminogen activator (uPA) gene driven by an albumin (Alb)-promoter/enhancer); Alb-rtTA2S-M2/SCID/bg Mice; Alb-HB-EGF precursor mice (Saito et al. 2001, Nature Biotechnol., 19:746-750) or models as described in Rhim et al., 1994 Science, 263:1149-1152; Grompe et al., 1995, Nat. Genet., 10:453-460; Braun et al., 2000, Nat. Med., 6:320-326; Mignon et al., 1998, Nat. Med., 4:1185-1188; Song et al., 2009, Am. J. Pathol., 175:1975-1983.

For example the cells can be transplanted into NRG/FAH mice. Fah−/− mice are maintained with drinking water containing 2-(2-nitro-4-trifluoromethylbenzoyl)-1,3-cyclohexanedione (NTBC) of 7.5 g/mL, as Fah−/− mice have and depend on continuous medicinal treatment with NTBC, as the fumarylacetoacetate hydrolase deficiency affects liver and kidney and without treatment would die of liver failure (Overturf et al., 1996). This animal model is described for example by Grompe et al. 1995, Nature Genetics 10:453-460; Overturf et al. 1996, Nat. Genet. 12(3):266-73. After cell transplantation the NTBC treatment is discontinued. Liver samples are harvested at 6 and 10 weeks after cell transplantation and histologically examined. Mice are either sacrificed or anesthetized in the case that only biopsies are obtained. Alternatively, the serum of the mice can be assayed for the presence of human liver-specific proteins, such as albumin, alpha-1-antitrypsin, and alpha-fetoprotein by ELISA.

For the cell transplantation, cells are resuspended in a injection buffer (e.g. 50% Matrigel BD Biosciences #356234, 50% DMEM) and placed on ice until injection. Cells may be injected into four to eight weeks old newborn mice, and the liver functionally evaluated 6-10 weeks later.

As a specific example, isolated human liver stem cells were genetically modified (e.g., by viral infection using a lentivral or a retroviral vector) to express a heterologous gene, before the modified liver stem cells were introduced into a NOD scid gamma (NSG) mouse host. The example here shows that cloned human liver stem cells are capable of expressing the heterologous gene GFP after differentiating into liver tissue.

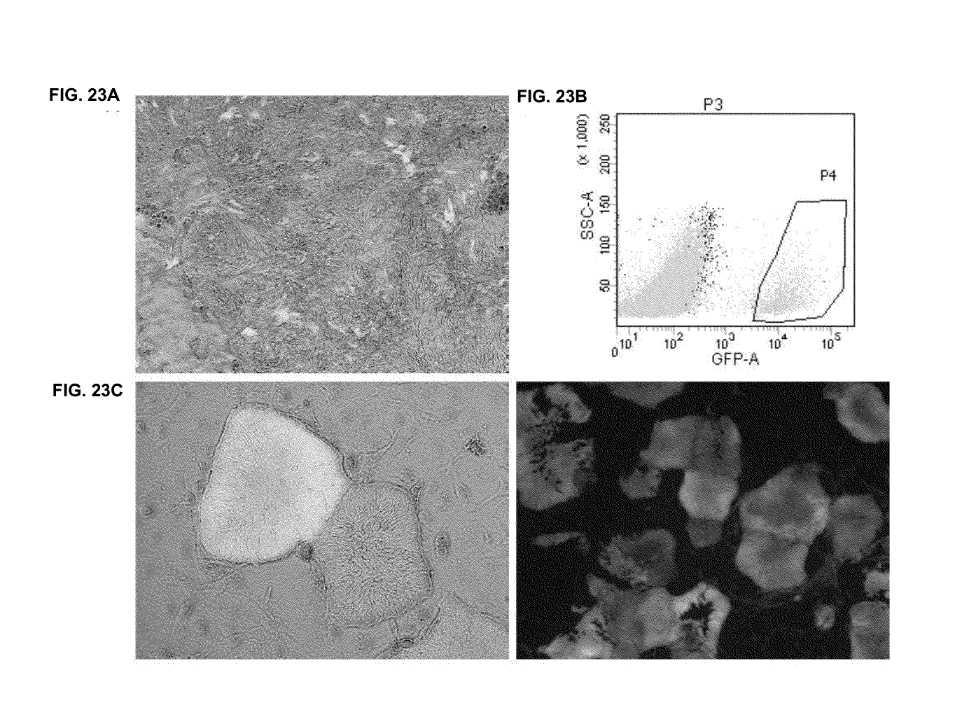

Specifically, the isolated liver stem cells were modified with GFP, and the GFP-positive cells were separated from the GFP-negative cell by FACS sorting (see FIGS. 23A and 23B). Continued culturing of the GFP-positive liver stem cells in the subject culturing system was achieved without observing any abnormality in the proliferation or differentiation capacity of these cells (FIG. 23C). The GFP-labeled (heterologous gene-expressing) liver stem cells remained immature, and can be readily expanded to large populations while maintaining this immature phenotype. Further, these GFP expressing cells can be differentiated in vitro to hepatocyte-like cells expressing Albumin (data not shown).

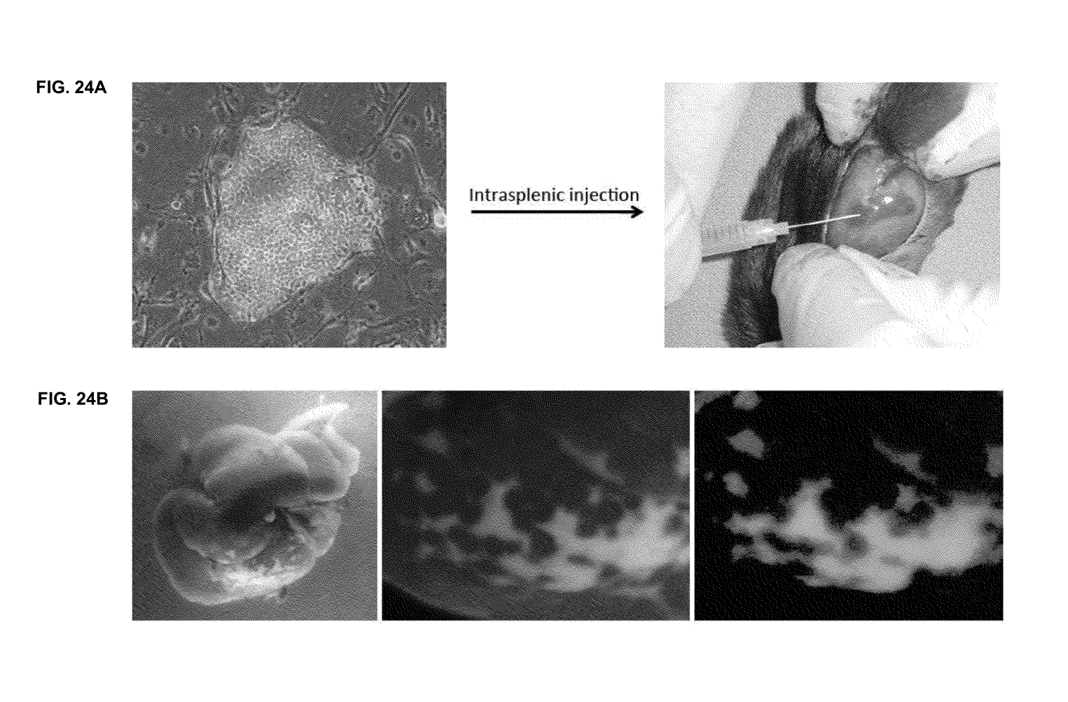

To demonstrate that the heterologous gene-expressing liver stem cells can differentiate in vivo into liver tissue, thus be reconstituted into the liver, the GFP-labeled human liver stem cells were introduced into an immuno-compromised mice, such as a NSG mice in this example, via injection of the GFP-labeled stem cells into the spleens of the NSG mice. The radiation of these cells to the hepatic ducts were readily observed 7 days post injection (FIGS. 24A and 24B).

This example demonstrates that cloned/isolated liver stem cells can be engineered to express a heterologous gene without losing its stem cell characteristics in culture; that the engineered stem cells can be expanded to large amounts in vitro under normal cultural conditions according to the method of the invention; that the engineered liver stem cells can home to the correct tissue (i.e., liver) from which the stem cells were initially isolated (albeit from a different species); and that the engineered liver stem cells can properly differentiate into the correct tissue. Thus the isolated/cloned adult stem cells can be used in regenerative medicine to, for example, repair or regenerate damaged or diseased tissues/organs.

In addition, the example also demonstrates that a xenograft animal model can be established to study diseases, such as a xenograft mouse model for studying liver damage. Indeed, an immuno-compromised mouse model has been shown, upon induced incipient liver failure, to provide an environmental niche allowing efficient repopulation by human hepatocytes and to a lower extent, in vitro derived cells (Liu et al., 2011). The xenograft mouse model in this experiment provides an equivalent or better alternative for studying tissue repair, would healing, and/or correction of a genetic defect associated with a human disease.

For example, the cloned liver stem cells can be engineered to be able to protect against hepatitis viruses, to correct gene defects implicated in a liver disease, or to develop mice with “humanized” livers for testing transplantation, differentiation, and proof-of-concept for anti-viral and gene correction technologies.

### Example 16

### Cloning of Cancer Stem Cells (CSCs) from Human Tumors and Chemotherapy-Resistant Cells Therefrom

This example demonstrates that the subject adult stem cell cloning methods can also be used to clone cancer stem cells (CSCs), as well as those of their precursor lesions, from cancerous tissues/cells. The example shows that the method of the invention can be used to clone large numbers of CSCs from each of several high-grade ovarian cancers. In addition, such CSC “libraries” were used to identify preexisting CSCs that are resistant to the chemotherapy drugs typically used to treat patients with high-grade ovarian cancer.

High-grade ovarian cancer (HGOC) is the most lethal of all gynecological cancers. Unlike other cancers affecting women, the five-year survival rate for high-grade ovarian cancer has not changed in the last 30 years. Worldwide, there are 225,000 new cases of ovarian cancer diagnosed annually, and an estimated 140,000 disease-related deaths. The lethality of this disease is attributed, in part, to the ability of metastatic tumor cells to propagate undetected in the peritoneum to large numbers, and the frequent late diagnosis of the disease at relatively advanced Stages III and IV.

The initial results of debulking surgery and cisplatin/paclitaxel chemotherapy are typically nothing short of spectacular, with many cases showing negligible or undetectable tumor within six months of treatment. Despite this initial good response to therapy, however, about 70-80% of these patients eventually show a recurrence of tumor after one year, and most of these recurrent tumors, unfortunately, will be resistant to further treatments with cisplatin and paclitaxel.

Thus it is generally believed that these lethal recurrences are the product of very small number of tumor cells (“cancer stem cells”) that survive the initial rounds of chemotherapy, and that ultimately expand their numbers over the six months to two years or so after the initial therapy. Thus the problem with the existing ovarian cancer treatment is not how to eliminate the bulk of the tumor cells (which are readily killed by the initial chemotherapy), but is likely how to eradicate the small number of resistant cancer stem cells lurking in the naïve tumor cell population.

Unfortunately, prior to the instant invention, there are no known methods for high-efficiency cloning of tumor cells from cancerous tumor tissues to test the notion of pre-existing chemotherapy-resistant cells, or more importantly, to assess therapies that would target this small population of cells that escape the standard-of-care regimens.

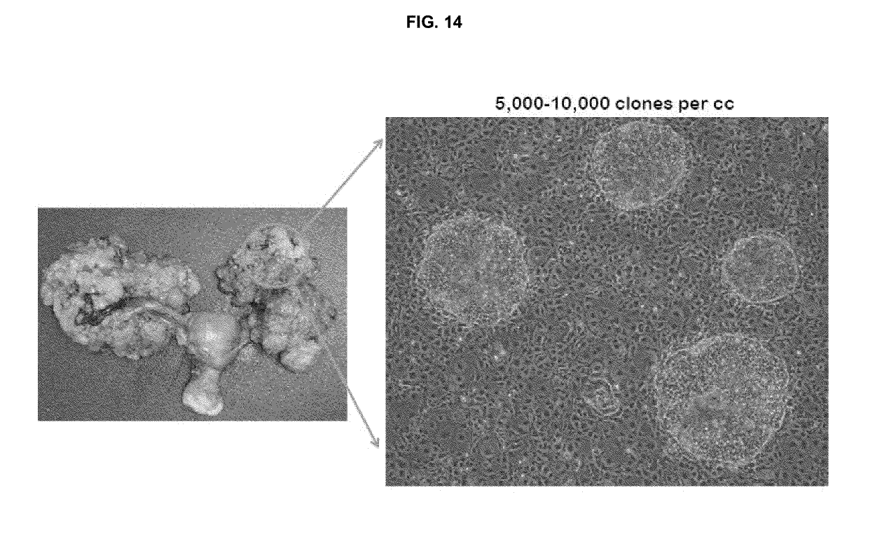

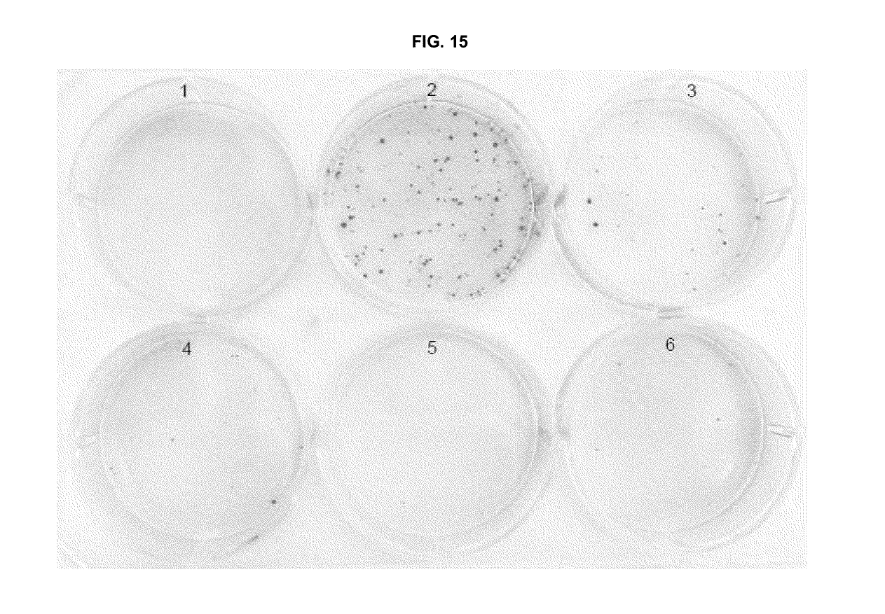

Date presented herein demonstrates that the methods of the invention can be used for rapid cloning of multiple tumor cells from a single high-grade ovarian cancer patient (FIG. 14). That is, the methods of the invention enable rapid cloning of about 5,000-10,000 discrete tumor cell colonies from each cubic centimeter (cm³) of a resected tumor tissue (FIG. 14). The media conditions that support optimal cloning of high-grade ovarian cancer are identical to the “six-factor” media shown to support human somatic stem cells from an array of regenerative tissues, while other permutations are considerably less efficient (FIG. 15; Table 3).

TABLE 3
Media Tested for Cancer Stem Cell Cultures

| | |
|---|---|
| 1 | SBM |
| 2 | SCM |
| 3 | SBM + SB431542 + GSK3i + Rocki + Nico |
| 4 | SBM + R-spondin + jagged-1 + Noggin + Rocki |
| 5 | SBM + Rocki |
| 6 | SBM + Rocki + R-spondin |

*SBM is modified media based on cFAD media of H. Green and colleagues (active ingredient EGF); SCM is SBM plus the “six-factors” described in the instant application (e.g., a Base Medium supplemented with Jagged-1 as a Notch agonist, Y-27632 as a ROCK inhibitor, Noggin as a BMP antagonist, R-spondin 1 as a Wnt agonist, SB431542 as TGF-β receptor inhibitor, EGF as a mitogenic growth factor, nicotinamide, and insulin); SB-431542: TGF-beta signaling inhibitor; GSK3i: GSK3 inhibitor; ROCKi: inhibitor of the Rho-associated protein kinase p160ROCK; Nico: nicotinamide.

In brief, a piece of human tumor tissue was enzymatically digested and seeded on the irradiated 3T3-J2 feeder (originally obtained from Prof. Howard Green's laboratory at the Harvard Medical School, Boston, Mass., USA) in the presence of a modified growth medium. The stem cells selectively grow under these conditions and can be passaged indefinitely in vitro.

The day prior to receiving the human tissues, irradiated 3T3-J2 cells were seeded on Matrigel coated plates (BD Matrigel™, Basement Membrane Matrix, Growth Factor Reduced (GFR), cat. no. 354230). For this the Matrigel was thawed on ice and diluted in cold 3T3-J2 medium at the concentration of 10%. The 3T3-J2 growth medium contains DMEM (Invitrogen cat. no. 11960; high glucose (4.5 g/L), no L-glutamine, no sodium pyruvate), 10% bovine calf serum (not heat inactivated), 1% penicillin-streptomycin and 1% L-glutamine. The tissue culture plates were pre-cooled at −20° C. for 15 min, then diluted Matrigel was added on the cold plates, and the plates were swirled to evenly distribute the diluted Matrigel, then superfluous Matrigel was removed. Subsequently the plates were incubated for 15 min in a 37° C. incubator to allow the Matrigel layer to solidify.

Frozen irradiated 3T3-J2 cells were thawed and plated on the top of the Matrigel in the presence of 3T3-J2 growth medium. The next morning the 3T3-J2 medium was replaced by basic growth medium before being used as feeder layer for human cells. 1 L of basic growth medium contains 675 ml DMEM (Invitrogen cat. no. 11960; high glucose (4.5 g/L), no L-glutamine, no sodium pyruvate), 225 ml F12 (F-12 nutrient mixture (HAM), Invitrogen cat. no. 11765; containing L-glutamine), 100 ml FBS (Hyclone cat. no. SV30014.03; not heat inactivated), 6.75 ml of 200 mM L-glutamine (GIBCO cat. no. 25030), 10 ml adenine (Calbiochem cat. no. 1152; for the stock solution 243 mg of adenine were added to 100 ml of 0.05 M HCl and stirred for about one hour at RT until the solution was dissolved before filter sterilization. The solution can be stored at −20° C. until use), 1 ml of a 5 mg/ml stock solution of insulin (Sigma cat. no. 1-5500), 1 ml of 2×10⁻⁶ M T3 (3,3′,5-Triiodo-L-Thyronine) solution (Sigma cat. no. T-2752; for the stock solution 13.6 mg T3 were dissolved in 15 ml of 0.02N NaOH, and adjusted to 100 ml with phosphate buffered saline (PBS), resulting in a concentrated stock of 2×10⁻⁴ M, that can be stored at −20° C. 0.1 ml of the concentrated stock were diluted to 10 ml with PBS to create a working stock of 2×10⁻⁶ M), 2 ml of 200 μg/ml hydrocortisone (Sigma cat. no. H-0888), 1 ml of 10 μg/ml EGF (Upstate Biotechnology cat. no. 01-107), and 10 ml Penicillin-Streptomycin containing 10,000 units of penicillin and 10,000 μg of streptomycin per ml (GIBCO cat. no. 15140).

Human tumor tissues (transferred from hospital in cold wash buffer on ice) were washed vigorously using 30 ml cold wash buffer (F12: DMEM 1:1; 1.0% penicillin-streptomycin; 0.1% fungizone and 2.5 ml of 100 μg/ml gentamycin) for two times, minced and digested using 2 mg/mL collagenase type IV (Gibco, cat. no. 17104-109) and incubated at 37° C. for 1-2 h while gently shaking. The digested tissues were pelleted and washed five times with 30 mL cold wash buffer each. After the final wash, the samples were spun down and resuspended in modified growth medium and seeded on the feeder. The modified growth medium for human cancer stem cells SCM consisted of basic growth medium and the following factors: rock inhibitor (R)-(+)-trans-N-(4-Pyridyl)-4-(1-aminoethyl)-cyclohexanecarboxamide (Y-27632, Rho Kinase Inhibitor VI, Calbiochem, cat. no. 688000) at a working concentration of 2.5 μM; recombinant R-spondin 1 protein (R&D, cat. no. 4645-RS) at a working concentration of 125 ng/ml; recombinant noggin protein (Peprotech, cat. no. 120-10c) at a working concentration of 100 ng/ml; Jagged-1 peptide (188-204) (AnaSpec Inc., cat. no. 61298) at a working concentration of 1 μM; SB431542: 4-(4-(benzo[d][1,3]dioxol-5-yl)-5-(pyridin-2-yl)-1H-imidazol-2-yl)benzamide (Cayman chemical company, cat. no. 13031) at a working concentration of 2 μM; nicotinamide (Sigma, cat. no. N0636-100G) at a working concentration of 10 mM. After three to four days the first cancer stem cell colonies were detectable. Then cells were trypsinized with warm 0.25% trypsin (Invitrogen, cat. no 25200056) for 10 min, neutralized, resuspended in the modified growth medium, passed through 40 micron cell strainer and seeded as single cells onto a new plate containing a 3T3-J2 feeder layer. The medium was changed every two days. 3 days later, individual clones of human cancer stem cells were observed.

A single colony was be picked using a cloning ring and expanded to develop a pedigree cell line, i.e. a cell line that has been derived from a single cell.

Alternatively, single cells from the dissociated single cell suspension derived from these colonies can be selected using a glass pipette under a microscope and individually transferred to 96 well plates previously coated with 10% Matrigel and seeded with the feeder cells. Once the single cell forms colony in the 96 well plates, the colony can be expanded to develop a pedigree cell line.

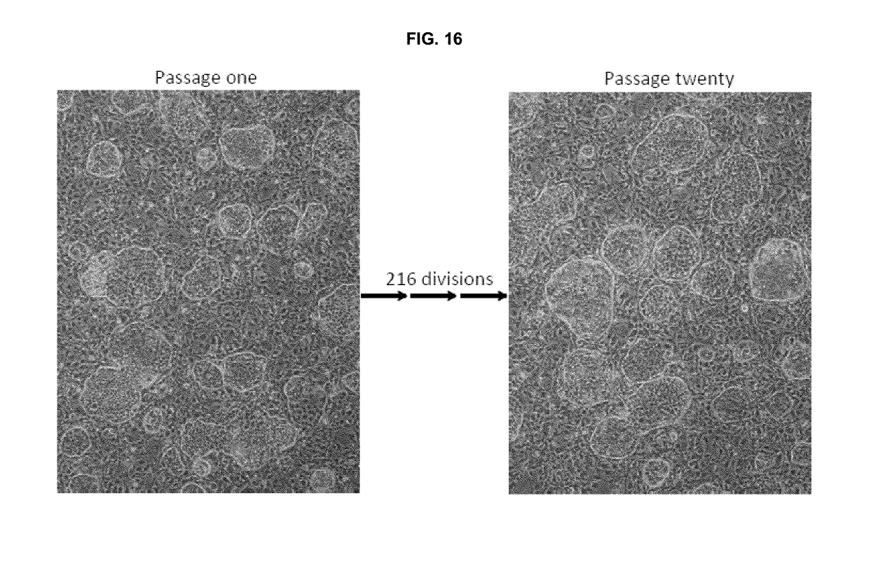

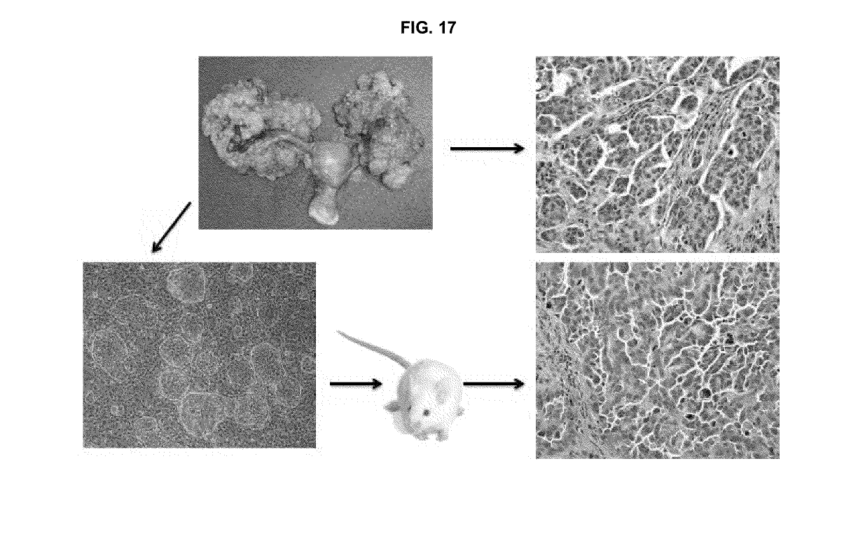

Furthermore, the data presented herein shows that each of these independent tumor cell colonies, which can be separately propagated to great numbers (FIG. 16), can support tumor development in highly immunosuppressed mice. The histology of these tumors grown in mice is indistinguishable from that of surgical resections from the same patient (FIG. 17).

In brief, between 10,000 to 1000,000 tumor stem cells originated from a single tumor stem cell were injected subcutaneously into the immunodeficient mice. In approximately three weeks to eight weeks, palpable tumors were detected, dissected, fixed and sectioned for histology analysis.

Given that these colonies were derived from single tumor cells, and can be independently and indefinitely propagated, and the fact that each pedigree can support high-grade ovarian-cancer-like tumor growth in mice, these tumor cell clones behaved as cancer stem cells in accordance with accepted definitions for cancer stem cells in the field.

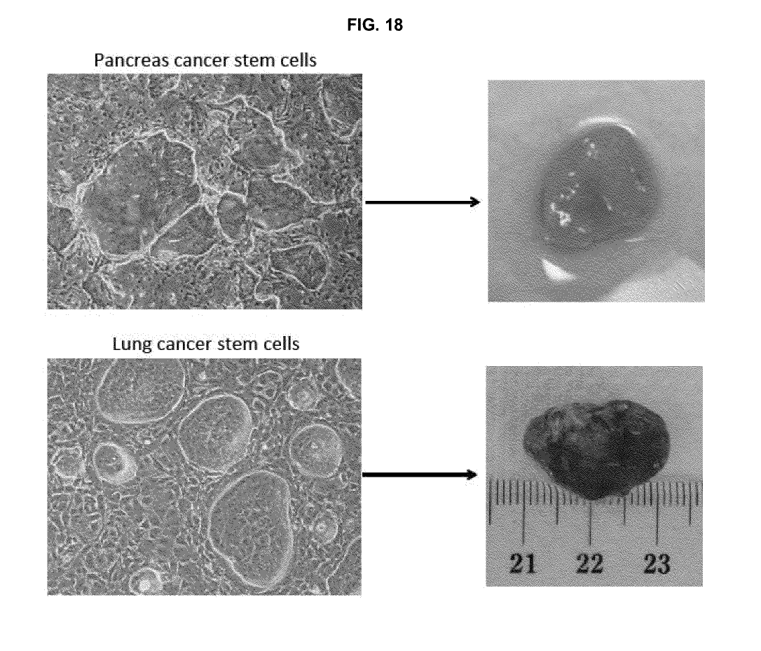

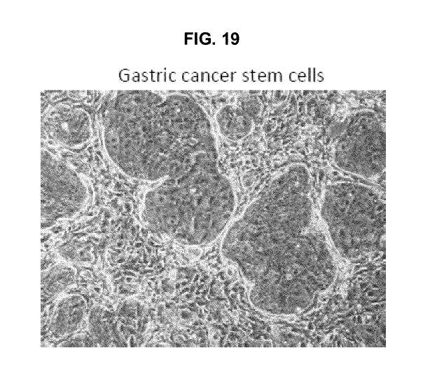

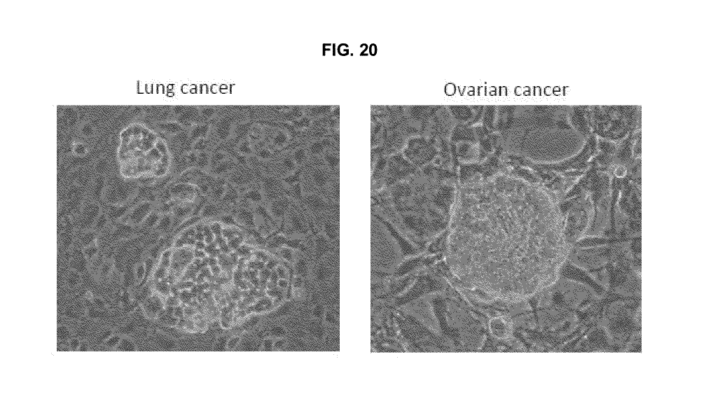

Using the same methods and substantially the same conditions, cancer stem cell clones were obtained from other primary resections of cancers, including pancreatic, lung, breast, esophagus and gastric cancers (FIGS. 18 and 19 and data not shown), as well as from human tumors (e.g., lung cancer, ovarian cancer and breast cancer) grown first in highly immunosuppressed mice in the PDX (patient-derived xenografts) models (FIG. 20 and data not shown).

### Example 17

### Identification of Mechanisms by which Tumor Cells Resist Chemotherapeutics

The libraries of CSCs established from a single patient using the methods of the invention enable interrogation of previously unapproachable questions such as tumor cell heterogeneity and, more importantly, screens and selections to identify chemotherapy-resistant variants that underlie the clinical development of lethal recurrences.

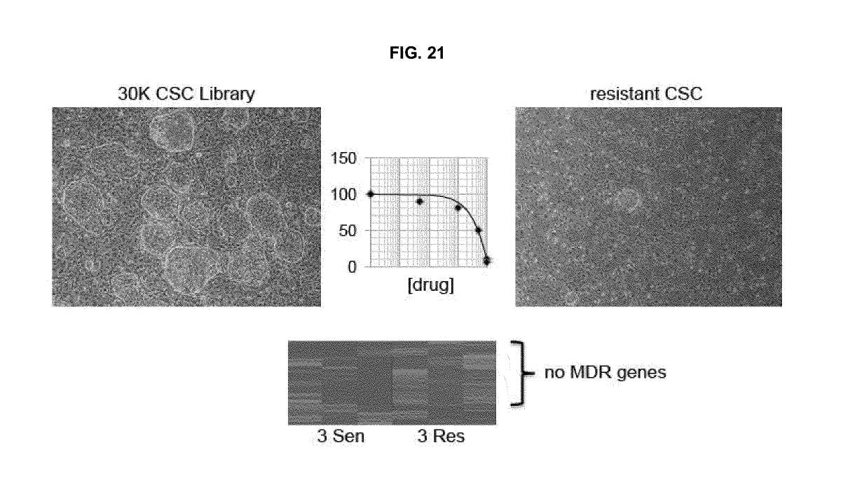

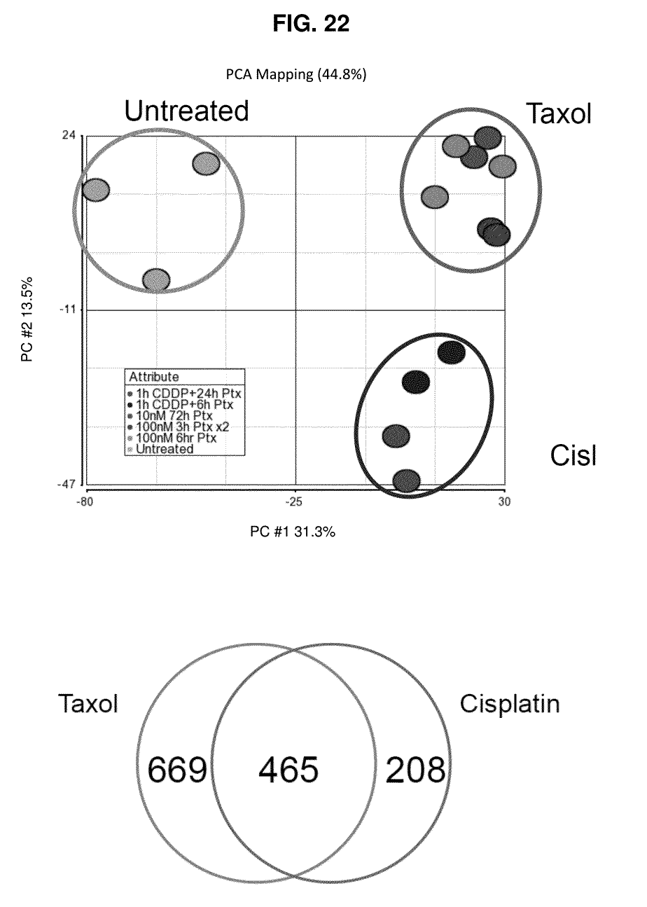

Described herein is the use of patient-specific CSC libraries in selections with standard-of-care chemotherapeutics to isolate and expand CSCs that not only resist the initial challenge with chemotherapeutics but also stably maintain this resistance to subsequent challenges by these drugs (FIG. 21). Finally, FIG. 22 shows our initial analyses of the gene expression differences between CSCs that are sensitive to cisplatin and paclitaxel, and those that are resistant to cisplatin, paclitaxel, or both.

Two general trends emerge based on the preliminary analysis of the resistant cells from patient tumor sample. One appears to contradict a supposed key mechanism underlying cancer resistance, as expected from many earlier studies. Specifically, earlier studies have suggested that overexpression of multiple drug resistance (MDR) genes is a key mechanism underlying cancer resistance. However, accumulating evidence in more recent studies seem to contradict this theory. Data presented herein supports the later findings, in that no MDR gene overexpression has been observed in the cloned cancer stem cells.

Another finding appears to shed light on the mechanism of resistance by cancer cells against distinct drugs having obvious differences in their mechanisms by which these drugs are thought to act. Specifically, there appears to be considerable overlap between gene sets over-represented in cisplatin and paclitaxel resistance CSC clones, despite the different cancer-killing mechanisms by which these drugs are thought to act. See FIG. 22.

### Example 18

### Cloning of Hippocampus Stem Cells

Adult neurogenesis, or the creation of new neurons in adult organisms, depends on the function of neural stem cells. The subgranular zone of the adult hippocampus is one area where hippocampal neural stem cells generate new neurons that functionally integrate into existing neuronal circuitry. The hippocampus plays a role in learning and memory consolidation, and is vulnerable to neurological diseases and conditions, such as Alzheimer's disease.

This example shows that the subject methods can be used to clone neural stem cells from hippocampus (FIG. 2). These cells can be cultured in vitro for numerous generations, and can differentiate into neurons upon induction. The cells are highly proliferative, and express stem cell markers such as SOX2 and PAX6 (data not shown). Upon induced differentiation, they express more differentiation markers such as Nestin (data not shown). This method of controlling the self-renewal and differentiation of hippocampal neural stem cells is a critical first step in developing novel disease treatment methods.

Specifically, the night before the following procedure, irradiated 3T3 cells were seeded on 10-20% MATRIGEL®-coated plate. The next morning, the culture medium was changed to fresh 3T3 culture medium. One hour before seeding hippocampal cells, the medium was again changed to the SBM medium (see above). The following steps were then carried out:

1. Isolate both sides of cortex containing hippocampus from mice (B1/6) or rat under dissection microscope. Put the tissues in the cold wash-buffer and keep on the ice immediately after dissection.

2. In the tissue culture hood, on a petri dish, mince tissue into fine pieces with sterile and disposable blade.

3. Digest the tissue by enzymes, such as papain or collagenase, by gently rocking at 37° C. for about 30 to 60 mins.

4. Break tissue into single cells by pipetting up and down gently for about 20 times;

5. Spin at 1000 rpm for about 5 mins;

6. Remove supernatant carefully without disturbing the pellet, and rinse cells with about 40 mL of washing buffer (same as other cell types). Then spin to remove the buffer. Repeat this step for 4 times in total as necessary. Try to remove all the wash medium carefully at the last step.

7. Resuspend cell pellet with about 10 mL of SCM (pre-warmed) by pipetting gently.

8. Filter the digested tissue through 100 micron cell strainer.

9. Seed the cells on MATRIGEL® and 3T3 fibroblast cells coated plate.

10. Changed medium every 2-3 days.

For Passaging and Replating Cells:

a. Wash cells with pre-warmed PBS or DMEM gently twice;

b. Add in pre-warmed 0.05% to 0.25% typsin, and incubate at 37° C. for not more than 10 mins when the cells are detached from the surface;

c. Stop trypsinization by adding 5 mL of SBM, pipet cells up and down, spin for about 5 min at 1000 rpm;

d. Remove supernatant carefully, and resuspend cells in SCM medium, transfer to a new 3T3 and MATRIGEL® coated dish.

For Cell Differentiation:

Seed the cells on 50% MATRIGEL® coated tissue culture dish in the presence of SBM medium.

### Example 19

### Cloning of Bladder Stem Cells

The bladder's inner lining is very unique. The multi-layered lining—known as urothelium—prevents leakage under pressure, fends off pathogens with a unique protein barrier, and protects underlying neurons, muscle, and blood vessels from toxins in the urine.

Cells in the barrier rarely divide, but acute damage from urinary tract infection or exposure to toxins induces rapid regeneration. The upper layer of the urothelium sloughs off, and the stem cells in the bladder form a new upper layer.

Multiple rounds of injury can compromise regeneration of the outer layer, resulting in permanent scarring, bladder dysfunction, and chronic pain. In chronic conditions such as bladder pain syndrome (aka interstitial cystitis), a disease that affects primarily women, underlying tissue including nerve endings is exposed, and that is thought to be a cause of chronic pain. In the most severe cases, the treatment for bladder pain syndrome and other chronic diseases is removal of the bladder.

Another reason to remove bladder is surgical removal of cancer in the bladder.

Using the subject methods described above, Applicant has cloned bladder stem cells from both mouse and human. The bladder stem cells are responsible for making and regenerating the organ's inner lining. Cloned patient-derived bladder stem cells create new ways for treating chronic bladder pain, such as by producing new tissues for patients with damaged bladders.

The stem cells cloned from human and mouse bladders have unlimited proliferation ability, and express markers such as p63, Krt5, and Agr2 (data not shown).

These cloned stem cells can be used for, e.g., tissue engineering to repair or regenerate damaged bladder in patients; or for differentiation into mature urothelium in vitro as discovery tool for identifying new therapeutic options to cure infections.

### Example 20

### Clonogenic Stem Cells of Intestine are Genetically Stable

Having established multiple, defined pedigrees of the human small intestine colonies, Applicant tests the “stemness” of the stem cell clones. In this Example, Applicant used independent pedigree cell lines (or “pedigrees” for short) for serial transfer and propagation over a five-month period. These pedigrees were grown and differentiated in an “air-liquid” interface known to trigger the differentiation of epithelial cells from a range of sources (see Example 14). Serial transfer and propagation of these three pedigrees were halted after five months, as the derived colonies maintained complete immaturity, despite having completed an estimated 400 divisions.

While it is well established that murine cells undergo “immortalization” and even transformation upon extended periods of proliferation in culture, human cells appear to be resistant to these processes. Applicant did not observe the morphological changes that typically accompany processes of immortalization or transformation in the subject stem cell pedigrees of the small intestine despite months of growth in continuous culture.

To further test the genetic stability of these intestinal stem cell pedigrees, Applicant performed copy number variation and exome sequencing analyses of these pedigrees at multiple time points during their months-long expansion in continuous culture. Significantly, these stem cell pedigrees proved to be remarkably stable as evidenced by the absence of obvious chromosome duplications or amplifications by CNV analysis and the acquisition of fewer than five non-synonymous mutations on average in genes without obvious impact on cell growth (data not shown). For example, exome sequencing showed that only one nonsynonymous mutation is gained after 20 passages or 70 cell divisions, supporting the notion that the cultured intestine stem cells are quite genomically stable. The same experiment further showed the absence of gaining more structural variation in the later passages, further supporting the genome stability of these cells.

Thus, in a very broad sense, these intestinal stem cell clones can be propagated for extended periods of time in culture while maintaining apparently normal genotype and phenotype.

### Example 21

### Cloning and Defining Regio-Specificity of Stem Cells of the Small Intestine and Colon

Given that many features of certain diseases, such as inflammatory bowel disease (IBD), appear to be confined to specific regions of the gastrointestinal tract, Applicant sought to define this “regio-specificity” at the level of gastrointestinal tract stem cells.

To this end, Applicant has obtained, under IRB approval, the entire gastrointestinal tract from a 22-week-old fetus (resulting from a failed pregnancy). Multiple regions of the duodenum, jejunum, ileum, ascending, transverse, and descending colon as well as rectum were excised for assessing the regional histology as well as for corresponding stem cell cloning. At 22 weeks, the histology of the gastrointestinal tract is well differentiated into the respective regions, and it was possible to successfully establish stem cell colonies and subsequent pedigrees using tissues from each region. Specifically, stem cells were cloned from Duodenum, Illeium, Jejunum, descending colon, ascending colon, and transverse colon, and were culture in the presence of SCM medium and feeder layer. The morphology of the stem cell clones looked remarkably similar—the cells all look very immature, with small size and high nucleus/cytoplasm ratio (data not shown).

Expression microarrays showed that the stem cells from the different regions of the small intestine were indeed distinct in approximately 100 genes as expected from the corresponding histology of the 22-week-old small intestine from which they were derived. Whole genome transcriptome analysis also showed that stem cells derived from different parts of small intestine are distinct from each other and also are different from stem cells derived from colon. For instance, small intestine stem cells express higher levels of CLDN18 and MSMD, and colon stem cells express higher level of HOXB9.

While the stem cells of the different regions of the colon proved to be more similar to one another (the stem cells derived from distinct part of the colon displayed their unique gene expression signature consisting of less than 60 genes), heatmap comparison of the different regions of the colon showed that these too could be distinguished from one another.

Consistent with these findings, the different regions of the gastrointestinal tract differentiate in air-liquid interface cultures into structures with distinct properties typical to the origin of the stem cells. For instance, when colon stem cells are differentiated in 3-D culture, they yield a mature epithelium remarkably distinct from the patterned villi associated with stem cells from the small intestine stem cells and marked by broad expanses of goblet cells reminiscent of the colonic epithelium.

Using the methods of the invention, regiospecific colon stem cells were also obtained from patients with Crohn's disease. Specifically, under IRB approval and through informed consent, Applicant has obtained a series of 1 mm biopsies from colonoscopies of multiple cases of pediatric Crohn's disease, functional control cases, and cases in various stages of remission following treatment. For each case, one or more 1 mm biopsies were obtained from the ileum as well as the ascending, transverse, and descending colon (data not shown). Multiple single stem cell pedigrees were derived from each, and these pedigrees were expanded for differentiation assays, copy number variation analysis, and exome sequencing.

Whole genome expression analyses of the stem cells of the ileum and three regions of the colon have been completed in the initial case of pediatric Crohn's disease, in one “functional” ulcerative colitis case without mucosal symptoms (control case), and in one Crohn's case for which the patient has been under standard treatment. All of the stem cells analyzed had been in continuous culture for at least six weeks, and the gene expression profiles of the stem cells of the Crohn's patient and those of the functional control patient have been obtained.

These Crohn's patient stem cells looked immature in vitro, and displayed the same morphology as the colon stem cells derived from healthy individual or fetal tissues (data not shown).

### Example 22

### Isolation, Cloning and Culturing of Human Biliary Tree Stem Cells

The biliary tree is composed of intrahepatic and extrahepatic bile ducts, lined by mature epithelial cells called cholangiocytes, and contains peribiliary glands deep within the duct walls. The perbiliary glands at the branch points, such as the cystic duct, perihilar and periampullar regions, contain multipotent stem cells, which can self-replicate and can differentiate into hepatocytes, cholangiocytes or pancreatic islets, depending on the microenvironment.

Using the subject method described herein, stem cells were cloned from fetal tissue containing bile ducts. The cloned bile duct stem cells express pluripotency markers such as SOX2, proliferation markers such as Ki67, and early hepato-pancreatic markers such as SOX9, SOX17, PDX1. Biliary tree-derived cells behaved as stem cells in this culture system, they can divide indefinitely and remain morphologically immature (data not shown). The shared common stem cell characteristics indicate a common embryological origin for the liver, biliary tree and pancreas, which has implications for regenerative medicine as well as the pathophysiology and oncogenesis of midgut organs.

### Example 23

### Lung Regeneration Using p63⁺/Krt5⁺ Distal Airway Stem Cells (DASC)

The potential for lung regeneration was long discounted due to the irreversible character of chronic lung diseases. However, patients who sustain massive loss of lung tissue during acute infections often recover full pulmonary function. This example demonstrates lung regeneration in mice following H1N1 influenza infections, and implicates p63⁺Krt5⁺ distal airway stem cells (DASC^{p63/K5}) in this process. Specifically, it was shown that rare, preexisting DASC^{p63/K5} cells undergo a proliferative expansion in response to H1N1 influenza infection, and can be lineage-traced to nascent alveoli assembled at sites of interstitial necrosis. Ablation of DASC^{p63/K5} in vivo prevents the regeneration of lung tissue following H1N1 influenza infection. In addition, single-cell derived pedigrees of DASC^{p63/K5} can be indefinitely expanded in culture, and, after being transplanted to lungs of H1N1 influenza-infected mice (e.g., after being introduced to and subsequently homing to the damaged lungs), can regenerate the damaged lung. Thus, these exogenous stem cells readily contribute to lung regeneration, and may have significant potential for mitigating acute and chronic lung diseases.

Using this method, human distal airway epithelial stem cell can be cloned in a robust way, and can form alveoli structures in in vitro assay. In addition, the culture system described herein allows cloning of lung epithelial stem cells from about 1 mm biopsy obtained from bronchoscope and expanding them to unlimited cell number for transplantation purpose.

### Background

Lung regeneration has long been difficult, as evidenced in part by the inexorable and progressive decline of pulmonary function in patients with chronic obstructive pulmonary disease (COPD) and pulmonary fibrosis. However, clinical reports of acute lung damage, especially pediatric cases of necrotizing pneumonia, detail events of extensive liquefaction of lung tissue that completely resolve both functionally and radiologically over several months. Similarly, survivors of acute respiratory distress syndrome (ARDS), which also can involve extensive destruction of lung tissue, often recover normal pulmonary function within six months of discharge.

A similar phenomenon is also present in mice infected with sub-lethal doses of murine-adapted H1N1 influenza A virus Like human lung during H1N1 or H5N1 influenza infections or other triggers of ARDS, the lungs of these mice developed broad zones of interstitial leukocyte infiltration marked by wholesale loss of distal airway epithelial cells including type I and type II pneumocytes in alveoli and Clara cells in bronchioli. Over the next six to eight weeks, however, these lungs return to their pre-infection status without evidence of such interstitial lesions. Paralleling this dramatic regenerative process was the appearance, at seven days post-infection (7 dpi), of large numbers of p63⁺Krt5⁺ cells in bronchioles and their sudden migration at 11 dpi to interstitial regions harboring dense leukocyte infiltrates. Once there, these p63⁺Krt5⁺ cells assembled into pod-like structures that ultimately assume the size, appearance, and gene expression profiles of alveoli. This assembly of alveoli from p63⁺Krt5⁺ cells in the lung is paralleled by the differentiation of cloned p63⁺Krt5⁺ cells to alveolar structures in vitro 11.

Cloned p63⁺Krt5⁺ cells are highly undifferentiated, capable of long-term self-renewal and differentiating into both Clara and alveolar cell types, and are thus referred to here as p63⁺Krt5⁺ distal airway stem cells (DASC^{p63/K5}).

Provided here is genetic lineage-tracing data that demonstrates the existence of DASC^{p63/K5} prior to H1N1 influenza infections, and that these pre-existing cells undergo proliferative expansion in response to lung damage and subsequently migrate to sites of damage where they differentiate to alveoli. Also described here is a novel mouse model that enables the conditional ablation of activated DASC^{p63/K5} and demonstrate that DASC^{p63/K5} are essential for lung regeneration following massive acute lung injury. Finally, it was demonstrated that cloned, syngeneic DASC^{p63/K5} can readily assemble into nascent alveoli in damaged lung following transplantation.

Lineage-Tracing of Pre-Infection DASC^{p63/K5} to Regenerating Lung

Applicant has previously shown that a sub-lethal dose of PR8 H1N1 influenza A virus induces a cycle of leukocyte lung infiltration that peaks between 9-15 days post-infection (dpi) and is followed by a gradual clearing and replacement by new alveoli of over the next several weeks in a process of lung regeneration. To clarify the fate of lung tissue infiltrated by leukocytes, infected and sham-infected control lungs at 15 dpi were examined by whole mount and serial sectioning. On a gross level, the H1N1 influenza virus triggers leukocyte infiltration and lung damage in a pattern radiating from the airway conduits. In cleared, whole-mount lung tissue, the control lungs show a highly ordered pattern of distal bronchioles with associated alveoli, whereas the infected lung show regions of obvious disruption that extend even to the most distal of bronchoalveolar networks (data not shown). Histology sections through these same anomalous regions show densely packed cells that stain positive for CD45, a general leukocyte marker including neutrophils and macrophages implicated in ARDS-associated lung damage (data not shown). Conspicuously absent from these regions of leukocyte infiltration is both the structural features of alveoli seen in unaffected regions of the same lung or even markers of type I (PDPN+) and type II (SPC+) pneumocytes (data not shown). These data are consistent with pathological findings in the lungs of ARDS patients in which lung tissue undergoes a breakdown in endothelial alveolar barriers and resulting edema, followed by a generalized necrotic phase involving tissue dissolution by leukocytes.

Despite the local destruction of alveoli in these zones of leukocyte infiltration, by 15 dpi these same zones show large numbers of discrete clusters of epithelial cells that co-express p63 and Krt5 and likely represent the early stages of de novo alveoli formation (data not shown). 3-D reconstructions of Krt5 staining in serial sections of 15 dpi lungs reveal broad, peri-bronchiolar patterns of the so-called p63/Krt5 “pods” during the regenerative process, suggesting their extension along the axis of the bronchioles (data not shown).

Similar processes were occurring in lungs of patients who succumbed to H1N1 influenza. While a majority of these patients expired within a week of infection, and, like the mice examined before 10 dpi, did not show interstitial p63/Krt5 pods, two patients who survived more than two weeks showed an pattern of these peri-bronchiolar p63/Krt5 pods that closely matched the murine model. In particular, laser capture microdissection and expression microarray analysis revealed that these peri-bronchiolar pods were distinct from squamous metaplasia and damaged lung and had an expression profile that most closely matched that of alveoli (data not shown).

Genetic lineage-tracing of Krt5⁺ cells was performed, starting before infection and followed the fate of these cells through the cycle of influenza-induced lung damage and regeneration. Mice expressing a Tamoxifen-dependent Cre recombinase under the control of the Krt5 promoter and having Cre-dependent lacZ expression [Tg (KRT5-Cre/ERT2) ROSA26-stop-lacZ] were treated with Tamoxifen at 9, 6, and 3 days before intratracheal delivery of 25 pfu of H1N1 influenza and processed for lacZ (E. coli (3-galactosidase) activity at various times post infection (data not shown). From 9 dpi to 60 dpi, the whole mount lacZ activity goes from subtle and restricted to the conducting airways (data not shown), to become more extensively distributed along the conducting airways and surrounding interstitial regions, suggesting a process that progressed from 15 to 60 dpi (data not shown). Importantly, no lacZ activity was detected in the lungs of Tamoxifen-treated mice in the absence of H1N1 influenza infection, suggesting the robust signal observed in the infected lung is response to the lung damage accompanying this infection (data not shown). Histological analysis of the lacZ-positive regions of lung from infected mice showed broad interstitial areas of staining that correspond to alveoli containing both type I and type II pneumocytes (data not shown). Together, the data suggests that rare, pre-existing Krt5⁺ stem cells contribute to the epithelial component of de novo alveoli produced in response to influenza-induced lung damage.

In view of this, the Krt5⁺p63⁺ cells in normal, uninfected lung were examined using immunofluorescence. These cells were indeed rare in normal lung (approx. 0.003% of total cells), and exist in bronchiolar regions as single or small clusters of cells and are distinct and more basally situated from the more common Clara cell expressing CC10 (data not shown).

Using the method of the invention, a single clone type that has long-term self-renewal ability was obtained, and all of these clones are co-label with antibodies to p63 and Krt5 (data not shown). Finally, in addition to type I and type II pneumocytes, LacZ⁺Krt5⁺ cells also gave rise to CC10⁺ bronchiolar cells in murine lung following pre-infection lineage tracing of Krt5⁺ cells (data not shown), suggesting that DASC^{p63/K5} cells give rise to Clara cells. The data suggests that p63⁺Krt5⁺ cells are pre-existing DASCs in the lung that undergo a proliferative response, and contribute to de novo alveoli during acute lung damage.

Conditional Ablation of Activated DASC^{p63/Krt5}

The experiment demonstrates that selective ablation of DASCp63/Krt5 cells suppress or eliminate the regenerative response in lung.

Our previous studies revealed that DASCp63/Krt5 responding to acute lung injury begin to express keratin 6 (Krt6), a marker of epidermal stem cells responding to injury, just prior to and through the first several days of their migration to interstitial regions of lung damage. Thus, Applicant engineered the Krt6a locus in embryonic stem cells to generate a mouse strain that constitutively expresses DTR from one of the Krt6a alleles (data not shown). DASCp63/Krt5 cells responding to influenza infection indeed expressed the DTR transgene at the same time these cells assume expression of the Krt6 gene (data not shown). Then cloned DASCp63/Krt5 cells from Krt6-DTR mice at 15 dpi (when Krt6 is expressed) were found to die within four days of diphtheria toxin, whereas control clones continued to proliferate and expand in size (data not shown). For the in vivo analysis of this mouse model, the Krt6-DTR mice were infected with a sub-lethal dose (25 pfu) of H1N1 influenza virus and at 8 dpi with diphtheria toxin. Diphtheria toxin resulted in a rapid loss of interstitial clusters of Krt5⁺Krt6⁺ cells by 15 dpi (data not shown), suggesting a highly efficient ablation model. As expected, the Krt5⁺Krt6⁻ cells within the bronchiolar epithelium survived the diphtheria toxin treatment (data not shown). Compared to wild type controls, Krt6⁻ DTR mice lose 90 percent of Krt5⁺ cells and greater than 99% of Krt6⁺ cells following diphtheria toxin treatment (data not shown).

Applicant also followed the fate of mice treated with diphtheria toxin following H1N1 influenza infection. At 30 dpi, when wild type mice show significant recovery of lung damage as evidenced by reduction in interstitial densities, the lungs of Krt6-DTR mice show more and broader areas of unresolved damage similar to the damage that was present at 15 dpi (data not shown). This difference in persistent lung damage is even more evident from a comparison of whole genome expression analyses of wild type and the KRT-DTR lungs, which reveals a strong bias towards alveolar gene expression in the wild type animals (data not shown). The basis for this bias was revealed by histological comparisons of the persistent densities, which showed that even though the 30 dpi wild type lung still had about 30% of the damage evident at 15 dpi, nearly all of the remaining densities consist of networks of type I pneumocytes lacking SPC type II pneumocytes (data not shown).

As lineage tracing of Krt5 cells labels both type I and type II cells in the regenerating regions at two months, we anticipate events between one and two months result in mature networks of alveoli including type II pneumocytes. Given the absence of regenerative events in 30 dpi lungs following ablation of DASCp63Krt5/Krt6, we asked if these lungs showed evidence of chronic degeneration. At the same 30 dpi time point, the densities in the Krt6-DTR lungs are completely devoid of these type I pneumocytes networks (data not shown). In fact the persistent densities in the 30 dpi wild type mouse were due to assembly of new lung tissue rather than concentrated leukocytes which had resolved, whereas the densities of the 30 dpi Krt6-DTR mice reflected the continued presence of CD45⁺ leukocytes (data not shown).

Given the absence of regenerative events in 30 dpi lungs following ablation of DASCp63Krt5/Krt6, we asked if these lungs showed evidence of chronic degeneration. Significantly, the persistent densities in the 30 dpi showed staining for smooth muscle actin (αSMA), a marker of myofibroblasts previously implicated in a pre-fibrotic state of the lung. These same interstitial regions showed weak but detectable staining with Masson's Trichrome blue, a marker of fibrosis (data not shown). Consistently, a comparison of gene expression profiles of wild type and DASC-ablated lung at 30 dpi revealed a lung fibrosis gene signature including vimentin, FSP129, and collagen genes (data not shown). Expression of fibrosis-related genes including collagen and vimentin is also observed in the Krt6-DTR lung (data not shown). Additional histological examination showed large numbers of myofibroblasts expressing alpha-smooth muscle actin, an early marker of fibrosis. Such cells were not present in dense regions of 30 dpi wild type lung. Together these data demonstrate that the ablation of DASCp63/Krt5/Krt6 that arise during the response to acute lung injury results in a failure of the regenerative process and the development of prefibrotic events at sites of lung damage.

Incorporation of Exogenous Stem Cell Pedigrees into Regenerating Lung

This experiment demonstrates that pre-existing DASCp63/Krt5 could indeed participate in the regenerative process following in vitro cloning, expansion, and transplantation.

Using a syngeneic strain of mice marked by lacZ expressed from the ubiquitous ROSA26 locus (ROSA26-lacZ19), airway stem cells from both the upper airways (tracheobronchiolar stem cells; TBSClacZ) and the lung (DASClacZ) were cloned (data not shown). Using the methods of the invention, pedigrees of both TBSClacZ and DASClacZ were then generated from single cells for expansion and parallel analyses.

In their immature stem cell state, the TBSClacZ and DASClacZ pedigrees are both positive for Krt5 and p63 while negative for known differentiation markers. They are highly similar at the transcriptome level but distinguishable by a signature set of genes even after long-term serial passaging (>6 months; data not shown). Upon differentiation in 3-D culture, TBSClacZ and DASClacZ give rise to upper airway epithelium and alveolar structures (data not shown), respectively, and express correspondingly divergent sets of genes (data not shown).

To test the fate of these cells upon transplantation, one million immature cells derived from TBSClacZ or DASClacZ pedigrees were intratracheally delivered to mice infected with 25 pfu H1N1 influenza virus five days earlier and followed over time (data not shown). In uninfected controls, neither TBSClacZ nor DASClacZ showed incorporation into lung at any time within 90 days (data not shown). However, at 40 dpi (35 days post-delivery), TBSClacZ localized to the major airways in a pattern that did not change at 90 dpi and consistent with their tracheobronchial origin (data not shown). In contrast, DASClacZ showed a broad distribution of lacZ activity involving smaller airways and interstitial regions of the 40 dpi lung (data not shown). At 90 dpi, DASClacZ showed a more homogenous pattern in interstitial spaces compared to those assayed at 40 dpi (data not shown).

Histological sections of lungs seeded with DASClacZ revealed lacZ staining in patterns in interstitial lung typical of alveoli, and these same regions co-stained for markers of type I and type II alveoli (data not shown). Gene expression analysis of the lacZ-positive regions of these lungs using laser-capture microdissection showed a typical alveoli gene signature very different from that of damage lung (data not shown). Finally, Clara cells could also be generated by transplanted DASClacZ as shown by lacZ staining in CC10⁺ bronchioles (data not shown).

Together these findings demonstrate that pedigree lines of distal airway stem cells derived from single cells can be expanded by proliferation in vitro indefinitely and readily incorporate into damaged lung to contribute to the regeneration of lung tissue.

### Materials and Methods

Krt5-CRE/Rosa26-LacZ mice were used for lineage tracing. Tamoxifen (Tam) was resolved in corn oil and applied to mice at 200 mg/Kg through IP injection. For post-infection tracing, Tam was applied at 5, 6, 7 dpi. For pre-infection tracing, Tam was applied at −9, −6, −3 dpi. H1N1 virus dose is 50 pfu.

Mouse lungs were collected at indicated dpis, and subjected to x-gal whole-mount staining overnight. Representative lobes were made transparent by BABB to show clear blue signal. FFPE sections were stained by Nuclear Red and IF. Blue signals indicate the LacZ labeled cells. The specificity of x-gal staining was verified by bacteria-specific beta-gal antibody staining.

For post-infection tracing, one day after Tam (8 dpi), some basal cells were successfully labeled in bronchioles. After 2-3 months, significant airway structure regeneration by the LacZ labeled cells was observed. Those blue cells include CC10⁺ secretory cells in bronchioles, 1H8/11D6⁺ pneumocytes (including SPC⁺ Type II alveoli cells).

For pre-infection tracing, similar pattern is observed as in post-infection tracing. Those blue cells include CC10⁺ secretory cells and acetyl-Tubulin⁺ Ciliated cells in main stem bronchus, CC10⁺ secretory cells in bronchioles, 1H8⁺ pneumocytes (including SPC⁺ Type II alveoli cells).

The non-infection control mouse showed only trace amount of blue signal 3 months after tamoxifen, which could be due to the normal turnover of lung cells.

To isolate airway stem cells, trachea and lung were collected from one adult C57/B6 mouse and digested by dispase and trypsin and then seeded onto matrigel coated dish with 3T3 feeder cells. After 4 consecutive passages, single colonies were picked up by cloning ring and cultured. TASC and DASC colony morphology look similar, and both are Krt5 and p63 positive. All of the lineage markers (Pdpn, CC10 and SPC) are negative (data not shown). Up to now, these colonies have been passaged up to one year with no observable properties change.

Matrigel differentiation assay was performed as described in previous report (Kumar et. al 2011). FGF10 (50 ng/mL) was included in medium to favor distal airway differentiation. Under this condition, DASCs clustered and grew into sphere-like structure. The sphere is hollow inside with one or two layer of cells on the surface. TASCs also clustered but showed little growth and formed no regular structure. IF staining showed DASC but not TASC matrigel structures express some alveoli markers such as Aqp5 and SPC. Representative images are taken on Day 9.

Furthermore, microarray analysis on DASC, TASC and their matrigel structures was performed. By PCA analysis, it was found that DASC but not TASC matrigel structure was similar to mouse embryonic lung in terms of transcriptome. And the “stem cell to matrigel structure” differentiation process recapitulates the mouse embryonic development process.

In order to compare matrigel structure with real mouse trachea and lung, LCM was used to dissected mouse trachea, bronchioles and alveoli and microarray analysis was performed. By doing this, mouse tracheal, bronchiolar and alveolar gene expression signatures were developed respectively. Further analysis showed TASC matrigel structure has higher tracheal signature while DASC matrigel structure has higher bronchiolar and alveolar signature.

For ALI differentiation, FGF10 was excluded and retinoid acid was included in medium to favor proximal airway differentiation. Under ALI condition, TASC forms stratified structure while DASC forms single layer structure. IF staining showed TASC ALI structure has Krt5⁺ basal cell layer and luminal ciliated and secretory cell layer.

To perform orthotropic transplantation of stem cells, Applicant developed intra-tracheal delivery system and first tested it using retro-GFP labeled DASC. C57/B6 mice were infected by 75 pfu H1N1 virus, and transplantation (1×10⁷ cells) was performed at 5 dpi. 24 hours after transplantation, GFP⁺ cells were found incorporated into multiple lung regions including bronchioles, BADJ and damaged interstitial regions. Some of the cells maintain strong Krt5 expression similar to endogenous Krt5⁺ stem cells. No GFP⁺ cells were found in trachea because its tube-like shape can hardly retain exogenous cells.

1 week after transplantation (12 dpi), GFP⁺ cells form clusters which mimic endogenous Krt5 pods but no lineage marker is expressed (data not shown). 2 weeks after transplantation (20 dpi), GFP⁺ cells form clusters which mimic bigger Krt5 pods which express Pdpn.

LacZ labeled cells were used for long-term transplantation experiments. Stem cells from adult K5-CRE/Rosa26-LacZ mice were cloned. Cells were treated by 4OH-Tmx in vitro for 4 days to induce CRE activity. LacZ expression was verified by IF staining with bacteria-specific beta-gal antibody which shows >90% stem cells are LacZ positive. 1 month after transplantation (40 dpi), whole mount lacZ staining showed regeneration of airway structure by transplanted DASC but not TASC. After sectioning, it was found that transplanted 42% DASC forms bronchioles at 40 dpi, while 19% forms alveoli structures. 3 months after transplantation, whole mount LacZ staining showed significant regeneration of airway structure by transplanted DASC but not TASC. 5% DASC formed bronchioles and 83% DASC formed alveoli (alveoli cells are in larger numbers for healthy lung). IF staining showed the transplanted DASC form 1H8⁺ pneumocytes (including SPC⁺ type II and Hop⁺ type I alveoli cells). In contrast, TASC formed only a few irregular structure that somewhat resembled lung tumor.

To verify the Krt6-DTR mouse model, the DTR expression in 12 dpi lung was verified, which showed good DTR and Krt6 co-expression. Then DT was shown to be able to kill Krt6-DTR cells in vitro. Stem cells were isolated from post-infection (23 dpi) WT and Krt6-DTR mouse lung followed by DT treatment (0.02 ug/mL). Krt6-DTR colony dies after 4 days while the WT colony looks normal even when the DT dose is increased to 10 fold. The Krt6 negative endothelial cells isolated from Krt6-DTR mice is also insensitive to DT.

DT effect was also tested in vivo. At 8 dpi, DT was given through both IP (50 ug) and intratracheal (100 ug) way. Mouse lungs were collected at 12 dpi. Krt6 and Krt5 cell numbers were counted. The IF staining results showed that comparing to WT, in Krt6-DT mouse treated with DT the Krt6⁺ cell number was nearly 90% reduced, and Krt5⁺ cells number was also 70% reduced.

Clonogenic assay was performed for the 12 dpi lung, and a 70-80% reduction of clonogenic cell number reduction was found in Krt6-DT mouse in comparison with WT mouse with DT. This number is consistent with the loss of Krt5/Krt6 cells by IF staining.

12 and 30 dpi lungs were collected for H&E staining. Damaged area of lung (loss of airway structure, with dense immune cell infiltration) was measured. The results showed that at 12 dpi WT and Krt6-DT lungs were similarly damaged (around 30%); 30 dpi WT lung was half repaired while Krt6-DT lung was not. Mouse body weight curve was largely consistent with histology.

1. A method for isolating a non-embryonic stem cell from a non-embryonic tissue, the method comprising:
(1) culturing dissociated epithelial cells from the non-embryonic tissue, in contact with a first population of lethally irradiated feeder cells and a basement membrane matrix, to form epithelial cell clones, in a medium comprising:
(a) a Notch agonist;
(b) a ROCK (Rho Kinase) inhibitor;
(c) a Bone Morphogenetic Protein (BMP) antagonist;
(d) a Wnt agonist;
(e) a mitogenic growth factor; and,
(f) insulin or IGF;
said medium optionally further comprising at least one of:
(g) a TGFβ receptor inhibitor; and,
(h) nicotinamide or an analog thereof;
(2) isolating single cells from said epithelial cell clones, and,
(3) culturing isolated single cells from step (2) individually to form single cell clones, in contact with a second population of lethally irradiated feeder cells and a second basement membrane matrix in said medium;
wherein each of said single cell clones represents a clonal expansion of said non-embryonic stem cell, thereby isolating said non-embryonic stem cell.

2-4. (canceled)

5. The method of claim 1, further comprising isolating single non-embryonic stem cell from said single cell clones, and optionally further comprising culturing one of said single cell clones to generate a pedigree cell line of said non-embryonic stem cell.

6-9. (canceled)

10. The method of claim 1, wherein the non-embryonic tissue is obtained from or originates in lung, esophagus, stomach, small intestine, colon, intestinal metaplasia, fallopian tube, kidney, pancreas, bladder, or liver, or a portion/section thereof.

11. The method of claim 1, wherein the non-embryonic tissue is a disease tissue, a disorder tissue, an abnormal condition tissue, or a tissue from a patient having said disease, disorder, or abnormal condition, such as, for example, wherein the disease, disorder, or abnormal condition comprises an adenoma, a carcinoma, an adenocarcinoma, a cancer, a solid tumor, an inflammatory bowel disease (e.g., Crohn's disease, ulcerative colitis), ulcer, gastropathy, gastritis, oesophagitis, cystitis, glomerulonephritis, polycystic kidney disease, hepatitis, pancreatitis, an inflammatory disorder (e.g., type I diabetes, diabetic nephropathy) and autoimmune disorder.

12-14. (canceled)

15. The method of claim 1, wherein in step (1) the (epithelial) cells are dissociated from the non-embryonic tissue through enzymatic digestion with an enzyme, such as collagenase, protease, dispase, pronase, elastase, hyaluronidase, accutase and/or trypsin.

16. (canceled)

19. The method of claim 1, wherein the basement membrane matrix is a laminin-containing basement membrane matrix (e.g., MATRIGEL™ basement membrane matrix (BD Biosciences)), preferably growth factor-reduced, preferably wherein the basement membrane matrix does not support 3-dimensional growth, or does not form a 3-dimensional matrix necessary to support 3-dimensional growth.

20-21. (canceled)

22. The method of claim 1, characterized by one or more of the following:
the Notch agonist comprises Jagged-1,
the ROCK inhibitor comprises Rho Kinase Inhibitor VI (Y-27632, (R)-(+)-trans-N-(4-Pyridyl)-4-(1-aminoethyl)-cyclohexanecarboxamide)), Fasudil or HA1071 (5-(1,4-diazepan-1-ylsulfonyl)isoquinoline), or H1152 ((S)-(+)-2-methyl-1-[(4-methyl-5-isoquinolinyl) sulfonyl]-hexahydro-1H-1,4-diazepine dihydrochloride),
the BMP antagonist comprises Noggin, DAN, a DAN-like proteins comprising a DAN cystine-knot domain (e.g., Cerberus and Gremlin), Chordin, a chordin-like protein comprising a chordin domain, Follistatin, a follistatin-related protein comprising a follistatin domain, sclerostin/SOST, decorin, or α-2 macroglobulin,
the Wnt agonist comprises R-spondin 1, R-spondin 2, R-spondin 3, R-spondin 4, an R-spondin mimic, a Wnt family protein (e.g., Wnt-3a, Wnt 5, Wnt-6a), Norrin, or a GSK-inhibitor (e.g., CHIR99021),
the mitogenic growth factor comprises EGF (and/or Keratinocyte Growth Factor, TGFα, BDNF, HGF, bFGF),
the TGFβ receptor inhibitor comprises SB431542 (4-(4-(benzo[d][1,3]dioxol-5-yl)-5-(pyridin-2-yl)-1H-imidazol-2-yl)benzamide), A83-01, SB-505124, SB-525334, LY 364947, SD-208, or SJN 2511, and/or,
the TGFβ (signaling) inhibitor binds to and reduces the activity of one or more serine/threonine protein kinases selected from the group consisting of ALK5, ALK4, TGF-beta receptor kinase 1 and ALK7.

23-29. (canceled)

30. The method of claim 1, wherein the medium comprises: 5 μg/mL insulin; 2 nM of (3,3′,5-Triiodo-L-Thyronine); 400 ng/mL hydrocortisone; 24.3 μg/mL adenine; 10 ng/mL EGF; 10% fetal bovine serum (without heat inactivation); 1 μM Jagged-1; 100 ng/mL noggin; 125 ng/mL R-spondin 1; 2.5 μM Y-27632; and 1.35 mM L-glutamine in DMEM:F12 3:1 medium, optionally further comprising 0.1 nM cholera enterotoxin, and optionally further comprises one or more of 2 μM SB431542 and 10 mM nicotinamide.

31-37. (canceled)

38. The method of claim 30, wherein the non-embryonic tissue is fetal small intestine, and the medium further comprises: FGF receptor inhibitor; N-Acetyl-L-cysteine; a p38 inhibitor (e.g., SB-202190, SB-203580, VX-702, VX-745, PD-169316, RO-4402257 and BIRB-796); Gastrin; PGE2; an FGF receptor inhibitor; Shh; TGFβ; 10 mM nicotinamide and TGFβ; 10 mM nicotinamide and Wnt3a; 10 mM nicotinamide and GSK3 inhibitor; or 10 mM nicotinamide and 2 μM SB431542.

39. The method of claim 1, wherein the medium lacks at least one of: Wnt3a, p38 inhibitor (e.g., SB-202190, SB-203580, VX-702, VX-745, PD-169316, RO-4402257 and BIRB-796), N-Acetyl-L-cysteine, Gastrin, HGF, testosterone (e.g., (dihydro)testosterone), N2, B27, and PGE2.

40. (canceled)

41. The method of claim 1, wherein said non-embryonic stem cell, when isolated as single cell, is capable of self-renewal for greater than about 50 generations, 70 generations, 100 generations, 150 generations, 200 generations, 250 generations, 300 generations, 350 generations, or about 400 or more generations.

42. (canceled)

43. The method of claim 1, wherein said non-embryonic stem cell is a small intestine stem cell, and is capable of differentiating into a differentiated small intestine cell that (1) expresses a marker selected from MUC or PAS (goblet cell markers), CHGA (neuroendocrine cell marker), LYZ (Paneth cell marker), MUC7, MUC13, and KRT20; and/or (2) absorbs water and nutrients (such as by differentiated enterocytes), secretes mucus (such as by differentiated goblet cells), secretes intestinal hormones (such as by differentiated enteroendocrine cells), or secreting antibacterial substances (such as by differentiated Paneth cells).

44. The method of claim 1, wherein said non-embryonic stem cell expresses one or more stem cell markers selected from: SOX9, KRT19, KRT7, LGR5, CA9, FXYD2, CDH6, CLDN18, TSPAN8, BPIFB1, OLFM4, CDH17, and PPARGC1A, and preferably said non-embryonic stem cell is a small intestine stem cell, and expresses one or more markers selected from: OLFM4, SOX9, LGR5, CLDN18, CA9, BPIFB1, KRT19, CDH17, and TSPAN8.

45. (canceled)

46. The method of claim 1, wherein said non-embryonic stem cell substantially lacks expression of marker(s) associated with differentiated cell types in said non-embryonic tissue, preferably said non-embryonic stem cell is a small intestine stem cell, and lacks expression of markers associated with differentiated small intestine cells selected from: MUC or PAS (goblet cell markers), CHGA (neuroendocrine cell marker), LYZ (Paneth cell marker), MUC7, MUC13, and KRT20.

47-48. (canceled)

49. A non-embryonic stem cell isolated according to the method of claim 1.

50. The non-embryonic stem cell of claim 49, which is isolated from a cuboidal or columnar epithelial tissue (preferably an adult cuboidal or columnar epithelial tissue).

51-52. (canceled)

53. A single cell clone of a non-embryonic stem cell, wherein at least about 40%, 50%, 60%, 70%, or about 80% of cells within said single cell clone, when isolated as single cell, is capable of proliferation to produce single cell clone.

54-55. (canceled)

56. A single cell clone of a non-embryonic stem cell, wherein said non-embryonic stem cell expresses one or more stem cell markers selected from SOX9, KRT19, KRT7, LGR5, CA9, FXYD2, CDH6, CLDN18, TSPAN8, BPIFB1, OLFM4, CDH17, and PPARGC1A, such as a small intestine stem cell, which expresses one or more markers selected from: OLFM4, SOX9, LGR5, CLDN18, CA9, BPIFB1, KRT19, CDH17, and TSPAN8.

57-60. (canceled)

61. A medium for isolating and/or culturing non-embryonic stem cell, said medium comprising:
(a) a Notch agonist;
(b) a ROCK (Rho Kinase) inhibitor;
(c) a Bone Morphogenetic Protein (BMP) antagonist;
(d) a Wnt agonist;
(e) a mitogenic growth factor; and,
(f) insulin or IGF,
and optionally may also include:
(g) a TGFβ receptor inhibitor; and,
(h) nicotinamide or a precursor thereof.

62. (canceled)

63. A method of treating a subject having a disease, a disorder, or an abnormal condition and in need of treatment, comprising:
(1) using the method of claim 1, isolating an adult stem cell from a tissue corresponding to a tissue affected by said disease, disorder, or abnormal condition in said subject;
(2) altering the expression of at least one gene in said adult stem cell to produce an altered adult stem cell;
(3) reintroducing said altered adult stem cell or a clonal expansion thereof into the subject, wherein at least one adverse effect or symptom of said disease, disorder, or abnormal condition is alleviated in said subject.

64-70. (canceled)

71. A method of screening for a compound, said method comprising:
(1) using the method of claim 1, isolating an adult stem cell from a subject;
(2) producing a cell line of said adult stem cell via single cell clonal expansion;
(3) contacting test cells from the cell line with a plurality of candidate compounds; and,
(4) identifying one or more compounds that produces a pre-determined phenotype change in said test cells.
